SEQUENCE LISTING (383 sequences)

SEQ ID NO: 1 - DNA, 383 bp, synthetic construct
  misc_feature 1..383: note = synthetic primer
  source 1..383: mol_type = other DNA
accggtgtacattccgaggtccagctgcaacagtctggggctgagctggtgaagcctggggcttcagtgaagttgtcctgcaaggcttctggctacactttcaccagctactgggtgcactgggtgaggcagaggcctggacaaggccttgagtggattggaatgattcatcctaatagtggtagtactaactacaatgagaagttcaagagcaaggccacactgactgtagacaaatcctccagcacagcctacatgcaactcagcagcctgacatctgaggactctgcggtctattactgtgcaagttattactacggtagtagctattactttgactactggggccaaggcaccactctcacagtctcctcagcgtcgac

SEQ ID NO: 2 - DNA, 360 bp, synthetic construct
  misc_feature 1..360: note = synthetic primer
  source 1..360: mol_type = other DNA
accggtgtacattccgacattgtgatgacccagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagctggagctgaaacgtacg

SEQ ID NO: 3 - DNA, 371 bp, synthetic construct
  misc_feature 1..371: note = synthetic primer
  source 1..371: mol_type = other DNA
accggtgtacattccgaggtccagctgcaacagtctggggctgagctggtaaagcctggggcttcagtgaagctgtcctgcaaggcttctggctacactttcaccaactactggatgcactgggtgaagcagaggcctgggcaaggccttgagtggattggaatgattcatcctaatagtggtacttctaactacaatgagaagttcaagagcaaggccacactgactgtagacaaatcctccagcacagcctacatgcaactcagcagcctgacatctgaggactctgcggtcttttactgtacaagatctgactgggcctttgactactggggccaaggtacctctctcacagtctcctcagcgtcgac

SEQ ID NO: 4 - DNA, 430 bp, synthetic construct
  misc_feature 1..430: note = synthetic primer
  source 1..430: mol_type = other DNA
accggtgtacattccgacattgtgatgacccagtctccatcctccctgagtgtgtcagcaggagagaaggtcactatgagctgcaagtccagtcagagcctgttaaacagtggaaatcaaaagaactacttggcctggtaccagcagaaaccagggcagcctcctaaactgttgatctacggggcatccactagggaatctggggtccctgatcgcttcacaggcagtggatctggaaccgatttcactcttaccatcagtagtgtgcaggctgaagacctggcagtttattactgtcagaatgatcatagttatccgctcacgttcggtgctgggaccaagctggagctgaaacgtacgcctttgtatcatgctattgcttcccgtatggctttcattttctcctccttgtataaatcctggntgctgn

SEQ ID NO: 5 - DNA, 377 bp, synthetic construct
  misc_feature 1..377: note = synthetic primer
  source 1..377: mol_type = other DNA
accggtgtacattccgaggtccaactgcagcagtctggagctgagctggtgaggcctgggtcctcagtgaagatgtcctgcaagacttctggatatacattcacattctacggtataaactgggtgaagcagaggcctggacagggcctggaatggattggatatatttatgttggaaatggttattctgagtacaatgagaagttcaaggtcaaggccacactgacttcagacacatcctccagcacagcctacatgcagctcagcggcctgacatctgaggactctgcaatctatttctgtgcaagatcagatggttacaactactttgactactggggccaaggcaccactctcacagtctcctcaacgtcgac

SEQ ID NO: 6 - DNA, 339 bp, synthetic construct
  misc_feature 1..339: note = synthetic primer
  source 1..339: mol_type = other DNA
accggtgtacattcccaaattgttctcacccagtctccagcaatcctatctgcatctccaggggagaaagtcacaatgacttgcagggccagctcaagtttacattacatgcactggtaccagcagaagacaggatcctcccccaaaccctggatttatgccacatccaacctggcttctggagtccctgctcgcttcagtggcagtgggtctgggacctcttactctctcacaatcagcagagtggaggctgaagatactgccacttattactgccagcagtggagtagtaacccacccacgttcggtgctgggaccaagctggagctgaaacgtacg

SEQ ID NO: 7 - DNA, 380 bp, synthetic construct
  misc_feature 1..380: note = synthetic primer
  source 1..380: mol_type = other DNA
accggtgtacattccgaggtccaactgcagcagcctggggctgagctggtaaagcctggggcttcagtgaagttgtcctgcaaggcttctggctacactttcaccagctactggatgcactgggtgaagcagaggcctggacaaggccttgagtggattggattgattcatcctaatagtggtagtacttactacaatgagaagttcaagaacaaggccacactgactgtagacaaatcctccagcacagcctacatgcaactcagcagcctgacatctgaggactctgcggtctattactgtgcaagatgggatgattcctactggtacttcaaagtctggggcacagggaccacggtcaccgtctcctcagcgtcgac

SEQ ID NO: 8 - DNA, 339 bp, synthetic construct
  misc_feature 1..339: note = synthetic primer
  source 1..339: mol_type = other DNA
accggtgtacattcccaaattgttctcacccagtctccagcaatcctgtctgcatctccaggggagaaggtcacaatgacttgcagggccagttcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagtagcaacccgtacacgttcggaggggggaccaagctggaaataaaacgtacg

SEQ ID NO: 9 - DNA, 377 bp, synthetic construct
  misc_feature 1..377: note = synthetic primer
  source 1..377: mol_type = other DNA
accggtgtacattccgaggtccaactgcagcagtctggagctgagctggtgaggcctggggcttcagtgaagctgtcctgcaaggcttctggctacactttcactgactactatataaactgggtgaagcagaggcctggacagggactcgagtggattgcaaggatttatcctggaagtggtaatacttactacaatgagaagttcaagggcaaggccacactgactgcagaaaaatcctccagcactgcctacatgcagctcagcagcctgacatctgaggactctgctgtctatttctgtgcaagacggctaactgcgggatacttcgatgtctggggcacagggaccacggtcaccgtctcctcagcgtcgac

SEQ ID NO: 10 - DNA, 342 bp, synthetic construct
  misc_feature 1..342: note = synthetic primer
  source 1..342: mol_type = other DNA
accggtgtacattccgacatcaagatgacccagtctccatcttccatgtttgcatctctaggagagagagtcactatcacttgcaaggcgagtcaggacattaatagctatttatcctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttccgctcacgttcggtgctgggaccaagctggagctgaaacgtacg

SEQ ID NO: 11 - DNA, 386 bp, synthetic construct
  misc_feature 1..386: note = synthetic primer
  source 1..386: mol_type = other DNA
accggtgtacattccgaagtgatgctggtggagtctggggaagacttagtgaaacctggagggtccctgaaactctcctgtatagcctctggattcactttcagtagctatgccatgtcttgggttcgccagactccagagaagaggctggagtgggtcgcatatattagtagtggtggtgattacatctactatacagacactgtgaagggccgattcaccatctccagagacaatgccaggaacaccctgttcctacaaatgagcagtctgaagtctgaggacacagccatgtattactgtacaagagataccggttactacgtttctcggtacttcgatgtctggggcacagggaccacggtcaccgtctcctcagcgtcgac

SEQ ID NO: 12 - DNA, 342 bp, synthetic construct
  misc_feature 1..342: note = synthetic primer
  source 1..342: mol_type = other DNA
accggtgtacattccgacattgtgatgacccagtctcaaaaattcatgtccacatcagttggagacagggtcagtgtcacctgcaaggccagtcagaatgtgggaactaatgtagcctggtatcaacagaaaccaggacaatctcctaaagcactgatttactcggcatccttccggaacagtggagtccctgatcgcttcacaggcagtggatctgggacagatttcactctcaccatcagcaatgtgcagtctgaagacttggcagagtatttctgtcatcaatataacaactatcctctgacgttcggtggaggcaccaagctggaaatcaaacgtacg

SEQ ID NO: 13 - DNA, 389 bp, synthetic construct
  misc_feature 1..389: note = synthetic primer
  source 1..389: mol_type = other DNA
accggtgtacattccgaggtccaactgcagcagtctggacctgtgctggtgaagcctgggccttcagtgaagatatcctgtaaggcttctggattcacattcactgactactacatgcactgggtgaagcagagccatggaaagagccttgaatggattggacttgtttatccttacaatggtggtacttattacaaccagaagttcaagggcaaggccacattgactgtagacacatcctccagcacagcctacatggagctaaacagcctgacttctgaggactctgcggtctattactgtgtacgattagggaacggtagtagcaacgagtggtacttcgatgtctggggcacagggaccacggtcaccgtctcctcagcgtcgac

SEQ ID NO: 14 - DNA, 345 bp, synthetic construct
  misc_feature 1..345: note = synthetic primer
  source 1..345: mol_type = other DNA
accggtgtacattcccaaattgttctcacccagtctccagcactcatggctgcatctccaggggagaaggtcaccatcacctgcagtgtcagctcaagtataagttcctacaatttgcactggtaccagcagaagtcagaaacctcccccaaaccctggatttatggcacatccaacctggcttctggagtccctgttcgcttcagtggcagtggatctgggacctcttattctctcacaatcagcagtatggaggctgaagatgctgccacttattactgtcaacagtggagaacctacccgtggacgttcggtggaggcaccaagctggaaatcaaacgtacg

SEQ ID NO: 15 - DNA, 374 bp, synthetic construct
  misc_feature 1..374: note = synthetic primer
  source 1..374: mol_type = other DNA
accggtgtacattccgaggtccaactgcagcagcctggagctgagcttgtgaagcctggggcttcagtgaagctgtcctgcaaggcttctggctacaccttcaccaactactggatacactggatgaagcagaggcctggacgaggccttgagtggattggaaggattgagcctaatagcggtgatactaaatacaatgagaagatcaagagcagggccacactgactgtagacaaaccgtccagcacagcctacatgcagctcagcagcctgacatctgaggactctgcggtctattattgtgcaagatctgggtatgattaccctgaggcctggggccaaggcaccactctcacagtctcctcagcgtcgac

SEQ ID NO: 16 - DNA, 354 bp, synthetic construct
  misc_feature 1..354: note = synthetic primer
  source 1..354: mol_type = other DNA
accggtgtacattccgacattgtgctgacccaatctccagcttctttggctgtgtctccagggcagagagccaccatctcctgcagagccagtgaaggtgttgaatattatggcacaactttaatgcagtggtaccaacagaaaccaggacagccacccaaactcctcatctatgctgcatccaacgtagaatctggggtccctgccaggtttagtggcagtgggtctgggacagacttcagcctcaacatccatcctgtggaggaggatgatattgcaatgtatttctgtcagcaaagtaggaaggtttcttggacgttcggtggaggcaccaagctggaaatcaaacgtacg

SEQ ID NO: 17 - DNA, 374 bp, synthetic construct
  misc_feature 1..374: note = synthetic primer
  source 1..374: mol_type = other DNA
accggtgtacattccgaggtccagctgcaacagcctggggctgagcttgtgaagcctggggcttcagtgaagttgtcctgcaaggcttctggctataccttcaccaactactggatgcactgggtgaaacacaggcctggacgaggccttgagtggattggaaggattgatcctaatagtggtggtactaagtacaatgagaagttcaagaataaggccacactgactgtagacaatccctccagcacaggctacatgcagctcagcagcctgacatctgaggactctgcggtctattattgtacaagatctgggtatgattaccctgactactggggccaaggcaccactctcacagtctcctcagcgtcgac

SEQ ID NO: 18 - DNA, 354 bp, synthetic construct
  misc_feature 1..354: note = synthetic primer
  source 1..354: mol_type = other DNA
accggtgtacattccgacattgtgctgacccaatctccagcttctttggctgtgtctctagggcagagagccaccatctcctgcagagccagtgaaagtgttgaatattatggcacaagtttaatgcagtggtaccaacagaaaccaggacagccacccaaagtcctcatctatgctgcatccaacgtagattctggggtccctcccaggtttagtggcagtgggtctgggacagacttcagcctcaacttccatcctgtggaggaggatgatattgcaatgtatttctgtcagcaaagtaggaaggttccttggacgttcggtggaggcaccaagctggaaatcaaacgtacg

SEQ ID NO: 19 - DNA, 374 bp, synthetic construct
  misc_feature 1..374: note = synthetic primer
  source 1..374: mol_type = other DNA
accggtgtacattccgaggtccaactgcaacagcctggggctgagcttgtgaagcctggggcttcagtgaagctgtcctgcaaggcttctggctacaccttcaccagctactggatgcactgggtgaagcagaggcctggacgaggccttgagtggattggaaggattgatcctaatagtggtggtactaagtacaatgagaagttcaagagcaaggccacactgactgtagacaaaccctccagcacagcctacatgcagctcagcagcctgacatctgaggactctgcggtctattattgtgcaagatctgggtatgattaccctgactactggggccaaggcaccactctcacagtctcctcagcgtcgac

SEQ ID NO: 20 - DNA, 354 bp, synthetic construct
  misc_feature 1..354: note = synthetic primer
  source 1..354: mol_type = other DNA
accggtgtacattccgacattgtgctgacccaatctccagctcctttggctgtgtctctagggcagagagccaccatctcctgcagagccagtgaaggtgttgaatattatgacacaagtttaatgcagtggtaccaacagaaaccaggacagccacccaaactcctcatctatgctgcatccaacgtagaacctggggttcctgccaggtttggtggcagtgggtctgggacagacttcagcctcaacatccatcctgtggaggaggatgatattgcaatgtatttctgtcagcaaagtaggaaggttccttggacgttcggtggaggcaccaagctggaaatcaaacgtacg

SEQ ID NO: 21 - DNA, 365 bp, synthetic construct
  misc_feature 1..365: note = synthetic primer
  source 1..365: mol_type = other DNA
accggtgtacattccgaggtccaactgcaacagcctggggctgagctggtaaagcctgggacttcagtgaagttgtcctgcaaggcttctggctacactttcaccagctactggatgcactgggtgaagcagaggcctggacaaggccttgagtggattggaatgattcatcctaatagtggtagtactaactacaatgagatgttcgagagcaaggcctcactgactgtagacaaatcctccagcacagcctacatgcaactcagcagcctgacatctgaggactctgcggtctattactgtgcaagaatcggatactccggctggggtcaaggaacctcagtcaccgtctcctcagcgtcgac

SEQ ID NO: 22 - DNA, 360 bp, synthetic construct
  misc_feature 1..360: note = synthetic primer
  source 1..360: mol_type = other DNA
accggtgtacattccgacattgtgatgacacagtctccatcctccctgagtgtgtcagcaggagagaaggtcactatgagctgcaagtccagtcagagtctgttaaatggtggaaatcaaaagaactacttggcctggtaccagcagaaaccagggcagcctcctaaagtgttgatcaacggggcatccactagggaatctggggtccctgatcgcttcacaggcaatggatctggaaccgatttcactcttaccatcagcagtgtgcaggctgaagacctggcagtttattactgtcagaatgatcatacttatccgctcacgttcggtgctgggaccaagctggagctgaaacgtacg

SEQ ID NO: 23 - DNA, 377 bp, synthetic construct
  misc_feature 1..377: note = synthetic primer
  source 1..377: mol_type = other DNA
accggtgtacattccgaggtccaactgcagcagcctggaggtgagctggtgaggcctgggtcctcagtgaagatgtcctgcaagacttctggatatacattcacattctacggtataaactgggtgaagcagaggcctggacagggcctggaatggattggatatatttatgttggaaatggttatactgagtacaatgagaagttcaaggtcaaggccacactgacttcagacacatcctccagcacagcctacatgcagctcagcggcctgacatctgaggactctgcaatctatttctgtgcaagatccgatggttacaactacttcgactactggggccaaggcaccactctcacagtctcctcagcgtcgac

SEQ ID NO: 24 - DNA, 339 bp, synthetic construct
  misc_feature 1..339: note = synthetic primer
  source 1..339: mol_type = other DNA
accggtgtacattcccaaattgttctcacccagtctccagcaatcctgactgcatccccaggggagaaagtcacaatgacttgcagggccagttcaagtgtacattacatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatactgccacttattactgccatcagtggagtagtaacccacccacgttcggtgctgggacaaagttggaaataaaacgtacg

SEQ ID NO: 25 - DNA, 380 bp, synthetic construct; identical to SEQ ID NO: 7
  misc_feature 1..380: note = synthetic primer
  source 1..380: mol_type = other DNA

SEQ ID NO: 26 - DNA, 339 bp, synthetic construct; identical to SEQ ID NO: 8
  misc_feature 1..339: note = synthetic primer
  source 1..339: mol_type = other DNA

SEQ ID NO: 27 - DNA, 380 bp, synthetic construct
  misc_feature 1..380: note = synthetic primer
  source 1..380: mol_type = other DNA
accggtgtacattccgaggtccagctgcaacagtctggacctgagctggtgaggcctggggcttcagtgaagatatcctgcaaggcttctggctacaccttcactgactactatatacactggctgaagcagaggcctggacagggacttgagtggattggattgatttttcctggaagtggtagtatttactgtaatgagaagttcaagggcaaggctacacttactgtagacaaatcctccaccacagcctacatgttgctcagcagcctgacctctgaggactctgcggtctacttctgtgcaagatgggagactacggcgtggtacttcgatgtctggggcacagggaccacggtcaccgtctcctcagcgtcgac

SEQ ID NO: 28 - DNA, 363 bp, synthetic construct
  misc_feature 1..363: note = synthetic primer
  source 1..363: mol_type = other DNA
accggtgtacattcccaaattgttctcacccagtctccccaaattgttctcacccagtctccagcaatcctgtctgcatctccaggggagaaggtcacaatgacttgcagggccagtttaagtgtaagttacatgcactggtaccagcagaagccaggatcctcccccaaaccctggatttatgccacatccaacctggcttctggagtccctgcccgcttcagtggcagtgggtctgggacctcttactctctcacagtcagcagagtggaggccgaagatgctgccacttattactgccagcagtggaggagttacccgtacacgttcggagggggcaccaagctggaaatcaaacgtacg

SEQ ID NO: 29 - DNA, 386 bp, synthetic construct
  misc_feature 1..386: note = synthetic primer
  source 1..386: mol_type = other DNA
accggtgtacattccgaggtccaactgcaacagtctggacctgtgctggtgaagcctgggccttcaatgaagatatcctgtaaggcttctggattcacattcactgactactacatacactgggtgaggcagagccatggaaagagccttgagtggattggacttgtttctccttacaatggtggtacttactacaaccagaagttcaagggcaaggccacattgactgtagactcatcctccagcacagcctacatggagctaagcagcctgacttctgaggactctgcggtctattactgtgcaagattgggctactatggtgactggtactactttgactactggggccaaggcacccctctcacagtctcctcagcgtcgac

SEQ ID NO: 30 - DNA, 345 bp, synthetic construct
  misc_feature 1..345: note = synthetic primer
  source 1..345: mol_type = other DNA
accggtgtacattcccaaattgttctcacccagtctccagcactcatggctacatctccaggggaaaaggtcaccatcacctgtagtgtcagctcaagtataagttccagcaacttgaactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccatacacgttcggaggggggaccaagctggaaataaaacgtacg

SEQ ID NO: 31 - DNA, 389 bp, synthetic construct
  misc_feature 1..389: note = synthetic primer
  source 1..389: mol_type = other DNA
accggtgtacattccgaggtccagctgcaacagtctggggctgagctggtgaagcctggggcttcagtgaagttgtcctgcaaggcttctggctacactttcaccacctactggatgcactggatgaagcagtggcctggacaaggccttgagtggattggattgattcatcctaatagtggtagtacttactacaatgagaagttcaagagcaaggccacactgactgtagacaaatcctccagcacagcctacatgcaactcagcagcctgacatctgaggactctgcggtctattactgtgcaagatttgaggagctatggggaggttactggtacttcgatgtctggggcacagggaccacggtcaccgtctcctcagcgtcgac

SEQ ID NO: 32 - DNA, 345 bp, synthetic construct
  misc_feature 1..345: note = synthetic primer
  source 1..345: mol_type = other DNA
accggtgtacattcccaaattgttctcacccagtctccagcactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccgtacacgttcggaggggggaccaagctggaaataaaacgtacg

SEQ ID NO: 33 - DNA, 389 bp, synthetic construct
  misc_feature 1..389: note = synthetic primer
  source 1..389: mol_type = other DNA
accggtgtacattccgaggtccagctgcagcagcctggggctgagctggcaaagcctggggcttcagtgaagttgtcctgcaaggcttctggctacaatttcaccagctactggatgcactgggtgaagcagaggcctggacaaggccttgagtggattggattgattcatcctaatagtggtggtacttactacaatgagaagttcaagagcaaggccacactgactttagacagatcctccagcacagcctacatgcaactcagcagcctgacatctgaggactctgcggtctattattgtgcaagatttgaggaggaatggggagggtattggtacttcgatgtctggggcacggggaccacggtcaccgtctcctcagcgtcgac

SEQ ID NO: 34 - DNA, 345 bp, synthetic construct
  misc_feature 1..345: note = synthetic primer
  source 1..345: mol_type = other DNA
accggtgtacattcccaaattgttctcacccagtctccagcactcatggctgcatctccaggggaaaaggtcaccatcacctgcagtgtcagttcaagtataagttccagcaacttgcactggtaccaacagaagtcagaaacctcccccaaaccctggatttatggcacttccaacctggcttctggagtccctgttcgcttcagtggcagtggatctgggacctcttattctctcacaatcagcagcatggaggctgaagatgctgccacttattactgtcaacagtggagtagttacccgtacacgttcggaggggggaccaaactggaaataaaacgtacg

SEQ ID NO: 35 - DNA, 386 bp, synthetic construct
  misc_feature 1..386: note = synthetic primer
  source 1..386: mol_type = other DNA
accggtgtacattccgaggtccaactgcaacagtctggggctgagctggtaaagcctggggcttcagtgaagttgtcctgcaaggcttctggctacactttcaccagctactggatgcactgggtgaagcagaggcctggacaaggccttgagtggattggactgattcatcctaatagtagtagtacttactacaatgagaagttcaagaccagggccacactgactgtagacaagtcctccagcacagcctacatgcaactcagcagcctgacatctgaggactctgcggtctattactgtgcaagattgggctatggtaactcctactggtacttcgatgtctggggcacagggaccacggtcaccgtctcctcagcgtcgac

SEQ ID NO: 36 - DNA, 345 bp, synthetic construct
  misc_feature 1..345: note = synthetic primer
  source 1..345: mol_type = other DNA
accggtgtacattcccaaattgttctcacccagtctccagcactcatggctgcatctccaggggagaaggtcaccatcacctgcagtgtcagctcaagtattagttccagcaacttgcactggtaccagcagaagtcagaaacctcccccaaaccctggatttatggcacatccaacctggcttctggagtccctattcgcttcagtggcagtggatctgggacctcttattctctcacaatcagcagtttggaggctgaggatgctgccacttattactgtcaacagtggagaagttacccgtggacgttcggtggaggcaccaagctggaaatcaaacgtacg

SEQ ID NO: 37 - DNA, 380 bp, synthetic construct
  misc_feature 1..380: note = synthetic primer
  source 1..380: mol_type = other DNA
accggtgtacattccgaggtccaactgcagcagtctggggctgagctggtaaagcctggggcttcagtgaagttgtcctgcaaggcttctggctacactttcaccagctactggatgcactgggtgcagcagaggcctggacaaggccttgagtggattggattgattcatcctattggtggtggtactcactacaatgagaagttcaagaacaaggccacactgactgtagacaaatcctccagcacagcctacatgcaactcagcagcctgacatctgaggactctgcggtctattactgtgcaagactcggaactggtccgtactactttgactactggggccaaggcaccactctcacagtctcctcagcgtcgac

SEQ ID NO: 38 - DNA, 345 bp, synthetic construct
  misc_feature 1..345: note = synthetic primer
  source 1..345: mol_type = other DNA
accggtgtacattcccaaattgttctcacccagtctccagcactcatggctgcatctccaggggagaaggtcaccatcacctgcagtgtcagctcaagtataagttccagcaccttggactggtaccagcagaagtcagaaacctcccccaaaccctgggtttatggcacatccaacctggcttctggagtccctgttcgcttcagtggcagtggatctgggacctcttattctctcacaatcagcagcatggaggctgaagatgctgccacttattactgtcaacagtggagtagttacccgtggacgttcggtggaggcaccaagctggaaatcaaacgtacg

SEQ ID NO: 39 - DNA, 380 bp, synthetic construct
  misc_feature 1..380: note = synthetic primer
  source 1..380: mol_type = other DNA
accggtgtacattccgaggtccagctgcagcagtctggacctgtgctggtgaagcctgggccttcagtgaatatatcctgtaaggcttctggattcacattcactgactactacattcactgggtgaaacagagccatggaaagagccttgagtggattggacttgtttatccttacagtggtggtacttactacaaccagaagttcaagggcaaggccacattgactgtagacgcatcctccagcacagcctacatggagctaggcagcctgacttctgaggactctgcggtctattactgtgcaagattgggcgacaacttctactactttgactactggggccaaggcaccactctcacagtctcctcagcgtcgac

SEQ ID NO: 40 - DNA, 345 bp, synthetic construct
  misc_feature 1..345: note = synthetic primer
  source 1..345: mol_type = other DNA
accggtgtacattcccaaattgttctcacccagtctccaacactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacatcttattctctcacaatcagcagcatggaggctgaagatgctgccacttattactgtcaacagtggagaagttacccgtacacgttcggaggggggaccaagctggaaataaaacgtacg

SEQ ID NO: 41 - DNA, 386 bp, synthetic construct
  misc_feature 1..386: note = synthetic primer
  source 1..386: mol_type = other DNA
accggtgtacattccgaggtccaactgcaacagcctggacctgtgctggtgaagcctgggccttcagtgaagatatcctgtaaggcttctggattcacattcactgactactacatgcactgggtgaaacagagccatggaaagagccttgagtggattggtcttgtttctccttacaatggtggtactttctacaaccagaagttcaagggcaaggccacattgactgtagacacatcctccagcacagcctacatggaactaaacagcctgacttctgaggactctgcggtctattactgtgcaagagtgggtaatagctacgtccattatgctatggactactggggtcaaggaacctcagtcaccgtctcctcagcgtcgac

SEQ ID NO: 42 - DNA, 345 bp, synthetic construct
  misc_feature 1..345: note = synthetic primer
  source 1..345: mol_type = other DNA
accggtgtacattcccaaattgttctcacccagtctccagcactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtaattacccgtacacgttcggaggggggaccaagctggaaataaaacgtacg

SEQ ID NO: 43 - DNA, 386 bp, synthetic construct
  misc_feature 1..386: note = synthetic primer
  source 1..386: mol_type = other DNA
accggtgtacattccgaagtgaagctggtggagtctggggaaggcttagtgaagcctggagggtccctgaaactctcctgtgcagcctctggattcactttcagtagctatgccatgtcttgggttcgccagactccagagaagaggctggactgggtcgcatacattagtagtggtggtgatcacatctactatgcagacactgtgaagggccgattcaccatctccagagacaatgccaggaacaccctgtacctgcaaatgagcagtctgaagtctgaggacacagccatgtattactgtacaagagataccggttactacgtctctcggtacttcgatgtctggggcacagggaccacggtcaccgtctcctcagcgtcgac

SEQ ID NO: 44 - DNA, 342 bp, synthetic construct
  misc_feature 1..342: note = synthetic primer
  source 1..342: mol_type = other DNA
accggtgtacattccgacattgtgatgacccagtctcaaaaattcatgtccacatcagtaggagacagggtcagcgtcacctgcaaggccagtcagaatgtggttactaatgttgcctggtatcaacagaaatcagggcaatctcctaaagtagtgatttattcggcatccttccggtccagtggagtccctgatcgcttcacaggcagtggatctgggacagatttcactctcaccatcagcaatgtgcagtctgaagacttggcagagtatttctgtcaccaatataacagccatcctctgacgttcggtggaggcaccaagctggaaatcaaacgtacg

SEQ ID NO: 45 - DNA, 386 bp, synthetic construct
  misc_feature 1..386: note = synthetic primer
  source 1..386: mol_type = other DNA
accggtgtacattccgaggtccagctgcagcagcctggacctgaattggtgaagcccgggtcctcactgaagatatcctgcaaggcttctggttacaccttcactgactactttataaactgggtgaaacagaggcctggacagggacttgactggattggatggatttttcctggaagtggtagtacttactacaatgacaagttcaagggcaaggccacacttactgtagacaaatcctccagcactgcctacatgttgctcagcagcctgacctctgaggcctctgcggtctatttctgtgcaagatgggactccgatagtacctactggtacttcgatgtctggggcacagggaccacggtcaccgtctcctcagcgtcgac

SEQ ID NO: 46 - DNA, 345 bp, synthetic construct
  misc_feature 1..345: note = synthetic primer
  source 1..345: mol_type = other DNA
accggtgtacattcccaaattgttctcacccagtctccagcactcatggctgcatctccaggggagaaggtcaccatcacctgcagtgtcaattcaagtataagttccagcaccttgcactggtaccagcagaagtcacaaacctcccccaaaccctggatttatggtacatccaatttggcttctggagtccctattcgcttcagtggcagtggatctgggacctcttattctctcacaatcagcagcatggaggctgaagatgctgccacttattactgtcaacagtggagtacttacccgtacacgttcggaggtggcaccaagctggaaatcaaacgtacg

SEQ ID NO: 47 - DNA, 374 bp, synthetic construct
  misc_feature 1..374: note = synthetic primer
  source 1..374: mol_type = other DNA
accggtgtacattccgaggtccaactgcaacagtctggagctgagctgatgaagcctggggcctcagtgaagctttcctgcaaggctactggctacacattcactggctactggatagagtggttaaagcagaggcctggacatggccttgagtggattggagagattttacctggaagtgataatactaactacaatgagaagttcaggggcaaggccacattcactgcagatacatcctccaacacagcctacatgcacctcagcagcctgacaactgaggactctgccatctattactgtgcaagagaagggggtttctactttgactactggggccaaggcaccactctcacagtctcctcagcgtcgac

SEQ ID NO: 48 - DNA, 389 bp, synthetic construct
  misc_feature 1..389: note = synthetic primer
  source 1..389: mol_type = other DNA
accggtgtacattcccaggctgttgtgactcaggaatctgcactcaccacatcacctggtgaaacagtcacactcacttgtcgctcaagtattggggctgttacaactagtaactacgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggccagcccaagtcttcgccatcagtcaccctgtttccgccctcgag

SEQ ID NO: 49 - DNA, 383 bp, synthetic construct
  misc_feature 1..383: note = synthetic primer
  source 1..383: mol_type = other DNA
accggtgtacattccgaggtccagctgcaacagtctggacctgtgctggtgaagcctgggccttcagtgaagatatcctgtaaggcttctggattcacattcactgactactacatgcactgggtgaagcagagccatggaaagagccttgaatggattggacttgtttatccttacaatggtggtactagctacaaccagaagttcaagggcaaggccacattgactgtagacacatcctccaccacagcctacatggagctaaacagcctgacttccgaggactctgcggtctattactgtgcaagattgggctcaggctacgagtactactttgactactggggccaaggcacctctctcacagtctcctcagcgtcgac

SEQ ID NO: 50 - DNA, 345 bp, synthetic construct
  misc_feature 1..345: note = synthetic primer
  source 1..345: mol_type = other DNA
accggtgtacattcccaaattgttctcacccagtctccagcactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaggatgctgccacttattactgtcaacagtggagaacttacccgtggacgttcggtggaggcaccaagctggaaatcaaacgtacg

SEQ ID NO: 51 - DNA, 386 bp, synthetic construct
  misc_feature 1..386: note = synthetic primer
  source 1..386: mol_type = other DNA
accggtgtacattccgaggtccaactgcagcagcctggacctgtgctggtgaagcctgggccttcagtgaagatatcctgtaaggcttctggattcacattcactgactactacatgcactgggtgaagcagagccatggagagagccttgagtggattggacttgtttctccttacaatggtggtactttctacaaccagaagttcaagggcaaggccacattgactgtagacacatcctccagcacagcctacatggaactaaacagcctgacttctgaggactctgcggtctattactgtgcaagagtgggtagtagttacgtccattatgctatggactactggggtcaaggaacctcagtcaccgtctcctcagcgtcgac

SEQ ID NO: 52 - DNA, 345 bp, synthetic construct
  misc_feature 1..345: note = synthetic primer
  source 1..345: mol_type = other DNA
accggtgtacattcccaaattgttctcacccagtctccagcactcacggctgcatctccaggggagagggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtaagtacccgtacacgttcggaggggggaccaagctggaaataaaacgtacg

SEQ ID NO: 53 - DNA, 380 bp, synthetic construct
  misc_feature 1..380: note = synthetic primer
  source 1..380: mol_type = other DNA
accggtgtacattccgaggtccaactgcaacagtctggggctgggctggtaaagcctggggcttcagtgaagttgccttgcaaggcttctggctacactttcaccagctactggatgcactgggtgcagcagaggcctggacaaggccttgagtggattggattgattcatcctattggtggtggtactcactacaatgagaagttcaagaacaaggccacactgactgtggacaaatcctccagcacagcctacatgcaactcagcagcctgacatctgaggactctgcggtctattactgtgcaagactcggaactggtccgtactactttgactactggggccaaggcaccactctcacagtctcctcagcgtcgac

SEQ ID NO: 54 - DNA, 345 bp, synthetic construct
  misc_feature 1..345: note = synthetic primer
  source 1..345: mol_type = other DNA
accggtgtacattcccaaattgttctcacccagtctccagcactcatggctgcatctccaggggagaaggtcaccatcacctgcagtgtcagctcaagtataagttccagcaacttgcactggtaccagcagaagtcagagacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccgtggacgttcggtggaggcaccaagctggaaatcaaacgtacg

SEQ ID NO: 55 - DNA, 380 bp, synthetic construct
  misc_feature 1..380: note = synthetic primer
  source 1..380: mol_type = other DNA
accggtgtacattccgaggtccagctgcagcagcctggacctgacgtggtgaagcctggggcttcagtgaagatttcctgcaaggcttctggctacaccttcactgactactatataaactgggtgaagcagaggcctggacagggacttgagtggattggatggatttttcctggaagtggtagtagttattacaatgagaagttcaaggacaaggccacatctactgtagacaaatcctccagcacagcctacatgttgctcagcagcctgacctctgaggactctgcggtctatttctgtgcaaaatggaaggattacgggtggtacttcgatgtctggggcacagggaccacggtcaccgtctcctcagcgtcgac

SEQ ID NO: 56 - DNA, 345 bp, synthetic construct
  misc_feature 1..345: note = synthetic primer
  source 1..345: mol_type = other DNA
accggtgtacattcccaaattgttctcacccagtctccagcactcatggctgcatctccaggggagaaggtcaccctcacctgcaatgtcagctcaagtctaagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggaggacttacccttacacgttcggaggggggaccaagctggaaataaaacgtacg

SEQ ID NO: 57 - DNA, 377 bp, synthetic construct
  misc_feature 1..377: note = synthetic primer
  source 1..377: mol_type = other DNA
accggtgtacattccgaggtccaactgcagcagtctggacctgtgctggtgaagcctgggccttcagtgaagatatcctgtaaggcttctggattcacattcactgactactacattcactgggtgaagcggagccatggaaagagccttgagtggattggacttgtttctccttacaatggtggtactttctacaaccagaagttcaagggcaaggccacattgactgtagatacatcctccaatacagcctacatggagctaaccagcctgacttctgaggactctgcggtctattactgtgcaagattgggggttaactggtacttcgatgtctggggcacagggaccacggtcaccgtctcctcagcgtcgac

SEQ ID NO: 58 - DNA, 345 bp, synthetic construct
  misc_feature 1..345: note = synthetic primer
  source 1..345: mol_type = other DNA
accggtgtacattcccaaattgttctcacccagtctccagcactcatggctgcatctccaggggaaaaggtcaccctcacctgcagtgtcagctcaagtataagttccagcaccttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtacttacccatacacgttcggaggggggaccaagctggaaataaaacgtacg

SEQ ID NO: 59 - DNA, 386 bp, synthetic construct
  misc_feature 1..386: note = synthetic primer
  source 1..386: mol_type = other DNA
accggtgtacattccgaggtccaactgcaacagcctggacctgtactggtgaagcctgggccttcaatgaagatatcctgtaaggcttctgggtacgctttcactgactacttcatacactgggtgagacagagccatggaaggagccttgagtggattggacttgtttctccttataatggtggtacttactacaaccagaagttcaagggcaaggcctcattgactgtagacacatcctccagcacagcctacatggaactaagcagcctgacttctgaggactctgcggtctattactgtgcaagattgggctactatggtaactggtacttctttgactactggggccaaggcacccctctcacagtctcctcagcgtcgac

SEQ ID NO: 60 - DNA, 345 bp, synthetic construct; identical to SEQ ID NO: 30
  misc_feature 1..345: note = synthetic primer
  source 1..345: mol_type = other DNA

SEQ ID NO: 61 - protein, 128 aa, synthetic construct
  REGION 1..128: note = synthetic primer
TGVHSEVQLQQSGAELVKPGASVKLSCKASGYTFTSYWVHWVRQRPGQGLEWIGMIHPNSGSTNYNEKFKSKATLTVDKSSSTAYMQLSSLTSEDSAVYYCASYYYGSSYYFDYWGQGTTLTVSSAST

SEQ ID NO: 62 - protein, 120 aa, synthetic construct
  REGION 1..120: note = synthetic primer
TGVHSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELKRT

SEQ ID NO: 63 - protein, 124 aa, synthetic construct
  REGION 1..124: note = synthetic primer
TGVHSEVQLQQSGAELVKPGASVKLSCKASGYTFTNYWMHWVKQRPGQGLEWIGMIHPNSGTSNYNEKFKSKATLTVDKSSSTAYMQLSSLTSEDSAVFYCTRSDWAFDYWGQGTSLTVSSAST

SEQ ID NO: 64 - protein, 144 aa, synthetic construct
  REGION 1..144: note = synthetic primer
TGVHSDIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHSYPLTFGAGTKLELKRTPLYHAIASRMAFIFSSLYKSWXLX

SEQ ID NO: 65 - protein, 126 aa, synthetic construct
  REGION 1..126: note = synthetic primer
TGVHSEVQLQQSGAELVRPGSSVKMSCKTSGYTFTFYGINWVKQRPGQGLEWIGYIYVGNGYSEYNEKFKVKATLTSDTSSSTAYMQLSGLTSEDSAIYFCARSDGYNYFDYWGQGTTLTVSSTST

SEQ ID NO: 66 - protein, 113 aa, synthetic construct
  REGION 1..113: note = synthetic primer
TGVHSQIVLTQSPAILSASPGEKVTMTCRASSSLHYMHWYQQKTGSSPKPWIYATSNLASGVPARFSGSGSGTSYSLTISRVEAEDTATYYCQQWSSNPPTFGAGTKLELKRT

SEQ ID NO: 67 - protein, 127 aa, synthetic construct
  REGION 1..127: note = synthetic primer
TGVHSEVQLQQPGAELVKPGASVKLSCKASGYTFTSYWMHWVKQRPGQGLEWIGLIHPNSGSTYYNEKFKNKATLTVDKSSSTAYMQLSSLTSEDSAVYYCARWDDSYWYFKVWGTGTTVTVSSAST

SEQ ID NO: 68 - protein, 113 aa, synthetic construct
  REGION 1..113: note = synthetic primer
TGVHSQIVLTQSPAILSASPGEKVTMTCRASSSVSYMHWYQQKPGSSPKPWIYATSNLASGVPARFSGSGSGTSYSLTISRVEAEDAATYYCQQWSSNPYTFGGGTKLEIKRT

SEQ ID NO: 69 - protein, 128 aa, synthetic construct
  REGION 1..128: note = synthetic primer
TGVHSEVQLQQSGAELVRPGASVKLSCKASGYTFTDYYINWVKQRPGQGLEXXWIARIYPGSGNTYYNEKFKGKATLTAEKSSSTAYMQLSSLTSEDSAVYFCARRLTAGYFDVWGTGTTVTVSSAST

SEQ ID NO: 70 - protein, 114 aa, synthetic construct
  REGION 1..114: note = synthetic primer
TGVHSDIKMTQSPSSMFASLGERVTITCKASQDINSYLSWFQQKPGKSPKTLIYRANRLVDGVPSRFSGSGSGQDYSLTISSLEYEDMGIYYCLQYDEFPLTFGAGTKLELKRT

SEQ ID NO: 71 - protein, 129 aa, synthetic construct
  REGION 1..129: note = synthetic primer
TGVHSEVMLVESGEDLVKPGGSLKLSCIASGFTFSSYAMSWVRQTPEKRLEWVAYISSGGDYIYYTDTVKGRFTISRDNARNTLFLQMSSLKSEDTAMYYCTRDTGYYVSRYFDVWGTGTTVTVSSAST

SEQ ID NO: 72 - protein, 114 aa, synthetic construct
  REGION 1..114: note = synthetic primer
TGVHSDIVMTQSQKFMSTSVGDRVSVTCKASQNVGTNVAWYQQKPGQSPKALIYSASFRNSGVPDRFTGSGSGTDFTLTISNVQSEDLAEYFCHQYNNYPLTFGGGTKLEIKRT

SEQ ID NO: 73 - protein, 130 aa, synthetic construct
  REGION 1..130: note = synthetic primer
TGVHSEVQLQQSGPVLVKPGPSVKISCKASGFTFTDYYMHWVKQSHGKSLEWIGLVYPYNGGTYYNQKFKGKATLTVDTSSSTAYMELNSLTSEDSAVYYCVRLGNGSSNEWYFDVWGTGTTVTVSSAST

SEQ ID NO: 74 - protein, 115 aa, synthetic construct
  REGION 1..115: note = synthetic primer
TGVHSQIVLTQSPALMAASPGEKVTITCSVSSSISSYNLHWYQQKSETSPKPWIYGTSNLASGVPVRFSGSGSGTSYSLTISSMEAEDAATYYCQQWRTYPWTFGGGTKLEIKRT

SEQ ID NO: 75 - protein, 125 aa, synthetic construct
  REGION 1..125: note = synthetic primer
TGVHSEVQLQQPGAELVKPGASVKLSCKASGYTFTNYWIHWMKQRPGRGLEWIGRIEPNSGDTKYNEKIKSRATLTVDKPSSTAYMQLSSLTSEDSAVYYCARSGYDYPEAWGQGTTLTVSSAST

SEQ ID NO: 76 - protein, 118 aa, synthetic construct
  REGION 1..118: note = synthetic primer
TGVHSDIVLTQSPASLAVSPGQRATISCRASEGVEYYGTTLMQWYQQKPGQPPKLLIYAASNVESGVPARFSGSGSGTDFSLNIHPVEEDDIAMYFCQQSRKVSWTFGGGTKLEIKRT

SEQ ID NO: 77 - protein, 125 aa, synthetic construct
  REGION 1..125: note = synthetic primer
TGVHSEVQLQQPGAELVKPGASVKLSCKASGYTFTNYWMHWVKHRPGRGLEWIGRIDPNSGGTKYNEKFKNKATLTVDNPSSTGYMQLSSLTSEDSAVYYCTRSGYDYPDYWGQGTTLTVSSAST

SEQ ID NO: 78 - protein, 118 aa, synthetic construct
  REGION 1..118: note = synthetic primer
TGVHSDIVLTQSPASLAVSLGQRATISCRASESVEYYGTSLMQWYQQKPGQPPKVLIYAASNVDSGVPPRFSGSGSGTDFSLNFHPVEEDDIAMYFCQQSRKVPWTFGGGTKLEIKRT

SEQ ID NO: 79 - protein, 125 aa, synthetic construct
  REGION 1..125: note = synthetic primer
TGVHSEVQLQQPGAELVKPGASVKLSCKASGYTFTSYWMHWVKQRPGRGLEWIGRIDPNSGGTKYNEKFKSKATLTVDKPSSTAYMQLSSLTSEDSAVYYCARSGYDYPDYWGQGTTLTVSSAST

SEQ ID NO: 80 - protein, 118 aa, synthetic construct
  REGION 1..118: note = synthetic primer
TGVHSDIVLTQSPAPLAVSLGQRATISCRASEGVEYYDTSLMQWYQQKPGQPPKLLIYAASNVEPGVPARFGGSGSGTDFSLNIHPVEEDDIAMYFCQQSRKVPWTFGGGTKLEIKRT

SEQ ID NO: 81 - protein, 122 aa, synthetic construct
  REGION 1..122: note = synthetic primer
TGVHSEVQLQQPGAELVKPGTSVKLSCKASGYTFTSYWMHWVKQRPGQGLEWIGMIHPNSGSTNYNEMFESKASLTVDKSSSTAYMQLSSLTSEDSAVYYCARIGYSGWGQGTSVTVSSAST

SEQ ID NO: 82 - protein, 120 aa, synthetic construct
  REGION 1..120: note = synthetic primer
TGVHSDIVMTQSPSSLSVSAGEKVTMSCKSSQSLLNGGNQKNYLAWYQQKPGQPPKVLINGASTRESGVPDRFTGNGSGTDFTLTISSVQAEDLAVYYCQNDHTYPLTFGAGTKLELKRT

SEQ ID NO: 83 - protein, 126 aa, synthetic construct
  REGION 1..126: note = synthetic primer
TGVHSEVQLQQPGGELVRPGSSVKMSCKTSGYTFTFYGINWVKQRPGQGLEWIGYIYVGNGYTEYNEKFKVKATLTSDTSSSTAYMQLSGLTSEDSAIYFCARSDGYNYFDYWGQGTTLTVSSAST

SEQ ID NO: 84 - protein, 113 aa, synthetic construct
  REGION 1..113: note = synthetic primer
TGVHSQIVLTQSPAILTASPGEKVTMTCRASSSVHYMHWYQQKPGSSPKPWIYATSNLASGVPARFSGSGSGTSYSLTISRVEAEDTATYYCHQWSSNPPTFGAGTKLEIKRT

SEQ ID NO: 85 - protein, 127 aa, synthetic construct; identical to SEQ ID NO: 67
  REGION 1..127: note = synthetic primer

SEQ ID NO: 86 - protein, 113 aa, synthetic construct; identical to SEQ ID NO: 68
  REGION 1..113: note = synthetic primer

SEQ ID NO: 87 - protein, 127 aa, synthetic construct
  REGION 1..127: note = synthetic primer
TGVHSEVQLQQSGPELVRPGASVKISCKASGYTFTDYYIHWLKQRPGQGLEWIGLIFPGSGSIYCNEKFKGKATLTVDKSSTTAYMLLSSLTSEDSAVYFCARWETTAWYFDVWGTGTTVTVSSAST

SEQ ID NO: 88 - protein, 121 aa, synthetic construct
  REGION 1..121: note = synthetic primer
TGVHSQIVLTQSPQIVLTQSPAILSASPGEKVTMTCRASLSVSYMHWYQQKPGSSPKPWIYATSNLASGVPARFSGSGSGTSYSLTVSRVEAEDAATYYCQQWRSYPYTFGGGTKLEIKRT

SEQ ID NO: 89 - protein, 129 aa, synthetic construct
  REGION 1..129: note = synthetic primer
TGVHSEVQLQQSGPVLVKPGPSMKISCKASGFTFTDYYIHWVRQSHGKSLEWIGLVSPYNGGTYYNQKFKGKATLTVDSSSSTAYMELSSLTSEDSAVYYCARLGYYGDWYYFDYWGQGTPLTVSSAST

SEQ ID NO: 90 - protein, 115 aa, synthetic construct
  REGION 1..115: note = synthetic primer
TGVHSQIVLTQSPALMATSPGEKVTITCSVSSSISSSNLNWYQQKSETSPKPWIYGTSNLASGVPVRFSGSGSGTSYSLTISSMEAEDAATYYCQQWSSYPYTFGGGTKLEIKRT

SEQ ID NO: 91 - protein, 130 aa, synthetic construct
  REGION 1..130: note = synthetic primer
TGVHSEVQLQQSGAELVKPGASVKLSCKASGYTFTTYWMHWMKQWPGQGLEWIGLIHPNSGSTYYNEKFKSKATLTVDKSSSTAYMQLSSLTSEDSAVYYCARFEELWGGYWYFDVWGTGTTVTVSSAST

SEQ ID NO: 92 - protein, 115 aa, synthetic construct
  REGION 1..115: note = synthetic primer
TGVHSQIVLTQSPALMAASPGEKVTITCSVSSSISSSNLHWYQQKSETSPKPWIYGTSNLASGVPVRFSGSGSGTSYSLTISSMEAEDAATYYCQQWSSYPYTFGGGTKLEIKRT

SEQ ID NO: 93 - protein, 130 aa, synthetic construct
  REGION 1..130: note = synthetic primer
TGVHSEVQLQQPGAELAKPGASVKLSCKASGYNFTSYWMHWVKQRPGQGLEWIGLIHPNSGGTYYNEKFKSKATLTLDRSSSTAYMQLSSLTSEDSAVYYCARFEEEWGGYWYFDVWGTGTTVTVSSAST

SEQ ID NO: 94 - protein, 115 aa, synthetic construct; identical to SEQ ID NO: 92
  REGION 1..115: note = synthetic primer

SEQ ID NO: 95 - protein, 129 aa, synthetic construct
  REGION 1..129: note = synthetic primer
TGVHSEVQLQQSGAELVKPGASVKLSCKASGYTFTSYWMHWVKQRPGQGLEWIGLIHPNSSSTYYNEKFKTRATLTVDKSSSTAYMQLSSLTSEDSAVYYCARLGYGNSYWYFDVWGTGTTVTVSSAST

SEQ ID NO: 96 - protein, 115 aa, synthetic construct
  REGION 1..115: note = synthetic primer
TGVHSQIVLTQSPALMAASPGEKVTITCSVSSSISSSNLHWYQQKSETSPKPWIYGTSNLASGVPIRFSGSGSGTSYSLTISSLEAEDAATYYCQQWRSYPWTFGGGTKLEIKRT

SEQ ID NO: 97 - protein, 127 aa, synthetic construct
  REGION 1..127: note = synthetic primer
TGVHSEVQLQQSGAELVKPGASVKLSCKASGYTFTSYWMHWVQQRPGQGLEWIGLIHPIGGGTHYNEKFKNKATLTVDKSSSTAYMQLSSLTSEDSAVYYCARLGTGPYYFDYWGQGTTLTVSSAST

SEQ ID NO: 98 - protein, 115 aa, synthetic construct
  REGION 1..115: note = synthetic primer
TGVHSQIVLTQSPALMAASPGEKVTITCSVSSSISSSTLDWYQQKSETSPKPWVYGTSNLASGVPVRFSGSGSGTSYSLTISSMEAEDAATYYCQQWSSYPWTFGGGTKLEIKRT

SEQ ID NO: 99 - protein, 127 aa, synthetic construct
  REGION 1..127: note = synthetic primer
TGVHSEVQLQQSGPVLVKPGPSVNISCKASGFTFTDYYIHWVKQSHGKSLEWIGLVYPYSGGTYYNQKFKGKATLTVDASSSTAYMELGSLTSEDSAVYYCARLGDNFYYFDYWGQGTTLTVSSAST

SEQ ID NO: 100 - protein, 115 aa, synthetic construct
  REGION 1..115: note = synthetic primer
TGVHSQIVLTQSPTLMAASPGEKVTITCSVSSSISSSNLHWYQQKSETSPKPWIYGTSNLASGVPVRFSGSGSGTSYSLTISSMEAEDAATYYCQQWRSYPYTFGGGTKLEIKRT

SEQ ID NO: 101 - protein, 129 aa, synthetic construct
  REGION 1..129: note = synthetic primer
TGVHSEVQLQQPGPVLVKPGPSVKISCKASGFTFTDYYMHWVKQSHGKSLEWIGLVSPYNGGTFYNQKFKGKATLTVDTSSSTAYMELNSLTSEDSAVYYCARVGNSYVHYAMDYWGQGTSVTVSSAST

SEQ ID NO: 102 - protein, 115 aa, synthetic construct
  REGION 1..115: note = synthetic primer
TGVHSQIVLTQSPALMAASPGEKVTITCSVSSSISSSNLHWYQQKSETSPKPWIYGTSNLASGVPVRFSGSGSGTSYSLTISSMEAEDAATYYCQQWSNYPYTFGGGTKLEIKRT

SEQ ID NO: 103 - protein, 129 aa, synthetic construct
  REGION 1..129: note = synthetic primer
TGVHSEVKLVESGEGLVKPGGSLKLSCAASGFTFSSYAMSWVRQTPEKRLDWVAYISSGGDHIYYADTVKGRFTISRDNARNTLYLQMSSLKSEDTAMYYCTRDTGYYVSRYFDVWGTGTTVTVSSAST

SEQ ID NO: 104 - protein, 114 aa, synthetic construct
  REGION 1..114: note = synthetic primer
TGVHSDIVMTQSQKFMSTSVGDRVSVTCKASQNVVTNVAWYQQKSGQSPKVVIYSASFRSSGVPDRFTGSGSGTDFTLTISNVQSEDLAEYFCHQYNSHPLTFGGGTKLEIKRT

SEQ ID NO: 105 - protein, 129 aa, synthetic construct
  REGION 1..129: note = synthetic primer
TGVHSEVQLQQPGPELVKPGSSLKISCKASGYTFTDYFINWVKQRPGQGLDWIGWIFPGSGSTYYNDKFKGKATLTVDKSSSTAYMLLSSLTSEASAVYFCARWDSDSTYWYFDVWGTGTTVTVSSAST

SEQ ID NO: 106 - protein, 115 aa, synthetic construct
  REGION 1..115: note = synthetic primer
TGVHSQIVLTQSPALMAASPGEKVTITCSVNSSISSSTLHWYQQKSQTSPKPWIYGTSNLASGVPIRFSGSGSGTSYSLTISSMEAEDAATYYCQQWSTYPYTFGGGTKLEIKRT

SEQ ID NO: 107 - protein, 125 aa, synthetic construct
  REGION 1..125: note = synthetic primer
TGVHSEVQLQQSGAELMKPGASVKLSCKATGYTFTGYWIEWLKQRPGHGLEWIGEILPGSDNTNYNEKFRGKATFTADTSSNTAYMHLSSLTTEDSAIYYCAREGGFYFDYWGQGTTLTVSSAST

SEQ ID NO: 108 - protein, 130 aa, synthetic construct
  REGION 1..130: note = synthetic primer
TGVHSQAVVTQESALTTSPGETVTLTCRSSIGAVTTSNYANWVQEKPDHLFTGLIGGTNNRAPGVPARFSGSLIGDKAALTITGAQTEDEAIYFCALWYSNHWVFGGGTKLTVLGQPKSSPSVTLFPPSX

SEQ ID NO: 109 - protein, 128 aa, synthetic construct
  REGION 1..128: note = synthetic primer
TGVHSEVQLQQSGPVLVKPGPSVKISCKASGFTFTDYYMHWVKQSHGKSLEWIGLVYPYNGGTSYNQKFKGKATLTVDTSSTTAYMELNSLTSEDSAVYYCARLGSGYEYYFDYWGQGTSLTVSSAST

SEQ ID NO: 110 - protein, 115 aa, synthetic construct
  REGION 1..115: note = synthetic primer
TGVHSQIVLTQSPALMAASPGEKVTITCSVSSSISSSNLHWYQQKSETSPKPWIYGTSNLASGVPVRFSGSGSGTSYSLTISSMEAEDAATYYCQQWRTYPWTFGGGTKLEIKRT

SEQ ID NO: 111 - protein, 129 aa, synthetic construct
  REGION 1..129: note = synthetic primer
TGVHSEVQLQQPGPVLVKPGPSVKISCKASGFTFTDYYMHWVKQSHGESLEWIGLVSPYNGGTFYNQKFKGKATLTVDTSSSTAYMELNSLTSEDSAVYYCARVGSSYVHYAMDYWGQGTSVTVSSAST

SEQ ID NO: 112 - protein, 115 aa, synthetic construct
  REGION 1..115: note = synthetic primer
TGVHSQIVLTQSPALTAASPGERVTITCSVSSSISSSNLHWYQQKSETSPKPWIYGTSNLASGVPVRFSGSGSGTSYSLTISSMEAEDAATYYCQQWSKYPYTFGGGTKLEIKRT

SEQ ID NO: 113 - protein, 127 aa, synthetic construct
  REGION 1..127: note = synthetic primer
TGVHSEVQLQQSGAGLVKPGASVKLPCKASGYTFTSYWMHWVQQRPGQGLEWIGLIHPIGGGTHYNEKFKNKATLTVDKSSSTAYMQLSSLTSEDSAVYYCARLGTGPYYFDYWGQGTTLTVSSAST

SEQ ID NO: 114 - protein, 115 aa, synthetic construct
  REGION 1..115: note = synthetic primer
TGVHSQIVLTQSPALMAASPGEKVTITCSVSSSISSSNLHWYQQKSETSPKPWIYGTSNLASGVPVRFSGSGSGTSYSLTISSMEAEDAATYYCQQWSSYPWTFGGGTKLEIKRT

SEQ ID NO: 115 - protein, 127 aa, synthetic construct
  REGION 1..127: note = synthetic primer
TGVHSEVQLQQPGPDVVKPGASVKISCKASGYTFTDYYINWVKQRPGQGLEWIGWIFPGSGSSYYNEKFKDKATSTVDKSSSTAYMLLSSLTSEDSAVYFCAKWKDYGWYFDVWGTGTTVTVSSAST

SEQ ID NO: 116 - protein, 115 aa, synthetic construct
  REGION 1..115: note = synthetic primer
TGVHSQIVLTQSPALMAASPGEKVTLTCNVSSSLSSSNLHWYQQKSETSPKPWIYGTSNLASGVPVRFSGSGSGTSYSLTISSMEAEDAATYYCQQWRTYPYTFGGGTKLEIKRT

SEQ ID NO: 117 - protein, 126 aa, synthetic construct
  REGION 1..126: note = synthetic primer
TGVHSEVQLQQSGPVLVKPGPSVKISCKASGFTFTDYYIHWVKRSHGKSLEWIGLVSPYNGGTFYNQKFKGKATLTVDTSSNTAYMELTSLTSEDSAVYYCARLGVNWYFDVWGTGTTVTVSSAST

SEQ ID NO: 118 - protein, 115 aa, synthetic construct
  REGION 1..115: note = synthetic primer
TGVHSQIVLTQSPALMAASPGEKVTLTCSVSSSISSSTLHWYQQKSETSPKPWIYGTSNLASGVPVRFSGSGSGTSYSLTISSMEAEDAATYYCQQWSTYPYTFGGGTKLEIKRT

SEQ ID NO: 119 - protein, 129 aa, synthetic construct
  REGION 1..129: note = synthetic primer
TGVHSEVQLQQPGPVLVKPGPSMKISCKASGYAFTDYFIHWVRQSHGRSLEWIGLVSPYNGGTYYNQKFKGKASLTVDTSSSTAYMELSSLTSEDSAVYYCARLGYYGNWYFFDYWGQGTPLTVSSAST

SEQ ID NO: 120 - protein, 115 aa, synthetic construct; identical to SEQ ID NO: 90
  REGION 1..115: note = synthetic primer

SEQ ID NO: 121 - DNA, 446 bp, synthetic construct
  misc_feature 1..446: note = synthetic primer
  source 1..446: mol_type = other DNA
nnnnnnnnnnnnnnnnnnngnannnnnngtgaagttgtcctgcaaggcttctggctacactttcaccagctactggatgcactgggtgaagcagaggcctggacaaggccttgagtggattggaatgattcatcctaatagtggtagtactaactacaatgagaagttcaagagcaaggccacactgactgtagacaaatcctccagcacagcctacatgcaactcagcagcctgacatctgaggactctgcggtctattactgtgcaagttattactacggtagtagctattactttgactactggggccaaggcaccactctcacagtctcctcagccaaaacaacagccccatcggtctatccactggcccctgtgtgtggaggtacaactggctcctcggtgactctaggatgcctggtcaagggctattnnccctgancan

SEQ ID NO: 122 - DNA, 343 bp, synthetic construct
  misc_feature 1..343: note = synthetic primer
  source 1..343: mol_type = other DNA
nnncnnnnnnntnnncttggtcccagcaccgaacgtgagcggataactataatcattctgacagtaataaactgccaggtcttcagcctgcacactgctgatggtgagagtgaaatctgttccagatccactgcctgtgaagcgatcagggaccccagattccctagtggatgcccagtagatcaacagtttaggaggctgccctggtttctgctggtaccaggtcaagtagttcttttgatttccactgtttaacagactctgactggacttgcagctcatagtgaccttctctcctgctgtcacagtcagggaggatggagactgggtcnnnnnaatgtca

SEQ ID NO: 123 - DNA, 431 bp, synthetic construct
  misc_feature 1..431: note = synthetic primer
  source 1..431: mol_type = other DNA
gnnnnnnnnnnntnnnnnagnangttcntgaagttgtcctgcaaggcttctggctacactttcaccaactactggatgcactgggtgaagcataggcctggacaaggccttgattggattggaangattcatcctaatagtggtacttctaactacaatgagaagttcaagagcaaggccacactgactgtagacaaatcctccagcacagcctacatgcaactcagcagcctgacatctgaggactctgcggtcttttactgtacaagatctgactgggcctttgactactggggccaaggcacctctctcacagtctcctcacccaaaacgacacccccatctgtctatccactggcccctggatctgctgcccaaactaactccatggtgaccctgggatgcctggtcaagggctattnccctganca

SEQ ID NO: 124 - DNA, 342 bp, synthetic construct
  misc_feature 1..342: note = synthetic primer
  source 1..342: mol_type = other DNA
nnnnnnnnnnncnnnncttggtcnnncaccgaacgtgagcggaaactatgatcattctgacagtaataaactgccaggtcttcagcctgcacactactgatggtaagagtgaaatcggttccagatccactgcctgtgaagcgatcagggaccccagattccctagtggatgccccgtatatcaacagtttaggaggctgccctggtttctgctggtaccaggccaagtagttcttttgatttccactgtttaacaggctctgactggacttgcagctcatagtgaccttctctcctgctgacacactcagggaggatggagactgggtntntcaatgtcan

SEQ ID NO: 125 - DNA, 440 bp, synthetic construct
  misc_feature 1..440: note = synthetic primer
  source 1..440: mol_type = other DNA
nnnnnnnnnnnntnannnnngnantcnnagtgaagatgtcctgcaagacttctggatatacattcacattctacggtataaactgggtgaagcagaggcctggacagggcctggaatggattggatatatttatgttggaaatggttattctgagtacaatgagaagttcaaggtcaaggccacactgacttcagacacatcctccagcacagcctacatgcagctcagcggcctgacatctgaggactctgcaatctatttctgtgcaagatccgatggttacaactactttgactactggggccaaggcaccactctcacagtctcctcagccaaaacgacacccccatctgtctatccactggcccctggatctgctgcccaaactaactccatggtgaccctgggatgcctggtcaagggctantnccctgancan

SEQ ID NO: 126 - DNA, 326 bp, synthetic construct
  misc_feature 1..326: note = synthetic primer
  source 1..326: mol_type = other DNA
nnnncnnnncncnnnncttggtcccagcaccgaacgtgggtgggttactactccactgctggcagtaataagtggcagtatcttcagcctccactctgctgattgtgagagagtaagaggtcccagacccactgccactgaagcgagcagggactccagaagccaggttggatgtggcataaatccagggtttgggggaggatcctgtcttctgctggtaccagtgcatgtaatgtaaacttgagctggccctgcaagtcattgtgactttctcccctggagatgcagacaggattgctggagactgggnnnnnccaatgtcaana

SEQ ID NO: 127 - DNA, 445 bp, synthetic construct
  misc_feature 1..445: note = synthetic primer
  source 1..445: mol_type = other DNA
nnnnnnnnnnnnnnnnnnnnnnnangnttcagtgaagttgtcctgcaaggcttctggctacactttcaccagctactggatgcactgggtgaagcagaggcctggacaaggccttgagtggattggattgattcatcctaatagtggtagtacttactacaatgagaagttcaagaacaaggccacactgactgtagacaaatcctccagcacagcctacatgcaactcagcagcctgacatctgaggactctgcggtctattactgtgcaagatgggatgattcctactggtacttcaaagtctggggcacagggacctcggtcaccgtctcctcagccaaaacgacacccccatctgtctatccactggcccctggatctgctgcccaaactaactccatggtgaccctgggatgcctggtcaagggctattnccctgannan

SEQ ID NO: 128 - DNA, 326 bp, synthetic construct
  misc_feature 1..326: note = synthetic primer
  source 1..326: mol_type = other DNA
nnnnnnnnnnnntttnnncttggtcccccctccgaacgtgtacgggttgctactccactgctggcagtaataagtggcagcatcttcagcctccactctgctgattgtgagagagtaagaggtcccagacccactgccactgaagcgagcagggactccagaagccaggttggatgtggcataaatccagggtttgggggaggatcctggcttctgctggtaccagtgcatgtaacttacacttgaactggccctgcaagtcattgtgaccttctcccctggagatgcagacaggattgctggagattgtgtcagnacaatgtcan

SEQ ID NO: 129 - DNA, 450 bp, synthetic construct
  misc_feature 1..450: note = synthetic primer
  source 1..450: mol_type = other DNA
nnnnnnnnnnnnnnnnnnnnnnnnnnnnnnnnnncttcngtgaagctgtcctgcaaggcttctggctacactttcactgactactatataaactgggtgaagcagaggcctggacagggacttgagtggattgcaaggatttatcctggaagtggtaatacttactacaatgagaagttcaagggcaaggccacactgactgcagaaaaatcctccagcactgcctacatgcagctcagcagcctgacatctgaggactctgctgtctatttctgtgcaagacggctaactgcgggatacttcgatgtctggggcacagggaccacggtcaccgtctcctcagccaaaacgacacccccatctgtctatccactggcccctggatctgctgcccaaactaactccatggtgaccctgggatgcctggtcaagggctactnccctgancan

SEQ ID NO: 130 - DNA, 315 bp, synthetic construct
  misc_feature 1..315: note = synthetic primer
  source 1..315: mol_type = other DNA
nnnnnnnnnnnctnnncttggtcccagcaccgaacgtgagcggaaactcatcatactgtagacaataataaattcccatatcttcatactccaggctgctgatggtgagagaataatcttgcccagatccactgccactgaaccttgatgggaccccatctaccaatctgtttgcacgatagatcagggtcttaggagatttccctggtttctgctggaaccaggataaatagctattaatgtcctgactcgccttgcaagtgatagtgactctctctcctagagatgcaaacatggaagatggagactgtntnn

SEQ ID NO: 131 - DNA, 455 bp, synthetic construct
  misc_feature 1..455: note = synthetic primer
  source 1..455: mol_type = other DNA
nnnnnnnnnnnnnnnnnnnnnnnnnnnngnanggnccctgaactctcctgtacagcctctggattcactttcagtagctatgccatgtcttgggttcgccagactccagagaagaggctggagtgggtcgcatatattagtagtggtggtgattacatctactatacagacactgtgaagggccgattcaccatctccagagacaatgccaggaacaccctgttcctacaaatgagcagtctgaagtctgaggacacagccatgtattactgtacaagagataccggttactacgtttctcggtacttcgatgtctggggcacagggaccacggtcaccgtctcctcagccaaaacgacacccccatctgtctatccactggcccctggatctgctgcccaaactaactccatggtgaccctgggatgcctggtcaagggctacttccctgagca

SEQ ID NO: 132 - DNA, 331 bp, synthetic construct
  misc_feature 1..331: note = synthetic primer
  source 1..331: mol_type = other DNA
nnnnnnnnnntgnntnnnnncttnnnnnnnnnnnnnaacgtcagcaggatagttgttatattgatgacagaaatactctgccaagtcttcagactgcacattgctgatggtgagagtgaaatctgtcccagatccactgcctgtgaagcgatcagggactccactgttccggaaggatgccgagtaaatcagtgctttaggagattgtcctggtttctgttgataccaggctacattagttcccacattctgactggccttgcaggtgacgctgaccctgtctccaactgatgtggacatgaattttggagactgggtcatcacaatgtca

SEQ ID NO: 133 - DNA, 464 bp, synthetic construct
  misc_feature 1..464: note = synthetic primer
  source 1..464: mol_type = other DNA
nnnnnnnnnnnnnnnnnnnnnnnnnnnnnnnngnanccttcagtgaagatatcctgtaaggcttctggattcacattcactgactactacatgcactgggtgaagcagagccatggaaagagccttgaatggattggacttgtttatccttacaatggtggtacttattacaaccagaagttcaagggcaaggccacattgactgtagacacatcctccagcacagcctacatggagctaaacagcctgacttctgaggactctgcggtctattactgtgtacgattagggaacggtagtagcaacgagtggtacttcgatgtctggggcacagggaccacggtcactgtctcctcagccaaaacgacacccccatctgtctatccactggcccctggatctgctgcccaaactaactccatggtgaccctgggatgcctggtcaagggctacttccctganca

SEQ ID NO: 134 - DNA, 322 bp, synthetic construct
  misc_feature 1..322: note = synthetic primer
  source 1..322: mol_type = other DNA
nnnnncnnntnannnnngtttggtgcctccccgaacgtccacgggtaagttctccactgttgacagtaataagtggcagcatcttcagcctccatactgctgattgtgagagaataagaggtcccagatccactgccactgaagcgaacagggactccagaagccaggttggatgtgccataaatccagggtttgggggaggtttctgacttctgctggtaccagtgcaaattgtaggaacttatacttgagctgacactgcaggtgatggtgaccttctcccctggagatgcagccatgagtgctggagactgngtnannn

SEQ ID NO: 135 - DNA, 436 bp, synthetic construct
  misc_feature 1..436: note = synthetic primer
  source 1..436: mol_type = other DNA
nnnnnnnnnnngtnannntgnnngcttcngtgaagctgtcctgcaaggcttctggctacaccttcaccaactactggatacactggatgaagcagaggcctggacgaggccttgagtggattggaaggattgagcctaatagcggtgatactaaatacaatgagaagatcaagagcagggccacactgactgtagacaaaccgtccagcacagcctacatgcagctcagcagcctgacatctgaggactctgcggtctattattgtgcaagatctgggtatgattaccctgaggcctggggccaaggcaccactctcacagtctcctcagccaaaacgacacccccatctgtctatccactggcccctggatctgctgcccaaactaactccatggtgaccctgggatgcctggtcaagggctactnccctganna

SEQ ID NO: 136 - DNA, 343 bp, synthetic construct
  misc_feature 1..343: note = synthetic primer
  source 1..343: mol_type = other DNA
nnnnnnnnnnnnnnnnnnnnnttggtgcctccaccgaacgtccaaggaaccttcctactttgctgacagaaatacattgcaatatcatcctcctccacaggatggatgttgaggctgaagtctgtcccagacccactgccactaaacctggcagggaccccagattctacgttggatgcagcatagatgaggagtttgggtggctgtcctggtttctgttggtaccactgcattaaagttgtgccataatattcaacaccttcactggctctgcaggagatggtggctctctgccctagagacacagccaaagaagctggagattgggtcatcacaatgtcan

SEQ ID NO: 137 - DNA, 444 bp, synthetic construct
  misc_feature 1..444: note = synthetic primer
  source 1..444: mol_type = other DNA
nnnnnnnnnnnnnnnnnnntgancctgnnncttcagtgaagttgtcctgcaaggcttctggctataccttcaccaactactggatgcactgggtgaaacacaggcctggacgaggccttgagtggattggaaggattgatcctaatagtggtggtactaagtacaatgagaagttcaagaataaggccacactgactgtagacaatccctccagcacaggctacatgcagctcagcagcctgacatctgaggactctgcggtctattattgtacaagatctgggtatgattaccctgactactggggccaaggcaccactctcacagtctcctcagccaaaacgacacccccatctgtctatccactggcccctggatctgctgcccaaactaactccatggtgaccctgggatgcctggtcaagggctatttnccctganaan

SEQ ID NO: 138 - DNA, 343 bp, synthetic construct
  misc_feature 1..343: note = synthetic primer
  source 1..343: mol_type = other DNA
nnnnnnnnnnnnnntnnnngcttggtgcctccnccgaacgtccaaggaaccttcctactttgctgacagaaatacattgcaatatcatcctcctccacaggatggaagttgaggctgaagtctgtcccagacccactgccactaaacctgggagggaccccagaatctacgttggatgcagcatagatgaggactttgggtggctgtcctggtttctgttggtaccactgcattaaacttgtgccataatattcaacactttcactggctctgcaggagatggtggctctctgccctagagacacagccaaagaagctggagactgggtnannncaatgtcaa

SEQ ID NO: 139 - DNA, 444 bp, synthetic construct
  misc_feature 1..444: note = synthetic primer
  source 1..444: mol_type = other DNA
nnnnnnnnnnnnnnnnnnnnnnancnngnnngcttcagtgaagctgtcctgcaaggcttctggctacaccttcaccagctactggatgcactgggtgaagcagaggcctggacgaggccttgagtggattggaaggattgatcctaatagtggtggtactaagtacaatgagaagttcaagagcaaggccacactgactgtagacaaaccctccagcacagcctacatgcagctcagcagcctgacatctgaggactctgcggtctattattgtgcaagatctgggtatgattaccctgactactggggccaaggcaccactctcacagtctcctcagccaaaacgacacccccatctgtctatccactggcccctggatctgctgcccaaactaactccatggtgaccctgggatgcctggtcaagggctacttccctganca

SEQ ID NO: 140 - DNA, 327 bp, synthetic construct
  misc_feature 1..327: note = synthetic primer
  source 1..327: mol_type = other DNA
nnnnnnnnnatnnnncttggtgcctccccgaacgtccaaggaaccttcctactttgctgacagaaatacattgcaatatcatcctcctccacaggatggatgttgaggctgaagtctgtcccagacccactgccaccaaacctggcaggaaccccaggttctacgttggatgcagcatagatgaggagtttgggtggctgtcctggtttctgttggtaccactgcattaaacttgtgtcataatattcaacaccttcactggctctgcaggagatggtggctctctgccctagagacacagccaaagaagctggagnntgtnnnnnn

SEQ ID NO: 141 - DNA, 428 bp, synthetic construct
  misc_feature 1..428: note = synthetic primer
  source 1..428: mol_type = other DNA
nnnnnnnnnnnnnnnncntgannannattcngtgaagttgtcctgcaaggcttctggctacactttcaccatctactggatgcactgggtgaagcataggcctggacaaggccttgagtggattgtaatgattcatcctaatattggtattactaactacaatgagatgtcgagagcaaggcctcactgactgtagacaaatcctccagcacatcctacatgcaactcancagcctgacatctgaggactctgcggtctattactgtgcaagaatcggatactccggctggggtcaaggaacctcattcaccgtctcctcagccaaaacgacacccccatctgtctatccactggcccctggatctgctgcccaaactaactccatggtgaccctgggatgcctggtcaagggctactnccctganca

SEQ ID NO: 142 - DNA, 345 bp, synthetic construct
  misc_feature 1..345: note = synthetic primer
  source 1..345: mol_type = other DNA
gnnnnnnctnnctnnncttggtcccagcaccgaacgtgagcggataagtatgatcattctgacagtaataaactgccaggtcttcagcctgcacactgctgatggtaagagtgaaatcggttccagatccattgcctgtgaagcgatcagggaccccagattccctagtggatgccccgttgatcaacactttaggaggctgccctggtttctgctggtaccaggccaagtagttcttttgatttccaccatttaacagactctgactggacttgcagctcatagtgaccttctctcctgctgacacactcagggaggatggagactgggtntnacaatgtcana

SEQ ID NO: 143 - DNA, 445 bp, synthetic construct
  misc_feature 1..445: note = synthetic primer
  source 1..445: mol_type = other DNA
nnnnnnnnnnnanctggngnngcctgggtcctcagtgaagatgtcctgcaagacttctggatatacattcacattctacggtataaactgggtgaagcagaggcctggacagggcctggaatggattggatatatttatgttggaaatggttatactgagtacaatgagaagttcaaggtcaaggccacactgacttcagacacatcctccagcacagcctacatgcagctcagcggcctgacatctgaggactctgcaatctatttctgtgcaagatccgatggttacaactacttcgactactggggccaaggcaccactctcacagtctcctcagccaaaacgacacccccatctgtctatccactggcccctggatctgctgcccaaactaactccatggtgaccctgggatgcctggtcaagggctattnncctgancan

SEQ ID NO: 144 - DNA, 328 bp, synthetic construct
  misc_feature 1..328: note = synthetic primer
  source 1..328: mol_type = other DNA
nnnnnnnntntngctnnnnnttgnntcccngnaccgaacgtgggtgggttactactccactgatggcagtaataagtggcagtatcttcagcctccactctgctgattgtgagagagtaagaggtcccagacccactgccactgaagcgagcagggactccagaagccaggttggatgtggcataaatccagggtttgggggaggatcctggcttctgctggtaccagtgcatgtaatgtacacttgaactggccctgcaagtcattgtgactttctcccctggagatgcagtcaggattgctggagactgggtcatcacnatgtcaa

SEQ ID NO: 145 - DNA, 447 bp, synthetic construct
  misc_feature 1..447: note = synthetic primer
  source 1..447: mol_type = other DNA
nnnnnnnnnnnnnnnnnnnnnnnagnnnncnnnnntgaagttgtcctgcaaggcttctggctacactttcaccagctactggatgcactgggtgaagcagaggcctggacaaggccttgagtggattggattgattcatcctaatagtggtagtacttactacaatgagaagttcaagaacaaggccacactgactgtagacaaatcctccagcacagcctacatgcaactcagcagcctgacatctgaggactctgcggtctattactgtgcaagatgggatgattcctactggtacttcaaagtctggggcacagggacctcggtcaccgtctcctcagccaaaacgacacccccatctgtctatccactggcccctggatctgctgcccaaactaactccatggtgaccctgggatgcctggtcaagggctacttccctganca

SEQ ID NO: 146 - DNA, 322 bp, synthetic construct
  misc_feature 1..322: note = synthetic primer
  source 1..322: mol_type = other DNA
nnnnnnnnnnnnnncttggtcccccctccgaacgtgtacgggttgctactccactgctggcagtaataagtggcagcatcttcagcctccactctgctgattgtgagagagtaagaggtcccagacccactgccactgaagcgagcagggactccagaagccaggttggatgtggcataaatccagggtttgggggaggatcctggcttctgctggtaccagtgcatgtaacttacacttgaactggccctgcaagtcattgtgaccttctcccctggagatgcagacaggattgctggagactgggtnnnnncaatgtcan

SEQ ID NO: 147 - DNA, 453 bp, synthetic construct
  misc_feature 1..453: note = synthetic primer
  source 1..453: mol_type = other DNA
nnnncnnnnnnnnnnnnnntnnnnnnnnnngnnngcttcngtgaagatatcctgcaaggcttctggctacaccttcactgactactatatacactggctgaagcagaggcctggacagggacttgagtggattggattgatttttcctggaagtggtagtatttactgtaatgagaagttcaagggcaaggccacacttactgtagacaaatcctccaccacagcctacatgttgctcagcagcctgacctctgaggactctgcggtctatttctgtgcaagatgggagactacggcgtggtacttcgatgtctggggcacagggaccacggtcaccgtctcctcagccaaaacgacacccccatctgtctatccactggcccctggatctgctgcccaaactaactccatggtgaccctgggatgcctggtcaagggctacttccctgagca

SEQ ID NO: 148 - DNA, 324 bp, synthetic construct
  misc_feature 1..324: note = synthetic primer
  source 1..324: mol_type = other DNA
nnnnncnnnnnnnnnnnnttgnnnnnnctccgaacgtgtacgggtaactcctccactgctggcagtaataagtggcagcatcttcggcctccactctgctgactgtgagagagtaagaggtcccagacccactgccactgaagcgggcagggactccagaagccaggttggatgtggcataaatccagggtttgggggaggatcctggcttctgctggtaccagtgcatgtaacttacacttaaactggccctgcaagtcattgtgaccttctcccctggagatgcagacaggattgctggagactgggtcannacaatgtcan

SEQ ID NO: 149 - DNA, 445 bp, synthetic construct
  misc_feature 1..445: note = synthetic primer
  source 1..445: mol_type = other DNA
nnnnnnnggtgagcctgggccttcatgaagatatcctgtaaggcttctggattcacattcactgactactacatacactgggtgaggcagagccatggaaagagccttgagtggattggacttgtttctccttacaatggtggtacttactacaaccagaagttcaagggcaaggccacattgactgtagactcatcctccagcacagcctacatggagctaagcagcctgacttctgaggactctgcggtctattactgtgcaagattgggctactatggtgactggtactactttgactactggggccaaggcacccctctcacagtctcctcagccaaaacgacacccccatctgtctatccactggcccctggatctgctgcccaaactaactccatggtgaccctgggatgcctggtcaagggctattnnccctgancan

SEQ ID NO: 150 - DNA, 330 bp, synthetic construct
  misc_feature 1..330: note = synthetic primer
  source 1..330: mol_type = other DNA
nnnnnnnnnnnnnnnnttggtcccccctccgaacgtgtatgggtaactactccactgttgacagtaataagtggcagcatcttcagcctccatgctgctgattgtgagagaataagaggtcccagatccactgccactgaagcgaacagggactccagaagccaggttggatgtgccataaatccagggtttgggggaggtttctgacttctgctggtaccagttcaagttgctggaacttatacttgagctgacactacaggtgatggtgaccttttcccctggagatgtagccatgagtgctggagactgggtgannncaatgtcann

SEQ ID NO: 151 - DNA, 458 bp, synthetic construct
  misc_feature 1..458: note = synthetic primer
  source 1..458: mol_type = other DNA
nnnnnnnnnnnnnnnnnnnnnnnccngnnncttcagtgaagttgtcctgcaaggcttctggctacactttcaccacctactggatgcactggatgaagcagtggcctggacaaggccttgagtggattggattgattcatcctaatagtggtagtacttactacaatgagaagttcaagagcaaggccacactgactgtagacaaatcctccagcacagcctacatgcaactcagcagcctgacatctgaggactctgcggtctattactgtgcaagatttgaggagctatggggaggttactggtacttcgatgtctggggcacagggaccacggtcaccgtctcctcagccaaaacgacacccccatctgtctatccactggcccctggatctgctgcccaaactaactccatggtgaccctgggatgcctggtcaagggctatttccctgancan

SEQ ID NO: 152 - DNA, 330 bp, synthetic construct
  misc_feature 1..330: note = synthetic primer
  source 1..330: mol_type = other DNA
nnnnnnnnttnnnnnnncttggtccccctccgaacgtgtacgggtaactactccactgttgacagtaataagtggcagcatcttcagcctccatgctgctgattgtgagagaataagaggtcccagatccactgccactgaagcgaacagggactccagaagccaggttggatgtgccataaatccagggtttgggggaggtttctgacttctgctggtaccagtgcaagttgctggaacttatacttgagctgacactgcaggtgatggtgaccttctcccctggagatgcagccatgagtgctggagactgggtnnnnacaatgtcan

SEQ ID NO: 153 - DNA, 461 bp, synthetic construct
  misc_feature 1..461: note = synthetic primer
  source 1..461: mol_type = other DNA
nnnnnnnnnnnnnnnnnnnnnnnnnnnnngnnngcttcagtgaagttgtcctgcaaggcttctggctacaatttcaccagctactggatgcactgggtgaagcagaggcctggacaaggccttgagtggattggattgattcatcctaatagtggtggtacttactacaatgagaagttcaagagcaaggccacactgactgtagacagatcctccagcacagcctacatgcaactcagcagcctgacatctgaggactctgcggtctattattgtgcaagatttgaggaggaatggggagggtattggtacttcgatgtctggggcacagggaccacggtcaccgtctcctcagccaaaacgacacccccatctgtctatccactggcccctggatctgctgcccaaactaactccatggtgaccctgggatgcctggtcaagggctatttccctgagca

SEQ ID NO: 154 - DNA, 321 bp, synthetic construct
  misc_feature 1..321: note = synthetic primer
  source 1..321: mol_type = other DNA
nnnnnnnnntntnnnnnnttggtnnncctccgaacgtgtacgggtaactactccactgttgacagtaataagtggcagcatcttcagcctccatgctgctgattgtgagagaataagaggtcccagatccactgccactgaagcgaacagggactccagaagccaggttggaagtgccataaatccagggtttgggggaggtttctgacttctgttggtaccagtgcaagttgctggaacttatacttgaactgacactgcaggtgatggtgaccttttcccctggagatgcagccatgagtgctggagtntgggtcannn

SEQ ID NO: 155 - DNA, 460 bp, synthetic construct
  misc_feature 1..460: note = synthetic primer
  source 1..460: mol_type = other DNA
nnnnannnnnnnnnnnnnnnnnnnnnnnnnnnnnnncttcngtgaagttgtcctgcaaggcttctggctacactttcaccagctactggatgcactgggtgaagcagaggcctggacaaggccttgagtggattggactgattcatcctaatagtagtagtacttactacaatgagaagttcaagaccagggccacactgactgtagacaagtcctccagcacagcctacatgcaactcagcagcctgacatctgaggactctgcggtctattactgtgcaagattgggctatggtaactcctactggtacttcgatgtctggggcacagggaccacggtcaccgtctcctcagccaaaacgacacccccatctgtctatccactggcccctggatctgctgcccaaactaactccatggtgaccctgggatgcctggtcaagggctacttccctganca

SEQ ID NO: 156 - DNA, 329 bp, synthetic construct
  misc_feature 1..329: note = synthetic primer
  source 1..329: mol_type = other DNA
nnnnnnnnnnnnnnnngcttggtgcctccaccgaacgtccacgggtaacttctccactgttgacagtaataagtggcagcatcttcagcctccaaactgctgattgtgagagaataagaggtcccagatccactgccactgaagcgaatagggactccagaagccaggttggatgtgccataaatccagggtttgggggaggtttctgacttctgctggtaccagtgcaagttgctggaactaatacttgagctgacactgcaggtgatggtgaccttctcccctggagatgcagccatgagtgctggagactgggtnnnncaatgtca

SEQ ID NO: 157 - DNA, 447 bp, synthetic construct
  misc_feature 1..447: note = synthetic primer
  source 1..447: mol_type = other DNA
nnnnnnnnnnnnnnnnnnnnnnnnnnnnnnttcngtgaagttgtcctgcaaggcttctggctacactttcaccagctactggatgcactgggtgcagcagaggcctggacaaggccttgagtggattggattgattcatcctattggtggtggtactcactacaatgagaagttcaagaacaaggccacactgactgtagacaaatcctccagcacagcctacatgcaactcagcagcctgacatctgaggactctgcggtctattactgtgcaagactcggaactggtccgtactactttgactactggggccaaggcaccactctcacagtctcctcagccaaaacgacacccccatctgtctatccactggcccctggatctgctgcccaaactaactccatggtgaccctgggatgcctggtcaagggctatttccctganca

SEQ ID NO: 158 - DNA, 333 bp, synthetic construct
  misc_feature 1..333: note = synthetic primer
  source 1..333: mol_type = other DNA
nnnncnttnnntnnnnntttggtgcctccaccgaacgtccacgggtaactactccactgttgacagtaataagtggcagcatcttcagcctccatgctgctgattgtgagagaataagaggtcccagatccactgccactgaagcgaacagggactccagaagccaggttggatgtgccataaatccagggtttgggggaggtttctgacttctgctggtaccagtccaaggtgctggaacttatacttgagctgacactgcaggtgatggtgaccttctcccctggagatgcagccatgagtgctggagactgggtcnnnacaatgtcannn

SEQ ID NO: 159 - DNA, 455 bp, synthetic construct
  misc_feature 1..455: note = synthetic primer
  source 1..455: mol_type = other DNA
nnnnnnnnnnnnnnnnnnnnnnnnnnannnngnanccttcagtgaatatatcctgtaaggcttctggattcacattcactgactactacattcactgggtgaaacagagccatggaaagagccttgagtggattggacttgtttatccttacagtggtggtacttactacaaccagaagttcaagggcaaggccacattgactgtagacacatcctccagcacagcctacatggagctaggcagcctgacttctgaggactctgcggtctattactgtgcaagattgggcgacaacttctactactttgactactggggccaaggcaccactctcacagtctcctcagccaaaacgacacccccatctgtctatccactggcccctggatctgctgcccaaactaactccatggtgaccctgggatgcctggtcaagggctatttccctgagcan

SEQ ID NO: 160 - DNA, 329 bp, synthetic construct
  misc_feature 1..329: note = synthetic primer
  source 1..329: mol_type = other DNA
nnnnnnnnnnnttnngcttggtcnncctccgaacgtgtacgggtaacttctccactgttgacagtaataagtggcagcatcttcagcctccatgctgctgattgtgagagaataagatgtcccagatccactgccactgaagcgaacagggactccagaagccaggttggatgtgccataaatccagggtttgggggaggtttctgacttctgctggtaccagtgcaagttgctggaacttatacttgagctgacactgcaggtgatggtgaccttctcccctggagatgcagccatgagtgttggagtntgngnnannacaatgtcan

SEQ ID NO: 161 - DNA, 454 bp, synthetic construct
  misc_feature 1..454: note = synthetic primer
  source 1..454: mol_type = other DNA
gnnnnnnnnnnnnnnnnnnnnnnnnnnnnncttcagtgaagatatcctgtaaggcttctggattcacattcactgactactacatgcactgggtgaaacagagccatggaaagagccttgagtggattggtcttgtttctccttacaatggtggtactttctacaaccagaagttcaagggcaaggccacattgactgtagacacatcctccagcacagcctacatggaactaaacagcctgacttctgaggactctgcggtctattactgtgcaagagtgggtaatagctacgtccattatgctatggactactggggtcaaggaacctcagtcaccgtctcctcagccaaaacgacacccccatctgtctatccactggcccctggatctgctgcccaaactaactccatggtgaccctgggatgcctggtcaagggctacttccctganca

SEQ ID NO: 162 - DNA, 331 bp, synthetic construct
  misc_feature 1..331: note = synthetic primer
  source 1..331: mol_type = other DNA
nnnncnnnnnnnnnnncttggtcccccctccgaacgtgtacgggtaattactccactgttgacagtaataagtggcagcatcttcagcctccatgctgctgattgtgagagaataagaggtcccagatccactgccactgaagcgaacagggactccagaagccaggttggatgtgccataaatccagggtttgggggaggtttctgacttctgctggtaccagtgcaagttgctggaacttatacttgagctgacactgcaggtgatggtgaccttctcccctggagatgcagccatgagtgctggagactgggtgannncaatgtcann

SEQ ID NO: 163 - DNA, 464 bp, synthetic construct
  misc_feature 1..464: note = synthetic primer
  source 1..464: mol_type = other DNA
nnnnnnnnnnnnnnnnnnnnnnnnnnnnnnnnnnanggtccctgnnnctctcctgtgcagcctctggattcactttcagtagctatgccatgtcttgggttcgccagactccagagaagaggctggactgggtcgcatacattagtagtggtggtgatcacatctactatgcagacactgtgaagggccgattcaccatctccagagacaatgccaggaacaccctgtacctgcaaatgagcagtctgaagtctgaggacacagccatgtattactgtacaagagataccggttactacgtttctcggtacttcgatgtctggggcacagggaccacggtcaccgtctcctcagccaaaacgacacccccatctgtctatccactggcccctggatctgctgcccaaactaactccntggtgaccctgggatgcctggtcaagggctanttccctgancaaata

SEQ ID NO: 164 - DNA, 333 bp, synthetic construct
  misc_feature 1..333: note = synthetic primer
  source 1..333: mol_type = other DNA
nnnnnnnnnnntntgnnnnnnnanttgntgcctccnccnancgtcagaggatggctgttatattggtgacagaaatactctgccaagtcttcagactgcacattgctgatggtgagagtgaaatctgtcccagatccactgcctgtgaagcgatcagggactccactggaccggaaggatgccgaataaatcactactttaggagattgccctgatttctgttgataccaggcaacattagtaaccacattctgactggccttgcaggtgacgctgaccctgtctcctactgatgtggacatgaattttggagactgggtcatcacaatgtca

SEQ ID NO: 165 - DNA, 459 bp, synthetic construct
  misc_feature 1..459: note = synthetic primer
  source 1..459: mol_type = other DNA
nnnnnnnnnnnnnnnnnnnnnnnnnnnnnnnnantcttcactgaagatatcctgcaaggcttctggctacaccttcactgactactttataaactgggtgaaacagaggcctggacagggacttgactggattggatggatttttcctggaagtggtagtacttactacaatgacaagttcaagggcaaggccacacttactgtagacaaatcctccagcactgcctacatgttgctcagcagcctgacctctgaggcctctgcggtctatttctgtgcaagatgggactccgatagtacctactggtacttcgatgtctggggcacagggaccacggtcaccgtctcctcagccaaaacgacacccccatctgtctatccactggcccctggatctgctgcccaaactaactccatggtgaccctgggatgcctggtcaagggctatttccctganca

SEQ ID NO: 166 - DNA, 330 bp, synthetic construct
  misc_feature 1..330: note = synthetic primer
  source 1..330: mol_type = other DNA
nnnnnncnnnnntnnnnncttggtnnncctccgaacgtgtacgggtaagtactccactgttgacagtaataagtggcagcatcttcagcctccatgctgctgattgtgagagaataagaggtcccagatccactgccactgaagcgaatagggactccagaagccaaattggatgtgccataaatccagggtttgggggaggtttgtgacttctgctggtaccagtgcaaggtgctggaacttatacttgaattgacactgcaggtgatggtgaccttctcccctggagatgcagccatgagtgctggagtctgggtcannncaatgtca

SEQ ID NO: 167 - DNA, 440 bp, synthetic construct
  misc_feature 1..440: note = synthetic primer
  source 1..440: mol_type = other DNA
nnnnnnnnncnnnnnnnnnnnnnnangcctcagtgaagctttcctgcaaggctactggctacacattcactggctactggatagagtggttaaagcagaggcctggacatggccttgagtggattggagagattttacctggaagtgataatactaactacaatgagaagttcaggggcaaggccacattcactgcagatacatcctccaacacagcctacatgcacctcagcagcctgacaactgaggactctgccatctattactgtgcaagagaagggggtttctactttgactactggggccaaggcaccactctcactgtctcctcagccaaaacgacacccccatctgtctatccactggcccctggatctgctgcccaaactaactccatggtgaccctgggatgcctggtcaagggctactnccctgancaa

SEQ ID NO: 168 - DNA, 349 bp, synthetic construct
  misc_feature 1..349: note = synthetic primer
  source 1..349: mol_type = other DNA
nnnnnnnnnntggtganngtcacactcacttgtcgctcaagtattggggctgttacaactagtaactacgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggccagcccaagtcttcgccatcagtcaccctgttcccaccttccnctgaagaga

SEQ ID NO: 169 - DNA, 453 bp, synthetic construct
  misc_feature 1..453: note = synthetic primer
  source 1..453: mol_type = other DNA
nnnnnnnnnnnnnnnnnnnnannccngnnnccttcngtgaagatatcctgtaaggcttctggattcacattcactgactactacatgcactgggtgaagcagagccatggaaagagccttgaatggattggacttgtttatccttacaatggtggtactagctacaaccagaagttcaagggcaaggccacattgactgtagacacatcctccaccacagcctacatggagctaaacagcctgacttctgaggactctgcggtctattactgtgcaagattgggctcaggctacgagtactactttgactactggggccaaggcacctctctcacagtctcctcagccaaaacgacacccccatctgtctatccactggcccctggatctgctgcccaaactaactccatggtgaccctgggatgcctggtcaagggctactnccctgancan

SEQ ID NO: 170 - DNA, 321 bp, synthetic construct
  misc_feature 1..321: note = synthetic primer
  source 1..321: mol_type = other DNA
nnnncnnnnnnttnnngcttggtgcctccnccgaacgtccacgggtaagttctccactgttgacagtaataagtggcagcatcctcagcctccatgctgctgattgtgagagaataagaggtcccagatccactgccactgaagcgaacagggactccagaagccaggttggatgtgccataaatccagggtttgggggaggtttctgacttctgctggtaccagtgcaagttgctggaacttatacttgagctgacactgcaggtgatggtgaccttctcccctggagatgcagccatgagtgctggagnctgggtntnn

SEQ ID NO: 171 - DNA, 461 bp, synthetic construct
  misc_feature 1..461: note = synthetic primer
  source 1..461: mol_type = other DNA
nnnnnnnnnnnnnnnnnnnnnnnnnnnnnnnnnnnnnnntncagtgananntcctgtaaggcttctggattcacattcactgactactacatgcactgggtgaagcagagccatggagagagccttgagtggattggacttgtttctccttacaatggtggtactttctacaaccagaagttcaagggcaaggccacattgactgtagacacatcctccagcacagcctacatggaactaaacagcctgacttctgaggactctgcggtctattactgtgcaagagtgggtagtagttacgtccattatgctatggactactggggtcaaggaacctcagtcaccgtctcctcagccaaaacgacacccccatctgtctatccactggcccctggatctgctgcccaaactaactccatggtgaccctgggatgcctggtcaagggctacttccctgagca

SEQ ID NO: 172 - DNA, 339 bp, synthetic construct
  misc_feature 1..339: note = synthetic primer
  source 1..339: mol_type = other DNA
nnnnnnnnnnnnnnnnnnnnnntnnnncttggtcnccctccgaacgtgtacgggtacttactccactgttgacagnaataagtggcagcatcttcagcctccatgctgctgattgtgagagaataanaggtcccagatccactgccactgaagcgaacagggactccagaagccaggttggatgtgccataaatccagggtttgggggaggtttctgacttctgctggtaccagtgcaagttgctggaacttatacttgagctgacactgcaggtgatggtgaccctctcccctggagatgcagccatgagtgctggagactgggtcatcacaatgtca

SEQ ID NO: 173 - DNA, 453 bp, synthetic construct
  misc_feature 1..453: note = synthetic primer
  source 1..453: mol_type = other DNA
nnnnnnnnnnnnnnnnnnnnnnnnnnnnnnnangcttcagtgaagttgtcctgcanggcttctggctacactttcaccagctactggatgcactgggtgcagcagaggcctggacaaggccttgagtggattggattgattcatcctattggtggtggtactcactacaatgagaagttcaagaacaaggccacactgactgtagacaaatcctccagcacagcctacatgcaactcagcagcctgacatctgaggactctgcggtctattactgtgcaagactcggaactggtccgtactactttgactactggggccaaggcaccactctcacagtctcctcacccaaaacgacacccccatctgtctatccactggcccctggatctgctgcccaaactaactccatggtgaccctgggatgcctggtcaagggctatttccctgannan

SEQ ID NO: 174 - DNA, 320 bp, synthetic construct
  misc_feature 1..320: note = synthetic primer
  source 1..320: mol_type = other DNA
ngnnngntngatttcngcttggtgcctccaccgaacgtccacgggtaactactccactgttgacagtaataagtggcagcatcttcagcctccatgctgctgattgtgagagaataagaggtcccagatccactgccactgaagcgaacagggactccagaagccaggttggatgtgccataaatccagggtttgggggaggtttctgacttctgctggtaccagtgcaagttgctggaacttatacttgagctgacactgcaggtgatggtgaccttctcccctggagatgcagccatgagtgctggagactgggtgtn

SEQ ID NO: 175 - DNA, 445 bp, synthetic construct
  misc_feature 1..445: note = synthetic primer
  source 1..445: mol_type = other DNA
nnnnnnnnnnnnnnnnnnnnnnnnnnntnctgtgaanatttcctgcaaggcttctggctacaccttcactgactactatataaactgggtgaatcagaggcctggacagggacttgattggattggatggatttttcctggaantgnttttatttattacaatganaagttcaaggacaaggtcacttctactgtatacaaatcctccancacattctacttgttgctcancagcctgacctctgaggactctgcggtctatttctgtgcaanatggaaggattacgggtggtacttctatgtctggggcacagggaccacggtcaccgtctcctcatccaaaacgacacccccatctgtctatccactggcccctggatctgctgcccaaactaactccatggtgaccctgggatgcctggtcaagggctatttcccttancan

SEQ ID NO: 176 - DNA, 328 bp, synthetic construct
  misc_feature 1..328: note = synthetic primer
  source 1..328: mol_type = other DNA
nnnnnnnnnnnnnnncttggtcccccctccgaacgtgtaagggtaagtcctccactgttgacagtaataagtggcagcatcttcagcctccatgctgctgattgtgagagaataagaggtcccagatccactgccactgaagcgaacagggactccagaagccaggttggatgtgccataaatccagggtttgggggaggtttctgacttctgctggtaccagtgcaagttgctggaacttagacttgagctgacattgcaggtgagggtgaccttctcccctggagatgcagccatgagtgctggagactgggtgannncaatgtca

SEQ ID NO: 177 - DNA, 448 bp, synthetic construct
  misc_feature 1..448: note = synthetic primer
  source 1..448: mol_type = other DNA
nnnnnnnnnnnnnnnnnnnnnnnnnnnagnanccttcagtgaagatatcctgtaaggcttctggattcacattcactgactactacattcactgggtgaagcggagccatggaaagagccttgagtggattggacttgtttctccttacaatggtggtactttctacaaccagaagttcaagggcaaggccacattgactgtagatacatcctccaatacagcctacatggagctaaccagcctgacttctgaggactctgcggtctattactgtgcaagattgggggttaactggtacttcgatgtctggggcacagggaccacggtcaccgtctcctcagccaaaacgacacccccatctgtctatccactggcccctggatctgctgcccaaactaactccatggtgaccctgggatgcctggtcaagggctatttccctgannn

SEQ ID NO: 178 - DNA, 338 bp, synthetic construct
  misc_feature 1..338: note = synthetic primer
  source 1..338: mol_type = other DNA
nnnnnnnnnnnnnnntnttcnncttgtttcncccctcctancaagtggnnntnnaaactcctctgntgganaacaatannntanncatcnnntnccncctccctgcnnatgaagngnnataananntccnncnnnnnctgcgctnntgnanacaagnnctactcannaanngnnagnngtgccntaaatcnacngtttggnggaggtttctgacttctgctggtaccanngcnangtgctggaacttatactnctgntgacactgcggnnnagnaggaccttttcccccccnggatgcanccatgagtgctntgnantgtgtcatcacaatatcanca

SEQ ID NO: 179 - DNA, 451 bp, synthetic construct
  misc_feature 1..451: note = synthetic primer
  source 1..451: mol_type = other DNA
nnnnnnnnctgnntnnnnnnngnancnttcaatgaagatatcctgtaaggcttctgggttcactttcactgactacttcatacactgggtgagacagagccatggaaagagccttgagtggattggacttgtttctccttataatggtggtacttactacaaccagaagttcaagggcaaggcctcattgactgtagacacatcctccagcacagcctacatggaactaagcagcctgacttctgaggactctgcggtctattactgtgcaagattgggctactatggtaactggtacttctttgactactggggccaaggcacccctctcacagtctcctcagccaaaacgacacccccatctgtctatccactggcccctggatctgctgcccaaactaactccatggtgaccctgggatgcctggtcaagggctattnccctgagcan

SEQ ID NO: 180 - DNA, 332 bp, synthetic construct
  misc_feature 1..332: note = synthetic primer
  source 1..332: mol_type = other DNA
nnnncnnnnnnnnnnncttggtcccccctccgaacgtgtatgggtaactactccactgttgacagtaataagtggcagcatcttcagcctccatgctgctgattgtgagagaataagaggtcccagatccactgccactgaagcgaacagggactccagaagccaggttggatgtgccataaatccagggtttgggggaggtttctgacttctgctggtaccagttcaagttgctggaacttatacttgagctgacactacaggtgatggtgaccttttcccctggagatgtagccatgagtgctggagactgggtgannncaatgtcanna

SEQ ID NO: 181 - DNA, 917 bp, synthetic construct
  misc_feature 1..917: note = synthetic primer
  source 1..917: mol_type = other DNA
nnnnnnnnnnnnngtcactgcacctcggttctatcgattgaattccaccatgggatggtcatgtatcatcctttttctagtagcaactgcaaccggtgtacattccgaggtccagctgcaacagtctggggctgagctggtgaagcctggggcttcagtgaagttgtcctgcaaggcttctggctacactttcaccagctactgggtgcactgggtgaggcagaggcctggacaaggccttgagtggattggaatgattcatcctaatagtggtagtactaactacaatgagaagttcaagagcaaggccacactgactgtagacaaatcctccagcacagcctacatgcaactcagcagcctgacatctgaggactctgcggtctattactgtgcaagttattactacggtagtagctattactttgactactggggccaaggcaccactctcacagtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagnnnnnngagcccaaatcttgtgacaaaactcacacatgcccaccgtgcccagcacctgaactcctggggggaccgtcagtcttcctcttccccccaaaacccaaggacaccctcatgatctcccgganccctgaggtcacatgcgtggnggtggacgtgagcn

SEQ ID NO: 182 - DNA, 953 bp, synthetic construct
  misc_feature 1..953: note = synthetic primer
  source 1..953: mol_type = other DNA
nnnnnnnnnnnnnnntcactgcacctcggttctatcgattgaattccaccatgggatggtcatgtatcatcctttttctagtagcaactgcaaccggtgtacattccgacattgtgatgacccagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgag

SEQ ID NO: 183 - DNA, 904 bp, synthetic construct
  misc_feature 1..904: note = synthetic primer
  source 1..904: mol_type = other DNA
nnnnnnnnnnnnnngnnnntgcacctcggttctatcgattgaattccaccatgggatggtcatgtatcatcctttttctagtagcaactgcaaccggtgtacattccgaggtccagctgcaacagtctggggctgagctggtaaagcctggggcttcagtgaagctgtcctgcaaggcttctggctacactttcaccaactactggatgcactgggtgaagcagaggcctgggcaaggccttgagtggattggaatgattcatcctaatagtggtacttctaactacaatgagaagttcaagagcaaggccacactgactgtagacaaatcctccagcacagcctacatgcaactcagcagcctgacatctgaggactctgcggtcttttactgtacaagatctgactgggcctttgactactggggccaaggtacctctctcacagtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgan

SEQ ID NO: 184 - DNA, 943 bp, synthetic construct
  misc_feature 1..943: note = synthetic primer
  source 1..943: mol_type = other DNA
nnnnnnnnnnnnnnnnnnnctgcacctcggttctatcgattgaattccaccatgggatggtcatgtatcatcctttttctagtagcaactgcaaccggtgtacattccgacattgtgatgacccagtctccatcctccctgagtgtgtcagcaggagagaaggtcactatgagctgcaagtccagtcagagcctgttaaacagtggaaatcaaaagaactacttggcctggtaccagcagaaaccagggcagcctcctaaactgttgatctacggggcatccactagggaatctggggtccctgatcgcttcacaggcagtggatctggaaccgatttcactcttaccatcagtagtgtgcaggctgaagacctggcagtttattactgtcagaatgatcatagttatccgctcacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggntgctgn

SEQ ID NO: 185 - DNA, 885 bp, synthetic construct
  misc_feature 1..885: note = synthetic primer
  source 1..885: mol_type = other DNA
nnnnnnnnncngncactgcacctcggttctatcgattgaattccaccatgggatggtcatgtatcatcctttttctagtagcaactgcaaccggtgtacattccgaggtccaactgcagcagtctggagctgagctggtgaggcctgggtcctcagtgaagatgtcctgcaagacttctggatatacattcacattctacggtataaactgggtgaagcagaggcctggacagggcctggaatggattggatatatttatgttggaaatggttattctgagtacaatgagaagttcaaggtcaaggccacactgacttcagacacatcctccagcacagcctacatgcagctcagcggcctgacatctgaggactctgcaatctatttctgtgcaagatcagatggttacaactactttgactactggggccaaggcaccactctcacagtctcctcaacgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggncn

SEQ ID NO: 186 - DNA, 975 bp, synthetic construct
  misc_feature 1..975: note = synthetic primer
  source 1..975: mol_type = other DNA
nnnnnnnnnnnnnngnnnntgcacctcggttctatcgattgaattccaccatgggatggtcatgtatcatcctttttctagtagcaactgcaaccggtgtacattcccaaattgttctcacccagtctccagcaatcctatctgcatctccaggggagaaagtcacaatgacttgcagggccagctcaagtttacattacatgcactggtaccagcagaagacaggatcctcccccaaaccctggatttatgccacatccaacctggcttctggagtccctgctcgcttcagtggcagtgggtctgggacctcttactctctcacaatcagcagagtggaggctgaagatactgccacttattactgccagcagtggagtagtaacccacccacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcnnnnnnnncaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggnccgttgtcaggnaacgtggcgtggtgtgcactg

SEQ ID NO: 187 - DNA, 903 bp, synthetic construct
  misc_feature 1..903: note = synthetic primer
  source 1..903: mol_type = other DNA
nnnnnnnnnnngncactgcacctcggttctatcgattgaattccaccatgggatggtcatgtatcatcctttttctagtagcaactgcaaccggtgtacattccgaggtccaactgcagcagcctggggctgagctggtaaagcctggggcttcagtgaagttgtcctgcaaggcttctggctacactttcaccagctactggatgcactgggtgaagcagaggcctggacaaggccttgagtggattggattgattcatcctaatagtggtagtacttactacaatgagaagttcaagaacaaggccacactgactgtagacaaatcctccagcacagcctacatgcaactcagcagcctgacatctgaggactctgcggtctattactgtgcaagatgggatgattcctactggtacttcaaagtctggggcacagggaccacggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgnnncacatgcgtggtggtgga

SEQ ID NO: 188 - DNA, 923 bp, synthetic construct
  misc_feature 1..923: note = synthetic primer
  source 1..923: mol_type = other DNA
nnnnnnnnnnnnnnnngnnnnntgcacctcggttctatcgattgaattccaccatgggatggtcatgtatcatcctttttctagtagcaactgcaaccggtgtacattcccaaattgttctcacccagtctccagcaatcctgtctgcatctccaggggagaaggtcacaatgacttgcagggccagttcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagtagcaacccgtacacgttcggaggggggaccaagctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggntgctn

SEQ ID NO: 189 - DNA, 907 bp, synthetic construct
  misc_feature 1..907: note = synthetic primer
  source 1..907: mol_type = other DNA
nnnnnnnnnnnnnngncactgcacctcggttctatcgattgaattccaccatgggatggtcatgtatcatcctttttctagtagcaactgcaaccggtgtacattccgaggtccaactgcagcagtctggagctgagctggtgaggcctggggcttcagtgaagctgtcctgcaaggcttctggctacactttcactgactactatataaactgggtgaagcagaggcctggacagggactcgagtggattgcaaggatttatcctggaagtggtaatacttactacaatgagaagttcaagggcaaggccacactgactgcagaaaaatcctccagcactgcctacatgcagctcagcagcctgacatctgaggactctgctgtctatttctgtgcaagacggctaactgcgggatacttcgatgtctggggcacagggaccacggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgantcacatgcgtggtggtggacnnn

SEQ ID NO: 190 - DNA, 942 bp, synthetic construct
  misc_feature 1..942: note = synthetic primer
  source 1..942: mol_type = other DNA
nnnnnnnnnnngtcactgcacctcggttctatcgattgaattccaccatgggatggtcatgtatcatcctttttctagtagcaactgcaaccggtgtacattccgacatcaagatgacccagtctccatcttccatgtttgcatctctaggagagagagtcactatcacttgcaaggcgagtcaggacattaatagctatttatcctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttccgctcacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctcnntgtataaatcctggttgctgtctctttatgaggagttgtggn

SEQ ID NO: 191 - DNA, 895 bp, synthetic construct
  misc_feature 1..895: note = synthetic primer
  source 1..895: mol_type = other DNA
nnnnnnnnnnnnnngnnnntgcacctcggttctatcgattgaattccaccatgggatggtcatgtatcatcctttttctagtagcaactgcaaccggtgtacattccgaagtgatgctggtggagtctggggaagacttagtgaaacctggagggtccctgaaactctcctgtatagcctctggattcactttcagtagctatgccatgtcttgggttcgccagactccagagaagaggctggagtgggtcgcatatattagtagtggtggtgattacatctactatacagacactgtgaagggccgattcaccatctccagagacaatgccaggaacaccctgttcctacaaatgagcagtctgaagtctgaggacacagccatgtattactgtacaagagataccggttactacgtttctcggtacttcgatgtctggggcacagggaccacggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgangn

SEQ ID NO: 192 - DNA, 950 bp, synthetic construct
  misc_feature 1..950: note = synthetic primer
  source 1..950: mol_type = other DNA
nnnnnnnnnnnnnngnnnntgcacctcggttctatcgattgaattccaccatgggatggtcatgtatcatcctttttctagtagcaactgcaaccggtgtacattccgacattgtgatgacccagtctcaaaaattcatgtccacatcagttggagacagggtcagtgtcacctgcaaggccagtcagaatgtgggaactaatgtagcctggtatcaacagaaaccaggacaatctcctaaagcactgatttactcggcatccttccggaacagtggagtccctgatcgcttcacaggcagtggatctgggacagatttcactctcaccatcagcaatgtgcagtctgaagacttggcagagtatttctgtcatcaatataacaactatcctctgacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccnnn

SEQ ID NO: 193 - DNA, 847 bp, synthetic construct
  misc_feature 1..847: note = synthetic primer
  source 1..847: mol_type = other DNA
nnnnnnnnnnngncactgcacctcggttctatcgattgaattccaccatgggatggtcatgtatcatcctttttctagtagcaactgcaaccggtgtacattccgaggtccaactgcagcagtctggacctgtgctggtgaagcctgggccttcagtgaagatatcctgtaaggcttctggattcacattcactgactactacatgcactgggtgaagcagagccatggaaagagccttgaatggattggacttgtttatccttacaatggtggtacttattacaaccagaagttcaagggcaaggccacattgactgtagacacatcctccagcacagcctacatggagctaaacagcctgacttctgaggactctgcggtctattactgtgtacgattagggaacggtagtagcaacgagtggtacttcgatgtctggggcacagggaccacggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcnn

SEQ ID NO: 194 - DNA, 915 bp, synthetic construct
  misc_feature 1..915: note = synthetic primer
  source 1..915: mol_type = other DNA
nnnnnnnnnnnnnngnnnntgcacctcggttctatcgattgaattccaccatgggatggtcatgtatcatcctttttctagtagcaactgcaaccggtgtacattcccaaattgttctcacccagtctccagcactcatggctgcatctccaggggagaaggtcaccatcacctgcagtgtcagctcaagtataagttcctacaatttgcactggtaccagcagaagtcagaaacctcccccaaaccctggatttatggcacatccaacctggcttctggagtccctgttcgcttcagtggcagtggatctgggacctcttattctctcacaatcagcagtatggaggctgaagatgctgccacttattactgtcaacagtggagaacctacccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaan

SEQ ID NO: 195 - DNA, 849 bp, synthetic construct
  misc_feature 1..849: note = synthetic primer
  source 1..849: mol_type = other DNA
nnnnnnnnnngtcactgcacctcggttctatcgattgaattccaccatgggatggtcatgtatcatcctttttctagtagcaactgcaaccggtgtacattccgaggtccaactgcagcagcctggagctgagcttgtgaagcctggggcttcagtgaagctgtcctgcaaggcttctggctacaccttcaccaactactggatacactggatgaagcagaggcctggacgaggccttgagtggattggaaggattgagcctaatagcggtgatactaaatacaatgagaagatcaagagcagggccacactgactgtagacaaaccgtccagcacagcctacatgcagctcagcagcctgacatctgaggactctgcggtctattattgtgcaagatctgggtatgattaccctgaggcctggggccaaggcaccactctcacagtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggancgtcagtcttcctcttccccccaaaacccaanna

SEQ ID NO: 196 - DNA, 1045 bp, synthetic construct
  misc_feature 1..1045: note = synthetic primer
  source 1..1045: mol_type = other DNA
nnnnnnnnnnnnngtnactgcacctcggttctatcgattgaattccaccatgggatggtcatgtatcatcctttttctagtagcaactgcaaccggtgtacattccgacattgtgctgacccaatctccagcttctttggctgtgtctccagggcagagagccaccatctcctgcagagccagtgaaggtgttgaatattatggcacaactttaatgcagtggtaccaacagaaaccaggacagccacccaaactcctcatctatgctgcatccaacgtagaatctggggtccctgccaggtttagtggcagtgggtctgggacagacttcagcctcaacatccatcctgtggaggaggatgatattgcaatgtatttctgtcagcaaagtaggaaggtttcttggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggntgctgtctctttatgagngttgtgggcccgttgtcnggcaacntggcgnggngtgcactgtgtttgctgacgcaacccccactggttggggcatnnnnactcnnctgtcagcnnct

SEQ ID NO: 197 - DNA, 865 bp, synthetic construct
  misc_feature 1..865: note = synthetic primer
  source 1..865: mol_type = other DNA
nnnnnnnnnnnngtcactgcacctcggttctatcgattgaattccaccatgggatggtcatgtatcatcctttttctagtagcaactgcaaccggtgtacattccgaggtccagctgcaacagcctggggctgagcttgtgaagcctggggcttcagtgaagttgtcctgcaaggcttctggctataccttcaccaactactggatgcactgggtgaaacacaggcctggacgaggccttgagtggattggaaggattgatcctaatagtggtggtactaagtacaatgagaagttcaagaataaggccacactgactgtagacaatccctccagcacaggctacatgcagctcagcagcctgacatctgaggactctgcggtctattattgtacaagatctgggtatgattaccctgactactggggccaaggcaccactctcacagtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatct

SEQ ID NO: 198 - DNA, 1041 bp, synthetic construct
  misc_feature 1..1041: note = synthetic primer
  source 1..1041: mol_type = other DNA
nnnnnnnnnnnnnnnngnnnnntgnacctcggttctatcgattgaattccaccatgggatggtcatgtatcatcctttttctagtagcaactgcaaccggtgtacattccgacattgtgctgacccaatctccagcttctttggctgtgtctctagggcagagagccaccatctcctgcagagccagtgaaagtgttgaatattatggcacaagtttaatgcagtggtaccaacagaaaccaggacagccacccaaagtcctcatctatgctgcatccaacgtagattctggggtccctcccaggtttagtggcagtgggtctgggacagacttcagcctcaacttccatcctgtggaggaggatgatattgcaatgtatttctgtcagcaaagtaggaaggttccttggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgangagttgtggcccgttgtcaggnacgtggcgtggtgtgcactgtgnttgctgacncaaccccccactggttggggcattgccaccnacctgn

SEQ ID NO: 199 - DNA, 924 bp, synthetic construct
  misc_feature 1..924: note = synthetic primer
  source 1..924: mol_type = other DNA
nnnnnnnnccnngnnnntgcacctcggttctatcgattgaattccaccatgggatggtcatgtatcatcctttttctagtagcaactgcaaccggtgtacattccgaggtccaactgcaacagcctggggctgagcttgtgaagcctggggcttcagtgaagctgtcctgcaaggcttctggctacaccttcaccagctactggatgcactgggtgaagcagaggcctggacgaggccttgagtggattggaaggattgatcctaatagtggtggtactaagtacaatgagaagttcaagagcaaggccacactgactgtagacaaaccctccagcacagcctacatgcagctcagcagcctgacatctgaggactctgcggtctattattgtgcaagatctgggtatgattaccctgactactggggccaaggcaccactctcacagtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggangtgagccacgaagancctgaggtn

SEQ ID NO: 200 - DNA, 996 bp, synthetic construct
  misc_feature 1..996: note = synthetic primer
  source 1..996: mol_type = other DNA
nnnnnnnnnnngtcactgcacctcggttctatcgattgaattccaccatgggatggtcatgtatcatcctttttctagtagcaactgcaaccggtgtacattccgacattgtgctgacccaatctccagctcctttggctgtgtctctagggcagagagccaccatctcctgcagagccagtgaaggtgttgaatattatgacacaagtttaatgcagtggtaccaacagaaaccaggacagccacccaaactcctcatctatgctgcatccaacgtagaacctggggttcctgccaggtttggtggcagtgggtctgggacagacttcagcctcaacatccatcctgtggaggaggatgatattgcaatgtatttctgtcagcaaagtaggaaggttccttggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgagnagttgtggcccgttgtcnnnaacgtggcgtggtgtgnnactgtgtttgctg

SEQ ID NO: 201 - DNA, 895 bp, synthetic construct
  misc_feature 1..895: note = synthetic primer
  source 1..895: mol_type = other DNA
nnnnncnnnnnnnnngtnactgcacctcggttctatcgattgaattccaccatgggatggtcatgtatcatcctttttctagtagcaactgcaaccggtgtacattccgaggtccaactgcaacagcctggggctgagctggtaaagcctgggacttcagtgaagttgtcctgcaaggcttctggctacactttcaccagctactggatgcactgggtgaagcagaggcctggacaaggccttgagtggattggaatgattcatcctaatagtggtagtactaactacaatgagatgttcgagagcaaggcctcactgactgtagacaaatcctccagcacagcctacatgcaactcagcagcctgacatctgaggactctgcggtctattactgtgcaagaatcggatactccggctggggtcaaggaacctcagtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacg

SEQ ID NO: 202 - DNA, 926 bp, synthetic construct
  misc_feature 1..926: note = synthetic primer
  source 1..926: mol_type = other DNA
nnnnnnnnnnnnnngtnactgcacctcggttctatcgattgaattccaccatgggatggtcatgtatcatcctttttctagtagcaactgcaaccggtgtacattccgacattgtgatgacacagtctccatcctccctgagtgtgtcagcaggagagaaggtcactatgagctgcaagtccagtcagagtctgttaaatggtggaaatcaaaagaactacttggcctggtaccagcagaaaccagggcagcctcctaaagtgttgatcaacggggcatccactagggaatctggggtccctgatcgcttcacaggcaatggatctggaaccgatttcactcttaccatcagcagtgtgcaggctgaagacctggcagtttattactgtcagaatgatcatacttatccgctcacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtat

SEQ ID NO: 203 - DNA, 872 bp, synthetic construct
  misc_feature 1..872: note = synthetic primer
  source 1..872: mol_type = other DNA
nnnnnnnnnnnnnnngtcactgcacctcggttctatcgattgaattccaccatgggatggtcatgtatcatcctttttctagtagcaactgcaaccggtgtacattccgaggtccaactgcagcagcctggaggtgagctggtgaggcctgggtcctcagtgaagatgtcctgcaagacttctggatatacattcacattctacggtataaactgggtgaagcagaggcctggacagggcctggaatggattggatatatttatgttggaaatggttatactgagtacaatgagaagttcaaggtcaaggccacactgacttcagacacatcctccagcacagcctacatgcagctcagcggcctgacatctgaggactctgcaatctatttctgtgcaagatccgatggttacaactacttcgactactggggccaaggcaccactctcacagtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctc

SEQ ID NO: 204 - DNA, 1048 bp, synthetic construct
  misc_feature 1..1048: note = synthetic primer
  source 1..1048: mol_type = other DNA
nnnnnnnnnnnnnnnnnnncactgcacctcggttctatcgattgaattccaccatgggatggtcatgtatcatcctttttctagtagcaactgcaaccggtgtacattcccaaattgttctcacccagtctccagcaatcctgactgcatccccaggggagaaagtcacaatgacttgcagggccagttcaagtgtacattacatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatactgccacttattactgccatcagtggagtagtaacccacccacgttcggtgctgggacaaagttggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggntgctgtctctttatgaggagttgtgggcccgttgtcnnnaacgtggcgtggtgtgcactgtgtttgctgacgcaacccccactggttggggcattgccaccacctgtcagctcctttccgggactttcgc

SEQ ID NO: 205 - DNA, 849 bp, synthetic construct
  misc_feature 1..849: note = synthetic primer
  source 1..849: mol_type = other DNA
nnnnnnnnnnngncactgcacctcggttctatcgattgaattccaccatgggatggtcatgtatcatcctttttctagtagcaactgcaaccggtgtacattccgaggtccaactgcagcagcctggggctgagctggtaaagcctggggcttcagtgaagttgtcctgcaaggcttctggctacactttcaccagctactggatgcactgggtgaagcagaggcctggacaaggccttgagtggattggattgattcatcctaatagtggtagtacttactacaatgagaagttcaagaacaaggccacactgactgtagacaaatcctccagcacagcctacatgcaactcagcagcctgacatctgaggactctgcggtctattactgtgcaagatgggatgattcctactggtacttcaaagtctggggcacagggaccacggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaan

SEQ ID NO: 206 - DNA, 985 bp, synthetic construct
  misc_feature 1..985: note = synthetic primer
  source 1..985: mol_type = other DNA
nnnnnnnnnnnnnngtcactgcncctcggttctatcgattgaattccaccatgggatggtcatgtatcatcctttttctagtagcaactgcaaccggtgtacattcccaaattgttctcacccagtctccagcaatcctgtctgcatctccaggggagaaggtcacaatgacttgcagggccagttcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagtagcaacccgtacacgttcggaggggggaccaagctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggntgctgtctctttatgaggagttgtggcccgttgtcnnnaacgtgggcgtggngtgcactgtgtttgctga

SEQ ID NO: 207 - DNA, 912 bp, synthetic construct
  misc_feature 1..912: note = synthetic primer
  source 1..912: mol_type = other DNA
nnnnnnnnnnnnnngtnactgcacctcggttctatcgattgaattccaccatgggatggtcatgtatcatcctttttctagtagcaactgcaaccggtgtacattccgaggtccagctgcaacagtctggacctgagctggtgaggcctggggcttcagtgaagatatcctgcaaggcttctggctacaccttcactgactactatatacactggctgaagcagaggcctggacagggacttgagtggattggattgatttttcctggaagtggtagtatttactgtaatgagaagttcaagggcaaggctacacttactgtagacaaatcctccaccacagcctacatgttgctcagcagcctgacctctgaggactctgcggtctacttctgtgcaagatgggagactacggcgtggtacttcgatgtctggggcacagggaccacggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggancgtcagtcttcctcttccccccaaaacccaaggacaccctcatgatctcccggacccctgaggtcacatgcgtggtggtggacgtga

SEQ ID NO: 208 - DNA, 1013 bp, synthetic construct
  misc_feature 1..1013: note = synthetic primer
  source 1..1013: mol_type = other DNA
nnnnnnnnnnnnnnncactgcacctcggttctatcgattgaattccaccatgggatggtcatgtatcatcctttttctagtagcaactgcaaccggtgtacattcccaaattgttctcacccagtctccccaaattgttctcacccagtctccagcaatcctgtctgcatctccaggggagaaggtcacaatgacttgcagggccagtttaagtgtaagttacatgcactggtaccagcagaagccaggatcctcccccaaaccctggatttatgccacatccaacctggcttctggagtccctgcccgcttcagtggcagtgggtctgggacctcttactctctcacagtcagcagagtggaggccgaagatgctgccacttattactgccagcagtggaggagttacccgtacacgttcggagggggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtgggcccgttgtcagnaacgtggngtggngtgcactgtgtttgctgacgcaa

SEQ ID NO: 209 - DNA, 951 bp, synthetic construct
  misc_feature 1..951: note = synthetic primer
  source 1..951: mol_type = other DNA
nnnnnnnnnnnnngtnactgcacctcggttctatcgattgaattccaccatgggatggtcatgtatcatcctttttctagtagcaactgcaaccggtgtacattccgaggtccaactgcaacagtctggacctgtgctggtgaagcctgggccttcaatgaagatatcctgtaaggcttctggattcacattcactgactactacatacactgggtgaggcagagccatggaaagagccttgagtggattggacttgtttctccttacaatggtggtacttactacaaccagaagttcaagggcaaggccacattgactgtagactcatcctccagcacagcctacatggagctaagcagcctgacttctgaggactctgcggtctattactgtgcaagattgggctactatggtgactggtactactttgactactggggccaaggcacccctctcacagtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgnntcacatgcgtggtggtggacgtgagccacgaagancctgagnnnagttcaactggtacg

SEQ ID NO: 210 - DNA, 986 bp, synthetic construct
  misc_feature 1..986: note = synthetic primer
  source 1..986: mol_type = other DNA
nnnnnnnnnnnnnnnntnaactgcacctcggttctatcgattgaattccaccatgggatggtcatgtatcatcctttttctagtagcaactgcaaccggtgtacattcccaaattgttctcacccagtctccagcactcatggctacatctccaggggaaaaggtcaccatcacctgtagtgtcagctcaagtataagttccagcaacttgaactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccatacacgttcggaggggggaccaagctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagantgactggtattcttaactatgttgctccttttacgctatgtggatacgctgctttaatgcctttgtatcatgctattgcttcccgtatggctttcattttctcctccttgtataaatcctggttgctgtctctttatgaaggagttgtggcccgttgtcaggnaacgtgggcgnggtnngcactgn

SEQ ID NO: 211 - DNA, 871 bp, synthetic construct
  misc_feature 1..871: note = synthetic primer
  source 1..871: mol_type = other DNA
nnnnnnnnnnnnncactgcacctcggttctatcgattgaattccaccatgggatggtcatgtatcatcctttttctagtagcaactgcaaccggtgtacattccgaggtccagctgcaacagtctggggctgagctggtgaagcctggggcttcagtgaagttgtcctgcaaggcttctggctacactttcaccacctactggatgcactggatgaagcagtggcctggacaaggccttgagtggattggattgattcatcctaatagtggtagtacttactacaatgagaagttcaagagcaaggccacactgactgtagacaaatcctccagcacagcctacatgcaactcagcagcctgacatctgaggactctgcggtctattactgtgcaagatttgaggagctatggggaggttactggtacttcgatgtctggggcacagggaccacggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcangactctactccctcagcancgtggtgaccgtgccctccagcagcttgggcacccagacctacatctgcnacgtgaatcacaagcccagcaacaccaaggtggacaaganagttgagcccaaatcttgtgacaannctcacacatgcccaccgtgcccancacctgaactcctggggggaccgncagtcttcctcttccccccaaaacccangacnnccnn

SEQ ID NO: 212 - DNA, 969 bp, synthetic construct
  misc_feature 1..969: note = synthetic primer
  source 1..969: mol_type = other DNA
nnnnnnnnnnnngtcactgcacctcggttctatcgattgaattccaccatgggatggtcatgtatcatcctttttctagtagcaactgcaaccggtgtacattcccaaattgttctcacccagtctccagcactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccgtacacgttcggaggggggaccaagctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggntgctgtctctttatgaggagttgtggcccgttgtcaggnaacgtggngtg

SEQ ID NO: 213 - DNA, 961 bp, synthetic construct
  misc_feature 1..961: note = synthetic primer
  source 1..961: mol_type = other DNA
nnnnnnnnnnnncnggtnactgcacctcggttctatcgattgaattccaccatgggatggtcatgtatcatcctttttctagtagcaactgcaaccggtgtacattccgaggtccagctgcagcagcctggggctgagctggcaaagcctggggcttcagtgaagttgtcctgcaaggcttctggctacaatttcaccagctactggatgcactgggtgaagcagaggcctggacaaggccttgagtggattggattgattcatcctaatagtggtggtacttactacaatgagaagttcaagagcaaggccacactgactttagacagatcctccagcacagcctacatgcaactcagcagcctgacatctgaggactctgcggtctattattgtgcaagatttgaggaggaatggggagggtattggtacttcgatgtctggggcacggggaccacggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagancctgaggncaagttcaactggtacgnnn

SEQ ID NO: 214 - DNA, 905 bp, synthetic construct
  misc_feature 1..905: note = synthetic primer
  source 1..905: mol_type = other DNA
nnnnnnnnnnnnnnnnntgcacctcggttctatcgattgaattccaccatgggatggtcatgtatcatcctttttctagtagcaactgcaaccggtgtacattcccaaattgttctcacccagtctccagcactcatggctgcatctccaggggaaaaggtcaccatcacctgcagtgtcagttcaagtataagttccagcaacttgcactggtaccaacagaagtcagaaacctcccccaaaccctggatttatggcacttccaacctggcttctggagtccctgttcgcttcagtggcagtggatctgggacctcttattctctcacaatcagcagcatggaggctgaagatgctgccacttattactgtcaacagtggagtagttacccgtacacgttcggaggggggaccaaactggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctcntt

SEQ ID NO: 215 - DNA, 922 bp, synthetic construct
  misc_feature 1..922: note = synthetic primer
  source 1..922: mol_type = other DNA
nnnnnnnnnnnnngtcactgcacctcggttctatcgattgaattccaccatgggatggtcatgtatcatcctttttctagtagcaactgcaaccggtgtacattccgaggtccaactgcaacagtctggggctgagctggtaaagcctggggcttcagtgaagttgtcctgcaaggcttctggctacactttcaccagctactggatgcactgggtgaagcagaggcctggacaaggccttgagtggattggactgattcatcctaatagtagtagtacttactacaatgagaagttcaagaccagggccacactgactgtagacaagtcctccagcacagcctacatgcaactcagcagcctgacatctgaggactctgcggtctattactgtgcaagattgggctatggtaactcctactggtacttcgatgtctggggcacagggaccacggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaagncaccctcatgatctcccggacccctgaggtcacatgcgtggtggtggacgtgagccnnn

SEQ ID NO: 216 - DNA, 1010 bp, synthetic construct
  misc_feature 1..1010: note = synthetic primer
  source 1..1010: mol_type = other DNA
nnnnnnnnnnncnggtnnactgcacctcggttctatcgattgaattccaccatgggatggtcatgtatcatcctttttctagtagcaactgcaaccggtgtacattcccaaattgttctcacccagtctccagcactcatggctgcatctccaggggagaaggtcaccatcacctgcagtgtcagctcaagtattagttccagcaacttgcactggtaccagcagaagtcagaaacctcccccaaaccctggatttatggcacatccaacctggcttctggagtccctattcgcttcagtggcagtggatctgggacctcttattctctcacaatcagcagtttggaggctgaggatgctgccacttattactgtcaacagtggagaagttacccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggntgctgtctctttatgaggagttgtgggcccgttgtcangnacgnngnngtggtgtgcactgtgtttgctgacgcannnccnnnnnggn

SEQ ID NO: 217 - DNA, 926 bp, synthetic construct
  misc_feature 1..926: note = synthetic primer
  source 1..926: mol_type = other DNA
nnnnnnnnnncnggtcactgcacctcggttctatcgattgaattccaccatgggatggtcatgtatcatcctttttctagtagcaactgcaaccggtgtacattccgaggtccaactgcagcagtctggggctgagctggtaaagcctggggcttcagtgaagttgtcctgcaaggcttctggctacactttcaccagctactggatgcactgggtgcagcagaggcctggacaaggccttgagtggattggattgattcatcctattggtggtggtactcactacaatgagaagttcaagaacaaggccacactgactgtagacaaatcctccagcacagcctacatgcaactcagcagcctgacatctgaggactctgcggtctattactgtgcaagactcggaactggtccgtactactttgactactggggccaaggcaccactctcacagtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgantcacatgcgtggtggtggacgtgagccacgaagancctga

SEQ ID NO: 218 - DNA, 992 bp, synthetic construct
  misc_feature 1..992: note = synthetic primer
  source 1..992: mol_type = other DNA
nnnnnnnnnnnnnnnnnnnnctgcacctcggttctatcgattgaattccaccatgggatggtcatgtatcatcctttttctagtagcaactgcaaccggtgtacattcccaaattgttctcacccagtctccagcactcatggctgcatctccaggggagaaggtcaccatcacctgcagtgtcagctcaagtataagttccagcaccttggactggtaccagcagaagtcagaaacctcccccaaaccctgggtttatggcacatccaacctggcttctggagtccctgttcgcttcagtggcagtggatctgggacctcttattctctcacaatcagcagcatggaggctgaagatgctgccacttattactgtcaacagtggagtagttacccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggntttcattttctcctccttgtataaatcctggntgctgtctctttnatgaggagttgtggcccgtngncaggnaacgnggcgtggtgtgcactgnntttgct

SEQ ID NO: 219 - DNA, 914 bp, synthetic construct
  misc_feature 1..914: note = synthetic primer
  source 1..914: mol_type = other DNA
nnnnnnnnnnnngtcactgcacctcggttctatcgattgaattccaccatgggatggtcatgtatcatcctttttctagtagcaactgcaaccggtgtacattccgaggtccagctgcagcagtctggacctgtgctggtgaagcctgggccttcagtgaatatatcctgtaaggcttctggattcacattcactgactactacattcactgggtgaaacagagccatggaaagagccttgagtggattggacttgtttatccttacagtggtggtacttactacaaccagaagttcaagggcaaggccacattgactgtagacgcatcctccagcacagcctacatggagctaggcagcctgacttctgaggactctgcggtctattactgtgcaagattgggcgacaacttctactactttgactactggggccaaggcaccactctcacagtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccn

SEQ ID NO: 220 - DNA, 979 bp, synthetic construct
  misc_feature 1..979: note = synthetic primer
  source 1..979: mol_type = other DNA
nnnnnnnnnnnnnnnnnnntgcacctcggttctatcgattgaattccaccatgggatggtcatgtatcatcctttttctagtagcaactgcaaccggtgtacattcccaaattgttctcacccagtctccaacactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacatcttattctctcacaatcagcagcatggaggctgaagatgctgccacttattactgtcaacagtggagaagttacccgtacacgttcggaggggggaccaagctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggnngctgtctctttatgaggagttgtgggcccgttgtcaggnaacgtggcgtggngtgnn

SEQ ID NO: 221 - DNA, 908 bp, synthetic construct
  misc_feature 1..908: note = synthetic primer
  source 1..908: mol_type = other DNA
nnnnnnnnnnnnnngtcactgcacctcggttctatcgattgaattccaccatgggatggtcatgtatcatcctttttctagtagcaactgcaaccggtgtacattccgaggtccaactgcaacagcctggacctgtgctggtgaagcctgggccttcagtgaagatatcctgtaaggcttctggattcacattcactgactactacatgcactgggtgaaacagagccatggaaagagccttgagtggattggtcttgtttctccttacaatggtggtactttctacaaccagaagttcaagggcaaggccacattgactgtagacacatcctccagcacagcctacatggaactaaacagcctgacttctgaggactctgcggtctattactgtgcaagagtgggtaatagctacgtccattatgctatggactactggggtcaaggaacctcagtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgangtcacatgcgtggnn

SEQ ID NO: 222 - DNA, 995 bp, synthetic construct
  misc_feature 1..995: note = synthetic primer
  source 1..995: mol_type = other DNA
nnnnnnnnnnnnnccnggtnactgcacctcggttctatcgattgaattccaccatgggatggtcatgtatcatcctttttctagtagcaactgcaaccggtgtacattcccaaattgttctcacccagtctccagcactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtaattacccgtacacgttcggaggggggaccaagctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgnataaatcctggntgctgtctctttatgangagttgtggncccgttgtcngnnaacgtggcgtggtgtgcactgtgnttgctgn

SEQ ID NO: 223 - DNA, 921 bp, synthetic construct
  misc_feature 1..921: note = synthetic primer
  source 1..921: mol_type = other DNA
nnnnnnnnnnncngnnnntgcacctcggttctatcgattgaattccaccatgggatggtcatgtatcatcctttttctagtagcaactgcaaccggtgtacattccgaagtgaagctggtggagtctggggaaggcttagtgaagcctggagggtccctgaaactctcctgtgcagcctctggattcactttcagtagctatgccatgtcttgggttcgccagactccagagaagaggctggactgggtcgcatacattagtagtggtggtgatcacatctactatgcagacactgtgaagggccgattcaccatctccagagacaatgccaggaacaccctgtacctgcaaatgagcagtctgaagtctgaggacacagccatgtattactgtacaagagataccggttactacgtctctcggtacttcgatgtctggggcacagggaccacggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccgganccctgaggtcacatgcgtggtggtggacgtgagcca

SEQ ID NO: 224 - DNA, 961 bp, synthetic construct
  misc_feature 1..961: note = synthetic primer
  source 1..961: mol_type = other DNA
nnnnnnnnnnnnngnnnntgcacctcggttctatcgattgaattccaccatgggatggtcatgtatcatcctttttctagtagcaactgcaaccggtgtacattccgacattgtgatgacccagtctcaaaaattcatgtccacatcagtaggagacagggtcagcgtcacctgcaaggccagtcagaatgtggttactaatgttgcctggtatcaacagaaatcagggcaatctcctaaagtagtgatttattcggcatccttccggtccagtggagtccctgatcgcttcacaggcagtggatctgggacagatttcactctcaccatcagcaatgtgcagtctgaagacttggcagagtatttctgtcaccaatataacagccatcctctgacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggntgctgnctcttnantnnnnganntgtggcccgttgtcaggnaac

SEQ ID NO: 225 - DNA, 922 bp, synthetic construct
  misc_feature 1..922: note = synthetic primer
  source 1..922: mol_type = other DNA
nnnnnnnnnnnnngtcactgcacctcggttctatcgattgaattccaccatgggatggtcatgtatcatcctttttctagtagcaactgcaaccggtgtacattccgaggtccagctgcagcagcctggacctgaattggtgaagcccgggtcctcactgaagatatcctgcaaggcttctggttacaccttcactgactactttataaactgggtgaaacagaggcctggacagggacttgactggattggatggatttttcctggaagtggtagtacttactacaatgacaagttcaagggcaaggccacacttactgtagacaaatcctccagcactgcctacatgttgctcagcagcctgacctctgaggcctctgcggtctatttctgtgcaagatgggactccgatagtacctactggtacttcgatgtctggggcacagggaccacggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccac

SEQ ID NO: 226 - DNA, 1062 bp, synthetic construct
  misc_feature 1..1062: note = synthetic primer
  source 1..1062: mol_type = other DNA
nnnnnnnnnnnncnnnnnnnnntgcacctcggttctatcgattgaattccaccatgggatggtcatgtatcatcctttttctagtagcaactgcaaccggtgtacattcccaaattgttctcacccagtctccagcactcatggctgcatctccaggggagaaggtcaccatcacctgcagtgtcaattcaagtataagttccagcaccttgcactggtaccagcagaagtcacaaacctcccccaaaccctggatttatggtacatccaatttggcttctggagtccctattcgcttcagtggcagtggatctgggacctcttattctctcacaatcagcagcatggaggctgaagatgctgccacttattactgtcaacagtggagtacttacccgtacacgttcggaggtggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtgggcccgttgtcaggcaacgtggngnggngtgcactgtgtttgctgacgcaaccccnctggtnggggcattgccaccacctgtcagctcctttccgggactttcgnttnnnnnn

SEQ ID NO: 227 - DNA, 910 bp, synthetic construct
  misc_feature 1..910: note = synthetic primer
  source 1..910: mol_type = other DNA
nnnnnnnnnnnnnnggtcactgcacctcggttctatcgattgaattccaccatgggatggtcatgtatcatcctttttctagtagcaactgcaaccggtgtacattccgaggtccaactgcaacagtctggagctgagctgatgaagcctggggcctcagtgaagctttcctgcaaggctactggctacacattcactggctactggatagagtggttaaagcagaggcctggacatggccttgagtggattggagagattttacctggaagtgataatactaactacaatgagaagttcaggggcaaggccacattcactgcagatacatcctccaacacagcctacatgcacctcagcagcctgacaactgaggactctgccatctattactgtgcaagagaagggggtttctactttgactactggggccaaggcaccactctcacagtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggancgtcagtcttcctcttccccccaaaacccaaggacaccctcatgatctcccggacccctgaggtcacatgcgtggtggtggacgtgancn

SEQ ID NO: 228 - DNA, 1054 bp, synthetic construct
  misc_feature 1..1054: note = synthetic primer
  source 1..1054: mol_type = other DNA
nnnnnnnnnnnnnnnnnnnnnnncctcggttctatcgattgaattccaccatgggatggtcatgtatcatcctttttctagtagcaactgcaaccggtgtacattcccaggctgttgtgactcaggaatctgcactcaccacatcacctggtgaaacagtcacactcacttgtcgctcaagtattggggctgttacaactagtaactacgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggccagcccaagtcttcgccatcagtcaccctgtttccgccctcgagtgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatagaagcttgatcctcaacctctggattacaaaatttgtgaaagattgactggtattcttaactatgttgctccttttacgctatgtggatacgctgctttaatgcctttgtatcatgctattgctncccgtatggctttcattttctcctccttgtataaatcctggttgctgtctctttatgaggagttgtggcccgttgtcaggnaacgtggcgtggngtgcactgngtttgctgacgcaacccccactggtnggggcattgcnnccacctgtcanctnctttccgggactttcgcttn

SEQ ID NO: 229 - DNA, 909 bp, synthetic construct
  misc_feature 1..909: note = synthetic primer
  source 1..909: mol_type = other DNA
nnnnnnnnnnnnnccnngnnnactgcacctcggttctatcgattgaattccaccatgggatggtcatgtatcatcctttttctagtagcaactgcaaccggtgtacattccgaggtccagctgcaacagtctggacctgtgctggtgaagcctgggccttcagtgaagatatcctgtaaggcttctggattcacattcactgactactacatgcactgggtgaagcagagccatggaaagagccttgaatggattggacttgtttatccttacaatggtggtactagctacaaccagaagttcaagggcaaggccacattgactgtagacacatcctccaccacagcctacatggagctaaacagcctgacttccgaggactctgcggtctattactgtgcaagattgggctcaggctacgagtactactttgactactggggccaaggcacctctctcacagtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccgganccctgaggtcacatgcgtggtg

SEQ ID NO: 230 - DNA, 969 bp, synthetic construct
  misc_feature 1..969: note = synthetic primer
  source 1..969: mol_type = other DNA
nnnnnnnnnnnnnngtnactgcacctcggttctatcgattgaattccaccatgggatggtcatgtatcatcctttttctagtagcaactgcaaccggtgtacattcccaaattgttctcacccagtctccagcactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaggatgctgccacttattactgtcaacagtggagaacttacccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggntttcattttctcctccttgtataaatcctggttgctgnctctttatnnngnagttgnnggcccgtnnnnnnnancgtnnn

SEQ ID NO: 231 - DNA, 934 bp, synthetic construct
  misc_feature 1..934: note = synthetic primer
  source 1..934: mol_type = other DNA
nnnnnnnnnnnnngncactgcacctcggttctatcgattgaattccaccatgggatggtcatgtatcatcctttttctagtagcaactgcaaccggtgtacattccgaggtccaactgcagcagcctggacctgtgctggtgaagcctgggccttcagtgaagatatcctgtaaggcttctggattcacattcactgactactacatgcactgggtgaagcagagccatggagagagccttgagtggattggacttgtttctccttacaatggtggtactttctacaaccagaagttcaagggcaaggccacattgactgtagacacatcctccagcacagcctacatggaactaaacagcctgacttctgaggactctgcggtctattactgtgcaagagtgggtagtagttacgtccattatgctatggactactggggtcaaggaacctcagtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtgganngagagttgagcccaaatcttgtgacaaactcacacatgcccaccgtgcccagcacctgaactcctggggggancgtcngtcttcctcttcccccaaaacccnanganaccctcatgatctcccggacccctgaggtcncntgcgtggtggtggangtgannccnnnnaannncnntnan

SEQ ID NO: 232 - DNA, 937 bp, synthetic construct
  misc_feature 1..937: note = synthetic primer
  source 1..937: mol_type = other DNA
nnnnnnnnnnncnngtcactgcacctcggttctatcgattgaattccaccatgggatggtcatgtatcatcctttttctagtagcaactgcaaccggtgtacattcccaaattgttctcacccagtctccagcactcacggctgcatctccaggggagagggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtaagtacccgtacacgttcggaggggggaccaagctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatga

SEQ ID NO: 233 - DNA, 880 bp, synthetic construct
  misc_feature 1..880: note = synthetic primer
  source 1..880: mol_type = other DNA
nnnnnnnnnnnnnngncactgcacctcggttctatcgattgaattccaccatgggatggtcatgtatcatcctttttctagtagcaactgcaaccggtgtacattccgaggtccaactgcaacagtctggggctgggctggtaaagcctggggcttcagtgaagttgccttgcaaggcttctggctacactttcaccagctactggatgcactgggtgcagcagaggcctggacaaggccttgagtggattggattgattcatcctattggtggtggtactcactacaatgagaagttcaagaacaaggccacactgactgtggacaaatcctccagcacagcctacatgcaactcagcagcctgacatctgaggactctgcggtctattactgtgcaagactcggaactggtccgtactactttgactactggggccaaggcaccactctcacagtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggan

SEQ ID NO: 234 - DNA, 1081 bp, synthetic construct
  misc_feature 1..1081: note = synthetic primer
  source 1..1081: mol_type = other DNA
nnnnnnnnnnnnnnnngnnnactgcacctcggttctatcgattgaattccaccatgggatggtcatgtatcatcctttttctagtagcaactgcaaccggtgtacattcccaaattgttctcacccagtctccagcactcatggctgcatctccaggggagaaggtcaccatcacctgcagtgtcagctcaagtataagttccagcaacttgcactggtaccagcagaagtcagagacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgnctttgtatcatgctattgcttcccgtatggctttcattttctcctccttgnnnnnatcctggntgctgtctctttatgaggagttgtgggcccgttgtcaggcaacnnngcgtggngtgcactgtgtttgctgacgcaacccccnactggnnnnnnnattgccaccnnncngtcagcnnctttncgggactttcgctttncccntncntattnnnnnnnn

SEQ ID NO: 235 - DNA, 925 bp, synthetic construct
  misc_feature 1..925: note = synthetic primer
  source 1..925: mol_type = other DNA
nnnnnnnnnnnnngtcactgcacctcggttctatcgattgaattccaccatgggatggtcatgtatcatcctttttctagtagcaactgcaaccggtgtacattccgaggtccagctgcagcagcctggacctgacgtggtgaagcctggggcttcagtgaagatttcctgcaaggcttctggctacaccttcactgactactatataaactgggtgaagcagaggcctggacagggacttgagtggattggatggatttttcctggaagtggtagtagttattacaatgagaagttcaaggacaaggccacatctactgtagacaaatcctccagcacagcctacatgttgctcagcagcctgacctctgaggactctgcggtctatttctgtgcaaaatggaaggattacgggtggtacttcgatgtctggggcacagggaccacggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccgganccctgnntcacatgcgtggtggtggacgtgagccacgaagacctna

SEQ ID NO: 236 - DNA, 1056 bp, synthetic construct
  misc_feature 1..1056: note = synthetic primer
  source 1..1056: mol_type = other DNA
nnnnnnnnnnnnnnnnngnnnnctgnacctcggttctatcgattgaattccaccatgggatggtcatgtatcatcctnntttctagtagcaactgcaaccggtgtacattcccaaattgttctcacccagtctccagcactcatggctgcatctccaggggagaaggtcaccctcacctgcaatgtcagctcaagtctaagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggaggacttacccttacacgttcggaggggggaccaagctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagantgactggtattcttaactatgttgctccttttacgctatgtggatacgctgctttaatgcctttgtatcatgctattgcttcccgtatggctttcattttctcctccttgtataatcctggttgctgtctctttatgaggagttgtggnccgttgtcaggcaacgtggcgtggtgtgcactgtgtttgcnngacnncacccccnnctggttggggnattgccaccnncctgtcagctnntttccgggannntn

SEQ ID NO: 237 - DNA, 869 bp, synthetic construct
  misc_feature 1..869: note = synthetic primer
  source 1..869: mol_type = other DNA
nnnnnnnnnnnngtcactgcacctcggttctatcgattgaattccaccatgggatggtcatgtatcatcctttttctagtagcaactgcaaccggtgtacattccgaggtccaactgcagcagtctggacctgtgctggtgaagcctgggccttcagtgaagatatcctgtaaggcttctggattcacattcactgactactacattcactgggtgaagcggagccatggaaagagccttgagtggattggacttgtttctccttacaatggtggtactttctacaaccagaagttcaagggcaaggccacattgactgtagatacatcctccaatacagcctacatggagctaaccagcctgacttctgaggactctgcggtctattactgtgcaagattgggggttaactggtacttcgatgtctggggcacagggaccacggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacnnntgcccaccgtgcccagcacctgaactcctggggggancgtcagtcttcctcttccccccaaaacccaaggacaccctcatgatctc

SEQ ID NO: 238 - DNA, 981 bp, synthetic construct
  misc_feature 1..981: note = synthetic primer
  source 1..981: mol_type = other DNA
nnnnnnnnnnnnnncnngtcactgcacctcggttctatcgattgaattccaccatgggatggtcatgtatcatcctttttctagtagcaactgcaaccggtgtacattcccaaattgttctcacccagtctccagcactcatggctgcatctccaggggaaaaggtcaccctcacctgcagtgtcagctcaagtataagttccagcaccttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtacttacccatacacgttcggaggggggaccaagctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggntgctgtctctttatgaaggagttgtgggcccgttgtcnnnaacgtgggcgtggtgtg

SEQ ID NO: 239 - DNA, 926 bp, synthetic construct
  misc_feature 1..926: note = synthetic primer
  source 1..926: mol_type = other DNA
nnnnnnnnnnnnnngnnnntgcacctcggttctatcgattgaattccaccatgggatggtcatgtatcatcctttttctagtagcaactgcaaccggtgtacattccgaggtccaactgcaacagcctggacctgtactggtgaagcctgggccttcaatgaagatatcctgtaaggcttctgggtacgctttcactgactacttcatacactgggtgagacagagccatggaaggagccttgagtggattggacttgtttctccttataatggtggtacttactacaaccagaagttcaagggcaaggcctcattgactgtagacacatcctccagcacagcctacatggaactaagcagcctgacttctgaggactctgcggtctattactgtgcaagattgggctactatggtaactggtacttctttgactactggggccaaggcacccctctcacagtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggancgtcagtcttcctcttcccccaaaacccaaggacaccctcatgatctcccggacccctgagtcacatgcgtggtggtggacgtgagccacganna

SEQ ID NO: 240 - DNA, 1000 bp, synthetic construct
  misc_feature 1..1000: note = synthetic primer
  source 1..1000: mol_type = other DNA
nnnnnnnnnnnnnnnnnnctgcacctcggttctatcgattgaattccaccatgggatggtcatgtatcatcctttttctagtagcaactgcaaccggtgtacattcccaaattgttctcacccagtctccagcactcatggctacatctccaggggaaaaggtcaccatcacctgtagtgtcagctcaagtataagttccagcaacttgaactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccatacacgttcggaggggggaccaagctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttgatcctcaacctctggattacaaaatttgtgaaagattgactggtattcttaactatgttgctccttttacgctatgtggatacgctgctttaatgcctttgtatcatgctattgcttcccgtatggctttcattttctcctccntgtataaatcctgnngctgnctctttatgagganttgnggcccgttgtcaggnaacgtggcgnggtgtgcactgtgnttgcnngacnnnnnncn

SEQ ID NO: 241 - protein, 126 aa, Escherichia coli
TGVHSEVQLQQSGAEQVRPGASVKMSCKASGYTFTTYNMNWMKQTPGQGLEWIGVIYPGNGDISYNQKFKGKATLTVDKSSSTAYMQLSSLTSEDSAVYFCAREGDYGPWFAYWGQGTLVTVSAAS

SEQ ID NO: 242 - protein, 115 aa, Escherichia coli
TGVHSQIVLTQSPAIMSASPGEKVTMTCRASSSVSSSYLHWYQQKSGASPKFWIYSTSKLASGVPARFSGSGSGTSYSLTISSVEAEDAATYYCQHYSRYPLTFGGGTKLEIKRT

SEQ ID NO: 243 - protein, 126 aa, Escherichia coli
TGVHSEVQLQQSGPVLVKPGPSVKISCKASGFTFTDYYIHWVKQSHGKSPEWIGLVSPYNGGTYYNQKFMGRATLTEDTSSNTAYMELNSLTSEDSAVYYCARLLRGYWYFDVWGTGTTVTVSSAS

SEQ ID NO: 244 - protein, 115 aa, Escherichia coli
TGVHSQIVLTQSPALMAASPGEMVTITCSVSSSISSSNLHWYQQKSETSPKPWIYGTSNLASGVPVRFSGSGSGTSYSLTISSMEAEDAATYYCQQWRTYPYTFGGGTKLEIKRT

SEQ ID NO: 245 - protein, 126 aa, Escherichia coli
TGVHSEVQLQQPGAELVRPGASVKMSCKASGYTFTIYNLHWVKQTPRQGLEWIGTIYPGDGDTSYNQKFKGKATLTVDKSSSTAYMQLSNLTSEDSAVYFCAREGDYGPWFAYWGQGTLVTVSAAS

SEQ ID NO: 246 - protein, 115 aa, Escherichia coli
TGVHSQIVLTQSPAIMSASPGEKVTMTCRASSSVSSSYLHWYQQKSGASPKLWIYSSSNLASGVPARFSGSGSGTSYSLTINSVEAEDAATYYCQHYGSYPLTFGGGTKLEIKRT

SEQ ID NO: 247 - protein, 124 aa, Escherichia coli
TGVHSEVQLQQSGPELVKPGASVKISCKASGYAFSSSWMNWVKQRPGKGLKWIGRIYPGDGDTNYNGKFKGKATLTADKSSXTAYMQLSSLTSEDSAVYFCAREGNLXFDYWGQGTTLTVSSAS

SEQ ID NO: 248 - protein, 118 aa, Escherichia coli
TGVHSDIVLTQSPASLDVSLGQRATISCRASQSVSTSTYSYMHWYQQKPGQSPKVLIKYASNLEAGVPARVSGSGSGTDFTLNIHPVEEEDTATYYCQHSWEIPPTFGGGTKLEIKRT

SEQ ID NO: 249 - protein, 127 aa, Escherichia coli
TGVHSQVQLKQSGPGLVAPSQSLSITCTVSGFSLTNYAISWVRQPPGKGLEWLGIIWTGGGTLYNSALKSRLSISKDNSKSQVFLKMNSLQTDDTARYYCARAYYSNYDWYFDVWDTGTTVTVSSAS

SEQ ID NO: 250 - protein, 114 aa, Escherichia coli
TGVHSDIQMTQSPSSLSASLGDRVTISCSASQGISNYLNWYQQKPDGTVKLLIYYTSSLHSGVPSRFSGSGSGTDYSLTISNLEPEDIATYYCQQYSQVPYTFGGGTKLEIKRT

SEQ ID NO: 251 - protein, 124 aa, Escherichia coli
TGVHSEVQLQQSGAELVKPGASVKVSCKASGYSFTNYWVHWVKQRPGQGLEWIGRIYSSDGDSNYNQKFKGKATLTVDKSSSTAYMQLSSLTSEDSAVYYCAIEGNLYFDYWGQGTTLTVSSAS

SEQ ID NO: 252 - protein, 118 aa, Escherichia coli
TGVHSDIVLTQSPASLAVSLGQRATISCRASQSVSTSSYSYMHWYQQKPGQPPKFLIKYASNLESGVPARFSGSGSGTDFTLNIHPVEEEDTATYYCQHSWEIPLTFGAGTKLKLKRT

SEQ ID NO: 253 - protein, 126 aa, Escherichia coli
TGVHSEVQLQQPGPVLVKPGPSVKISCKASGFTFTDYFIHWVKQSHGKSLEWIGLVSPYNGGTYYNQQFKGKATLTVDTSSSTAYMDLNSLTSEDSAVYYCTRLLRGYWYFDVWGTGTTVTVSSAS

SEQ ID NO: 254 - protein, 115 aa, Escherichia coli
TGVHSQIVLTQSPALMAASPGEKVTITCSVSSSIGSSNLHWYQQKSETSPKPWIYGTSNLASGVPVRFSGSGSGTSYSLTISSMEAEDAATYYCQQWSTYPYTFGGGTKLEIKRT

SEQ ID NO: 255 - protein, 127 aa, Escherichia coli
TGVHSQVQLKESGPGLVAPSQSLSITCTVSGFSLTNYAISWVRQPPGKGLEWLGIIWTGGGTLYNSALKSRPSISKDNSKSQVFLKMNSLQTDDTARYYCARAYYSNYDWYFDVWDTGTTVTVSSAS

SEQ ID NO: 256 - protein, 114 aa, Escherichia coli; identical to SEQ ID NO: 250

SEQ ID NO: 257 - protein, 126 aa, Escherichia coli
TGVHSEVQLQQSGPVLVKPGPSVKISCKASGFTFTDYFVHWVKQSHGKSLEWIGLVSPYNGGTYYNQQFKGKATLTVDTSSSTAYMDLNSLTSEDSAVYYCARLLRGYWYFDVWGTGTTVTVSSAS

SEQ ID NO: 258 - protein, 115 aa, Escherichia coli
TGVHSQIVLTQSPALMAASPGEKVTITCSVSSSISSSTLHWYQQKSETSPKPWIYGTSNLASGVPVRFGGSGSGTSYSLTISSMEAEDAATYYCQQWSTYPYTFGGGTKLEIKRT

SEQ ID NO: 259 - DNA, 380 bp, Escherichia coli
  source 1..380: mol_type = other DNA
accggtgtacattccgaggtccaactgcaacagtctggggctgagcaggtgaggcctggggcctcagtgaagatgtcctgcaaggcttctggctacacatttaccacttacaatatgaactggatgaagcagacacctggacagggcctggaatggattggagttatttatccaggaaatggtgatatttcctacaatcagaagttcaagggcaaggccacactgactgtagacaaatcctccagcacagcctacatgcagctcagcagcctgacatctgaagactctgcggtctatttctgtgcaagagagggagattatgggccctggtttgcttactggggccaagggactctggtcactgtctctgcagcgtcgac

SEQ ID NO: 260 - DNA, 345 bp, Escherichia coli
  source 1..345: mol_type = other DNA
accggtgtacattcccaaattgttctcacccagtctccagcaatcatgtctgcatctccaggggaaaaggtcaccatgacctgcagggccagctcaagtgtaagttccagttacttgcactggtaccagcagaagtcaggtgcctcccccaaattctggatttatagtacatccaagttggcttctggagtccctgctcgcttcagtggcagtgggtctgggacctcttactctctcacaatcagcagtgtggaggctgaagatgctgccacttattactgccagcactacagtcgttacccactcacgttcggaggggggaccaagctggaaataaaacgtacg

SEQ ID NO: 261 - DNA, 380 bp, Escherichia coli
  source 1..380: mol_type = other DNA
accggtgtacattccgaggtccagctgcaacagtctggacctgtgctggtgaagcctgggccttcagtgaagatatcctgtaaggcttctggattcacattcactgactactacatacactgggtgaagcagagccatggaaagagccctgagtggattggacttgtttctccttacaatggtggtacttactacaaccagaagttcatgggcagggccacattgactgaagacacatcctccaacacagcctacatggagctaaacagcctgacttctgaggactctgcggtctattactgtgcgaggttactacggggctactggtacttcgatgtctggggcacagggaccacggtcaccgtctcctcagcgtcgac

SEQ ID NO: 262 - DNA, 345 bp, Escherichia coli
  source 1..345: mol_type = other DNA
accggtgtacattcccaaattgttctcacccagtctccagcactcatggctgcatctccaggggagatggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggcgtacttacccgtacacgttcggaggggggaccaagctggaaataaaacgtacg

SEQ ID NO: 263 - DNA, 380 bp, Escherichia coli
  source 1..380: mol_type = other DNA
accggtgtacattccgaggtccagctgcaacagcctggggctgagctggtgaggcctggggcctcagtgaagatgtcctgcaaggcttctggctacacatttaccatttacaatttgcactgggtaaagcagacacctagacagggcctggaatggattggaactatttatccaggagatggtgatacttcctacaatcagaagttcaagggcaaggccacactgactgtagacaaatcctccagcacagcctacatgcagctcagcaacctgacatctgaagactctgcggtctatttctgtgcaagagagggagattatgggccctggtttgcttactggggccaagggactctggtcactgtctctgcagcgtcgac

SEQ ID NO: 264 - DNA, 345 bp, Escherichia coli
  source 1..345: mol_type = other DNA
accggtgtacattcccaaattgttctcacccagtctccagcaatcatgtctgcatctccaggggaaaaggtcaccatgacctgcagggccagctcaagtgtaagttccagttatttgcactggtaccagcagaagtcaggtgcctcccccaaactctggatctatagctcatccaacttggcttctggagtccctgctcgcttcagtggcagtgggtctgggacctcttactctctcacaatcaacagtgtggaggctgaagatgcagccacttattactgccagcactacggtagttacccactcacgttcggaggggggaccaagctggaaataaaacgtacg

SEQ ID NO: 265 - DNA, 374 bp, Escherichia coli
  source 1..374: mol_type = other DNA
accggtgtacattccgaggtccaactgcaacagtctggacctgagctggtgaagcctggggcctcagtgaagatttcctgcaaggcttctggctacgcattcagtagctcctggatgaactgggtgaagcagaggcctggaaagggtcttaagtggattggacggatttatcctggagatggagatactaattacaatgggaagttcaagggcaaggccacactgactgcagacaaatcctccngcacagcctacatgcaactcagcagcctgacatctgaggactctgcggtctacttctgtgcaagagagggtaacttgnattttgactactggggccaaggcaccactctcacagtctcctcagcgtcgac

SEQ ID NO: 266 - DNA, 354 bp, Escherichia coli
  source 1..354: mol_type = other DNA
accggtgtacattccgacattgtgctgacccaatctcctgcttccttagatgtatctctggggcagagggccaccatctcatgcagggccagccaaagtgtcagtacatctacctatagttatatgcactggtaccaacagaaaccaggacagtcacccaaagtcctcatcaagtatgcatccaacctagaagctggggtccctgccagggtcagtggcagtgggtctgggacagacttcaccctcaacatccatcctgtggaggaggaggatactgcaacatattactgtcagcacagttgggagattcctccgacgttcggtggaggcaccaagctggaaatcaaacgtacg

SEQ ID NO: 267 - DNA, 383 bp, Escherichia coli
  source 1..383: mol_type = other DNA
accggtgtacattcccaggtgcagctgaagcagtcaggacctggcctggtggcgccctcacagagcctgtccatcacatgcactgtctctgggttctcattaaccaactatgctataagctgggttcgccagccaccaggaaagggtctggagtggcttggaataatatggactggtggaggcacactttataattcagctctcaaatccagactgagcatcagcaaagacaactccaagagtcaagttttcttaaaaatgaacagtctgcaaactgatgacacagccaggtactactgtgccagagcctactatagtaactacgactggtacttcgatgtctgggacacagggaccacggtcaccgtctcctcagcgtcgac

SEQ ID NO: 268 - DNA, 342 bp, Escherichia coli
  source 1..342: mol_type = other DNA
accggtgtacattccgacatccagatgactcagtctccatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcaatatagtcaggttccgtacacgttcggaggggggaccaagctggaaataaaacgtacg

SEQ ID NO: 269 - DNA, 374 bp, Escherichia coli
  source 1..374: mol_type = other DNA
accggtgtacattccgaggtccaactgcagcagtctggggctgaactggtgaagcctggggcttcagtgaaggtgtcctgcaaggcttctggctactccttcaccaactactgggtgcactgggtgaagcagaggcctggccaaggccttgagtggattggaaggatttattcttctgatggtgattctaattacaatcaaaagttcaagggcaaggccacattgactgtagacaaatcctccagcacagcctacatgcagctcagcagcctgacatctgaggactctgcggtctattactgtgcaatagagggaaacttgtactttgactactggggccaaggcaccactctcacagtctcctcagcgtcgac

SEQ ID NO: 270 - DNA, 354 bp, Escherichia coli
  source 1..354: mol_type = other DNA
accggtgtacattccgacattgtgctgacccaatctcctgcttccttagctgtatctctggggcagagggccaccatctcatgcagggccagccaaagtgtcagtacatctagctatagttatatgcactggtaccaacagaaaccaggacagccgcccaaattcctcatcaagtatgcatccaacctagaatctggggtccctgccaggttcagtggcagtgggtctgggacagacttcaccctcaatatccatcctgtggaggaggaggatactgcaacatattactgtcagcacagttgggagattcctctcacgttcggtgctgggaccaagctgaagctgaaacgtacg

SEQ ID NO: 271 - DNA, 380 bp, Escherichia coli
  source 1..380: mol_type = other DNA
accggtgtacattccgaggtccaactgcaacagcctggacctgtgctggtgaagcctgggccttcagtgaagatatcctgtaaggcttctggatttacattcactgactacttcatacactgggtgaagcagagccatggaaagagccttgagtggattggacttgtttctccttacaatggtggtacttactacaaccagcagttcaagggcaaggccacattgactgtagacacatcctccagcacagcctacatggacctaaacagcctgacttctgaggactctgcggtctattactgtacgaggttactacggggctactggtacttcgatgtctggggcacagggaccacggtcaccgtctcctcagcgtcgac

SEQ ID NO: 272 - DNA, 345 bp, Escherichia coli
  source 1..345: mol_type = other DNA
accggtgtacattcccaaattgttctcacccagtctccagcgctcatggctgcatctccaggggagaaggtcaccatcacctgcagtgtcagctcgagtataggttccagcaacttgcattggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtacttacccgtacacgttcggaggggggaccaagctggaaataaaacgtacg

SEQ ID NO: 273 - DNA, 383 bp, Escherichia coli
  source 1..383: mol_type = other DNA
accggtgtacattcccaggtgcagctgaaggagtcaggacctggcctggtggcgccctcacagagcctgtccatcacatgcactgtctctgggttctcattaaccaactatgctataagctgggttcgccagccaccaggaaagggtctggagtggcttggaataatatggactggtggaggcacactttataattcagctctcaaatccagaccgagcatcagtaaagacaactccaagagtcaagttttcttaaaaatgaacagtctgcaaactgatgacacagccaggtactactgtgccagagcctactatagtaactacgactggtacttcgatgtctgggacacagggaccacggtcaccgtctcctcagcgtcgac

SEQ ID NO: 274 - DNA, 341 bp, Escherichia coli
  source 1..341: mol_type = other DNA
accggtgtacattccgacatccagatgactcagtctccatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctcttaccatcagcaacctggaacctgaagatattgccacttactattgtcagcaatatagtcaggttccgtacacgttcggaggggggaccaagctggaaataaaacgtac

SEQ ID NO: 275 - DNA, 380 bp, Escherichia coli
  source 1..380: mol_type = other DNA
accggtgtacattccgaggtccagctgcaacagtctggacctgtgctggtgaagcctgggccttcagtgaagatatcctgtaaggcttctggattcacattcactgactacttcgtacactgggtgaagcagagccatggaaagagccttgagtggattggacttgtttctccttacaatggtggtacttactacaaccagcagttcaagggcaaggccacattgactgtagacacatcctccagcacagcctacatggacctaaacagcctgacttctgaggactctgcggtctattactgtgcgaggttactacggggctactggtacttcgatgtctggggcacagggaccacggtcaccgtctcctcagcgtcgac

SEQ ID NO: 276 - DNA, 345 bp, Escherichia coli
  source 1..345: mol_type = other DNA
accggtgtacattcccaaattgttctcacccagtctccagcgctcatggctgcatctccaggggagaaggtcaccatcacctgcagtgtcagctcgagtattagttccagcaccttgcactggtaccagcagaagtcagaaacctcccccaaaccctggatttatggcacatccaacctggcttctggagtccctgttcgcttcggtggcagtggatctgggacctcttattctctcacaatcagcagcatggaggctgaagatgctgccacttattactgtcaacagtggagtacttacccgtacacgttcggaggggggaccaagctggaaataaaacgtacg

SEQ ID NO: 277 - protein, 160 aa, Escherichia coli
FSCNVDGGSSIGAGTTSVYVNLDPVIQPGQNLVVDLSQHISCWNDYGGWYDTDHINLVQGSAFAGSLQSYKGSLYWNNVTYPFPLTTNTNVLDIGDKTPMPLPLKLYITPVGAAGGVVIKAGEVIARIHMYKIATLGSGNPRNFTWNIISNNSVVMPTGG

SEQ ID NO: 278 - protein, 160 aa, Escherichia coli
FACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 279 - protein, 160 aa, Escherichia coli
FACKTATGATIPIGGGSANVYVNLTPAVNVGQNLVVDLSTQIFCHNDYPETITDVVTLQRGSAYGGVLSSFSGTVKYNGTSYPFPTTTETARVIYDSRTDKPWPAVLYLTPVSTAGGVAITAGSLIAVLILHQTNNYNSDSFQFIWNIYANNDVVVPTGG

SEQ ID NO: 280 - RNA, 31 nt, synthetic construct
  source 1..31: mol_type = other RNA
gggaattcgaggtgcagctgcaggagtctgg

SEQ ID NO: 281 - RNA, 24 nt, synthetic construct
  source 1..24: mol_type = other RNA
agggggctctcgcaggagacgagg

SEQ ID NO: 282 - RNA, 23 nt, synthetic construct
  source 1..23: mol_type = other RNA
gaaagttcacggtggttatatcc

SEQ ID NO: 283 - RNA, 31 nt, synthetic construct; identical to SEQ ID NO: 280
  source 1..31: mol_type = other RNA

SEQ ID NO: 284 - RNA, 24 nt, synthetic construct
  source 1..24: mol_type = other RNA
agggggaagacatttgggaaggac

SEQ ID NO: 285 - RNA, 25 nt, synthetic construct
  source 1..25: mol_type = other RNA
tgccgaaagggaagtaatcgtgaat

SEQ ID NO: 286 - RNA, 62 nt, synthetic construct
  source 1..62: mol_type = other RNA
atcctttttctagtagcaactgcaaccggtgtacattccgaggtccarctgcarcagyctgg

SEQ ID NO: 287 - RNA, 57 nt, synthetic construct
  source 1..57: mol_type = other RNA
cctttttctagtagcaactgcaaccggtgtacattcccaggtgcagctgaagsagtc

SEQ ID NO: 288 - RNA, 59 nt, synthetic construct
  source 1..59: mol_type = other RNA
cctttttctagtagcaactgcaaccggtgtacattccgaagtgaagcttgargwgtctg

SEQ ID NO: 289 - RNA, 55 nt, synthetic construct
  source 1..55: mol_type = other RNA
cctttttctagtagcaactgcaaccggtgtacattccgaggttcagctgcagcag

SEQ ID NO: 290 - RNA, 46 nt, synthetic construct
  source 1..46: mol_type = other RNA
gaagaccgatgggcccttggtcgacgctgaggagacggtgaccgtg

SEQ ID NO: 291 - RNA, 43 nt, synthetic construct
  source 1..43: mol_type = other RNA
gaagaccgatgggcccttggtcgacgctgaggagactgtgaga

SEQ ID NO: 292 - RNA, 47 nt, synthetic construct
  source 1..47: mol_type = other RNA
gaagaccgatgggcccttggtcgacgctgcagagacagtgaccagag

SEQ ID NO: 293 - RNA, 46 nt, synthetic construct
  source 1..46: mol_type = other RNA
gaagaccgatgggcccttggtcgacgctgaggagacggtgactgag

SEQ ID NO: 294 - RNA, 21 nt, synthetic construct
  source 1..21: mol_type = other RNA
atgccatagcattttttatcc

SEQ ID NO: 295 - RNA, 18 nt, synthetic construct
  source 1..18: mol_type = other RNA
gatttaatctgtatcagg

SEQ ID NO: 296 - RNA, 24 nt, synthetic construct
  source 1..24: mol_type = other RNA
gcctgtgataccgccaatggtacc

SEQ ID NO: 297 - RNA, 23 nt, synthetic construct
  source 1..23: mol_type = other RNA
ggcggtatcacaggcgaatgacc

SEQ ID NO: 298 - RNA, 24 nt, synthetic construct
  source 1..24: mol_type = other RNA
gcctgtaaaaccgccaaaggtacc

SEQ ID NO: 299 - RNA, 24 nt, synthetic construct
  source 1..24: mol_type = other RNA
gatagcggtacctttggcggtttt

SEQ ID NO: 300 - RNA, 25 nt, synthetic construct
  source 1..25: mol_type = other RNA
ggtaccgatatccctattggcggtg

SEQ ID NO: 301 - RNA, 27 nt, synthetic construct
  source 1..27: mol_type = other RNA
gccaatagggatatcggtaccattggc

SEQ ID NO: 302 - RNA, 24 nt, synthetic construct
  source 1..24: mol_type = other RNA
gctatccctaaaggcggtggcagc

SEQ ID NO: 303 - RNA, 22 nt, synthetic construct
  source 1..22: mol_type = other RNA
ccaccgcctttagggatagcgg

SEQ ID NO: 304 - RNA, 33 nt, synthetic construct
  source 1..33: mol_type = other RNA
ggtggcaaagccaatgtttatgtaaaccttgcg

SEQ ID NO: 305 - RNA, 36 nt, synthetic construct
  source 1..36: mol_type = other RNA
gtttacataaacattggctttgccaccgccaatagg

SEQ ID NO: 306 - RNA, 28 nt, synthetic construct
  source 1..28: mol_type = other RNA
gcagcgccaaagtttatgtaaaccttgc

SEQ ID NO: 307 - RNA, 27 nt, synthetic construct
  source 1..27: mol_type = other RNA
gtttacataaactttggcgctgccacc

SEQ ID NO: 308 - RNA, 27 nt, synthetic construct
  source 1..27: mol_type = other RNA
gccaatgttgatgtaaaccttgcgccc

SEQ ID NO: 309 - RNA, 27 nt, synthetic construct
  source 1..27: mol_type = other RNA
gtttacatcaacattggcgctgccacc

SEQ ID NO: 310 - RNA, 27 nt, synthetic construct
  source 1..27: mol_type = other RNA
gtttatgtaaaacttgcgcccgtcgtg

SEQ ID NO: 311 - RNA, 30 nt, synthetic construct
  source 1..30: mol_type = other RNA
gacgggcgcaagttttacataaacattggc

SEQ ID NO: 312 - RNA, 30 nt, synthetic construct
  source 1..30: mol_type = other RNA
gtaaaccttgatcccgtcgtgaatgtgggg

SEQ ID NO: 313 - RNA, 33 nt, synthetic construct
  source 1..33: mol_type = other RNA
cacgacgggatcaaggtttacataaacattggc

SEQ ID NO: 314 - RNA, 20 nt, synthetic construct
  source 1..20: mol_type = other RNA
ccttgcgcccgacgtgaatg

SEQ ID NO: 315 - RNA, 19 nt, synthetic construct
  source 1..19: mol_type = other RNA
ccacattcacgtcgggcgc

SEQ ID NO: 316 - RNA, 19 nt, synthetic construct
  source 1..19: mol_type = other RNA
gcccgtcgtgaaagtgggg

SEQ ID NO: 317 - RNA, 22 nt, synthetic construct
  source 1..22: mol_type = other RNA
ggttttgccccactttcacgac

SEQ ID NO: 318 - RNA, 24 nt, synthetic construct
  source 1..24: mol_type = other RNA
cccgtcgtgaatgatgggcaaaac

SEQ ID NO: 319 - RNA, 25 nt, synthetic construct
  source 1..25: mol_type = other RNA
cgaccaggttttgcccatcattcac

SEQ ID NO: 320 - RNA, 26 nt, synthetic construct
  source 1..26: mol_type = other RNA
cgtgaatgtggggcaaaaactggtcg

SEQ ID NO: 321 - RNA, 27 nt, synthetic construct
  source 1..27: mol_type = other RNA
cgaaagatccacgaccagtttttgccc

SEQ ID NO: 322 - RNA, 19 nt, synthetic construct
  source 1..19: mol_type = other RNA
cctggtcgtgaaactttcg

SEQ ID NO: 323 - RNA, 19 nt, synthetic construct
  source 1..19: mol_type = other RNA
gcgtcgaaagtttcacgac

SEQ ID NO: 324 - RNA, 22 nt, synthetic construct
  source 1..22: mol_type = other RNA
cgtggatctttcgaagcaaatc

SEQ ID NO: 325 - RNA, 22 nt, synthetic construct
  source 1..22: mol_type = other RNA
ggcaaaagatttgcttcgaaag

SEQ ID NO: 326 - RNA, 24 nt, synthetic construct
  source 1..24: mol_type = other RNA
cgcaaatcgattgccataacgatt

SEQ ID NO: 327 - RNA, 24 nt, synthetic construct
  source 1..24: mol_type = other RNA
ggcaatcgatttgcgtcgaaagat

SEQ ID NO: 328 - RNA, 23 nt, synthetic construct
  source 1..23: mol_type = other RNA
gccataacgatgatccggaaacc

SEQ ID NO: 329 - RNA, 29 nt, synthetic construct
  source 1..29: mol_type = other RNA
ctgtaatggtttccggatcatcgttatgg

SEQ ID NO: 330 - RNA, 28 nt, synthetic construct
  source 1..28: mol_type = other RNA
gccataacgattatccgaaaaccattac

SEQ ID NO: 331 - RNA, 25 nt, synthetic construct
  source 1..25: mol_type = other RNA
gtctgtaatggttttcggataatcg

SEQ ID NO: 332 - RNA, 24 nt, synthetic construct
  source 1..24: mol_type = other RNA
ccattacagacgatgtcacactgc

SEQ ID NO: 333 - RNA, 24 nt, synthetic construct
  source 1..24: mol_type = other RNA
gtgacatcgtctgtaatggtttcc

SEQ ID NO: 334 - RNA, 28 nt, synthetic construct
  source 1..28: mol_type = other RNA
cactgcaaaaaggctcggcttatggcgg

SEQ ID NO: 335 - RNA, 29 nt, synthetic construct
  source 1..29: mol_type = other RNA
gccgagcctttttgcagtgtgacatagtc

SEQ ID NO: 336 - RNA, 22 nt, synthetic construct
  source 1..22: mol_type = other RNA
gcaacgaggcaaggcttatggc

SEQ ID NO: 337 - RNA, 24 nt, synthetic construct
  source 1..24: mol_type = other RNA
gccgttcctcgttgcagtgtgaca

SEQ ID NO: 338 - RNA, 23 nt, synthetic construct
  source 1..23: mol_type = other RNA
ctcggctgatggcggcgtgttat

SEQ ID NO: 339 - RNA, 22 nt, synthetic construct
  source 1..22: mol_type = other RNA
ccatcagccgagcctcgttgca

SEQ ID NO: 340 - RNA, 27 nt, synthetic construct
  source 1..27: mol_type = other RNA
ggcggcgatttatctaatttttccggg

SEQ ID NO: 341 - RNA, 27 nt, synthetic construct
  source 1..27: mol_type = other RNA
ttagataaatcgccgccataagccgag

SEQ ID NO: 342 - RNA, 23 nt, synthetic construct
  source 1..23: mol_type = other RNA
gttatctaaattttccgggaccg

SEQ ID NO: 343 - RNA, 23 nt, synthetic construct
  source 1..23: mol_type = other RNA
ggaaaatttagataacacgccgc

SEQ ID NO: 344 - RNA, 23 nt, synthetic construct
  source 1..23: mol_type = other RNA
ccgggaaagtaaaatatagtggc

SEQ ID NO: 345 - RNA, 28 nt, synthetic construct
  source 1..28: mol_type = other RNA
ctatattttactttcccggaaaaattag

SEQ ID NO: 346 - RNA, 29 nt, synthetic construct
  source 1..29: mol_type = other RNA
ccgggaccgtagattatagtggcagtagc

SEQ ID NO: 347 - RNA, 32 nt, synthetic construct
  source 1..32: mol_type = other RNA
gccactataatctacggtcccggaaaaattag

SEQ ID NO: 348 - RNA, 27 nt, synthetic construct
  source 1..27: mol_type = other RNA
cgtaaaatataaaggcagtagctatcc

SEQ ID NO: 349 - RNA, 23 nt, synthetic construct
  source 1..23: mol_type = other RNA
ctgcctttatattttacggtccc

SEQ ID NO: 350 - RNA, 22 nt, synthetic construct
  source 1..22: mol_type = other RNA
gtggcaaaagctatccatttcc

SEQ ID NO: 351 - RNA, 28 nt, synthetic construct
  source 1..28: mol_type = other RNA
ggatagcttttgccactatattttacgg

SEQ ID NO: 352 - RNA, 21 nt, synthetic construct
  source 1..21: mol_type = other RNA
cctaccaaaagcgaaacgccg

SEQ ID NO: 353 - RNA, 24 nt, synthetic construct
  source 1..24: mol_type = other RNA
cgtttcgcttttggtaggaaatgg

SEQ ID NO: 354 - RNA, 22 nt, synthetic construct
  source 1..22: mol_type = other RNA
cctaccaaaagcgaaacgccgc

SEQ ID NO: 355 - RNA, 22 nt, synthetic construct
  source 1..22: mol_type = other RNA
cggcgttttgctggtggtagga

SEQ ID NO: 356 - RNA, 23 nt, synthetic construct
  source 1..23: mol_type = other RNA
cgccggacgttgtttataattcg

SEQ ID NO: 357 - RNA, 22 nt, synthetic construct
  source 1..22: mol_type = other RNA
taaacaacgtccggcgtttcgc

SEQ ID NO: 358 - RNA, 24 nt, synthetic construct
  source 1..24: mol_type = other RNA
gcgttgtttataaatcgagaacgg

SEQ ID NO: 359 - RNA, 25 nt, synthetic construct
  source 1..25: mol_type = other RNA
ccgttctcgatttataaacaacgcg

SEQ ID NO: 360 - RNA, 27 nt, synthetic construct
  source 1..27: mol_type = other RNA
gtttataattcgaaaacggataagccg

SEQ ID NO: 361 - RNA, 29 nt, synthetic construct
  source 1..29: mol_type = other RNA
gcttatccgttttcgaattataaacaacg

SEQ ID NO: 362 - RNA, 27 nt, synthetic construct
  source 1..27: mol_type = other RNA
gtttataattcgagaaaggataagccg

SEQ ID NO: 363 - RNA, 27 nt, synthetic construct
  source 1..27: mol_type = other RNA
cggcttatcctttctcgaattataaac

SEQ ID NO: 364 - RNA, 25 nt, synthetic construct
  source 1..25: mol_type = other RNA
gagaacggatgatccgtggccggtg

SEQ ID NO: 365 - RNA, 26 nt, synthetic construct
  source 1..26: mol_type = other RNA
ccggccacggatcatccgttctcgaa

SEQ ID NO: 366 - RNA, 28 nt, synthetic construct
  source 1..28: mol_type = other RNA
ggtggcgctttatttgaagcctgtgagc

SEQ ID NO: 367 - RNA, 28 nt, synthetic construct
  source 1..28: mol_type = other RNA
cgcactgctcacaggcttcaaataaagc

SEQ ID NO: 368 - RNA, 27 nt, synthetic construct
  source 1..27: mol_type = other RNA
ggcgattgatgctggctcattaattgc

SEQ ID NO: 369 - RNA, 21 nt, synthetic construct
  source 1..21: mol_type = other RNA
gagccagcatcaatcgccacc

SEQ ID NO: 370 - RNA, 27 nt, synthetic construct
  source 1..27: mol_type = other RNA
cattaattgccgatcttattttgcgac

SEQ ID NO: 371 - RNA, 27 nt, synthetic construct
  source 1..27: mol_type = other RNA
caaaataagatcggcaattaatgagcc

SEQ ID NO: 372 - RNA, 25 nt, synthetic construct
  source 1..25: mol_type = other RNA
gcttattttgaaacagaccaacaac

SEQ ID NO: 373 - RNA, 24 nt, synthetic construct
  source 1..24: mol_type = other RNA
ggtctgtttcaaaataagcacggc

SEQ ID NO: 374 - RNA, 25 nt, synthetic construct
  source 1..25: mol_type = other RNA
caactataacaaagatgatttccag

SEQ ID NO: 375 - RNA, 27 nt, synthetic construct
  source 1..27: mol_type = other RNA
ggaaatcatctttgttatagttgttgg

SEQ ID NO: 376 - RNA, 31 nt, synthetic construct
  source 1..31: mol_type = other RNA
ctataacagcgatgatgaccagtttgtgtgg

SEQ ID NO: 377 - RNA, 31 nt, synthetic construct
  source 1..31: mol_type = other RNA
cacaaactggtcatcatcgctgttatagttg

SEQ ID NO: 378 - RNA, 26 nt, synthetic construct
  source 1..26: mol_type = other RNA
tttccagtttgattggaatatttacg

SEQ ID NO: 379 - RNA, 27 nt, synthetic construct
  source 1..27: mol_type = other RNA
gtaaatattccaatcaaactggaaatc

SEQ ID NO: 380 - RNA, 22 nt, synthetic construct
  source 1..22: mol_type = other RNA
cgccaataaagatgtggtggtg

SEQ ID NO: 381 - RNA, 22 nt, synthetic construct
  source 1..22: mol_type = other RNA
caccacatctttattggcgtaa

SEQ ID NO: 382 - RNA, 29 nt, synthetic construct
  source 1..29: mol_type = other RNA
ccaataatgatgtggatgtgcctactggc

SEQ ID NO: 383 - RNA, 27 nt, synthetic construct
  source 1..27: mol_type = other RNA
gtaggcacatccacatcattattggcg

(19) United States
(12) Patent Application Publication
(10) Pub. No.: US 2025/0304662 A1
(43) Pub. Date: Oct. 2, 2025

(54) FIMH INHIBITING COMPOSITIONS AND METHODS OF USE THEREOF

(71) Applicant: Washington University, St. Louis, MO (US)

(72) Inventors: Scott Hultgren, St. Louis, MO (US); Ali Ellebedy, St. Louis, MO (US); Edward Lopatto, St. Louis, MO (US); Karen Dodson, St. Louis, MO (US); Jerome Pinkner, St. Louis, MO (US); Morgan Timm, St. Louis, MO (US); Shaza Sayed Ahmed, St. Louis, MO (US); Isabella Fox, St. Louis, MO (US); Denise Sanick, St. Louis, MO (US); Nathaniel Gualberto, St. Louis, MO (US); Andrew Kau, St. Louis, MO (US); Jesus Santiago-Borges, St. Louis, MO (US); Aaron Schmitz, St. Louis, MO (US); Jackson Turner, St. Louis, MO (US)

(73) Assignee: Washington University, St. Louis, MO (US)

(21) Appl. No.: 19/097,433

(22) Filed: Apr. 1, 2025

Related U.S. Application Data
(60) Provisional application No. 63/572,509, filed on Apr. 1, 2024.

(51) Int. Cl. C07K 16/12; A61P 31/04; G01N 33/569

(52) U.S. Cl. CPC C07K 16/1232; A61P 31/04; C07K 16/1228; G01N 33/56916; G01N 2333/245; G01N 2333/26

(57) ABSTRACT

Among the various aspects of the present disclosure is the provision of FimH inhibiting compositions and methods of use thereof. FimH inhibiting compositions that target and inhibit FimH, including monoclonal antibodies, are described. Methods of identifying FimH inhibiting antibodies are also described. Further, a method of treating bacterial infections, including urinary tract infections, is described.

### CROSS-REFERENCE TO RELATED APPLICATIONS

This application claims the benefit of priority to U.S. Provisional Application Ser. No. 63/572,509 filed on Apr. 1, 2024, which is incorporated herein by reference in its entirety.

### STATEMENT REGARDING FEDERALLY SPONSORED RESEARCH OR DEVELOPMENT

This invention was made with government support under AI157797 and AI165915 awarded by the National Institutes of Health (NIH). The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name “020501-US-NP_SEQ_LISTING” created on 27 Mar. 2025; 544,225 bytes). The subject matter of the Sequence Listing is incorporated herein by reference in its entirety.

### FIELD OF THE INVENTION

The present disclosure generally relates to FimH inhibiting compositions and methods of use thereof.

### BACKGROUND OF THE INVENTION

Urinary tract infections (UTIs) afflict millions of people annually in the US alone and the two most prevalent UTI-causing pathogens are Escherichia coli and Klebsiella pneumoniae. Many UTIs are catheter-associated and come with an increased risk of severity and morbidity.

Uropathogenic Escherichia coli (UPEC) and Klebsiella pneumoniae are two primary causative agents of urinary tract infections (UTIs). Moreover, UPEC and K. pneumoniae are becoming alarmingly antibiotic-resistant, making these infections increasingly difficult to treat and new therapeutics are needed. UPEC and K. pneumoniae possess type 1 pili tipped with the mannose binding adhesin protein FimH which allows the pathogens to infect the urinary tract. The type 1 pilus is a prototypical example of a chaperone-usher pathway (CUP) pilus, requiring chaperone and usher proteins for assembly. 7 Pilin subunits are assembled into the pilus at the outer membrane usher. The FimC chaperone, which donates a β-strand to complete the immunoglobulin fold of the subunits, is replaced by a donor strand exchange (DSE) interaction with the N terminal extension of the next subunit. At the tip of the type 1 pilus is the adhesion FimH. These pili are essential virulence factors for UTIs and allow the pathogen to bind to exposed mannose residues on the surface of epithelial cells. After attachment to host cells, both UPEC and K. pneumoniae invade these tissues, replicate, and form clonal intracellular bacterial communities of >10⁴ bacteria while shielded from much of the host immune response and non-host cell permeant antibiotics.

### SUMMARY OF THE INVENTION

Among the various aspects of the present disclosure is the provision of FimH inhibiting compositions and methods of use thereof.

Briefly, therefore, the present disclosure is directed to compositions to treat a bacterial infection and methods of identifying compounds to treat a bacterial infection.

The present teachings include a composition comprising an agent that targets a FimH adhesion protein. In one aspect, the composition can be an antibody. In another aspect, the composition can target the FimH adhesin protein of K. pneumonia and E. coli bacteria. In another aspect, the composition can target the lectin domains of the FimH adhesin protein. In another aspect, the antibody comprises a heavy chain (HC) protein variable region and a light chain (LC) regio with the amino acid sequence independently selected from the sequences found in Table 2. In another aspect, the antibody comprises a heavy chain protein variable region and a light chain protein region encoded by a nucleotide sequence independently selected from the sequences found in Table 1. In another aspect, the antibody comprises a heavy chain protein and a light chain protein encoded by a nested nucleotide sequence independently selected from the sequences found in Table 3. In another aspect, the antibody comprises a heavy chain protein and a light chain protein encoded by a plasmid nucleotide sequence independently selected from the sequences found in Table 4. In another aspect, the composition can be used to treat a urinary tract infection (UTI).

The present teachings also include a method for identifying an antibody to treat a bacterial infection; the method can include performing an ELISA binding assay with monoclonal antibodies to identify antibodies that inhibit FimH binding in vitro. In another aspect, the ELISA assay can be performed using bacteria comprising E. coli and K. pneumonia FimH proteins to identify antibodies to treat a bacterial infection. In another aspect, the method can identify an antibody to treat a UTI. In another aspect, the method can further include administering antibodies identified in vitro to murine UTI models to characterize protection against UTI in vivo.

The present teachings also include a method of treating a bacterial infection. In one aspect, the method can include administering a therapeutically effective amount of a composition that targets and inhibits FimH. In another aspect, the treated bacterial infection can be a UTI. In another aspect, the composition can be an antibody. In another aspect, the antibody binds to a lectin domain of FimH. In another aspect, the antibody comprises a heavy chain protein variable region with the amino acid sequence selected from SEQ ID NOS: 241, 243, 245, 247, 249, 251, 253, 255, and 257 and the light chain protein variable region comprises the amino acid sequence independently selected from SEQ ID NOS: 242, 244, 246, 248, 250, 252, 254, 256, and 258. In another aspect, the antibody comprises a heavy chain protein variable region encoded by a nucleotide sequence selected from 259, 261, 263, 265, 267, 269, 271, 273, and 275, and the light chain protein variable region is encoded by a nucleotide sequence independently selected from SEQ ID NOS: 260, 262, 264, 266, 268, 270, 272, 274, and 276.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is a schematic showing the structure of E. coli FimHLD (PDB 1KLF).

FIG. 1B is a schematic showing the structure of K. pneumoniae FimHLD (PDB 9AT9).

FIG. 1C is a schematic showing the structure of E. coli FimHLD (PDB 6AOW).

FIG. 1D is a chart showing ELISA EC50 values for each mAb to the listed protein. White cells with no values indicate EC50 values were above the range measured in the assay.

FIG. 1E is a set of schematics of E. coli FimHLD and a corresponding chart showing epitope mapping of mAbs (top labels) to a panel of FimH mutants (right labels). Binding classes were determined by shared residues that abrogated mAb binding which are highlighted in purple on the surface of E. coli FimHLD (above) (PDB 1KLF) and the table (below).

FIG. 2A is a cryo-EM density map of the Fab Kp1 2H04 (teal) complexed with FimHLD (red).

FIG. 2B is a cryo-EM density map of the Fab Ec1 E7 (cyan) complexed with FimHLD (red).

FIG. 2C is a cryo-EM density map of the Fabs Kp1 2H04 (teal) and Ec1 E7 (cyan) maps superimposed on each other and complexed with FimHLD (red).

FIG. 2D is a s cryo-EM density map of the Fab Kp2 2C07 (yellow) complexed with FimHLD (red).

FIG. 2E is a cryo-EM density map of the Fab Ec3 B7 (green complexed with FimHLD (red).

FIG. 2F is a schematic showing the binding epitopes of the Fab Kp1 2H04 (teal) on FimHLD.

FIG. 2G is a schematic showing the binding epitopes of the Fab Ec1 E7 (cyan) on FimHLD.

FIG. 2H is a schematic showing the binding epitopes of the Fab Kp2 2C07 (yellow) on FimHLD.

FIG. 2I is a schematic showing the binding epitopes of the Fab Ec3 B7 (green) on FimHLD.

FIG. 2J is a schematic of a density map overlaid on model residue interactions of Kp1 2H04 (cyan) with FimH P26. mAb heavy chain residues are labeled “HC” and light chain residues are labeled “LC”.

FIG. 2K is a schematic of a density map overlaid on model residue interactions of Ec3 B7 (green) with FimH Y64. mAb heavy chain residues are labeled “HC” and light chain residues are labeled “LC”.

FIG. 3A is a graph showing FimH mAb binding to UT189 (black) bacteria and the FimS LIR mutants UT189 LON (pink) and UT189 LON ΔfimH (teal).

FIG. 3B is a graph showing UT189 overexpressing conformationally shifted FimH variants: UT189 LON FimH A27V/V163A (ΔfimBE; pink), UT189 LON FimH WT ((ΔfimBE; black), and UT189 LON FimH A62S (ΔfimBE; teal). n=3, error bars represent SEM.

FIG. 3C is a set of graphs showing representative binding curves of Kp1 2H04, Ec1 F7, and Kp2 2C07 Fab binding FimHLD (left) and FimGNTE (right). Results are from kinetic measurements of dilution series (500 nM, 250 nM, 125 nM, 62.5 nM, 31.2 nM) of one experiment.

FIG. 3D is a chart showing the observed biolayer interferometry (BLI) binding kinetics to Ec FimGNTEH and Ec FimHLD. N.D. means not determined due to the off rate being below the detection limit.

FIG. 4A is a chart showing inhibition of FimHLD binding to BSM at a 5:1 molar ratio of mAb to protein (n≥3).

FIG. 4B is a graph showing the inhibition of UT189 guinea pig erythrocyte hemagglutination (n≥2).

FIG. 4C is a set of representative immunofluorescent images showing mAb inhibition of FimHLD binding to C3H/HeN mouse bladders. mAb was pre-incubated with FimHLD at a 10:1 molar ratio. Sections were stained with DNA dye Hoechst (blue), Ec FimHLD (red) and antibody to uroplakin III (green). n=4-5 bladder sections with n=2 technical replicates.

FIG. 4D is a set of representative immunofluorescent images showing mAb inhibition of FimHLD binding to C3H/HeN mouse bladders. mAb was pre-incubated with FimHLD at a 10:1 molar ratio. Sections were stained with DNA dye Hoechst (blue), Kp FimHLD (red) and antibody to uroplakin III (green). n=4-5 bladder sections with n=2 technical replicates.

FIG. 5A is a schematic showing an experimental timeline. 6-7 week old C3H/HeN mice were pretreated with 0.5 mg of mAb 24 h before infection with UT189.

FIG. 5B is a graph of UT189 CFU in the bladder 24 hpi after infection. For 1B03 and 2E08 n=5 with 1 independent replicate, for B7 n=8 with 1 independent replicate, for 2C07 n=10 with 2 independent replicates, for control IgG, F7, 2H04, 1A02, 2E02 n=13-33 with three independent replicates. Statistical comparisons were made using Kruskal-Wallis test (nonparametric ANOVA) with Dunn's comparisons to the control group correcting for multiple comparisons. *P≤0.05, **P≤0.01.

FIG. 5C is a graph of UT189 CFU in the kidney 24 hpi after infection. For 1B03 and 2E08 n=5 with 1 independent replicate, for B7 n=8 with 1 independent replicate, for 2C07 n=10 with 2 independent replicates, for control IgG, F7, 2H04, 1A02, 2E02 n=13-33 with three independent replicates. Statistical comparisons were made using Kruskal-Wallis test (nonparametric ANOVA) with Dunn's comparisons to the control group correcting for multiple comparisons. *P≤0.05, **P≤0.01.

FIG. 5D is a graph of IBC counts at 6 hpi (n=16 for control IgG, n=14 for Kp1 2H04 with two independent replicates) of IBCs (green) in splayed mouse bladders for control IgG and Kp1 2H04 treatments. A Mann-Whitney U test was used to evaluate statistical significance.

FIG. 5E is a representative fluorescent image of IBC counts at 6 hpi. 5× magnification images of IBCs (green) in splayed mouse bladders for control IgG treatments. Scale bar represents 200 μm.

FIG. 5F is a representative fluorescent image of IBC counts at 6 hpi. 5× magnification images of IBCs (green) in splayed mouse bladders for Kp1 2H04 treatments. Scale bar represents 200 μm.

FIG. 5G is a graph showing CFU in the bladder 24 hpi from the prophylactic model testing control IgG, 2H04, and 2H04_{LALAPG} (n=21 for control group, n=20 for 2H04, and n=19 for 2H04_{LALAPG} with three independent replicates). Statistical comparisons were made using Kruskal-Wallis test (nonparametric ANOVA) with Dunn's comparisons to the control group correcting for multiple comparisons. **P≤0.01, ****P≤0.00001.

FIG. 5H is a graph showing CFU in the kidneys 24 hpi from the prophylactic model testing control IgG, 2H04, and 2H04_{LALAPG} (n=21 for control group, n=20 for 2H04, and n=19 for 2H04LALAPG with three independent replicates). Statistical comparisons were made using Kruskal-Wallis test (nonparametric ANOVA) with Dunn's comparisons to the control group correcting for multiple comparisons. **P≤0.01, ****P≤0.00001.

FIG. 6A is a schematic of structural regions of FimHLD in the relaxed conformation (PDB 5jr4). The regions are colored as follows: swing loop in blue (residues 22-35) and linker to pilin domain in orange (residues 157-160).

FIG. 6B is a schematic of structural regions of FimHLD in the tense conformation (PDB 5jqi). The regions are colored as follows: clamp loop in limon (residues 8-16), peripheral α-helix in green (residues 59-72), and insertion loop in magenta (residues 109-124).

FIG. 6C is a topology diagram of FimHLD with β-sheet lettered in dark blue.

FIG. 6D is an image of amino acid sequence alignment of E. coli FmIHLD (EcFmIH), E. coli FimHLD (EcFimH), and K. pneumoniae FimHLD (KpFimH).

FIG. 7A is an image of a local resolution map of the Kp1 2H04-FimH complex, colored from high resolution (red) to low resolution (blue).

FIG. 7B is an image of a local resolution map of the Ec3 B7-FimH complex, colored from high resolution (red) to low resolution (blue).

FIG. 7C is an image of a local resolution map of the Ec1 F7-FimH complex, colored from high resolution (red) to low resolution (blue).

FIG. 7D is an image of a local resolution map of the Kp2 2C07-FimH complex, colored from high resolution (red) to low resolution (blue).

FIG. 8 is a flow diagram of the cryoEM processing of Fabs-FimH complexes. For each Fab-FimH complex, data processing is tracked by arrows starting at the top of the diagram and finishing with final density maps at the bottom.

FIG. 9A is a cryo-EM density map showing a ˜20° binding angle shift in the Kp1 2H04 (teal) and Ec1 F7 (cyan) Fabs relative to the binding site on FimH (red). Bars represent a 20° angle.

FIG. 9B is a schematic showing Ec1 F7 (cyan) coordinated to multiple aromatic residues around FimH (red). Density map is overlaid on model. mAb heavy chain residues are labeled “HC” and light chain residues are labeled “LC”.

FIG. 9C is a cryo-EM density map showing composite surface models of Kp1 2H04 (teal), Ec1 F7 (cyan), Kp2 2C07 (yellow), and Ec3 B7 (green) Fabs on FimH (red).

FIG. 10 is a Western blot showing anti-type 1 pili (FimA ˜17 kDa) to cell lysates used in bacterial cell ELISA (normalized by OD600).

FIG. 11A is a set of graphs showing BLI binding curves of Fabs to E. coli FimGnteH. Results are from kinetic measurements of dilution series of one experiment.

FIG. 11B is a set of graphs showing BLI binding curves of Fabs to E. coli FimGnteH. Results are from kinetic measurements of dilution series of one experiment.

FIG. 12A is a set of graphs showing BLI binding curves of Fabs to E. coli FimLD. Results are from kinetic measurements of dilution series of one experiment.

FIG. 12B is a set of graphs showing BLI binding curves of Fabs to E. coli FimLD. Results are from kinetic measurements of dilution series of one experiment.

FIG. 13 is a graph showing the presence of FimH mAbs in urine during cystitis. 0.5 mg of selected mAbs were injected via IP. Levels of hIgG mAbs in urine (1:10 dilution) at −24 (Pre-IP), 0 (Pre-infection), 3, 6 and 24 hrs for each of the FimH-specific mAbs and IgG control (n=3 to 5 per group). Mann-Whitney test used to determine significance.

FIG. 14 is a graph showing ELISA binding of Kp1 2H04 (black) and Kp1 2H04LALAPG (pink) mAbs to E. coli FimHLD (n=3).

FIG. 15A is a graph showing the presence of protective Kp1 2H04 in serum two weeks after intraperitoneal injection. Titers in urine were measured during the two-week infection period. Horizontal dashed lines represent limit of detection (LOD) of UT189 titers in urine and tissues.

FIG. 15B is a graph showing the presence of protective Kp1 2H04 in serum two weeks after intraperitoneal injection. Bacterial loads in bladder and kidney tissues (B) were assessed at sacrifice from mice pretreated with 0.5 mg of Kp1 2H04 (n=10) or control IgG (n=10) 24 hours before infection with UT189. Horizontal dashed lines represent limit of detection (LOD) of UT189 titers in urine and tissues.

FIG. 15C is a graph showing the presence of protective Kp1 2H04 in serum two weeks after intraperitoneal injection. hIgG mAb levels in serum (C, 1:100 dilution) of mice pretreated with 0.5 mg of hIgG mAb before infection. Mann-Whitney test was used to determine significance.

FIG. 15D is a graph showing the presence of protective Kp1 2H04 in serum two weeks after intraperitoneal injection. hIgG mAb levels in urine (1:10 dilution), and bladder (at sacrifice in day 14) homogenates (1:2 dilution) of mice pretreated with 0.5 mg of hIgG mAb before infection. Mann-Whitney test was used to determine significance.

FIG. 16A contains a top and side view of a cryoEM density map of singular uroplakin complex (singular unit in blue; EMD-36340).

FIG. 16B contains a top and side view of a cryoEM density map of singular uroplakin complex (singular unit in blue; EMD-36340) modeled with bound FimH lectin domain (red). FimH is modeled to bind to a glycan off N169. Structural clashes between uroplakin complex density and Fab model are noted, despite FimH being in a high-affinity state.

FIG. 16C contains a top and side view of a cryoEM density map of singular uroplakin complex (singular unit in blue; EMD-36340) modeled with bound FimH lectin domain (red) and FimH lectin domain-Kp1 2H04 Fab complex (Kp1 2H04 Fab; teal). FimH is modeled to bind to a glycan off N169. Structural clashes between uroplakin complex density and Fab model are noted, despite FimH being in a high-affinity state.

FIG. 17A is a graph showing the binding curves of the top Kp FimH mAbs.

FIG. 17B is a graph showing the binding curves of the E. coli mAb F7.

FIG. 17C is a chart showing that Kp FimH mAbs bind with high affinity to Kp and E. coli y1 CUP adhesins.

FIG. 17D is a chart showing the binding affinity of E. coli mAbs.

FIG. 18A is a graph showing the in vitro inhibition of Kp Fim1033 (black) and Kp 2H04 (green).

FIG. 18B is a graph showing the in vitro inhibition of Kp Fim1033 (black) and 1E04 (red).

FIG. 18C is a chart showing E. coli FimHLD inhibition at a 5:1 (mAb:FimH) molar ratio.

FIG. 18D is a chart showing TOP52 FimHLD inhibition at a 5:1 (mAb:FimH) molar ratio.

FIG. 19A is a graph showing the in vitro inhibition of E. coli mAbs.

FIG. 19B is a chart showing T2b inhibition at a 5:1 (mAb:FimH) molar ratio.

FIG. 19C is a chart showing TOP52 FimHLD inhibition at a 5:1 (mAb:FimH) molar ratio.

FIG. 20 is a graph showing the binding of 2H04 with UT189 (triangle; red), UT189 LON (triangle; black), TOP52 (circle; blue), and TOP52 LON (circle; black).

FIG. 21A is an image of an electron micrograph showing that 2H04 mAb can bind to native UT189 FimH.

FIG. 21B is an image of an electron micrograph showing that 2H04 mAb can bind to native TOP52 FimH.

FIG. 22 is a chart and corresponding graph of mAb inhibition on red blood cell hemagglutination.

FIG. 23A is a schematic of an experimental timeline of C3H/HeN mice pretreated with 0.5 mg of mAb 3 h before infection with UT189.

FIG. 23B is a set of graphs of UT189 CFU in the bladder (left) and kidneys (right) 3 hours after injection with a mAb.

FIG. 24A is a schematic of an experimental timeline of C3H/HeN mice pretreated with 0.5 mg of mAb 24 h before infection with UT189.

FIG. 24B is a graph showing 2H04 and F7 protection against UT189 in vivo with 10⁸ CFU inoculum in the bladder.

FIG. 24C is a graph showing 2H04 and F7 protection against UT189 in vivo with 10⁸ CFU inoculum in the kidneys.

FIG. 25 is a set of 3D diagrams showing 2H04 binding to the base of the FimHLD.

FIG. 26 is a table showing mAb class and epitope descriptions between tense and relaxed states.

### DETAILED DESCRIPTION OF THE INVENTION

The present disclosure is based, at least in part, on the discovery that FimH inhibiting antibodies can be generated that bind with high affinity to bacterial FimH lectin domains and protect against UTI in vivo. As shown herein, monoclonal antibodies targeting the FimH adhesin that protect against UTI in a murine model are described.

Monoclonal antibodies that inhibit the function of the FimH adhesin protein on Escherichia coli and Klebsiella pneumonia have been developed, and the monoclonal antibodies are demonstrated to be useful in preventing urinary tract infection in a mouse model.

One aspect of the present disclosure provides for compositions of FimH inhibiting antibodies.

The E. coli and K. pneumoniae FimH amino acid sequence is highly conserved among bacterial isolates. In one aspect, monoclonal antibodies (mAbs) to E. coli and/or K. pneumoniae FimH lectin domains can be generated to identify mAbs that inhibit FimH binding and prevent infection in vivo. Using ELISA binding assays, mAbs that bind with high affinity to the antigenic FimH can be identified. In addition, it was found that a subset of mAbs cross-reacted to multiple bacterial FimH proteins and related chaperone usher pili galactose binding adhesin FmIH, which contributes to attachment in chronic UTI. Further, monoclonal antibodies can be identified that inhibited E. coli and/or K. pneumoniae FimH binding in vitro by performing binding inhibition ELISAs. The highest inhibiting mAbs can be tested in an acute murine model of UPEC UTI. One out of three mAbs tested significantly reduced bacterial titers in the urine and bladders of infected mice. Together, these results suggest that monoclonal antibodies inhibiting FimH function are an encouraging antibiotic-sparing therapeutic strategy for K. pneumoniae UTIs.

In various aspects, it is to be noted that the monoclonal antibodies inhibiting FimH function disclosed herein are useful in the treatment or prevention of UTIs associated with E. coli and K. pneumonia and potentially other bacteria species. In additional aspects, the monoclonal antibodies inhibiting FimH function disclosed herein may also have therapeutic value against additional bacteria-related diseases including, but not limited to, Crohn's disease, sepsis, lung infection, and any other bacterial infection-related disorders.

### FimH Modulation Agents

As described herein, FimH expression has been implicated in various diseases, disorders, and conditions. As such, modulation of FimH (e.g., modulation of bacterial FimH adhesin proteins) can be used for the treatment of such conditions. A FimH modulation agent can modulate FimH response or induce or inhibit FimH. FimH modulation can comprise modulating the expression of FimH on cells, modulating the quantity of cells that express FimH, or modulating the quality of the FimH-expressing cells.

FimH modulation agents can be any composition or method that can modulate FimH expression on cells (e.g., blocking mannose-binding FimH adhesin proteins). For example, a FimH modulation agent can be an activator, an inhibitor, an agonist, or an antagonist. As another example, the FimH modulation can be the result of gene editing.

In various aspects, the FimH modulation agent can be an anti-FimH antibody (e.g., a monoclonal antibody to FimH). In various aspects, the monoclonal antibody to FimH comprises a heavy chain protein variable region and a light chain protein variable region configured to bind to and deactivate the binding of the FimH lectin domain protein of K. pneumoniae to exposed mannose residues on the surface of epithelial cells.

In some aspects, the heavy chain protein variable region comprises the amino acid sequence selected from SEQ ID NOS: 61, 63, 65, 67, 69, 71, 73, 75, 77, 79, 81, 83, 85, 87, 89, 91, 93, 95, 97, 99, 101, 103, 105, 107, 109, 111, 113, 115, 117, and 119 and the light chain protein variable region comprises the amino acid sequence selected from SEQ ID NOS: 62, 64, 66, 68, 70, 72, 74, 76, 78, 80, 82, 84, 86, 88, 90, 92, 94, 96, 98, 100, 102, 104, 106, 108, 110, 112, 114, 116, 118, and 120 as listed in Table 2. In other aspects, the heavy chain protein variable region is encoded by a nucleotide sequence selected from SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, and 59 and the light chain protein variable region is encoded by a nucleotide sequence selected from SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, and 60, shown listed in Table 1. In additional aspects, the heavy chain protein variable region is encoded by a nested nucleotide sequence (encoding the specific variable region) selected from SEQ ID NOS: 121, 123, 125, 127, 129, 131, 133, 135, 137, 139, 141, 143, 145, 147, 149, 151, 153, 155, 157, 159, 161, 163, 165, 167, 169, 171, 173, 175, 177, and 179 and the light chain protein variable region is encoded by a nested nucleotide sequence (encoding the specific variable region) selected from SEQ ID NOS: 122, 124, 126, 128, 130, 132, 134, 136, 138, 140, 142, 144, 146, 148, 150, 152, 154, 156, 158, 160, 162, 164, 166, 168, 170, 172, 174, 176, 178, and 180, shown listed in Table 3. In additional aspects, the heavy chain protein variable region is encoded by a plasmid nucleotide sequence (encoding the specific variable region and adjoining regions) selected from SEQ ID NOS: 181, 183, 185, 187, 189, 191, 193, 195, 197,199, 201, 203, 205, 207, 209, 211, 213, 215, 217, 219, 221, 223, 225, 227, 229, 231, 233, 235, 237, and 239, and the light chain protein variable region is encoded by a plasmid nucleotide sequence (encoding the specific variable region and adjoining regions) selected from SEQ ID NOS: 182, 184, 186, 188, 190, 192, 194, 196, 198, 200, 202, 204, 206, 208, 210, 212, 214, 216, 218, 220, 222, 224, 226, 228, 230, 232, 234, 236, 238, and 240, shown listed in Table 4.

TABLE 1
Nucleotide sequences of heavy and light chain protein variable region

| SEQ ID NO | Label | Sequence |
|---|---|---|
| 1 | 1E04_ | ACCGGTGTACATTCCGAGGTCCAGCTGCAACAGTCTGGGGCTGAGCTGGTGAA |
| | HC | GCCTGGGGCTTCAGTGAAGTTGTCCTGCAAGGCTTCTGGCTACACTTTCACCAG |
| | | CTACTGGGTGCACTGGGTGAGGCAGAGGCCTGGACAAGGCCTTGAGTGGATTG |
| | | GAATGATTCATCCTAATAGTGGTAGTACTAACTACAATGAGAAGTTCAAGAGCAA |
| | | GGCCACACTGACTGTAGACAAATCCTCCAGCACAGCCTACATGCAACTCAGCAG |
| | | CCTGACATCTGAGGACTCTGCGGTCTATTACTGTGCAAGTTATTACTACGGTAGT |
| | | AGCTATTACTTTGACTACTGGGGCCAAGGCACCACTCTCACAGTCTCCTCAGCGT |
| | | CGAC |
| 2 | 1E04_ | ACCGGTGTACATTCCGACATTGTGATGACCCAGTCTCCATCCTCCCTGACTGTGA |
| | LC | CAGCAGGAGAGAAGGTCACTATGAGCTGCAAGTCCAGTCAGAGTCTGTTAAACA |
| | | GTGGAAATCAAAAGAACTACTTGACCTGGTACCAGCAGAAACCAGGGCAGCCTC |
| | | CTAAACTGTTGATCTACTGGGCATCCACTAGGGAATCTGGGGTCCCTGATCGCTT |
| | | CACAGGCAGTGGATCTGGAACAGATTTCACTCTCACCATCAGCAGTGTGCAGGC |
| | | TGAAGACCTGGCAGTTTATTACTGTCAGAATGATTATAGTTATCCGCTCACGTTC |
| | | GGTGCTGGGACCAAGCTGGAGCTGAAACGTACG |
| 3 | 1C08_ | ACCGGTGTACATTCCGAGGTCCAGCTGCAACAGTCTGGGGCTGAGCTGGTAAAG |
| | HC | CCTGGGGCTTCAGTGAAGCTGTCCTGCAAGGCTTCTGGCTACACTTTCACCAAC |
| | | TACTGGATGCACTGGGTGAAGCAGAGGCCTGGGCAAGGCCTTGAGTGGATTGG |
| | | AATGATTCATCCTAATAGTGGTACTTCTAACTACAATGAGAAGTTCAAGAGCAAG |
| | | GCCACACTGACTGTAGACAAATCCTCCAGCACAGCCTACATGCAACTCAGCAGC |
| | | CTGACATCTGAGGACTCTGCGGTCTTTTACTGTACAAGATCTGACTGGGCCTTTG |
| | | ACTACTGGGGCCAAGGTACCTCTCTCACAGTCTCCTCAGCGTCGAC |
| 4 | 1C08_ | ACCGGTGTACATTCCGACATTGTGATGACCCAGTCTCCATCCTCCCTGAGTGTGT |
| | LC | CAGCAGGAGAGAAGGTCACTATGAGCTGCAAGTCCAGTCAGAGCCTGTTAAACA |
| | | GTGGAAATCAAAAGAACTACTTGGCCTGGTACCAGCAGAAACCAGGGCAGCCTC |
| | | CTAAACTGTTGATCTACGGGGCATCCACTAGGGAATCTGGGGTCCCTGATCGCT |
| | | TCACAGGCAGTGGATCTGGAACCGATTTCACTCTTACCATCAGTAGTGTGCAGG |
| | | CTGAAGACCTGGCAGTTTATTACTGTCAGAATGATCATAGTTATCCGCTCACGTT |
| | | CGGTGCTGGGACCAAGCTGGAGCTGAAACGTACGCCTTTGTATCATGCTATTGC |
| | | TTCCCGTATGGCTTTCATTTTCTCCTCCTTGTATAAATCCTGGNTGCTGN |
| 5 | 1B02_ | ACCGGTGTACATTCCGAGGTCCAACTGCAGCAGTCTGGAGCTGAGCTGGTGAG |
| | HC | GCCTGGGTCCTCAGTGAAGATGTCCTGCAAGACTTCTGGATATACATTCACATTC |
| | | TACGGTATAAACTGGGTGAAGCAGAGGCCTGGACAGGGCCTGGAATGGATTGG |
| | | ATATATTTATGTTGGAAATGGTTATTCTGAGTACAATGAGAAGTTCAAGGTCAAGG |
| | | CCACACTGACTTCAGACACATCCTCCAGCACAGCCTACATGCAGCTCAGCGGCC |
| | | TGACATCTGAGGACTCTGCAATCTATTTCTGTGCAAGATCAGATGGTTACAACTA |
| | | CTTTGACTACTGGGGCCAAGGCACCACTCTCACAGTCTCCTCAACGTCGAC |
| 6 | 1B02_ | ACCGGTGTACATTCCCAAATTGTTCTCACCCAGTCTCCAGCAATCCTATCTGCAT |
| | LC | CTCCAGGGGAGAAAGTCACAATGACTTGCAGGGCCAGCTCAAGTTTACATTACAT |
| | | GCACTGGTACCAGCAGAAGACAGGATCCTCCCCCAAACCCTGGATTTATGCCAC |
| | | ATCCAACCTGGCTTCTGGAGTCCCTGCTCGCTTCAGTGGCAGTGGGTCTGGGAC |
| | | CTCTTACTCTCTCACAATCAGCAGAGTGGAGGCTGAAGATACTGCCACTTATTAC |
| | | TGCCAGCAGTGGAGTAGTAACCCACCCACGTTCGGTGCTGGGACCAAGCTGGA |
| | | GCTGAAACGTACG |
| 7 | 1A09_ | ACCGGTGTACATTCCGAGGTCCAACTGCAGCAGCCTGGGGCTGAGCTGGTAAA |
| | HC | GCCTGGGGCTTCAGTGAAGTTGTCCTGCAAGGCTTCTGGCTACACTTTCACCAG |
| | | CTACTGGATGCACTGGGTGAAGCAGAGGCCTGGACAAGGCCTTGAGTGGATTG |
| | | GATTGATTCATCCTAATAGTGGTAGTACTTACTACAATGAGAAGTTCAAGAACAAG |
| | | GCCACACTGACTGTAGACAAATCCTCCAGCACAGCCTACATGCAACTCAGCAGC |
| | | CTGACATCTGAGGACTCTGCGGTCTATTACTGTGCAAGATGGGATGATTCCTACT |
| | | GGTACTTCAAAGTCTGGGGCACAGGGACCACGGTCACCGTCTCCTCAGCGTCGA |
| | | C |
| 8 | 1A09_ | ACCGGTGTACATTCCCAAATTGTTCTCACCCAGTCTCCAGCAATCCTGTCTGCAT |
| | LC | CTCCAGGGGAGAAGGTCACAATGACTTGCAGGGCCAGTTCAAGTGTAAGTTACA |
| | | TGCACTGGTACCAGCAGAAGCCAGGATCCTCCCCCAAACCCTGGATTTATGCCA |
| | | CATCCAACCTGGCTTCTGGAGTCCCTGCTCGCTTCAGTGGCAGTGGGTCTGGGA |
| | | CCTCTTACTCTCTCACAATCAGCAGAGTGGAGGCTGAAGATGCTGCCACTTATTA |
| | | CTGCCAGCAGTGGAGTAGCAACCCGTACACGTTCGGAGGGGGGACCAAGCTGG |
| | | AAATAAAACGTACG |
| 9 | 2A03_ | ACCGGTGTACATTCCGAGGTCCAACTGCAGCAGTCTGGAGCTGAGCTGGTGAG |
| | HC | GCCTGGGGCTTCAGTGAAGCTGTCCTGCAAGGCTTCTGGCTACACTTTCACTGA |
| | | CTACTATATAAACTGGGTGAAGCAGAGGCCTGGACAGGGACTCGAGXXXXXXTG |
| | | GATTGCAAGGATTTATCCTGGAAGTGGTAATACTTACTACAATGAGAAGTTCAAG |
| | | GGCAAGGCCACACTGACTGCAGAAAAATCCTCCAGCACTGCCTACATGCAGCTC |
| | | AGCAGCCTGACATCTGAGGACTCTGCTGTCTATTTCTGTGCAAGACGGCTAACTG |
| | | CGGGATACTTCGATGTCTGGGGCACAGGGACCACGGTCACCGTCTCCTCAGCG |
| | | TCGAC |
| 10 | 2A03_ | ACCGGTGTACATTCCGACATCAAGATGACCCAGTCTCCATCTTCCATGTTTGCAT |
| | LC | CTCTAGGAGAGAGAGTCACTATCACTTGCAAGGCGAGTCAGGACATTAATAGCTA |
| | | TTTATCCTGGTTCCAGCAGAAACCAGGGAAATCTCCTAAGACCCTGATCTATCGT |
| | | GCAAACAGATTGGTAGATGGGGTCCCATCAAGGTTCAGTGGCAGTGGATCTGGG |
| | | CAAGATTATTCTCTCACCATCAGCAGCCTGGAGTATGAAGATATGGGAATTTATTA |
| | | TTGTCTACAGTATGATGAGTTTCCGCTCACGTTCGGTGCTGGGACCAAGCTGGA |
| | | GCTGAAACGTACG |
| 11 | 2C09_ | ACCGGTGTACATTCCGAAGTGATGCTGGTGGAGTCTGGGGAAGACTTAGTGAAA |
| | HC | CCTGGAGGGTCCCTGAAACTCTCCTGTATAGCCTCTGGATTCACTTTCAGTAGCT |
| | | ATGCCATGTCTTGGGTTCGCCAGACTCCAGAGAAGAGGCTGGAGTGGGTCGCAT |
| | | ATATTAGTAGTGGTGGTGATTACATCTACTATACAGACACTGTGAAGGGCCGATT |
| | | CACCATCTCCAGAGACAATGCCAGGAACACCCTGTTCCTACAAATGAGCAGTCT |
| | | GAAGTCTGAGGACACAGCCATGTATTACTGTACAAGAGATACCGGTTACTACGTT |
| | | TCTCGGTACTTCGATGTCTGGGGCACAGGGACCACGGTCACCGTCTCCTCAGCG |
| | | TCGAC |
| 12 | 2C09_ | ACCGGTGTACATTCCGACATTGTGATGACCCAGTCTCAAAAATTCATGTCCACAT |
| | LC | CAGTTGGAGACAGGGTCAGTGTCACCTGCAAGGCCAGTCAGAATGTGGGAACTA |
| | | ATGTAGCCTGGTATCAACAGAAACCAGGACAATCTCCTAAAGCACTGATTTACTC |
| | | GGCATCCTTCCGGAACAGTGGAGTCCCTGATCGCTTCACAGGCAGTGGATCTGG |
| | | GACAGATTTCACTCTCACCATCAGCAATGTGCAGTCTGAAGACTTGGCAGAGTAT |
| | | TTCTGTCATCAATATAACAACTATCCTCTGACGTTCGGTGGAGGCACCAAGCTGG |
| | | AAATCAAACGTACG |
| 13 | 2G02_ | ACCGGTGTACATTCCGAGGTCCAACTGCAGCAGTCTGGACCTGTGCTGGTGAAG |
| | HC | CCTGGGCCTTCAGTGAAGATATCCTGTAAGGCTTCTGGATTCACATTCACTGACT |
| | | ACTACATGCACTGGGTGAAGCAGAGCCATGGAAAGAGCCTTGAATGGATTGGAC |
| | | TTGTTTATCCTTACAATGGTGGTACTTATTACAACCAGAAGTTCAAGGGCAAGGC |
| | | CACATTGACTGTAGACACATCCTCCAGCACAGCCTACATGGAGCTAAACAGCCT |
| | | GACTTCTGAGGACTCTGCGGTCTATTACTGTGTACGATTAGGGAACGGTAGTAG |
| | | CAACGAGTGGTACTTCGATGTCTGGGGCACAGGGACCACGGTCACCGTCTCCTC |
| | | AGCGTCGAC |
| 14 | 2G02_ | ACCGGTGTACATTCCCAAATTGTTCTCACCCAGTCTCCAGCACTCATGGCTGCAT |
| | LC | CTCCAGGGGAGAAGGTCACCATCACCTGCAGTGTCAGCTCAAGTATAAGTTCCT |
| | | ACAATTTGCACTGGTACCAGCAGAAGTCAGAAACCTCCCCCAAACCCTGGATTTA |
| | | TGGCACATCCAACCTGGCTTCTGGAGTCCCTGTTCGCTTCAGTGGCAGTGGATC |
| | | TGGGACCTCTTATTCTCTCACAATCAGCAGTATGGAGGCTGAAGATGCTGCCACT |
| | | TATTACTGTCAACAGTGGAGAACCTACCCGTGGACGTTCGGTGGAGGCACCAAG |
| | | CTGGAAATCAAACGTACG |
| 15 | 1D09_ | ACCGGTGTACATTCCGAGGTCCAACTGCAGCAGCCTGGAGCTGAGCTTGTGAAG |
| | HC | CCTGGGGCTTCAGTGAAGCTGTCCTGCAAGGCTTCTGGCTACACCTTCACCAAC |
| | | TACTGGATACACTGGATGAAGCAGAGGCCTGGACGAGGCCTTGAGTGGATTGGA |
| | | AGGATTGAGCCTAATAGCGGTGATACTAAATACAATGAGAAGATCAAGAGCAGG |
| | | GCCACACTGACTGTAGACAAACCGTCCAGCACAGCCTACATGCAGCTCAGCAGC |
| | | CTGACATCTGAGGACTCTGCGGTCTATTATTGTGCAAGATCTGGGTATGATTACC |
| | | CTGAGGCCTGGGGCCAAGGCACCACTCTCACAGTCTCCTCAGCGTCGAC |
| 16 | 1D09_ | ACCGGTGTACATTCCGACATTGTGCTGACCCAATCTCCAGCTTCTTTGGCTGTGT |
| | LC | CTCCAGGGCAGAGAGCCACCATCTCCTGCAGAGCCAGTGAAGGTGTTGAATATT |
| | | ATGGCACAACTTTAATGCAGTGGTACCAACAGAAACCAGGACAGCCACCCAAAC |
| | | TCCTCATCTATGCTGCATCCAACGTAGAATCTGGGGTCCCTGCCAGGTTTAGTGG |
| | | CAGTGGGTCTGGGACAGACTTCAGCCTCAACATCCATCCTGTGGAGGAGGATGA |
| | | TATTGCAATGTATTTCTGTCAGCAAAGTAGGAAGGTTTCTTGGACGTTCGGTGGA |
| | | GGCACCAAGCTGGAAATCAAACGTACG |
| 17 | 2B06_ | ACCGGTGTACATTCCGAGGTCCAGCTGCAACAGCCTGGGGCTGAGCTTGTGAAG |
| | HC | CCTGGGGCTTCAGTGAAGTTGTCCTGCAAGGCTTCTGGCTATACCTTCACCAACT |
| | | ACTGGATGCACTGGGTGAAACACAGGCCTGGACGAGGCCTTGAGTGGATTGGA |
| | | AGGATTGATCCTAATAGTGGTGGTACTAAGTACAATGAGAAGTTCAAGAATAAGG |
| | | CCACACTGACTGTAGACAATCCCTCCAGCACAGGCTACATGCAGCTCAGCAGCC |
| | | TGACATCTGAGGACTCTGCGGTCTATTATTGTACAAGATCTGGGTATGATTACCC |
| | | TGACTACTGGGGCCAAGGCACCACTCTCACAGTCTCCTCAGCGTCGAC |
| 18 | 2B06_ | ACCGGTGTACATTCCGACATTGTGCTGACCCAATCTCCAGCTTCTTTGGCTGTGT |
| | LC | CTCTAGGGCAGAGAGCCACCATCTCCTGCAGAGCCAGTGAAAGTGTTGAATATT |
| | | ATGGCACAAGTTTAATGCAGTGGTACCAACAGAAACCAGGACAGCCACCCAAAG |
| | | TCCTCATCTATGCTGCATCCAACGTAGATTCTGGGGTCCCTCCCAGGTTTAGTGG |
| | | CAGTGGGTCTGGGACAGACTTCAGCCTCAACTTCCATCCTGTGGAGGAGGATGA |
| | | TATTGCAATGTATTTCTGTCAGCAAAGTAGGAAGGTTCCTTGGACGTTCGGTGGA |
| | | GGCACCAAGCTGGAAATCAAACGTACG |
| 19 | 2A02_ | ACCGGTGTACATTCCGAGGTCCAACTGCAACAGCCTGGGGCTGAGCTTGTGAAG |
| | HC | CCTGGGGCTTCAGTGAAGCTGTCCTGCAAGGCTTCTGGCTACACCTTCACCAGC |
| | | TACTGGATGCACTGGGTGAAGCAGAGGCCTGGACGAGGCCTTGAGTGGATTGG |
| | | AAGGATTGATCCTAATAGTGGTGGTACTAAGTACAATGAGAAGTTCAAGAGCAAG |
| | | GCCACACTGACTGTAGACAAACCCTCCAGCACAGCCTACATGCAGCTCAGCAGC |
| | | CTGACATCTGAGGACTCTGCGGTCTATTATTGTGCAAGATCTGGGTATGATTACC |
| | | CTGACTACTGGGGCCAAGGCACCACTCTCACAGTCTCCTCAGCGTCGAC |
| 20 | 2A02_ | ACCGGTGTACATTCCGACATTGTGCTGACCCAATCTCCAGCTCCTTTGGCTGTGT |
| | LC | CTCTAGGGCAGAGAGCCACCATCTCCTGCAGAGCCAGTGAAGGTGTTGAATATT |
| | | ATGACACAAGTTTAATGCAGTGGTACCAACAGAAACCAGGACAGCCACCCAAACT |
| | | CCTCATCTATGCTGCATCCAACGTAGAACCTGGGGTTCCTGCCAGGTTTGGTGG |
| | | CAGTGGGTCTGGGACAGACTTCAGCCTCAACATCCATCCTGTGGAGGAGGATGA |
| | | TATTGCAATGTATTTCTGTCAGCAAAGTAGGAAGGTTCCTTGGACGTTCGGTGGA |
| | | GGCACCAAGCTGGAAATCAAACGTACG |
| 21 | 1B07_ | ACCGGTGTACATTCCGAGGTCCAACTGCAACAGCCTGGGGCTGAGCTGGTAAAG |
| | HC | CCTGGGACTTCAGTGAAGTTGTCCTGCAAGGCTTCTGGCTACACTTTCACCAGCT |
| | | ACTGGATGCACTGGGTGAAGCAGAGGCCTGGACAAGGCCTTGAGTGGATTGGA |
| | | ATGATTCATCCTAATAGTGGTAGTACTAACTACAATGAGATGTTCGAGAGCAAGG |
| | | CCTCACTGACTGTAGACAAATCCTCCAGCACAGCCTACATGCAACTCAGCAGCCT |
| | | GACATCTGAGGACTCTGCGGTCTATTACTGTGCAAGAATCGGATACTCCGGCTG |
| | | GGGTCAAGGAACCTCAGTCACCGTCTCCTCAGCGTCGAC |
| 22 | 1B07_ | ACCGGTGTACATTCCGACATTGTGATGACACAGTCTCCATCCTCCCTGAGTGTGT |
| | LC | CAGCAGGAGAGAAGGTCACTATGAGCTGCAAGTCCAGTCAGAGTCTGTTAAATG |
| | | GTGGAAATCAAAAGAACTACTTGGCCTGGTACCAGCAGAAACCAGGGCAGCCTC |
| | | CTAAAGTGTTGATCAACGGGGCATCCACTAGGGAATCTGGGGTCCCTGATCGCT |
| | | TCACAGGCAATGGATCTGGAACCGATTTCACTCTTACCATCAGCAGTGTGCAGG |
| | | CTGAAGACCTGGCAGTTTATTACTGTCAGAATGATCATACTTATCCGCTCACGTT |
| | | CGGTGCTGGGACCAAGCTGGAGCTGAAACGTACG |
| 23 | 2D04_ | ACCGGTGTACATTCCGAGGTCCAACTGCAGCAGCCTGGAGGTGAGCTGGTGAG |
| | HC | GCCTGGGTCCTCAGTGAAGATGTCCTGCAAGACTTCTGGATATACATTCACATTC |
| | | TACGGTATAAACTGGGTGAAGCAGAGGCCTGGACAGGGCCTGGAATGGATTGG |
| | | ATATATTTATGTTGGAAATGGTTATACTGAGTACAATGAGAAGTTCAAGGTCAAGG |
| | | CCACACTGACTTCAGACACATCCTCCAGCACAGCCTACATGCAGCTCAGCGGCC |
| | | TGACATCTGAGGACTCTGCAATCTATTTCTGTGCAAGATCCGATGGTTACAACTA |
| | | CTTCGACTACTGGGGCCAAGGCACCACTCTCACAGTCTCCTCAGCGTCGAC |
| 24 | 2D04_ | ACCGGTGTACATTCCCAAATTGTTCTCACCCAGTCTCCAGCAATCCTGACTGCAT |
| | LC | CCCCAGGGGAGAAAGTCACAATGACTTGCAGGGCCAGTTCAAGTGTACATTACA |
| | | TGCACTGGTACCAGCAGAAGCCAGGATCCTCCCCCAAACCCTGGATTTATGCCA |
| | | CATCCAACCTGGCTTCTGGAGTCCCTGCTCGCTTCAGTGGCAGTGGGTCTGGGA |
| | | CCTCTTACTCTCTCACAATCAGCAGAGTGGAGGCTGAAGATACTGCCACTTATTA |
| | | CTGCCATCAGTGGAGTAGTAACCCACCCACGTTCGGTGCTGGGACAAAGTTGGA |
| | | AATAAAACGTACG |
| 25 | 1E07_ | ACCGGTGTACATTCCGAGGTCCAACTGCAGCAGCCTGGGGCTGAGCTGGTAAA |
| | HC | GCCTGGGGCTTCAGTGAAGTTGTCCTGCAAGGCTTCTGGCTACACTTTCACCAG |
| | | CTACTGGATGCACTGGGTGAAGCAGAGGCCTGGACAAGGCCTTGAGTGGATTG |
| | | GATTGATTCATCCTAATAGTGGTAGTACTTACTACAATGAGAAGTTCAAGAACAAG |
| | | GCCACACTGACTGTAGACAAATCCTCCAGCACAGCCTACATGCAACTCAGCAGC |
| | | CTGACATCTGAGGACTCTGCGGTCTATTACTGTGCAAGATGGGATGATTCCTACT |
| | | GGTACTTCAAAGTCTGGGGCACAGGGACCACGGTCACCGTCTCCTCAGCGTCGA |
| | | C |
| 26 | 1E07_ | ACCGGTGTACATTCCCAAATTGTTCTCACCCAGTCTCCAGCAATCCTGTCTGCAT |
| | LC | CTCCAGGGGAGAAGGTCACAATGACTTGCAGGGCCAGTTCAAGTGTAAGTTACA |
| | | TGCACTGGTACCAGCAGAAGCCAGGATCCTCCCCCAAACCCTGGATTTATGCCA |
| | | CATCCAACCTGGCTTCTGGAGTCCCTGCTCGCTTCAGTGGCAGTGGGTCTGGGA |
| | | CCTCTTACTCTCTCACAATCAGCAGAGTGGAGGCTGAAGATGCTGCCACTTATTA |
| | | CTGCCAGCAGTGGAGTAGCAACCCGTACACGTTCGGAGGGGGGACCAAGCTGG |
| | | AAATAAAACGTACG |
| 27 | 2D07_ | ACCGGTGTACATTCCGAGGTCCAGCTGCAACAGTCTGGACCTGAGCTGGTGAGG |
| | HC | CCTGGGGCTTCAGTGAAGATATCCTGCAAGGCTTCTGGCTACACCTTCACTGACT |
| | | ACTATATACACTGGCTGAAGCAGAGGCCTGGACAGGGACTTGAGTGGATTGGAT |
| | | TGATTTTTCCTGGAAGTGGTAGTATTTACTGTAATGAGAAGTTCAAGGGCAAGGC |
| | | TACACTTACTGTAGACAAATCCTCCACCACAGCCTACATGTTGCTCAGCAGCCTG |
| | | ACCTCTGAGGACTCTGCGGTCTACTTCTGTGCAAGATGGGAGACTACGGCGTGG |
| | | TACTTCGATGTCTGGGGCACAGGGACCACGGTCACCGTCTCCTCAGCGTCGAC |
| 28 | 2D07_ | ACCGGTGTACATTCCCAAATTGTTCTCACCCAGTCTCCCCAAATTGTTCTCACCC |
| | LC | AGTCTCCAGCAATCCTGTCTGCATCTCCAGGGGAGAAGGTCACAATGACTTGCA |
| | | GGGCCAGTTTAAGTGTAAGTTACATGCACTGGTACCAGCAGAAGCCAGGATCCT |
| | | CCCCCAAACCCTGGATTTATGCCACATCCAACCTGGCTTCTGGAGTCCCTGCCC |
| | | GCTTCAGTGGCAGTGGGTCTGGGACCTCTTACTCTCTCACAGTCAGCAGAGTGG |
| | | AGGCCGAAGATGCTGCCACTTATTACTGCCAGCAGTGGAGGAGTTACCCGTACA |
| | | CGTTCGGAGGGGGCACCAAGCTGGAAATCAAACGTACG |
| 29 | 1A02_ | ACCGGTGTACATTCCGAGGTCCAACTGCAACAGTCTGGACCTGTGCTGGTGAAG |
| | HC | CCTGGGCCTTCAATGAAGATATCCTGTAAGGCTTCTGGATTCACATTCACTGACT |
| | | ACTACATACACTGGGTGAGGCAGAGCCATGGAAAGAGCCTTGAGTGGATTGGAC |
| | | TTGTTTCTCCTTACAATGGTGGTACTTACTACAACCAGAAGTTCAAGGGCAAGGC |
| | | CACATTGACTGTAGACTCATCCTCCAGCACAGCCTACATGGAGCTAAGCAGCCT |
| | | GACTTCTGAGGACTCTGCGGTCTATTACTGTGCAAGATTGGGCTACTATGGTGAC |
| | | TGGTACTACTTTGACTACTGGGGCCAAGGCACCCCTCTCACAGTCTCCTCAGCG |
| | | TCGAC |
| 30 | 1A02_ | ACCGGTGTACATTCCCAAATTGTTCTCACCCAGTCTCCAGCACTCATGGCTACAT |
| | LC | CTCCAGGGGAAAAGGTCACCATCACCTGTAGTGTCAGCTCAAGTATAAGTTCCA |
| | | GCAACTTGAACTGGTACCAGCAGAAGTCAGAAACCTCCCCCAAACCCTGGATTTA |
| | | TGGCACATCCAACCTGGCTTCTGGAGTCCCTGTTCGCTTCAGTGGCAGTGGATC |
| | | TGGGACCTCTTATTCTCTCACAATCAGCAGCATGGAGGCTGAAGATGCTGCCACT |
| | | TATTACTGTCAACAGTGGAGTAGTTACCCATACACGTTCGGAGGGGGGACCAAG |
| | | CTGGAAATAAAACGTACG |
| 31 | 2D10 | ACCGGTGTACATTCCGAGGTCCAGCTGCAACAGTCTGGGGCTGAGCTGGTGAA |
| | HC | GCCTGGGGCTTCAGTGAAGTTGTCCTGCAAGGCTTCTGGCTACACTTTCACCAC |
| | | CTACTGGATGCACTGGATGAAGCAGTGGCCTGGACAAGGCCTTGAGTGGATTGG |
| | | ATTGATTCATCCTAATAGTGGTAGTACTTACTACAATGAGAAGTTCAAGAGCAAG |
| | | GCCACACTGACTGTAGACAAATCCTCCAGCACAGCCTACATGCAACTCAGCAGC |
| | | CTGACATCTGAGGACTCTGCGGTCTATTACTGTGCAAGATTTGAGGAGCTATGG |
| | | GGAGGTTACTGGTACTTCGATGTCTGGGGCACAGGGACCACGGTCACCGTCTCC |
| | | TCAGCGTCGAC |
| 32 | 2D10 | ACCGGTGTACATTCCCAAATTGTTCTCACCCAGTCTCCAGCACTCATGGCTGCAT |
| | LC | CTCCAGGGGAGAAGGTCACCATCACCTGCAGTGTCAGCTCAAGTATAAGTTCCA |
| | | GCAACTTGCACTGGTACCAGCAGAAGTCAGAAACCTCCCCCAAACCCTGGATTT |
| | | ATGGCACATCCAACCTGGCTTCTGGAGTCCCTGTTCGCTTCAGTGGCAGTGGAT |
| | | CTGGGACCTCTTATTCTCTCACAATCAGCAGCATGGAGGCTGAAGATGCTGCCA |
| | | CTTATTACTGTCAACAGTGGAGTAGTTACCCGTACACGTTCGGAGGGGGGACCA |
| | | AGCTGGAAATAAAACGTACG |
| 33 | 2E08_ | ACCGGTGTACATTCCGAGGTCCAGCTGCAGCAGCCTGGGGCTGAGCTGGCAAA |
| | HC | GCCTGGGGCTTCAGTGAAGTTGTCCTGCAAGGCTTCTGGCTACAATTTCACCAG |
| | | CTACTGGATGCACTGGGTGAAGCAGAGGCCTGGACAAGGCCTTGAGTGGATTG |
| | | GATTGATTCATCCTAATAGTGGTGGTACTTACTACAATGAGAAGTTCAAGAGCAA |
| | | GGCCACACTGACTTTAGACAGATCCTCCAGCACAGCCTACATGCAACTCAGCAG |
| | | CCTGACATCTGAGGACTCTGCGGTCTATTATTGTGCAAGATTTGAGGAGGAATGG |
| | | GGAGGGTATTGGTACTTCGATGTCTGGGGCACGGGGACCACGGTCACCGTCTC |
| | | CTCAGCGTCGAC |
| 34 | 2E08_ | ACCGGTGTACATTCCCAAATTGTTCTCACCCAGTCTCCAGCACTCATGGCTGCAT |
| | LC | CTCCAGGGGAAAAGGTCACCATCACCTGCAGTGTCAGTTCAAGTATAAGTTCCA |
| | | GCAACTTGCACTGGTACCAACAGAAGTCAGAAACCTCCCCCAAACCCTGGATTTA |
| | | TGGCACTTCCAACCTGGCTTCTGGAGTCCCTGTTCGCTTCAGTGGCAGTGGATC |
| | | TGGGACCTCTTATTCTCTCACAATCAGCAGCATGGAGGCTGAAGATGCTGCCACT |
| | | TATTACTGTCAACAGTGGAGTAGTTACCCGTACACGTTCGGAGGGGGGACCAAA |
| | | CTGGAAATAAAACGTACG |
| 35 | 2H04_ | ACCGGTGTACATTCCGAGGTCCAACTGCAACAGTCTGGGGCTGAGCTGGTAAAG |
| | HC | CCTGGGGCTTCAGTGAAGTTGTCCTGCAAGGCTTCTGGCTACACTTTCACCAGC |
| | | TACTGGATGCACTGGGTGAAGCAGAGGCCTGGACAAGGCCTTGAGTGGATTGG |
| | | ACTGATTCATCCTAATAGTAGTAGTACTTACTACAATGAGAAGTTCAAGACCAGG |
| | | GCCACACTGACTGTAGACAAGTCCTCCAGCACAGCCTACATGCAACTCAGCAGC |
| | | CTGACATCTGAGGACTCTGCGGTCTATTACTGTGCAAGATTGGGCTATGGTAACT |
| | | CCTACTGGTACTTCGATGTCTGGGGCACAGGGACCACGGTCACCGTCTCCTCAG |
| | | CGTCGAC |
| 36 | 2H04_ | ACCGGTGTACATTCCCAAATTGTTCTCACCCAGTCTCCAGCACTCATGGCTGCAT |
| | LC | CTCCAGGGGAGAAGGTCACCATCACCTGCAGTGTCAGCTCAAGTATTAGTTCCA |
| | | GCAACTTGCACTGGTACCAGCAGAAGTCAGAAACCTCCCCCAAACCCTGGATTT |
| | | ATGGCACATCCAACCTGGCTTCTGGAGTCCCTATTCGCTTCAGTGGCAGTGGAT |
| | | CTGGGACCTCTTATTCTCTCACAATCAGCAGTTTGGAGGCTGAGGATGCTGCCA |
| | | CTTATTACTGTCAACAGTGGAGAAGTTACCCGTGGACGTTCGGTGGAGGCACCA |
| | | AGCTGGAAATCAAACGTACG |
| 37 | 1B03_ | ACCGGTGTACATTCCGAGGTCCAACTGCAGCAGTCTGGGGCTGAGCTGGTAAAG |
| | HC | CCTGGGGCTTCAGTGAAGTTGTCCTGCAAGGCTTCTGGCTACACTTTCACCAGC |
| | | TACTGGATGCACTGGGTGCAGCAGAGGCCTGGACAAGGCCTTGAGTGGATTGG |
| | | ATTGATTCATCCTATTGGTGGTGGTACTCACTACAATGAGAAGTTCAAGAACAAG |
| | | GCCACACTGACTGTAGACAAATCCTCCAGCACAGCCTACATGCAACTCAGCAGC |
| | | CTGACATCTGAGGACTCTGCGGTCTATTACTGTGCAAGACTCGGAACTGGTCCG |
| | | TACTACTTTGACTACTGGGGCCAAGGCACCACTCTCACAGTCTCCTCAGCGTCG |
| | | AC |
| 38 | 1B03_ | ACCGGTGTACATTCCCAAATTGTTCTCACCCAGTCTCCAGCACTCATGGCTGCAT |
| | LC | CTCCAGGGGAGAAGGTCACCATCACCTGCAGTGTCAGCTCAAGTATAAGTTCCA |
| | | GCACCTTGGACTGGTACCAGCAGAAGTCAGAAACCTCCCCCAAACCCTGGGTTT |
| | | ATGGCACATCCAACCTGGCTTCTGGAGTCCCTGTTCGCTTCAGTGGCAGTGGAT |
| | | CTGGGACCTCTTATTCTCTCACAATCAGCAGCATGGAGGCTGAAGATGCTGCCA |
| | | CTTATTACTGTCAACAGTGGAGTAGTTACCCGTGGACGTTCGGTGGAGGCACCA |
| | | AGCTGGAAATCAAACGTACG |
| 39 | 2H07_ | ACCGGTGTACATTCCGAGGTCCAGCTGCAGCAGTCTGGACCTGTGCTGGTGAAG |
| | HC | CCTGGGCCTTCAGTGAATATATCCTGTAAGGCTTCTGGATTCACATTCACTGACT |
| | | ACTACATTCACTGGGTGAAACAGAGCCATGGAAAGAGCCTTGAGTGGATTGGAC |
| | | TTGTTTATCCTTACAGTGGTGGTACTTACTACAACCAGAAGTTCAAGGGCAAGGC |
| | | CACATTGACTGTAGACGCATCCTCCAGCACAGCCTACATGGAGCTAGGCAGCCT |
| | | GACTTCTGAGGACTCTGCGGTCTATTACTGTGCAAGATTGGGCGACAACTTCTAC |
| | | TACTTTGACTACTGGGGCCAAGGCACCACTCTCACAGTCTCCTCAGCGTCGAC |
| 40 | 2H07_ | ACCGGTGTACATTCCCAAATTGTTCTCACCCAGTCTCCAACACTCATGGCTGCAT |
| | LC | CTCCAGGGGAGAAGGTCACCATCACCTGCAGTGTCAGCTCAAGTATAAGTTCCA |
| | | GCAACTTGCACTGGTACCAGCAGAAGTCAGAAACCTCCCCCAAACCCTGGATTT |
| | | ATGGCACATCCAACCTGGCTTCTGGAGTCCCTGTTCGCTTCAGTGGCAGTGGAT |
| | | CTGGGACATCTTATTCTCTCACAATCAGCAGCATGGAGGCTGAAGATGCTGCCA |
| | | CTTATTACTGTCAACAGTGGAGAAGTTACCCGTACACGTTCGGAGGGGGGACCA |
| | | AGCTGGAAATAAAACGTACG |
| 41 | 1C01_ | ACCGGTGTACATTCCGAGGTCCAACTGCAACAGCCTGGACCTGTGCTGGTGAAG |
| | HC | CCTGGGCCTTCAGTGAAGATATCCTGTAAGGCTTCTGGATTCACATTCACTGACT |
| | | ACTACATGCACTGGGTGAAACAGAGCCATGGAAAGAGCCTTGAGTGGATTGGTC |
| | | TTGTTTCTCCTTACAATGGTGGTACTTTCTACAACCAGAAGTTCAAGGGCAAGGC |
| | | CACATTGACTGTAGACACATCCTCCAGCACAGCCTACATGGAACTAAACAGCCTG |
| | | ACTTCTGAGGACTCTGCGGTCTATTACTGTGCAAGAGTGGGTAATAGCTACGTCC |
| | | ATTATGCTATGGACTACTGGGGTCAAGGAACCTCAGTCACCGTCTCCTCAGCGT |
| | | CGAC |
| 42 | 1C01_ | ACCGGTGTACATTCCCAAATTGTTCTCACCCAGTCTCCAGCACTCATGGCTGCAT |
| | LC | CTCCAGGGGAGAAGGTCACCATCACCTGCAGTGTCAGCTCAAGTATAAGTTCCA |
| | | GCAACTTGCACTGGTACCAGCAGAAGTCAGAAACCTCCCCCAAACCCTGGATTT |
| | | ATGGCACATCCAACCTGGCTTCTGGAGTCCCTGTTCGCTTCAGTGGCAGTGGAT |
| | | CTGGGACCTCTTATTCTCTCACAATCAGCAGCATGGAGGCTGAAGATGCTGCCA |
| | | CTTATTACTGTCAACAGTGGAGTAATTACCCGTACACGTTCGGAGGGGGGACCA |
| | | AGCTGGAAATAAAACGTACG |
| 43 | 2C07_ | ACCGGTGTACATTCCGAAGTGAAGCTGGTGGAGTCTGGGGAAGGCTTAGTGAAG |
| | HC | CCTGGAGGGTCCCTGAAACTCTCCTGTGCAGCCTCTGGATTCACTTTCAGTAGCT |
| | | ATGCCATGTCTTGGGTTCGCCAGACTCCAGAGAAGAGGCTGGACTGGGTCGCAT |
| | | ACATTAGTAGTGGTGGTGATCACATCTACTATGCAGACACTGTGAAGGGCCGATT |
| | | CACCATCTCCAGAGACAATGCCAGGAACACCCTGTACCTGCAAATGAGCAGTCT |
| | | GAAGTCTGAGGACACAGCCATGTATTACTGTACAAGAGATACCGGTTACTACGTC |
| | | TCTCGGTACTTCGATGTCTGGGGCACAGGGACCACGGTCACCGTCTCCTCAGCG |
| | | TCGAC |
| 44 | 2C07_ | ACCGGTGTACATTCCGACATTGTGATGACCCAGTCTCAAAAATTCATGTCCACAT |
| | LC | CAGTAGGAGACAGGGTCAGCGTCACCTGCAAGGCCAGTCAGAATGTGGTTACTA |
| | | ATGTTGCCTGGTATCAACAGAAATCAGGGCAATCTCCTAAAGTAGTGATTTATTC |
| | | GGCATCCTTCCGGTCCAGTGGAGTCCCTGATCGCTTCACAGGCAGTGGATCTGG |
| | | GACAGATTTCACTCTCACCATCAGCAATGTGCAGTCTGAAGACTTGGCAGAGTAT |
| | | TTCTGTCACCAATATAACAGCCATCCTCTGACGTTCGGTGGAGGCACCAAGCTG |
| | | GAAATCAAACGTACG |
| 45 | 2G04_ | ACCGGTGTACATTCCGAGGTCCAGCTGCAGCAGCCTGGACCTGAATTGGTGAAG |
| | HC | CCCGGGTCCTCACTGAAGATATCCTGCAAGGCTTCTGGTTACACCTTCACTGACT |
| | | ACTTTATAAACTGGGTGAAACAGAGGCCTGGACAGGGACTTGACTGGATTGGAT |
| | | GGATTTTTCCTGGAAGTGGTAGTACTTACTACAATGACAAGTTCAAGGGCAAGGC |
| | | CACACTTACTGTAGACAAATCCTCCAGCACTGCCTACATGTTGCTCAGCAGCCTG |
| | | ACCTCTGAGGCCTCTGCGGTCTATTTCTGTGCAAGATGGGACTCCGATAGTACCT |
| | | ACTGGTACTTCGATGTCTGGGGCACAGGGACCACGGTCACCGTCTCCTCAGCGT |
| | | CGAC |
| 46 | 2G04_ | ACCGGTGTACATTCCCAAATTGTTCTCACCCAGTCTCCAGCACTCATGGCTGCAT |
| | LC | CTCCAGGGGAGAAGGTCACCATCACCTGCAGTGTCAATTCAAGTATAAGTTCCA |
| | | GCACCTTGCACTGGTACCAGCAGAAGTCACAAACCTCCCCCAAACCCTGGATTT |
| | | ATGGTACATCCAATTTGGCTTCTGGAGTCCCTATTCGCTTCAGTGGCAGTGGATC |
| | | TGGGACCTCTTATTCTCTCACAATCAGCAGCATGGAGGCTGAAGATGCTGCCACT |
| | | TATTACTGTCAACAGTGGAGTACTTACCCGTACACGTTCGGAGGTGGCACCAAG |
| | | CTGGAAATCAAACGTACG |
| 47 | 1G01_ | ACCGGTGTACATTCCGAGGTCCAACTGCAACAGTCTGGAGCTGAGCTGATGAAG |
| | HC | CCTGGGGCCTCAGTGAAGCTTTCCTGCAAGGCTACTGGCTACACATTCACTGGC |
| | | TACTGGATAGAGTGGTTAAAGCAGAGGCCTGGACATGGCCTTGAGTGGATTGGA |
| | | GAGATTTTACCTGGAAGTGATAATACTAACTACAATGAGAAGTTCAGGGGCAAGG |
| | | CCACATTCACTGCAGATACATCCTCCAACACAGCCTACATGCACCTCAGCAGCCT |
| | | GACAACTGAGGACTCTGCCATCTATTACTGTGCAAGAGAAGGGGGTTTCTACTTT |
| | | GACTACTGGGGCCAAGGCACCACTCTCACAGTCTCCTCAGCGTCGAC |
| 48 | 1G01_ | ACCGGTGTACATTCCCAGGCTGTTGTGACTCAGGAATCTGCACTCACCACATCAC |
| | LC | CTGGTGAAACAGTCACACTCACTTGTCGCTCAAGTATTGGGGCTGTTACAACTAG |
| | | TAACTACGCCAACTGGGTCCAAGAAAAACCAGATCATTTATTCACTGGTCTAATA |
| | | GGTGGTACCAACAACCGAGCTCCAGGTGTTCCTGCCAGATTCTCAGGCTCCCTG |
| | | ATTGGAGACAAGGCTGCCCTCACCATCACAGGGGCACAGACTGAGGATGAGGC |
| | | AATATATTTCTGTGCTCTATGGTACAGCAACCATTGGGTGTTCGGTGGAGGAACC |
| | | AAACTGACTGTCCTAGGCCAGCCCAAGTCTTCGCCATCAGTCACCCTGTTTCCG |
| | | CCCTCGAG |
| 49 | 2A05_ | ACCGGTGTACATTCCGAGGTCCAGCTGCAACAGTCTGGACCTGTGCTGGTGAAG |
| | HC | CCTGGGCCTTCAGTGAAGATATCCTGTAAGGCTTCTGGATTCACATTCACTGACT |
| | | ACTACATGCACTGGGTGAAGCAGAGCCATGGAAAGAGCCTTGAATGGATTGGAC |
| | | TTGTTTATCCTTACAATGGTGGTACTAGCTACAACCAGAAGTTCAAGGGCAAGGC |
| | | CACATTGACTGTAGACACATCCTCCACCACAGCCTACATGGAGCTAAACAGCCTG |
| | | ACTTCCGAGGACTCTGCGGTCTATTACTGTGCAAGATTGGGCTCAGGCTACGAG |
| | | TACTACTTTGACTACTGGGGCCAAGGCACCTCTCTCACAGTCTCCTCAGCGTCGA |
| | | C |
| 50 | 2A05_ | ACCGGTGTACATTCCCAAATTGTTCTCACCCAGTCTCCAGCACTCATGGCTGCAT |
| | LC | CTCCAGGGGAGAAGGTCACCATCACCTGCAGTGTCAGCTCAAGTATAAGTTCCA |
| | | GCAACTTGCACTGGTACCAGCAGAAGTCAGAAACCTCCCCCAAACCCTGGATTT |
| | | ATGGCACATCCAACCTGGCTTCTGGAGTCCCTGTTCGCTTCAGTGGCAGTGGAT |
| | | CTGGGACCTCTTATTCTCTCACAATCAGCAGCATGGAGGCTGAGGATGCTGCCA |
| | | CTTATTACTGTCAACAGTGGAGAACTTACCCGTGGACGTTCGGTGGAGGCACCA |
| | | AGCTGGAAATCAAACGTACG |
| 51 | 2H05_ | ACCGGTGTACATTCCGAGGTCCAACTGCAGCAGCCTGGACCTGTGCTGGTGAAG |
| | HC | CCTGGGCCTTCAGTGAAGATATCCTGTAAGGCTTCTGGATTCACATTCACTGACT |
| | | ACTACATGCACTGGGTGAAGCAGAGCCATGGAGAGAGCCTTGAGTGGATTGGAC |
| | | TTGTTTCTCCTTACAATGGTGGTACTTTCTACAACCAGAAGTTCAAGGGCAAGGC |
| | | CACATTGACTGTAGACACATCCTCCAGCACAGCCTACATGGAACTAAACAGCCTG |
| | | ACTTCTGAGGACTCTGCGGTCTATTACTGTGCAAGAGTGGGTAGTAGTTACGTCC |
| | | ATTATGCTATGGACTACTGGGGTCAAGGAACCTCAGTCACCGTCTCCTCAGCGT |
| | | CGAC |
| 52 | 2H05_ | ACCGGTGTACATTCCCAAATTGTTCTCACCCAGTCTCCAGCACTCACGGCTGCAT |
| | LC | CTCCAGGGGAGAGGGTCACCATCACCTGCAGTGTCAGCTCAAGTATAAGTTCCA |
| | | GCAACTTGCACTGGTACCAGCAGAAGTCAGAAACCTCCCCCAAACCCTGGATTT |
| | | ATGGCACATCCAACCTGGCTTCTGGAGTCCCTGTTCGCTTCAGTGGCAGTGGAT |
| | | CTGGGACCTCTTATTCTCTCACAATCAGCAGCATGGAGGCTGAAGATGCTGCCA |
| | | CTTATTACTGTCAACAGTGGAGTAAGTACCCGTACACGTTCGGAGGGGGGACCA |
| | | AGCTGGAAATAAAACGTACG |
| 53 | 2B01_ | ACCGGTGTACATTCCGAGGTCCAACTGCAACAGTCTGGGGCTGGGCTGGTAAAG |
| | HC | CCTGGGGCTTCAGTGAAGTTGCCTTGCAAGGCTTCTGGCTACACTTTCACCAGC |
| | | TACTGGATGCACTGGGTGCAGCAGAGGCCTGGACAAGGCCTTGAGTGGATTGG |
| | | ATTGATTCATCCTATTGGTGGTGGTACTCACTACAATGAGAAGTTCAAGAACAAG |
| | | GCCACACTGACTGTGGACAAATCCTCCAGCACAGCCTACATGCAACTCAGCAGC |
| | | CTGACATCTGAGGACTCTGCGGTCTATTACTGTGCAAGACTCGGAACTGGTCCG |
| | | TACTACTTTGACTACTGGGGCCAAGGCACCACTCTCACAGTCTCCTCAGCGTCG |
| | | AC |
| 54 | 2B01_ | ACCGGTGTACATTCCCAAATTGTTCTCACCCAGTCTCCAGCACTCATGGCTGCAT |
| | LC | CTCCAGGGGAGAAGGTCACCATCACCTGCAGTGTCAGCTCAAGTATAAGTTCCA |
| | | GCAACTTGCACTGGTACCAGCAGAAGTCAGAGACCTCCCCCAAACCCTGGATTT |
| | | ATGGCACATCCAACCTGGCTTCTGGAGTCCCTGTTCGCTTCAGTGGCAGTGGAT |
| | | CTGGGACCTCTTATTCTCTCACAATCAGCAGCATGGAGGCTGAAGATGCTGCCA |
| | | CTTATTACTGTCAACAGTGGAGTAGTTACCCGTGGACGTTCGGTGGAGGCACCA |
| | | AGCTGGAAATCAAACGTACG |
| 55 | 1D01_ | ACCGGTGTACATTCCGAGGTCCAGCTGCAGCAGCCTGGACCTGACGTGGTGAA |
| | HC | GCCTGGGGCTTCAGTGAAGATTTCCTGCAAGGCTTCTGGCTACACCTTCACTGA |
| | | CTACTATATAAACTGGGTGAAGCAGAGGCCTGGACAGGGACTTGAGTGGATTGG |
| | | ATGGATTTTTCCTGGAAGTGGTAGTAGTTATTACAATGAGAAGTTCAAGGACAAG |
| | | GCCACATCTACTGTAGACAAATCCTCCAGCACAGCCTACATGTTGCTCAGCAGCC |
| | | TGACCTCTGAGGACTCTGCGGTCTATTTCTGTGCAAAATGGAAGGATTACGGGT |
| | | GGTACTTCGATGTCTGGGGCACAGGGACCACGGTCACCGTCTCCTCAGCGTCG |
| | | AC |
| 56 | 1D01_ | ACCGGTGTACATTCCCAAATTGTTCTCACCCAGTCTCCAGCACTCATGGCTGCAT |
| | LC | CTCCAGGGGAGAAGGTCACCCTCACCTGCAATGTCAGCTCAAGTCTAAGTTCCA |
| | | GCAACTTGCACTGGTACCAGCAGAAGTCAGAAACCTCCCCCAAACCCTGGATTT |
| | | ATGGCACATCCAACCTGGCTTCTGGAGTCCCTGTTCGCTTCAGTGGCAGTGGAT |
| | | CTGGGACCTCTTATTCTCTCACAATCAGCAGCATGGAGGCTGAAGATGCTGCCA |
| | | CTTATTACTGTCAACAGTGGAGGACTTACCCTTACACGTTCGGAGGGGGGACCA |
| | | AGCTGGAAATAAAACGTACG |
| 57 | 2E02_ | ACCGGTGTACATTCCGAGGTCCAACTGCAGCAGTCTGGACCTGTGCTGGTGAAG |
| | HC | CCTGGGCCTTCAGTGAAGATATCCTGTAAGGCTTCTGGATTCACATTCACTGACT |
| | | ACTACATTCACTGGGTGAAGCGGAGCCATGGAAAGAGCCTTGAGTGGATTGGAC |
| | | TTGTTTCTCCTTACAATGGTGGTACTTTCTACAACCAGAAGTTCAAGGGCAAGGC |
| | | CACATTGACTGTAGATACATCCTCCAATACAGCCTACATGGAGCTAACCAGCCTG |
| | | ACTTCTGAGGACTCTGCGGTCTATTACTGTGCAAGATTGGGGGTTAACTGGTACT |
| | | TCGATGTCTGGGGCACAGGGACCACGGTCACCGTCTCCTCAGCGTCGAC |
| 58 | 2E02_ | ACCGGTGTACATTCCCAAATTGTTCTCACCCAGTCTCCAGCACTCATGGCTGCAT |
| | LC | CTCCAGGGGAAAAGGTCACCCTCACCTGCAGTGTCAGCTCAAGTATAAGTTCCA |
| | | GCACCTTGCACTGGTACCAGCAGAAGTCAGAAACCTCCCCCAAACCCTGGATTT |
| | | ATGGCACATCCAACCTGGCTTCTGGAGTCCCTGTTCGCTTCAGTGGCAGTGGAT |
| | | CTGGGACCTCTTATTCTCTCACAATCAGCAGCATGGAGGCTGAAGATGCTGCCA |
| | | CTTATTACTGTCAACAGTGGAGTACTTACCCATACACGTTCGGAGGGGGGACCA |
| | | AGCTGGAAATAAAACGTACG |
| 59 | 1A05_ | ACCGGTGTACATTCCGAGGTCCAACTGCAACAGCCTGGACCTGTACTGGTGAAG |
| | HC | CCTGGGCCTTCAATGAAGATATCCTGTAAGGCTTCTGGGTACGCTTTCACTGACT |
| | | ACTTCATACACTGGGTGAGACAGAGCCATGGAAGGAGCCTTGAGTGGATTGGAC |
| | | TTGTTTCTCCTTATAATGGTGGTACTTACTACAACCAGAAGTTCAAGGGCAAGGC |
| | | CTCATTGACTGTAGACACATCCTCCAGCACAGCCTACATGGAACTAAGCAGCCTG |
| | | ACTTCTGAGGACTCTGCGGTCTATTACTGTGCAAGATTGGGCTACTATGGTAACT |
| | | GGTACTTCTTTGACTACTGGGGCCAAGGCACCCCTCTCACAGTCTCCTCAGCGT |
| | | CGAC |
| 60 | 1A05_ | ACCGGTGTACATTCCCAAATTGTTCTCACCCAGTCTCCAGCACTCATGGCTACAT |
| | LC | CTCCAGGGGAAAAGGTCACCATCACCTGTAGTGTCAGCTCAAGTATAAGTTCCA |
| | | GCAACTTGAACTGGTACCAGCAGAAGTCAGAAACCTCCCCCAAACCCTGGATTTA |
| | | TGGCACATCCAACCTGGCTTCTGGAGTCCCTGTTCGCTTCAGTGGCAGTGGATC |
| | | TGGGACCTCTTATTCTCTCACAATCAGCAGCATGGAGGCTGAAGATGCTGCCACT |
| | | TATTACTGTCAACAGTGGAGTAGTTACCCATACACGTTCGGAGGGGGGACCAAG |
| | | CTGGAAATAAAACGTACG |

TABLE 2
Amino acid sequences of heavy and light chain protein variable region

| SEQ ID NO | Label | Sequence |
|---|---|---|
| 61 | 1E04_ | TGVHSEVQLQQSGAELVKPGASVKLSCKASGYTFTSYWVHWVRQRPGQGLEWIG |
| | HC | MIHPNSGSTNYNEKFKSKATLTVDKSSSTAYMQLSSLTSEDSAVYYCASYYYGSSYY |
| | | FDYWGQGTTLTVSSAST |
| 62 | 1E04_ | TGVHSDIVMTQSPSSLTVTAGEKVTMSCKSSQSLLNSGNQKNYLTWYQQKPGQPP |
| | LC | KLLIYWASTRESGVPDRFTGSGSGTDFTLTISSVQAEDLAVYYCQNDYSYPLTFGAG |
| | | TKLELKRT |
| 63 | 1C08_ | TGVHSEVQLQQSGAELVKPGASVKLSCKASGYTFTNYWMHWVKQRPGQGLEWIG |
| | HC | MIHPNSGTSNYNEKFKSKATLTVDKSSSTAYMQLSSLTSEDSAVFYCTRSDWAFDY |
| | | WGQGTSLTVSSAST |
| 64 | 1C08_ | TGVHSDIVMTQSPSSLSVSAGEKVTMSCKSSQSLLNSGNQKNYLAWYQQKPGQPP |
| | LC | KLLIYGASTRESGVPDRFTGSGSGTDFTLTISSVQAEDLAVYYCQNDHSYPLTFGAG |
| | | TKLELKRTPLYHAIASRMAFIFSSLYKSWXLX |
| 65 | 1B02_ | TGVHSEVQLQQSGAELVRPGSSVKMSCKTSGYTFTFYGINWVKQRPGQGLEWIGYI |
| | HC | YVGNGYSEYNEKFKVKATLTSDTSSSTAYMQLSGLTSEDSAIYFCARSDGYNYFDY |
| | | WGQGTTLTVSSTST |
| 66 | 1B02_ | TGVHSQIVLTQSPAILSASPGEKVTMTCRASSSLHYMHWYQQKTGSSPKPWIYATSN |
| | LC | LASGVPARFSGSGSGTSYSLTISRVEAEDTATYYCQQWSSNPPTFGAGTKLELKRT |
| 67 | 1A09_ | TGVHSEVQLQQPGAELVKPGASVKLSCKASGYTFTSYWMHWVKQRPGQGLEWIGL |
| | HC | IHPNSGSTYYNEKFKNKATLTVDKSSSTAYMQLSSLTSEDSAVYYCARWDDSYWYF |
| | | KVWGTGTTVTVSSAST |
| 68 | 1A09_ | TGVHSQIVLTQSPAILSASPGEKVTMTCRASSSVSYMHWYQQKPGSSPKPWIYATS |
| | LC | NLASGVPARFSGSGSGTSYSLTISRVEAEDAATYYCQQWSSNPYTFGGGTKLEIKRT |
| 69 | 2A03_ | TGVHSEVQLQQSGAELVRPGASVKLSCKASGYTFTDYYINWVKQRPGQGLEXXWIA |
| | HC | RIYPGSGNTYYNEKFKGKATLTAEKSSSTAYMQLSSLTSEDSAVYFCARRLTAGYFD |
| | | VWGTGTTVTVSSAST |
| 70 | 2A03_ | TGVHSDIKMTQSPSSMFASLGERVTITCKASQDINSYLSWFQQKPGKSPKTLIYRAN |
| | LC | RLVDGVPSRFSGSGSGQDYSLTISSLEYEDMGIYYCLQYDEFPLTFGAGTKLELKRT |
| 71 | 2C09_ | TGVHSEVMLVESGEDLVKPGGSLKLSCIASGFTFSSYAMSWVRQTPEKRLEWVAYI |
| | HC | SSGGDYIYYTDTVKGRFTISRDNARNTLFLQMSSLKSEDTAMYYCTRDTGYYVSRYF |
| | | DVWGTGTTVTVSSAST |
| 72 | 2C09_ | TGVHSDIVMTQSQKFMSTSVGDRVSVTCKASQNVGTNVAWYQQKPGQSPKALIYS |
| | LC | ASFRNSGVPDRFTGSGSGTDFTLTISNVQSEDLAEYFCHQYNNYPLTFGGGTKLEIK |
| | | RT |
| 73 | 2G02_ | TGVHSEVQLQQSGPVLVKPGPSVKISCKASGFTFTDYYMHWVKQSHGKSLEWIGLV |
| | HC | YPYNGGTYYNQKFKGKATLTVDTSSSTAYMELNSLTSEDSAVYYCVRLGNGSSNEW |
| | | YFDVWGTGTTVTVSSAST |
| 74 | 2G02_ | TGVHSQIVLTQSPALMAASPGEKVTITCSVSSSISSYNLHWYQQKSETSPKPWIYGTS |
| | LC | NLASGVPVRFSGSGSGTSYSLTISSMEAEDAATYYCQQWRTYPWTFGGGTKLEIKR |
| | | T |
| 75 | 1D09_ | TGVHSEVQLQQPGAELVKPGASVKLSCKASGYTFTNYWIHWMKQRPGRGLEWIGRI |
| | HC | EPNSGDTKYNEKIKSRATLTVDKPSSTAYMQLSSLTSEDSAVYYCARSGYDYPEAW |
| | | GQGTTLTVSSAST |
| 76 | 1D09_ | TGVHSDIVLTQSPASLAVSPGQRATISCRASEGVEYYGTTLMQWYQQKPGQPPKLLI |
| | LC | YAASNVESGVPARFSGSGSGTDFSLNIHPVEEDDIAMYFCQQSRKVSWTFGGGTKL |
| | | EIKRT |
| 77 | 2B06_ | TGVHSEVQLQQPGAELVKPGASVKLSCKASGYTFTNYWMHWVKHRPGRGLEWIGR |
| | HC | IDPNSGGTKYNEKFKNKATLTVDNPSSTGYMQLSSLTSEDSAVYYCTRSGYDYPDY |
| | | WGQGTTLTVSSAST |
| 78 | 2B06_ | TGVHSDIVLTQSPASLAVSLGQRATISCRASESVEYYGTSLMQWYQQKPGQPPKVLI |
| | LC | YAASNVDSGVPPRFSGSGSGTDFSLNFHPVEEDDIAMYFCQQSRKVPWTFGGGTK |
| | | LEIKRT |
| 79 | 2A02_ | TGVHSEVQLQQPGAELVKPGASVKLSCKASGYTFTSYWMHWVKQRPGRGLEWIGR |
| | HC | IDPNSGGTKYNEKFKSKATLTVDKPSSTAYMQLSSLTSEDSAVYYCARSGYDYPDY |
| | | WGQGTTLTVSSAST |
| 80 | 2A02_ | TGVHSDIVLTQSPAPLAVSLGQRATISCRASEGVEYYDTSLMQWYQQKPGQPPKLLI |
| | LC | YAASNVEPGVPARFGGSGSGTDFSLNIHPVEEDDIAMYFCQQSRKVPWTFGGGTKL |
| | | EIKRT |
| 81 | 1B07_ | TGVHSEVQLQQPGAELVKPGTSVKLSCKASGYTFTSYWMHWVKQRPGQGLEWIG |
| | HC | MIHPNSGSTNYNEMFESKASLTVDKSSSTAYMQLSSLTSEDSAVYYCARIGYSGWG |
| | | QGTSVTVSSAST |
| 82 | 1B07_ | TGVHSDIVMTQSPSSLSVSAGEKVTMSCKSSQSLLNGGNQKNYLAWYQQKPGQPP |
| | LC | KVLINGASTRESGVPDRFTGNGSGTDFTLTISSVQAEDLAVYYCQNDHTYPLTFGAG |
| | | TKLELKRT |
| 83 | 2D04_ | TGVHSEVQLQQPGGELVRPGSSVKMSCKTSGYTFTFYGINWVKQRPGQGLEWIGYI |
| | HC | YVGNGYTEYNEKFKVKATLTSDTSSSTAYMQLSGLTSEDSAIYFCARSDGYNYFDY |
| | | WGQGTTLTVSSAST |
| 84 | 2D04_ | TGVHSQIVLTQSPAILTASPGEKVTMTCRASSSVHYMHWYQQKPGSSPKPWIYATS |
| | LC | NLASGVPARFSGSGSGTSYSLTISRVEAEDTATYYCHQWSSNPPTFGAGTKLEIKRT |
| 85 | 1E07_ | TGVHSEVQLQQPGAELVKPGASVKLSCKASGYTFTSYWMHWVKQRPGQGLEWIGL |
| | HC | IHPNSGSTYYNEKFKNKATLTVDKSSSTAYMQLSSLTSEDSAVYYCARWDDSYWYF |
| | | KVWGTGTTVTVSSAST |
| 86 | 1E07_ | TGVHSQIVLTQSPAILSASPGEKVTMTCRASSSVSYMHWYQQKPGSSPKPWIYATS |
| | LC | NLASGVPARFSGSGSGTSYSLTISRVEAEDAATYYCQQWSSNPYTFGGGTKLEIKRT |
| 87 | 2D07_ | TGVHSEVQLQQSGPELVRPGASVKISCKASGYTFTDYYIHWLKQRPGQGLEWIGLIF |
| | HC | PGSGSIYCNEKFKGKATLTVDKSSTTAYMLLSSLTSEDSAVYFCARWETTAWYFDV |
| | | WGTGTTVTVSSAST |
| 88 | 2D07_ | TGVHSQIVLTQSPQIVLTQSPAILSASPGEKVTMTCRASLSVSYMHWYQQKPGSSPK |
| | LC | PWIYATSNLASGVPARFSGSGSGTSYSLTVSRVEAEDAATYYCQQWRSYPYTFGG |
| | | GTKLEIKRT |
| 89 | 1A02_ | TGVHSEVQLQQSGPVLVKPGPSMKISCKASGFTFTDYYIHWVRQSHGKSLEWIGLV |
| | HC | SPYNGGTYYNQKFKGKATLTVDSSSSTAYMELSSLTSEDSAVYYCARLGYYGDWYY |
| | | FDYWGQGTPLTVSSAST |
| 90 | 1A02_ | TGVHSQIVLTQSPALMATSPGEKVTITCSVSSSISSSNLNWYQQKSETSPKPWIYGTS |
| | LC | NLASGVPVRFSGSGSGTSYSLTISSMEAEDAATYYCQQWSSYPYTFGGGTKLEIKRT |
| 91 | 2D10 | TGVHSEVQLQQSGAELVKPGASVKLSCKASGYTFTTYWMHWMKQWPGQGLEWIG |
| | HC | LIHPNSGSTYYNEKFKSKATLTVDKSSSTAYMQLSSLTSEDSAVYYCARFEELWGGY |
| | | WYFDVWGTGTTVTVSSAST |
| 92 | 2D10 | TGVHSQIVLTQSPALMAASPGEKVTITCSVSSSISSSNLHWYQQKSETSPKPWIYGTS |
| | LC | NLASGVPVRFSGSGSGTSYSLTISSMEAEDAATYYCQQWSSYPYTFGGGTKLEIKRT |
| 93 | 2E08_ | TGVHSEVQLQQPGAELAKPGASVKLSCKASGYNFTSYWMHWVKQRPGQGLEWIGL |
| | HC | IHPNSGGTYYNEKFKSKATLTLDRSSSTAYMQLSSLTSEDSAVYYCARFEEEWGGY |
| | | WYFDVWGTGTTVTVSSAST |
| 94 | 2E08_ | TGVHSQIVLTQSPALMAASPGEKVTITCSVSSSISSSNLHWYQQKSETSPKPWIYGTS |
| | LC | NLASGVPVRFSGSGSGTSYSLTISSMEAEDAATYYCQQWSSYPYTFGGGTKLEIKRT |
| 95 | 2H04_ | TGVHSEVQLQQSGAELVKPGASVKLSCKASGYTFTSYWMHWVKQRPGQGLEWIGL |
| | HC | IHPNSSSTYYNEKFKTRATLTVDKSSSTAYMQLSSLTSEDSAVYYCARLGYGNSYWY |
| | | FDVWGTGTTVTVSSAST |
| 96 | 2H04_ | TGVHSQIVLTQSPALMAASPGEKVTITCSVSSSISSSNLHWYQQKSETSPKPWIYGTS |
| | LC | NLASGVPIRFSGSGSGTSYSLTISSLEAEDAATYYCQQWRSYPWTFGGGTKLEIKRT |
| 97 | 1B03_ | TGVHSEVQLQQSGAELVKPGASVKLSCKASGYTFTSYWMHWVQQRPGQGLEWIGL |
| | HC | IHPIGGGTHYNEKFKNKATLTVDKSSSTAYMQLSSLTSEDSAVYYCARLGTGPYYFD |
| | | YWGQGTTLTVSSAST |
| 98 | 1B03_ | TGVHSQIVLTQSPALMAASPGEKVTITCSVSSSISSSTLDWYQQKSETSPKPWVYGT |
| | LC | SNLASGVPVRFSGSGSGTSYSLTISSMEAEDAATYYCQQWSSYPWTFGGGTKLEIK |
| | | RT |
| 99 | 2H07_ | TGVHSEVQLQQSGPVLVKPGPSVNISCKASGFTFTDYYIHWVKQSHGKSLEWIGLVY |
| | HC | PYSGGTYYNQKFKGKATLTVDASSSTAYMELGSLTSEDSAVYYCARLGDNFYYFDY |
| | | WGQGTTLTVSSAST |
| 100 | 2H07_ | TGVHSQIVLTQSPTLMAASPGEKVTITCSVSSSISSSNLHWYQQKSETSPKPWIYGTS |
| | LC | NLASGVPVRFSGSGSGTSYSLTISSMEAEDAATYYCQQWRSYPYTFGGGTKLEIKR |
| | | T |
| 101 | 1C01_ | TGVHSEVQLQQPGPVLVKPGPSVKISCKASGFTFTDYYMHWVKQSHGKSLEWIGLV |
| | HC | SPYNGGTFYNQKFKGKATLTVDTSSSTAYMELNSLTSEDSAVYYCARVGNSYVHYA |
| | | MDYWGQGTSVTVSSAST |
| 102 | 1C01_ | TGVHSQIVLTQSPALMAASPGEKVTITCSVSSSISSSNLHWYQQKSETSPKPWIYGTS |
| | LC | NLASGVPVRFSGSGSGTSYSLTISSMEAEDAATYYCQQWSNYPYTFGGGTKLEIKR |
| | | T |
| 103 | 2C07_ | TGVHSEVKLVESGEGLVKPGGSLKLSCAASGFTFSSYAMSWVRQTPEKRLDWVAYI |
| | HC | SSGGDHIYYADTVKGRFTISRDNARNTLYLQMSSLKSEDTAMYYCTRDTGYYVSRYF |
| | | DVWGTGTTVTVSSAST |
| 104 | 2C07_ | TGVHSDIVMTQSQKFMSTSVGDRVSVTCKASQNVVTNVAWYQQKSGQSPKVVIYS |
| | LC | ASFRSSGVPDRFTGSGSGTDFTLTISNVQSEDLAEYFCHQYNSHPLTFGGGTKLEIK |
| | | RT |
| 105 | 2G04_ | TGVHSEVQLQQPGPELVKPGSSLKISCKASGYTFTDYFINWVKQRPGQGLDWIGWIF |
| | HC | PGSGSTYYNDKFKGKATLTVDKSSSTAYMLLSSLTSEASAVYFCARWDSDSTYWYF |
| | | DVWGTGTTVTVSSAST |
| 106 | 2G04_ | TGVHSQIVLTQSPALMAASPGEKVTITCSVNSSISSSTLHWYQQKSQTSPKPWIYGT |
| | LC | SNLASGVPIRFSGSGSGTSYSLTISSMEAEDAATYYCQQWSTYPYTFGGGTKLEIKR |
| | | T |
| 107 | 1G01_ | TGVHSEVQLQQSGAELMKPGASVKLSCKATGYTFTGYWIEWLKQRPGHGLEWIGEI |
| | HC | LPGSDNTNYNEKFRGKATFTADTSSNTAYMHLSSLTTEDSAIYYCAREGGFYFDYW |
| | | GQGTTLTVSSAST |
| 108 | 1G01_ | TGVHSQAVVTQESALTTSPGETVTLTCRSSIGAVTTSNYANWVQEKPDHLFTGLIGG |
| | LC | TNNRAPGVPARFSGSLIGDKAALTITGAQTEDEAIYFCALWYSNHWVFGGGTKLTVL |
| | | GQPKSSPSVTLFPPSX |
| 109 | 2A05_ | TGVHSEVQLQQSGPVLVKPGPSVKISCKASGFTFTDYYMHWVKQSHGKSLEWIGLV |
| | HC | YPYNGGTSYNQKFKGKATLTVDTSSTTAYMELNSLTSEDSAVYYCARLGSGYEYYF |
| | | DYWGQGTSLTVSSAST |
| 110 | 2A05_ | TGVHSQIVLTQSPALMAASPGEKVTITCSVSSSISSSNLHWYQQKSETSPKPWIYGTS |
| | LC | NLASGVPVRFSGSGSGTSYSLTISSMEAEDAATYYCQQWRTYPWTFGGGTKLEIKR |
| | | T |
| 111 | 2H05_ | TGVHSEVQLQQPGPVLVKPGPSVKISCKASGFTFTDYYMHWVKQSHGESLEWIGLV |
| | HC | SPYNGGTFYNQKFKGKATLTVDTSSSTAYMELNSLTSEDSAVYYCARVGSSYVHYA |
| | | MDYWGQGTSVTVSSAST |
| 112 | 2H05_ | TGVHSQIVLTQSPALTAASPGERVTITCSVSSSISSSNLHWYQQKSETSPKPWIYGTS |
| | LC | NLASGVPVRFSGSGSGTSYSLTISSMEAEDAATYYCQQWSKYPYTFGGGTKLEIKRT |
| 113 | 2B01_ | TGVHSEVQLQQSGAGLVKPGASVKLPCKASGYTFTSYWMHWVQQRPGQGLEWIG |
| | HC | LIHPIGGGTHYNEKFKNKATLTVDKSSSTAYMQLSSLTSEDSAVYYCARLGTGPYYF |
| | | DYWGQGTTLTVSSAST |
| 114 | 2B01_ | TGVHSQIVLTQSPALMAASPGEKVTITCSVSSSISSSNLHWYQQKSETSPKPWIYGTS |
| | LC | NLASGVPVRFSGSGSGTSYSLTISSMEAEDAATYYCQQWSSYPWTFGGGTKLEIKR |
| | | T |
| 115 | 1D01_ | TGVHSEVQLQQPGPDVVKPGASVKISCKASGYTFTDYYINWVKQRPGQGLEWIGWI |
| | HC | FPGSGSSYYNEKFKDKATSTVDKSSSTAYMLLSSLTSEDSAVYFCAKWKDYGWYFD |
| | | VWGTGTTVTVSSAST |
| 116 | 1D01_ | TGVHSQIVLTQSPALMAASPGEKVTLTCNVSSSLSSSNLHWYQQKSETSPKPWIYGT |
| | LC | SNLASGVPVRFSGSGSGTSYSLTISSMEAEDAATYYCQQWRTYPYTFGGGTKLEIK |
| | | RT |
| 117 | 2E02_ | TGVHSEVQLQQSGPVLVKPGPSVKISCKASGFTFTDYYIHWVKRSHGKSLEWIGLVS |
| | HC | PYNGGTFYNQKFKGKATLTVDTSSNTAYMELTSLTSEDSAVYYCARLGVNWYFDVW |
| | | GTGTTVTVSSAST |
| 118 | 2E02_ | TGVHSQIVLTQSPALMAASPGEKVTLTCSVSSSISSSTLHWYQQKSETSPKPWIYGT |
| | LC | SNLASGVPVRFSGSGSGTSYSLTISSMEAEDAATYYCQQWSTYPYTFGGGTKLEIKR |
| | | T |
| 119 | 1A05_ | TGVHSEVQLQQPGPVLVKPGPSMKISCKASGYAFTDYFIHWVRQSHGRSLEWIGLV |
| | HC | SPYNGGTYYNQKFKGKASLTVDTSSSTAYMELSSLTSEDSAVYYCARLGYYGNWYF |
| | | FDYWGQGTPLTVSSAST |
| 120 | 1A05_ | TGVHSQIVLTQSPALMATSPGEKVTITCSVSSSISSSNLNWYQQKSETSPKPWIYGTS |
| | LC | NLASGVPVRFSGSGSGTSYSLTISSMEAEDAATYYCQQWSSYPYTFGGGTKLEIKRT |

TABLE 3
Heavy and light chain protein variable region encoded by a nested
nucleotide sequence

| SEQ ID NO | Label | Sequence |
|---|---|---|
| 121 | 1E04_ | NNNNNNNNNNNNNNNNNNNGNANNNNNNGTGAAGTTGTCCTGCAAGGCTTCTG |
| | HC | GCTACACTTTCACCAGCTACTGGATGCACTGGGTGAAGCAGAGGCCTGGACAAG |
| | | GCCTTGAGTGGATTGGAATGATTCATCCTAATAGTGGTAGTACTAACTACAATGA |
| | | GAAGTTCAAGAGCAAGGCCACACTGACTGTAGACAAATCCTCCAGCACAGCCTA |
| | | CATGCAACTCAGCAGCCTGACATCTGAGGACTCTGCGGTCTATTACTGTGCAAGT |
| | | TATTACTACGGTAGTAGCTATTACTTTGACTACTGGGGCCAAGGCACCACTCTCA |
| | | CAGTCTCCTCAGCCAAAACAACAGCCCCATCGGTCTATCCACTGGCCCCTGTGT |
| | | GTGGAGGTACAACTGGCTCCTCGGTGACTCTAGGATGCCTGGTCAAGGGCTATT |
| | | NNCCCTGANCAN |
| 122 | 1E04_ | NNNCNNNNNNNTNNNCTTGGTCCCAGCACCGAACGTGAGCGGATAACTATAATC |
| | LC | ATTCTGACAGTAATAAACTGCCAGGTCTTCAGCCTGCACACTGCTGATGGTGAGA |
| | | GTGAAATCTGTTCCAGATCCACTGCCTGTGAAGCGATCAGGGACCCCAGATTCC |
| | | CTAGTGGATGCCCAGTAGATCAACAGTTTAGGAGGCTGCCCTGGTTTCTGCTGG |
| | | TACCAGGTCAAGTAGTTCTTTTGATTTCCACTGTTTAACAGACTCTGACTGGACTT |
| | | GCAGCTCATAGTGACCTTCTCTCCTGCTGTCACAGTCAGGGAGGATGGAGACTG |
| | | GGTCNNNNNAATGTCA |
| 123 | 1C08_ | GNNNNNNNNNNNTNNNNNAGNANGTTCNTGAAGTTGTCCTGCAAGGCTTCTGGC |
| | HC | TACACTTTCACCAACTACTGGATGCACTGGGTGAAGCATAGGCCTGGACAAGGC |
| | | CTTGATTGGATTGGAANGATTCATCCTAATAGTGGTACTTCTAACTACAATGAGAA |
| | | GTTCAAGAGCAAGGCCACACTGACTGTAGACAAATCCTCCAGCACAGCCTACAT |
| | | GCAACTCAGCAGCCTGACATCTGAGGACTCTGCGGTCTTTTACTGTACAAGATCT |
| | | GACTGGGCCTTTGACTACTGGGGCCAAGGCACCTCTCTCACAGTCTCCTCACCC |
| | | AAAACGACACCCCCATCTGTCTATCCACTGGCCCCTGGATCTGCTGCCCAAACTA |
| | | ACTCCATGGTGACCCTGGGATGCCTGGTCAAGGGCTATTNCCCTGANCA |
| 124 | 1C08_ | NNNNNNNNNNNCNNNNCTTGGTCNNNCACCGAACGTGAGCGGAAACTATGATCA |
| | LC | TTCTGACAGTAATAAACTGCCAGGTCTTCAGCCTGCACACTACTGATGGTAAGAG |
| | | TGAAATCGGTTCCAGATCCACTGCCTGTGAAGCGATCAGGGACCCCAGATTCCC |
| | | TAGTGGATGCCCCGTATATCAACAGTTTAGGAGGCTGCCCTGGTTTCTGCTGGTA |
| | | CCAGGCCAAGTAGTTCTTTTGATTTCCACTGTTTAACAGGCTCTGACTGGACTTG |
| | | CAGCTCATAGTGACCTTCTCTCCTGCTGACACACTCAGGGAGGATGGAGACTGG |
| | | GTNTNTCAATGTCAN |
| 125 | 1B02_ | NNNNNNNNNNNNTNANNNNNGNANTCNNAGTGAAGATGTCCTGCAAGACTTCTG |
| | HC | GATATACATTCACATTCTACGGTATAAACTGGGTGAAGCAGAGGCCTGGACAGG |
| | | GCCTGGAATGGATTGGATATATTTATGTTGGAAATGGTTATTCTGAGTACAATGA |
| | | GAAGTTCAAGGTCAAGGCCACACTGACTTCAGACACATCCTCCAGCACAGCCTA |
| | | CATGCAGCTCAGCGGCCTGACATCTGAGGACTCTGCAATCTATTTCTGTGCAAGA |
| | | TCCGATGGTTACAACTACTTTGACTACTGGGGCCAAGGCACCACTCTCACAGTCT |
| | | CCTCAGCCAAAACGACACCCCCATCTGTCTATCCACTGGCCCCTGGATCTGCTG |
| | | CCCAAACTAACTCCATGGTGACCCTGGGATGCCTGGTCAAGGGCTANTNCCCTG |
| | | ANCAN |
| 126 | 1B02_ | NNNNCNNNNCNCNNNNCTTGGTCCCAGCACCGAACGTGGGTGGGTTACTACTC |
| | LC | CACTGCTGGCAGTAATAAGTGGCAGTATCTTCAGCCTCCACTCTGCTGATTGTGA |
| | | GAGAGTAAGAGGTCCCAGACCCACTGCCACTGAAGCGAGCAGGGACTCCAGAA |
| | | GCCAGGTTGGATGTGGCATAAATCCAGGGTTTGGGGGAGGATCCTGTCTTCTGC |
| | | TGGTACCAGTGCATGTAATGTAAACTTGAGCTGGCCCTGCAAGTCATTGTGACTT |
| | | TCTCCCCTGGAGATGCAGACAGGATTGCTGGAGACTGGGNNNNNCCAATGTCAA |
| | | NA |
| 127 | 1A09_ | NNNNNNNNNNNNNNNNNNNNNNNANGNTTCAGTGAAGTTGTCCTGCAAGGCTTC |
| | HC | TGGCTACACTTTCACCAGCTACTGGATGCACTGGGTGAAGCAGAGGCCTGGACA |
| | | AGGCCTTGAGTGGATTGGATTGATTCATCCTAATAGTGGTAGTACTTACTACAAT |
| | | GAGAAGTTCAAGAACAAGGCCACACTGACTGTAGACAAATCCTCCAGCACAGCC |
| | | TACATGCAACTCAGCAGCCTGACATCTGAGGACTCTGCGGTCTATTACTGTGCAA |
| | | GATGGGATGATTCCTACTGGTACTTCAAAGTCTGGGGCACAGGGACCTCGGTCA |
| | | CCGTCTCCTCAGCCAAAACGACACCCCCATCTGTCTATCCACTGGCCCCTGGAT |
| | | CTGCTGCCCAAACTAACTCCATGGTGACCCTGGGATGCCTGGTCAAGGGCTATT |
| | | NCCCTGANNAN |
| 128 | 1A09_ | NNNNNNNNNNNNTTTNNNCTTGGTCCCCCCTCCGAACGTGTACGGGTTGCTACT |
| | LC | CCACTGCTGGCAGTAATAAGTGGCAGCATCTTCAGCCTCCACTCTGCTGATTGTG |
| | | AGAGAGTAAGAGGTCCCAGACCCACTGCCACTGAAGCGAGCAGGGACTCCAGA |
| | | AGCCAGGTTGGATGTGGCATAAATCCAGGGTTTGGGGGAGGATCCTGGCTTCTG |
| | | CTGGTACCAGTGCATGTAACTTACACTTGAACTGGCCCTGCAAGTCATTGTGACC |
| | | TTCTCCCCTGGAGATGCAGACAGGATTGCTGGAGATTGTGTCAGNACAATGTCA |
| | | N |
| 129 | 2A03_ | NNNNNNNNNNNNNNNNNNNNNNNNNNNNNNNNNNCTTCNGTGAAGCTGTCCTG |
| | HC | CAAGGCTTCTGGCTACACTTTCACTGACTACTATATAAACTGGGTGAAGCAGAGG |
| | | CCTGGACAGGGACTTGAGTGGATTGCAAGGATTTATCCTGGAAGTGGTAATACTT |
| | | ACTACAATGAGAAGTTCAAGGGCAAGGCCACACTGACTGCAGAAAAATCCTCCA |
| | | GCACTGCCTACATGCAGCTCAGCAGCCTGACATCTGAGGACTCTGCTGTCTATTT |
| | | CTGTGCAAGACGGCTAACTGCGGGATACTTCGATGTCTGGGGCACAGGGACCA |
| | | CGGTCACCGTCTCCTCAGCCAAAACGACACCCCCATCTGTCTATCCACTGGCCC |
| | | CTGGATCTGCTGCCCAAACTAACTCCATGGTGACCCTGGGATGCCTGGTCAAGG |
| | | GCTACTNCCCTGANCAN |
| 130 | 2A03_ | NNNNNNNNNNNCTNNNCTTGGTCCCAGCACCGAACGTGAGCGGAAACTCATCAT |
| | LC | ACTGTAGACAATAATAAATTCCCATATCTTCATACTCCAGGCTGCTGATGGTGAG |
| | | AGAATAATCTTGCCCAGATCCACTGCCACTGAACCTTGATGGGACCCCATCTACC |
| | | AATCTGTTTGCACGATAGATCAGGGTCTTAGGAGATTTCCCTGGTTTCTGCTGGA |
| | | ACCAGGATAAATAGCTATTAATGTCCTGACTCGCCTTGCAAGTGATAGTGACTCT |
| | | CTCTCCTAGAGATGCAAACATGGAAGATGGAGACTGTNTNN |
| 131 | 2C09_ | NNNNNNNNNNNNNNNNNNNNNNNNNNNNGNANGGNCCCTGAACTCTCCTGTAC |
| | HC | AGCCTCTGGATTCACTTTCAGTAGCTATGCCATGTCTTGGGTTCGCCAGACTCCA |
| | | GAGAAGAGGCTGGAGTGGGTCGCATATATTAGTAGTGGTGGTGATTACATCTAC |
| | | TATACAGACACTGTGAAGGGCCGATTCACCATCTCCAGAGACAATGCCAGGAAC |
| | | ACCCTGTTCCTACAAATGAGCAGTCTGAAGTCTGAGGACACAGCCATGTATTACT |
| | | GTACAAGAGATACCGGTTACTACGTTTCTCGGTACTTCGATGTCTGGGGCACAG |
| | | GGACCACGGTCACCGTCTCCTCAGCCAAAACGACACCCCCATCTGTCTATCCAC |
| | | TGGCCCCTGGATCTGCTGCCCAAACTAACTCCATGGTGACCCTGGGATGCCTGG |
| | | TCAAGGGCTACTTCCCTGAGCA |
| 132 | 2C09_ | NNNNNNNNNNTGNNTNNNNNCTTNNNNNNNNNNNNNAACGTCAGCAGGATAGTT |
| | LC | GTTATATTGATGACAGAAATACTCTGCCAAGTCTTCAGACTGCACATTGCTGATG |
| | | GTGAGAGTGAAATCTGTCCCAGATCCACTGCCTGTGAAGCGATCAGGGACTCCA |
| | | CTGTTCCGGAAGGATGCCGAGTAAATCAGTGCTTTAGGAGATTGTCCTGGTTTCT |
| | | GTTGATACCAGGCTACATTAGTTCCCACATTCTGACTGGCCTTGCAGGTGACGCT |
| | | GACCCTGTCTCCAACTGATGTGGACATGAATTTTGGAGACTGGGTCATCACAATG |
| | | TCA |
| 133 | 2G02_ | NNNNNNNNNNNNNNNNNNNNNNNNNNNNNNNNGNANCCTTCAGTGAAGATATC |
| | HC | CTGTAAGGCTTCTGGATTCACATTCACTGACTACTACATGCACTGGGTGAAGCAG |
| | | AGCCATGGAAAGAGCCTTGAATGGATTGGACTTGTTTATCCTTACAATGGTGGTA |
| | | CTTATTACAACCAGAAGTTCAAGGGCAAGGCCACATTGACTGTAGACACATCCTC |
| | | CAGCACAGCCTACATGGAGCTAAACAGCCTGACTTCTGAGGACTCTGCGGTCTA |
| | | TTACTGTGTACGATTAGGGAACGGTAGTAGCAACGAGTGGTACTTCGATGTCTG |
| | | GGGCACAGGGACCACGGTCACTGTCTCCTCAGCCAAAACGACACCCCCATCTGT |
| | | CTATCCACTGGCCCCTGGATCTGCTGCCCAAACTAACTCCATGGTGACCCTGGG |
| | | ATGCCTGGTCAAGGGCTACTTCCCTGANCA |
| 134 | 2G02_ | NNNNNCNNNTNANNNNNGTTTGGTGCCTCCCCGAACGTCCACGGGTAAGTTCTC |
| | LC | CACTGTTGACAGTAATAAGTGGCAGCATCTTCAGCCTCCATACTGCTGATTGTGA |
| | | GAGAATAAGAGGTCCCAGATCCACTGCCACTGAAGCGAACAGGGACTCCAGAAG |
| | | CCAGGTTGGATGTGCCATAAATCCAGGGTTTGGGGGAGGTTTCTGACTTCTGCT |
| | | GGTACCAGTGCAAATTGTAGGAACTTATACTTGAGCTGACACTGCAGGTGATGGT |
| | | GACCTTCTCCCCTGGAGATGCAGCCATGAGTGCTGGAGACTGNGTNANNN |
| 135 | 1D09_ | NNNNNNNNNNNGTNANNNTGNNNGCTTCNGTGAAGCTGTCCTGCAAGGCTTCTG |
| | HC | GCTACACCTTCACCAACTACTGGATACACTGGATGAAGCAGAGGCCTGGACGAG |
| | | GCCTTGAGTGGATTGGAAGGATTGAGCCTAATAGCGGTGATACTAAATACAATGA |
| | | GAAGATCAAGAGCAGGGCCACACTGACTGTAGACAAACCGTCCAGCACAGCCTA |
| | | CATGCAGCTCAGCAGCCTGACATCTGAGGACTCTGCGGTCTATTATTGTGCAAG |
| | | ATCTGGGTATGATTACCCTGAGGCCTGGGGCCAAGGCACCACTCTCACAGTCTC |
| | | CTCAGCCAAAACGACACCCCCATCTGTCTATCCACTGGCCCCTGGATCTGCTGC |
| | | CCAAACTAACTCCATGGTGACCCTGGGATGCCTGGTCAAGGGCTACTNCCCTGA |
| | | NNA |
| 136 | 1D09_ | NNNNNNNNNNNNNNNNNNNNNTTGGTGCCTCCACCGAACGTCCAAGGAACCTT |
| | LC | CCTACTTTGCTGACAGAAATACATTGCAATATCATCCTCCTCCACAGGATGGATG |
| | | TTGAGGCTGAAGTCTGTCCCAGACCCACTGCCACTAAACCTGGCAGGGACCCCA |
| | | GATTCTACGTTGGATGCAGCATAGATGAGGAGTTTGGGTGGCTGTCCTGGTTTCT |
| | | GTTGGTACCACTGCATTAAAGTTGTGCCATAATATTCAACACCTTCACTGGCTCT |
| | | GCAGGAGATGGTGGCTCTCTGCCCTAGAGACACAGCCAAAGAAGCTGGAGATTG |
| | | GGTCATCACAATGTCAN |
| 137 | 2B06_ | NNNNNNNNNNNNNNNNNNNTGANCCTGNNNCTTCAGTGAAGTTGTCCTGCAAGG |
| | HC | CTTCTGGCTATACCTTCACCAACTACTGGATGCACTGGGTGAAACACAGGCCTG |
| | | GACGAGGCCTTGAGTGGATTGGAAGGATTGATCCTAATAGTGGTGGTACTAAGT |
| | | ACAATGAGAAGTTCAAGAATAAGGCCACACTGACTGTAGACAATCCCTCCAGCAC |
| | | AGGCTACATGCAGCTCAGCAGCCTGACATCTGAGGACTCTGCGGTCTATTATTGT |
| | | ACAAGATCTGGGTATGATTACCCTGACTACTGGGGCCAAGGCACCACTCTCACA |
| | | GTCTCCTCAGCCAAAACGACACCCCCATCTGTCTATCCACTGGCCCCTGGATCT |
| | | GCTGCCCAAACTAACTCCATGGTGACCCTGGGATGCCTGGTCAAGGGCTATTTN |
| | | CCCTGANAAN |
| 138 | 2B06_ | NNNNNNNNNNNNNNTNNNNGCTTGGTGCCTCCNCCGAACGTCCAAGGAACCTT |
| | LC | CCTACTTTGCTGACAGAAATACATTGCAATATCATCCTCCTCCACAGGATGGAAG |
| | | TTGAGGCTGAAGTCTGTCCCAGACCCACTGCCACTAAACCTGGGAGGGACCCCA |
| | | GAATCTACGTTGGATGCAGCATAGATGAGGACTTTGGGTGGCTGTCCTGGTTTCT |
| | | GTTGGTACCACTGCATTAAACTTGTGCCATAATATTCAACACTTTCACTGGCTCTG |
| | | CAGGAGATGGTGGCTCTCTGCCCTAGAGACACAGCCAAAGAAGCTGGAGACTG |
| | | GGTNANNNCAATGTCAA |
| 139 | 2A02_ | NNNNNNNNNNNNNNNNNNNNNNANCNNGNNNGCTTCAGTGAAGCTGTCCTGCA |
| | HC | AGGCTTCTGGCTACACCTTCACCAGCTACTGGATGCACTGGGTGAAGCAGAGGC |
| | | CTGGACGAGGCCTTGAGTGGATTGGAAGGATTGATCCTAATAGTGGTGGTACTA |
| | | AGTACAATGAGAAGTTCAAGAGCAAGGCCACACTGACTGTAGACAAACCCTCCA |
| | | GCACAGCCTACATGCAGCTCAGCAGCCTGACATCTGAGGACTCTGCGGTCTATT |
| | | ATTGTGCAAGATCTGGGTATGATTACCCTGACTACTGGGGCCAAGGCACCACTC |
| | | TCACAGTCTCCTCAGCCAAAACGACACCCCCATCTGTCTATCCACTGGCCCCTG |
| | | GATCTGCTGCCCAAACTAACTCCATGGTGACCCTGGGATGCCTGGTCAAGGGCT |
| | | ACTTCCCTGANCA |
| 140 | 2A02_ | NNNNNNNNNATNNNNCTTGGTGCCTCCCCGAACGTCCAAGGAACCTTCCTACTT |
| | LC | TGCTGACAGAAATACATTGCAATATCATCCTCCTCCACAGGATGGATGTTGAGGC |
| | | TGAAGTCTGTCCCAGACCCACTGCCACCAAACCTGGCAGGAACCCCAGGTTCTA |
| | | CGTTGGATGCAGCATAGATGAGGAGTTTGGGTGGCTGTCCTGGTTTCTGTTGGT |
| | | ACCACTGCATTAAACTTGTGTCATAATATTCAACACCTTCACTGGCTCTGCAGGA |
| | | GATGGTGGCTCTCTGCCCTAGAGACACAGCCAAAGAAGCTGGAGNNTGTNNNNN |
| | | N |
| 141 | 1B07_ | NNNNNNNNNNNNNNNNCNTGANNANNATTCNGTGAAGTTGTCCTGCAAGGCTTC |
| | HC | TGGCTACACTTTCACCATCTACTGGATGCACTGGGTGAAGCATAGGCCTGGACA |
| | | AGGCCTTGAGTGGATTGTAATGATTCATCCTAATATTGGTATTACTAACTACAATG |
| | | AGATGTCGAGAGCAAGGCCTCACTGACTGTAGACAAATCCTCCAGCACATCCTA |
| | | CATGCAACTCANCAGCCTGACATCTGAGGACTCTGCGGTCTATTACTGTGCAAGA |
| | | ATCGGATACTCCGGCTGGGGTCAAGGAACCTCATTCACCGTCTCCTCAGCCAAA |
| | | ACGACACCCCCATCTGTCTATCCACTGGCCCCTGGATCTGCTGCCCAAACTAACT |
| | | CCATGGTGACCCTGGGATGCCTGGTCAAGGGCTACTNCCCTGANCA |
| 142 | 1B07_ | GNNNNNNCTNNCTNNNCTTGGTCCCAGCACCGAACGTGAGCGGATAAGTATGAT |
| | LC | CATTCTGACAGTAATAAACTGCCAGGTCTTCAGCCTGCACACTGCTGATGGTAAG |
| | | AGTGAAATCGGTTCCAGATCCATTGCCTGTGAAGCGATCAGGGACCCCAGATTC |
| | | CCTAGTGGATGCCCCGTTGATCAACACTTTAGGAGGCTGCCCTGGTTTCTGCTG |
| | | GTACCAGGCCAAGTAGTTCTTTTGATTTCCACCATTTAACAGACTCTGACTGGAC |
| | | TTGCAGCTCATAGTGACCTTCTCTCCTGCTGACACACTCAGGGAGGATGGAGAC |
| | | TGGGTNTNACAATGTCANA |
| 143 | 2D04_ | NNNNNNNNNNNANCTGGNGNNGCCTGGGTCCTCAGTGAAGATGTCCTGCAAGA |
| | HC | CTTCTGGATATACATTCACATTCTACGGTATAAACTGGGTGAAGCAGAGGCCTGG |
| | | ACAGGGCCTGGAATGGATTGGATATATTTATGTTGGAAATGGTTATACTGAGTAC |
| | | AATGAGAAGTTCAAGGTCAAGGCCACACTGACTTCAGACACATCCTCCAGCACA |
| | | GCCTACATGCAGCTCAGCGGCCTGACATCTGAGGACTCTGCAATCTATTTCTGTG |
| | | CAAGATCCGATGGTTACAACTACTTCGACTACTGGGGCCAAGGCACCACTCTCA |
| | | CAGTCTCCTCAGCCAAAACGACACCCCCATCTGTCTATCCACTGGCCCCTGGAT |
| | | CTGCTGCCCAAACTAACTCCATGGTGACCCTGGGATGCCTGGTCAAGGGCTATT |
| | | NNCCTGANCAN |
| 144 | 2D04_ | NNNNNNNNTNTNGCTNNNNNTTGNNTCCCNGNACCGAACGTGGGTGGGTTACTA |
| | LC | CTCCACTGATGGCAGTAATAAGTGGCAGTATCTTCAGCCTCCACTCTGCTGATTG |
| | | TGAGAGAGTAAGAGGTCCCAGACCCACTGCCACTGAAGCGAGCAGGGACTCCA |
| | | GAAGCCAGGTTGGATGTGGCATAAATCCAGGGTTTGGGGGAGGATCCTGGCTTC |
| | | TGCTGGTACCAGTGCATGTAATGTACACTTGAACTGGCCCTGCAAGTCATTGTGA |
| | | CTTTCTCCCCTGGAGATGCAGTCAGGATTGCTGGAGACTGGGTCATCACNATGT |
| | | CAA |
| 145 | 1E07_ | NNNNNNNNNNNNNNNNNNNNNNNAGNNNNCNNNNNTGAAGTTGTCCTGCAAGG |
| | HC | CTTCTGGCTACACTTTCACCAGCTACTGGATGCACTGGGTGAAGCAGAGGCCTG |
| | | GACAAGGCCTTGAGTGGATTGGATTGATTCATCCTAATAGTGGTAGTACTTACTA |
| | | CAATGAGAAGTTCAAGAACAAGGCCACACTGACTGTAGACAAATCCTCCAGCACA |
| | | GCCTACATGCAACTCAGCAGCCTGACATCTGAGGACTCTGCGGTCTATTACTGT |
| | | GCAAGATGGGATGATTCCTACTGGTACTTCAAAGTCTGGGGCACAGGGACCTCG |
| | | GTCACCGTCTCCTCAGCCAAAACGACACCCCCATCTGTCTATCCACTGGCCCCT |
| | | GGATCTGCTGCCCAAACTAACTCCATGGTGACCCTGGGATGCCTGGTCAAGGGC |
| | | TACTTCCCTGANCA |
| 146 | 1E07_ | NNNNNNNNNNNNNNCTTGGTCCCCCCTCCGAACGTGTACGGGTTGCTACTCCAC |
| | LC | TGCTGGCAGTAATAAGTGGCAGCATCTTCAGCCTCCACTCTGCTGATTGTGAGA |
| | | GAGTAAGAGGTCCCAGACCCACTGCCACTGAAGCGAGCAGGGACTCCAGAAGC |
| | | CAGGTTGGATGTGGCATAAATCCAGGGTTTGGGGGAGGATCCTGGCTTCTGCTG |
| | | GTACCAGTGCATGTAACTTACACTTGAACTGGCCCTGCAAGTCATTGTGACCTTC |
| | | TCCCCTGGAGATGCAGACAGGATTGCTGGAGACTGGGTNNNNNCAATGTCAN |
| 147 | 2D07_ | NNNNCNNNNNNNNNNNNNNTNNNNNNNNNNGNNNGCTTCNGTGAAGATATCCT |
| | HC | GCAAGGCTTCTGGCTACACCTTCACTGACTACTATATACACTGGCTGAAGCAGAG |
| | | GCCTGGACAGGGACTTGAGTGGATTGGATTGATTTTTCCTGGAAGTGGTAGTATT |
| | | TACTGTAATGAGAAGTTCAAGGGCAAGGCCACACTTACTGTAGACAAATCCTCCA |
| | | CCACAGCCTACATGTTGCTCAGCAGCCTGACCTCTGAGGACTCTGCGGTCTATTT |
| | | CTGTGCAAGATGGGAGACTACGGCGTGGTACTTCGATGTCTGGGGCACAGGGA |
| | | CCACGGTCACCGTCTCCTCAGCCAAAACGACACCCCCATCTGTCTATCCACTGG |
| | | CCCCTGGATCTGCTGCCCAAACTAACTCCATGGTGACCCTGGGATGCCTGGTCA |
| | | AGGGCTACTTCCCTGAGCA |
| 148 | 2D07_ | NNNNNCNNNNNNNNNNNNTTGNNNNNNCTCCGAACGTGTACGGGTAACTCCTC |
| | LC | CACTGCTGGCAGTAATAAGTGGCAGCATCTTCGGCCTCCACTCTGCTGACTGTG |
| | | AGAGAGTAAGAGGTCCCAGACCCACTGCCACTGAAGCGGGCAGGGACTCCAGA |
| | | AGCCAGGTTGGATGTGGCATAAATCCAGGGTTTGGGGGAGGATCCTGGCTTCTG |
| | | CTGGTACCAGTGCATGTAACTTACACTTAAACTGGCCCTGCAAGTCATTGTGACC |
| | | TTCTCCCCTGGAGATGCAGACAGGATTGCTGGAGACTGGGTCANNACAATGTCA |
| | | N |
| 149 | 1A02_ | NNNNNNNGGTGAGCCTGGGCCTTCATGAAGATATCCTGTAAGGCTTCTGGATTC |
| | HC | ACATTCACTGACTACTACATACACTGGGTGAGGCAGAGCCATGGAAAGAGCCTT |
| | | GAGTGGATTGGACTTGTTTCTCCTTACAATGGTGGTACTTACTACAACCAGAAGT |
| | | TCAAGGGCAAGGCCACATTGACTGTAGACTCATCCTCCAGCACAGCCTACATGG |
| | | AGCTAAGCAGCCTGACTTCTGAGGACTCTGCGGTCTATTACTGTGCAAGATTGG |
| | | GCTACTATGGTGACTGGTACTACTTTGACTACTGGGGCCAAGGCACCCCTCTCA |
| | | CAGTCTCCTCAGCCAAAACGACACCCCCATCTGTCTATCCACTGGCCCCTGGAT |
| | | CTGCTGCCCAAACTAACTCCATGGTGACCCTGGGATGCCTGGTCAAGGGCTATT |
| | | NNCCCTGANCAN |
| 150 | 1A02_ | NNNNNNNNNNNNNNNNTTGGTCCCCCCTCCGAACGTGTATGGGTAACTACTCCA |
| | LC | CTGTTGACAGTAATAAGTGGCAGCATCTTCAGCCTCCATGCTGCTGATTGTGAGA |
| | | GAATAAGAGGTCCCAGATCCACTGCCACTGAAGCGAACAGGGACTCCAGAAGCC |
| | | AGGTTGGATGTGCCATAAATCCAGGGTTTGGGGGAGGTTTCTGACTTCTGCTGG |
| | | TACCAGTTCAAGTTGCTGGAACTTATACTTGAGCTGACACTACAGGTGATGGTGA |
| | | CCTTTTCCCCTGGAGATGTAGCCATGAGTGCTGGAGACTGGGTGANNNCAATGT |
| | | CANN |
| 151 | 2D10_ | NNNNNNNNNNNNNNNNNNNNNNNCCNGNNNCTTCAGTGAAGTTGTCCTGCAAG |
| | HC | GCTTCTGGCTACACTTTCACCACCTACTGGATGCACTGGATGAAGCAGTGGCCT |
| | | GGACAAGGCCTTGAGTGGATTGGATTGATTCATCCTAATAGTGGTAGTACTTACT |
| | | ACAATGAGAAGTTCAAGAGCAAGGCCACACTGACTGTAGACAAATCCTCCAGCA |
| | | CAGCCTACATGCAACTCAGCAGCCTGACATCTGAGGACTCTGCGGTCTATTACT |
| | | GTGCAAGATTTGAGGAGCTATGGGGAGGTTACTGGTACTTCGATGTCTGGGGCA |
| | | CAGGGACCACGGTCACCGTCTCCTCAGCCAAAACGACACCCCCATCTGTCTATC |
| | | CACTGGCCCCTGGATCTGCTGCCCAAACTAACTCCATGGTGACCCTGGGATGCC |
| | | TGGTCAAGGGCTATTTCCCTGANCAN |
| 152 | 2D10_ | NNNNNNNNTTNNNNNNNCTTGGTCCCCCTCCGAACGTGTACGGGTAACTACTCC |
| | LC | ACTGTTGACAGTAATAAGTGGCAGCATCTTCAGCCTCCATGCTGCTGATTGTGAG |
| | | AGAATAAGAGGTCCCAGATCCACTGCCACTGAAGCGAACAGGGACTCCAGAAGC |
| | | CAGGTTGGATGTGCCATAAATCCAGGGTTTGGGGGAGGTTTCTGACTTCTGCTG |
| | | GTACCAGTGCAAGTTGCTGGAACTTATACTTGAGCTGACACTGCAGGTGATGGT |
| | | GACCTTCTCCCCTGGAGATGCAGCCATGAGTGCTGGAGACTGGGTNNNNACAAT |
| | | GTCAN |
| 153 | 2E08_ | NNNNNNNNNNNNNNNNNNNNNNNNNNNNNGNNNGCTTCAGTGAAGTTGTCCTG |
| | HC | CAAGGCTTCTGGCTACAATTTCACCAGCTACTGGATGCACTGGGTGAAGCAGAG |
| | | GCCTGGACAAGGCCTTGAGTGGATTGGATTGATTCATCCTAATAGTGGTGGTACT |
| | | TACTACAATGAGAAGTTCAAGAGCAAGGCCACACTGACTGTAGACAGATCCTCCA |
| | | GCACAGCCTACATGCAACTCAGCAGCCTGACATCTGAGGACTCTGCGGTCTATT |
| | | ATTGTGCAAGATTTGAGGAGGAATGGGGAGGGTATTGGTACTTCGATGTCTGGG |
| | | GCACAGGGACCACGGTCACCGTCTCCTCAGCCAAAACGACACCCCCATCTGTCT |
| | | ATCCACTGGCCCCTGGATCTGCTGCCCAAACTAACTCCATGGTGACCCTGGGAT |
| | | GCCTGGTCAAGGGCTATTTCCCTGAGCA |
| 154 | 2E08_ | NNNNNNNNNTNTNNNNNNTTGGTNNNCCTCCGAACGTGTACGGGTAACTACTCC |
| | LC | ACTGTTGACAGTAATAAGTGGCAGCATCTTCAGCCTCCATGCTGCTGATTGTGAG |
| | | AGAATAAGAGGTCCCAGATCCACTGCCACTGAAGCGAACAGGGACTCCAGAAGC |
| | | CAGGTTGGAAGTGCCATAAATCCAGGGTTTGGGGGAGGTTTCTGACTTCTGTTG |
| | | GTACCAGTGCAAGTTGCTGGAACTTATACTTGAACTGACACTGCAGGTGATGGTG |
| | | ACCTTTTCCCCTGGAGATGCAGCCATGAGTGCTGGAGTNTGGGTCANNN |
| 155 | 2H04_ | NNNNANNNNNNNNNNNNNNNNNNNNNNNNNNNNNNNCTTCNGTGAAGTTGTCC |
| | HC | TGCAAGGCTTCTGGCTACACTTTCACCAGCTACTGGATGCACTGGGTGAAGCAG |
| | | AGGCCTGGACAAGGCCTTGAGTGGATTGGACTGATTCATCCTAATAGTAGTAGTA |
| | | CTTACTACAATGAGAAGTTCAAGACCAGGGCCACACTGACTGTAGACAAGTCCTC |
| | | CAGCACAGCCTACATGCAACTCAGCAGCCTGACATCTGAGGACTCTGCGGTCTA |
| | | TTACTGTGCAAGATTGGGCTATGGTAACTCCTACTGGTACTTCGATGTCTGGGGC |
| | | ACAGGGACCACGGTCACCGTCTCCTCAGCCAAAACGACACCCCCATCTGTCTAT |
| | | CCACTGGCCCCTGGATCTGCTGCCCAAACTAACTCCATGGTGACCCTGGGATGC |
| | | CTGGTCAAGGGCTACTTCCCTGANCA |
| 156 | 2H04_ | NNNNNNNNNNNNNNNNGCTTGGTGCCTCCACCGAACGTCCACGGGTAACTTCTC |
| | LC | CACTGTTGACAGTAATAAGTGGCAGCATCTTCAGCCTCCAAACTGCTGATTGTGA |
| | | GAGAATAAGAGGTCCCAGATCCACTGCCACTGAAGCGAATAGGGACTCCAGAAG |
| | | CCAGGTTGGATGTGCCATAAATCCAGGGTTTGGGGGAGGTTTCTGACTTCTGCT |
| | | GGTACCAGTGCAAGTTGCTGGAACTAATACTTGAGCTGACACTGCAGGTGATGG |
| | | TGACCTTCTCCCCTGGAGATGCAGCCATGAGTGCTGGAGACTGGGTNNNNCAAT |
| | | GTCA |
| 157 | 1B03_ | NNNNNNNNNNNNNNNNNNNNNNNNNNNNNNTTCNGTGAAGTTGTCCTGCAAGG |
| | HC | CTTCTGGCTACACTTTCACCAGCTACTGGATGCACTGGGTGCAGCAGAGGCCTG |
| | | GACAAGGCCTTGAGTGGATTGGATTGATTCATCCTATTGGTGGTGGTACTCACTA |
| | | CAATGAGAAGTTCAAGAACAAGGCCACACTGACTGTAGACAAATCCTCCAGCACA |
| | | GCCTACATGCAACTCAGCAGCCTGACATCTGAGGACTCTGCGGTCTATTACTGT |
| | | GCAAGACTCGGAACTGGTCCGTACTACTTTGACTACTGGGGCCAAGGCACCACT |
| | | CTCACAGTCTCCTCAGCCAAAACGACACCCCCATCTGTCTATCCACTGGCCCCT |
| | | GGATCTGCTGCCCAAACTAACTCCATGGTGACCCTGGGATGCCTGGTCAAGGGC |
| | | TATTTCCCTGANCA |
| 158 | 1B03_ | NNNNCNTTNNNTNNNNNTTTGGTGCCTCCACCGAACGTCCACGGGTAACTACTC |
| | LC | CACTGTTGACAGTAATAAGTGGCAGCATCTTCAGCCTCCATGCTGCTGATTGTGA |
| | | GAGAATAAGAGGTCCCAGATCCACTGCCACTGAAGCGAACAGGGACTCCAGAAG |
| | | CCAGGTTGGATGTGCCATAAATCCAGGGTTTGGGGGAGGTTTCTGACTTCTGCT |
| | | GGTACCAGTCCAAGGTGCTGGAACTTATACTTGAGCTGACACTGCAGGTGATGG |
| | | TGACCTTCTCCCCTGGAGATGCAGCCATGAGTGCTGGAGACTGGGTCNNNACAA |
| | | TGTCANNN |
| 159 | 2H07_ | NNNNNNNNNNNNNNNNNNNNNNNNNNANNNNGNANCCTTCAGTGAATATATCCT |
| | HC | GTAAGGCTTCTGGATTCACATTCACTGACTACTACATTCACTGGGTGAAACAGAG |
| | | CCATGGAAAGAGCCTTGAGTGGATTGGACTTGTTTATCCTTACAGTGGTGGTACT |
| | | TACTACAACCAGAAGTTCAAGGGCAAGGCCACATTGACTGTAGACACATCCTCCA |
| | | GCACAGCCTACATGGAGCTAGGCAGCCTGACTTCTGAGGACTCTGCGGTCTATT |
| | | ACTGTGCAAGATTGGGCGACAACTTCTACTACTTTGACTACTGGGGCCAAGGCA |
| | | CCACTCTCACAGTCTCCTCAGCCAAAACGACACCCCCATCTGTCTATCCACTGGC |
| | | CCCTGGATCTGCTGCCCAAACTAACTCCATGGTGACCCTGGGATGCCTGGTCAA |
| | | GGGCTATTTCCCTGAGCAN |
| 160 | 2H07_ | NNNNNNNNNNNTTNNGCTTGGTCNNCCTCCGAACGTGTACGGGTAACTTCTCCA |
| | LC | CTGTTGACAGTAATAAGTGGCAGCATCTTCAGCCTCCATGCTGCTGATTGTGAGA |
| | | GAATAAGATGTCCCAGATCCACTGCCACTGAAGCGAACAGGGACTCCAGAAGCC |
| | | AGGTTGGATGTGCCATAAATCCAGGGTTTGGGGGAGGTTTCTGACTTCTGCTGG |
| | | TACCAGTGCAAGTTGCTGGAACTTATACTTGAGCTGACACTGCAGGTGATGGTGA |
| | | CCTTCTCCCCTGGAGATGCAGCCATGAGTGTTGGAGTNTGNGNNANNACAATGT |
| | | CAN |
| 161 | 1C01_ | GNNNNNNNNNNNNNNNNNNNNNNNNNNNNNCTTCAGTGAAGATATCCTGTAAG |
| | HC | GCTTCTGGATTCACATTCACTGACTACTACATGCACTGGGTGAAACAGAGCCATG |
| | | GAAAGAGCCTTGAGTGGATTGGTCTTGTTTCTCCTTACAATGGTGGTACTTTCTA |
| | | CAACCAGAAGTTCAAGGGCAAGGCCACATTGACTGTAGACACATCCTCCAGCAC |
| | | AGCCTACATGGAACTAAACAGCCTGACTTCTGAGGACTCTGCGGTCTATTACTGT |
| | | GCAAGAGTGGGTAATAGCTACGTCCATTATGCTATGGACTACTGGGGTCAAGGA |
| | | ACCTCAGTCACCGTCTCCTCAGCCAAAACGACACCCCCATCTGTCTATCCACTGG |
| | | CCCCTGGATCTGCTGCCCAAACTAACTCCATGGTGACCCTGGGATGCCTGGTCA |
| | | AGGGCTACTTCCCTGANCA |
| 162 | 1C01_ | NNNNCNNNNNNNNNNNCTTGGTCCCCCCTCCGAACGTGTACGGGTAATTACTCC |
| | LC | ACTGTTGACAGTAATAAGTGGCAGCATCTTCAGCCTCCATGCTGCTGATTGTGAG |
| | | AGAATAAGAGGTCCCAGATCCACTGCCACTGAAGCGAACAGGGACTCCAGAAGC |
| | | CAGGTTGGATGTGCCATAAATCCAGGGTTTGGGGGAGGTTTCTGACTTCTGCTG |
| | | GTACCAGTGCAAGTTGCTGGAACTTATACTTGAGCTGACACTGCAGGTGATGGT |
| | | GACCTTCTCCCCTGGAGATGCAGCCATGAGTGCTGGAGACTGGGTGANNNCAAT |
| | | GTCANN |
| 163 | 2C07_ | NNNNNNNNNNNNNNNNNNNNNNNNNNNNNNNNNNANGGTCCCTGNNNCTCTCC |
| | HC | TGTGCAGCCTCTGGATTCACTTTCAGTAGCTATGCCATGTCTTGGGTTCGCCAGA |
| | | CTCCAGAGAAGAGGCTGGACTGGGTCGCATACATTAGTAGTGGTGGTGATCACA |
| | | TCTACTATGCAGACACTGTGAAGGGCCGATTCACCATCTCCAGAGACAATGCCA |
| | | GGAACACCCTGTACCTGCAAATGAGCAGTCTGAAGTCTGAGGACACAGCCATGT |
| | | ATTACTGTACAAGAGATACCGGTTACTACGTTTCTCGGTACTTCGATGTCTGGGG |
| | | CACAGGGACCACGGTCACCGTCTCCTCAGCCAAAACGACACCCCCATCTGTCTA |
| | | TCCACTGGCCCCTGGATCTGCTGCCCAAACTAACTCCNTGGTGACCCTGGGATG |
| | | CCTGGTCAAGGGCTANTTCCCTGANCAAATA |
| 164 | 2C07_ | NNNNNNNNNNNTNTGNNNNNNNANTTGNTGCCTCCNCCNANCGTCAGAGGATG |
| | LC | GCTGTTATATTGGTGACAGAAATACTCTGCCAAGTCTTCAGACTGCACATTGCTG |
| | | ATGGTGAGAGTGAAATCTGTCCCAGATCCACTGCCTGTGAAGCGATCAGGGACT |
| | | CCACTGGACCGGAAGGATGCCGAATAAATCACTACTTTAGGAGATTGCCCTGATT |
| | | TCTGTTGATACCAGGCAACATTAGTAACCACATTCTGACTGGCCTTGCAGGTGAC |
| | | GCTGACCCTGTCTCCTACTGATGTGGACATGAATTTTGGAGACTGGGTCATCACA |
| | | ATGTCA |
| 165 | 2G04_ | NNNNNNNNNNNNNNNNNNNNNNNNNNNNNNNNANTCTTCACTGAAGATATCCTG |
| | HC | CAAGGCTTCTGGCTACACCTTCACTGACTACTTTATAAACTGGGTGAAACAGAGG |
| | | CCTGGACAGGGACTTGACTGGATTGGATGGATTTTTCCTGGAAGTGGTAGTACTT |
| | | ACTACAATGACAAGTTCAAGGGCAAGGCCACACTTACTGTAGACAAATCCTCCAG |
| | | CACTGCCTACATGTTGCTCAGCAGCCTGACCTCTGAGGCCTCTGCGGTCTATTTC |
| | | TGTGCAAGATGGGACTCCGATAGTACCTACTGGTACTTCGATGTCTGGGGCACA |
| | | GGGACCACGGTCACCGTCTCCTCAGCCAAAACGACACCCCCATCTGTCTATCCA |
| | | CTGGCCCCTGGATCTGCTGCCCAAACTAACTCCATGGTGACCCTGGGATGCCTG |
| | | GTCAAGGGCTATTTCCCTGANCA |
| 166 | 2G04_ | NNNNNNCNNNNNTNNNNNCTTGGTNNNCCTCCGAACGTGTACGGGTAAGTACTC |
| | LC | CACTGTTGACAGTAATAAGTGGCAGCATCTTCAGCCTCCATGCTGCTGATTGTGA |
| | | GAGAATAAGAGGTCCCAGATCCACTGCCACTGAAGCGAATAGGGACTCCAGAAG |
| | | CCAAATTGGATGTGCCATAAATCCAGGGTTTGGGGGAGGTTTGTGACTTCTGCT |
| | | GGTACCAGTGCAAGGTGCTGGAACTTATACTTGAATTGACACTGCAGGTGATGG |
| | | TGACCTTCTCCCCTGGAGATGCAGCCATGAGTGCTGGAGTCTGGGTCANNNCAA |
| | | TGTCA |
| 167 | 1G01_ | NNNNNNNNNCNNNNNNNNNNNNNNANGCCTCAGTGAAGCTTTCCTGCAAGGCT |
| | HC | ACTGGCTACACATTCACTGGCTACTGGATAGAGTGGTTAAAGCAGAGGCCTGGA |
| | | CATGGCCTTGAGTGGATTGGAGAGATTTTACCTGGAAGTGATAATACTAACTACA |
| | | ATGAGAAGTTCAGGGGCAAGGCCACATTCACTGCAGATACATCCTCCAACACAG |
| | | CCTACATGCACCTCAGCAGCCTGACAACTGAGGACTCTGCCATCTATTACTGTGC |
| | | AAGAGAAGGGGGTTTCTACTTTGACTACTGGGGCCAAGGCACCACTCTCACTGT |
| | | CTCCTCAGCCAAAACGACACCCCCATCTGTCTATCCACTGGCCCCTGGATCTGC |
| | | TGCCCAAACTAACTCCATGGTGACCCTGGGATGCCTGGTCAAGGGCTACTNCCC |
| | | TGANCAA |
| 168 | 1G01_ | NNNNNNNNNNTGGTGANNGTCACACTCACTTGTCGCTCAAGTATTGGGGCTGTT |
| | LC | ACAACTAGTAACTACGCCAACTGGGTCCAAGAAAAACCAGATCATTTATTCACTG |
| | | GTCTAATAGGTGGTACCAACAACCGAGCTCCAGGTGTTCCTGCCAGATTCTCAG |
| | | GCTCCCTGATTGGAGACAAGGCTGCCCTCACCATCACAGGGGCACAGACTGAG |
| | | GATGAGGCAATATATTTCTGTGCTCTATGGTACAGCAACCATTGGGTGTTCGGTG |
| | | GAGGAACCAAACTGACTGTCCTAGGCCAGCCCAAGTCTTCGCCATCAGTCACCC |
| | | TGTTCCCACCTTCCNCTGAAGAGA |
| 169 | 2A05_ | NNNNNNNNNNNNNNNNNNNNANNCCNGNNNCCTTCNGTGAAGATATCCTGTAAG |
| | HO | GCTTCTGGATTCACATTCACTGACTACTACATGCACTGGGTGAAGCAGAGCCATG |
| | | GAAAGAGCCTTGAATGGATTGGACTTGTTTATCCTTACAATGGTGGTACTAGCTA |
| | | CAACCAGAAGTTCAAGGGCAAGGCCACATTGACTGTAGACACATCCTCCACCAC |
| | | AGCCTACATGGAGCTAAACAGCCTGACTTCTGAGGACTCTGCGGTCTATTACTGT |
| | | GCAAGATTGGGCTCAGGCTACGAGTACTACTTTGACTACTGGGGCCAAGGCACC |
| | | TCTCTCACAGTCTCCTCAGCCAAAACGACACCCCCATCTGTCTATCCACTGGCCC |
| | | CTGGATCTGCTGCCCAAACTAACTCCATGGTGACCCTGGGATGCCTGGTCAAGG |
| | | GCTACTNCCCTGANCAN |
| 170 | 2A05_ | NNNNCNNNNNNTTNNNGCTTGGTGCCTCCNCCGAACGTCCACGGGTAAGTTCTC |
| | LC | CACTGTTGACAGTAATAAGTGGCAGCATCCTCAGCCTCCATGCTGCTGATTGTGA |
| | | GAGAATAAGAGGTCCCAGATCCACTGCCACTGAAGCGAACAGGGACTCCAGAAG |
| | | CCAGGTTGGATGTGCCATAAATCCAGGGTTTGGGGGAGGTTTCTGACTTCTGCT |
| | | GGTACCAGTGCAAGTTGCTGGAACTTATACTTGAGCTGACACTGCAGGTGATGG |
| | | TGACCTTCTCCCCTGGAGATGCAGCCATGAGTGCTGGAGNCTGGGTNTNN |
| 171 | 2H05_ | NNNNNNNNNNNNNNNNNNNNNNNNNNNNNNNNNNNNNNNTNCAGTGANANNTC |
| | HC | CTGTAAGGCTTCTGGATTCACATTCACTGACTACTACATGCACTGGGTGAAGCAG |
| | | AGCCATGGAGAGAGCCTTGAGTGGATTGGACTTGTTTCTCCTTACAATGGTGGTA |
| | | CTTTCTACAACCAGAAGTTCAAGGGCAAGGCCACATTGACTGTAGACACATCCTC |
| | | CAGCACAGCCTACATGGAACTAAACAGCCTGACTTCTGAGGACTCTGCGGTCTA |
| | | TTACTGTGCAAGAGTGGGTAGTAGTTACGTCCATTATGCTATGGACTACTGGGGT |
| | | CAAGGAACCTCAGTCACCGTCTCCTCAGCCAAAACGACACCCCCATCTGTCTATC |
| | | CACTGGCCCCTGGATCTGCTGCCCAAACTAACTCCATGGTGACCCTGGGATGCC |
| | | TGGTCAAGGGCTACTTCCCTGAGCA |
| 172 | 2H05_ | NNNNNNNNNNNNNNNNNNNNNNTNNNNCTTGGTCNCCCTCCGAACGTGTACGG |
| | LC | GTACTTACTCCACTGTTGACAGNAATAAGTGGCAGCATCTTCAGCCTCCATGCTG |
| | | CTGATTGTGAGAGAATAANAGGTCCCAGATCCACTGCCACTGAAGCGAACAGGG |
| | | ACTCCAGAAGCCAGGTTGGATGTGCCATAAATCCAGGGTTTGGGGGAGGTTTCT |
| | | GACTTCTGCTGGTACCAGTGCAAGTTGCTGGAACTTATACTTGAGCTGACACTGC |
| | | AGGTGATGGTGACCCTCTCCCCTGGAGATGCAGCCATGAGTGCTGGAGACTGG |
| | | GTCATCACAATGTCA |
| 173 | 2B01_ | NNNNNNNNNNNNNNNNNNNNNNNNNNNNNNNANGCTTCAGTGAAGTTGTCCTG |
| | HC | CANGGCTTCTGGCTACACTTTCACCAGCTACTGGATGCACTGGGTGCAGCAGAG |
| | | GCCTGGACAAGGCCTTGAGTGGATTGGATTGATTCATCCTATTGGTGGTGGTACT |
| | | CACTACAATGAGAAGTTCAAGAACAAGGCCACACTGACTGTAGACAAATCCTCCA |
| | | GCACAGCCTACATGCAACTCAGCAGCCTGACATCTGAGGACTCTGCGGTCTATT |
| | | ACTGTGCAAGACTCGGAACTGGTCCGTACTACTTTGACTACTGGGGCCAAGGCA |
| | | CCACTCTCACAGTCTCCTCACCCAAAACGACACCCCCATCTGTCTATCCACTGGC |
| | | CCCTGGATCTGCTGCCCAAACTAACTCCATGGTGACCCTGGGATGCCTGGTCAA |
| | | GGGCTATTTCCCTGANNAN |
| 174 | 2B01_ | NGNNNGNTNGATTTCNGCTTGGTGCCTCCACCGAACGTCCACGGGTAACTACTC |
| | LC | CACTGTTGACAGTAATAAGTGGCAGCATCTTCAGCCTCCATGCTGCTGATTGTGA |
| | | GAGAATAAGAGGTCCCAGATCCACTGCCACTGAAGCGAACAGGGACTCCAGAAG |
| | | CCAGGTTGGATGTGCCATAAATCCAGGGTTTGGGGGAGGTTTCTGACTTCTGCT |
| | | GGTACCAGTGCAAGTTGCTGGAACTTATACTTGAGCTGACACTGCAGGTGATGG |
| | | TGACCTTCTCCCCTGGAGATGCAGCCATGAGTGCTGGAGACTGGGTGTN |
| 175 | 1D01_ | NNNNNNNNNNNNNNNNNNNNNNNNNNNTNCTGTGAANATTTCCTGCAAGGCTTC |
| | HC | TGGCTACACCTTCACTGACTACTATATAAACTGGGTGAATCAGAGGCCTGGACAG |
| | | GGACTTGATTGGATTGGATGGATTTTTCCTGGAANTGNTTTTATTTATTACAATGA |
| | | NAAGTTCAAGGACAAGGTCACTTCTACTGTATACAAATCCTCCANCACATTCTACT |
| | | TGTTGCTCANCAGCCTGACCTCTGAGGACTCTGCGGTCTATTTCTGTGCAANATG |
| | | GAAGGATTACGGGTGGTACTTCTATGTCTGGGGCACAGGGACCACGGTCACCGT |
| | | CTCCTCATCCAAAACGACACCCCCATCTGTCTATCCACTGGCCCCTGGATCTGCT |
| | | GCCCAAACTAACTCCATGGTGACCCTGGGATGCCTGGTCAAGGGCTATTTCCCT |
| | | TANCAN |
| 176 | 1D01_ | NNNNNNNNNNNNNNNCTTGGTCCCCCCTCCGAACGTGTAAGGGTAAGTCCTCCA |
| | LC | CTGTTGACAGTAATAAGTGGCAGCATCTTCAGCCTCCATGCTGCTGATTGTGAGA |
| | | GAATAAGAGGTCCCAGATCCACTGCCACTGAAGCGAACAGGGACTCCAGAAGCC |
| | | AGGTTGGATGTGCCATAAATCCAGGGTTTGGGGGAGGTTTCTGACTTCTGCTGG |
| | | TACCAGTGCAAGTTGCTGGAACTTAGACTTGAGCTGACATTGCAGGTGAGGGTG |
| | | ACCTTCTCCCCTGGAGATGCAGCCATGAGTGCTGGAGACTGGGTGANNNCAATG |
| | | TCA |
| 177 | 2E02_ | NNNNNNNNNNNNNNNNNNNNNNNNNNNAGNANCCTTCAGTGAAGATATCCTGTA |
| | HC | AGGCTTCTGGATTCACATTCACTGACTACTACATTCACTGGGTGAAGCGGAGCCA |
| | | TGGAAAGAGCCTTGAGTGGATTGGACTTGTTTCTCCTTACAATGGTGGTACTTTC |
| | | TACAACCAGAAGTTCAAGGGCAAGGCCACATTGACTGTAGATACATCCTCCAATA |
| | | CAGCCTACATGGAGCTAACCAGCCTGACTTCTGAGGACTCTGCGGTCTATTACT |
| | | GTGCAAGATTGGGGGTTAACTGGTACTTCGATGTCTGGGGCACAGGGACCACG |
| | | GTCACCGTCTCCTCAGCCAAAACGACACCCCCATCTGTCTATCCACTGGCCCCT |
| | | GGATCTGCTGCCCAAACTAACTCCATGGTGACCCTGGGATGCCTGGTCAAGGGC |
| | | TATTTCCCTGANNN |
| 178 | 2E02_ | NNNNNNNNNNNNNNNTNTTCNNCTTGTTTCNCCCCTCCTANCAAGTGGNNNTNN |
| | LC | AAACTCCTCTGNTGGANAACAATANNNTANNCATCNNNTNCCNCCTCCCTGCNNA |
| | | TGAAGNGNNATAANANNTCCNNCNNNNNCTGCGCTNNTGNANACAAGNNCTACT |
| | | CANNAANNGNNAGNNGTGCCNTAAATCNACNGTTTGGNGGAGGTTTCTGACTTC |
| | | TGCTGGTACCANNGCNANGTGCTGGAACTTATACTNCTGNTGACACTGCGGNNN |
| | | AGNAGGACCTTTTCCCCCCCNGGATGCANCCATGAGTGCTNTGNANTGTGTCAT |
| | | CACAATATCANCA |
| 179 | 1A05_ | NNNNNNNNCTGNNTNNNNNNNGNANCNTTCAATGAAGATATCCTGTAAGGCTTC |
| | HC | TGGGTTCACTTTCACTGACTACTTCATACACTGGGTGAGACAGAGCCATGGAAAG |
| | | AGCCTTGAGTGGATTGGACTTGTTTCTCCTTATAATGGTGGTACTTACTACAACCA |
| | | GAAGTTCAAGGGCAAGGCCTCATTGACTGTAGACACATCCTCCAGCACAGCCTA |
| | | CATGGAACTAAGCAGCCTGACTTCTGAGGACTCTGCGGTCTATTACTGTGCAAGA |
| | | TTGGGCTACTATGGTAACTGGTACTTCTTTGACTACTGGGGCCAAGGCACCCCTC |
| | | TCACAGTCTCCTCAGCCAAAACGACACCCCCATCTGTCTATCCACTGGCCCCTG |
| | | GATCTGCTGCCCAAACTAACTCCATGGTGACCCTGGGATGCCTGGTCAAGGGCT |
| | | ATTNCCCTGAGCAN |
| 180 | 1A05_ | NNNNCNNNNNNNNNNNCTTGGTCCCCCCTCCGAACGTGTATGGGTAACTACTCC |
| | LC | ACTGTTGACAGTAATAAGTGGCAGCATCTTCAGCCTCCATGCTGCTGATTGTGAG |
| | | AGAATAAGAGGTCCCAGATCCACTGCCACTGAAGCGAACAGGGACTCCAGAAGC |
| | | CAGGTTGGATGTGCCATAAATCCAGGGTTTGGGGGAGGTTTCTGACTTCTGCTG |
| | | GTACCAGTTCAAGTTGCTGGAACTTATACTTGAGCTGACACTACAGGTGATGGTG |
| | | ACCTTTTCCCCTGGAGATGTAGCCATGAGTGCTGGAGACTGGGTGANNNCAATG |
| | | TCANNA |

TABLE 4
Heavy and light chain protein variable region is
encoded by a plasmid nucleotide sequence

| SEQ | | |
|---|---|---|
| ID NO | Label | Sequence |
| 181 | 1E04_ | NNNNNNNNNNNNNGTCACTGCACCTCGGTTCTATCGATTGAATTCCACCATGGG |
| | HC | ATGGTCATGTATCATCCTTTTTCTAGTAGCAACTGCAACCGGTGTACATTCCGAG |
| | | GTCCAGCTGCAACAGTCTGGGGCTGAGCTGGTGAAGCCTGGGGCTTCAGTGAA |
| | | GTTGTCCTGCAAGGCTTCTGGCTACACTTTCACCAGCTACTGGGTGCACTGGGT |
| | | GAGGCAGAGGCCTGGACAAGGCCTTGAGTGGATTGGAATGATTCATCCTAATAG |
| | | TGGTAGTACTAACTACAATGAGAAGTTCAAGAGCAAGGCCACACTGACTGTAGAC |
| | | AAATCCTCCAGCACAGCCTACATGCAACTCAGCAGCCTGACATCTGAGGACTCT |
| | | GCGGTCTATTACTGTGCAAGTTATTACTACGGTAGTAGCTATTACTTTGACTACTG |
| | | GGGCCAAGGCACCACTCTCACAGTCTCCTCAGCGTCGACCAAGGGCCCATCGG |
| | | TCTTCCCCCTGGCACCCTCCTCCAAGAGCACCTCTGGGGGCACAGCGGCCCTG |
| | | GGCTGCCTGGTCAAGGACTACTTCCCCGAACCTGTGACGGTCTCGTGGAACTCA |
| | | GGCGCCCTGACCAGCGGCGTGCACACCTTCCCGGCTGTCCTACAGTCCTCAGG |
| | | ACTCTACTCCCTCAGCAGCGTGGTGACCGTGCCCTCCAGCAGCTTGGGCACCCA |
| | | GACCTACATCTGCAACGTGAATCACAAGCCCAGCAACACCAAGGTGGACAAGNN |
| | | NNNNGAGCCCAAATCTTGTGACAAAACTCACACATGCCCACCGTGCCCAGCACC |
| | | TGAACTCCTGGGGGGACCGTCAGTCTTCCTCTTCCCCCCAAAACCCAAGGACAC |
| | | CCTCATGATCTCCCGGANCCCTGAGGTCACATGCGTGGNGGTGGACGTGAGCN |
| 182 | 1E04_ | NNNNNNNNNNNNNNNTCACTGCACCTCGGTTCTATCGATTGAATTCCACCATGG |
| | LC | GATGGTCATGTATCATCCTTTTTCTAGTAGCAACTGCAACCGGTGTACATTCCGA |
| | | CATTGTGATGACCCAGTCTCCATCCTCCCTGACTGTGACAGCAGGAGAGAAGGT |
| | | CACTATGAGCTGCAAGTCCAGTCAGAGTCTGTTAAACAGTGGAAATCAAAAGAAC |
| | | TACTTGACCTGGTACCAGCAGAAACCAGGGCAGCCTCCTAAACTGTTGATCTACT |
| | | GGGCATCCACTAGGGAATCTGGGGTCCCTGATCGCTTCACAGGCAGTGGATCTG |
| | | GAACAGATTTCACTCTCACCATCAGCAGTGTGCAGGCTGAAGACCTGGCAGTTTA |
| | | TTACTGTCAGAATGATTATAGTTATCCGCTCACGTTCGGTGCTGGGACCAAGCTG |
| | | GAGCTGAAACGTACGGTGGCTGCACCATCTGTCTTCATCTTCCCGCCATCTGAT |
| | | GAGCAGTTGAAATCTGGAACTGCCTCTGTTGTGTGCCTGCTGAATAACTTCTATC |
| | | CCAGAGAGGCCAAAGTACAGTGGAAGGTGGATAACGCCCTCCAATCGGGTAACT |
| | | CCCAGGAGAGTGTCACAGAGCAGGACAGCAAGGACAGCACCTACAGCCTCAGC |
| | | AGCACCCTGACGCTGAGCAAAGCAGACTACGAGAAACACAAAGTCTACGCCTGC |
| | | GAAGTCACCCATCAGGGCCTGAGCTCGCCCGTCACAAAGAGCTTCAACAGGGG |
| | | AGAGTGTTAGAAGCTTGATCCTCAACCTCTGGATTACAAAATTTGTGAAAGATTGA |
| | | CTGGTATTCTTAACTATGTTGCTCCTTTTACGCTATGTGGATACGCTGCTTTAATG |
| | | CCTTTGTATCATGCTATTGCTTCCCGTATGGCTTTCATTTTCTCCTCCTTGTATAAA |
| | | TCCTGGTTGCTGTCTCTTTATGAG |
| 183 | 1C08_ | NNNNNNNNNNNNNNGNNNNTGCACCTCGGTTCTATCGATTGAATTCCACCATGG |
| | HC | GATGGTCATGTATCATCCTTTTTCTAGTAGCAACTGCAACCGGTGTACATTCCGA |
| | | GGTCCAGCTGCAACAGTCTGGGGCTGAGCTGGTAAAGCCTGGGGCTTCAGTGA |
| | | AGCTGTCCTGCAAGGCTTCTGGCTACACTTTCACCAACTACTGGATGCACTGGGT |
| | | GAAGCAGAGGCCTGGGCAAGGCCTTGAGTGGATTGGAATGATTCATCCTAATAG |
| | | TGGTACTTCTAACTACAATGAGAAGTTCAAGAGCAAGGCCACACTGACTGTAGAC |
| | | AAATCCTCCAGCACAGCCTACATGCAACTCAGCAGCCTGACATCTGAGGACTCT |
| | | GCGGTCTTTTACTGTACAAGATCTGACTGGGCCTTTGACTACTGGGGCCAAGGT |
| | | ACCTCTCTCACAGTCTCCTCAGCGTCGACCAAGGGCCCATCGGTCTTCCCCCTG |
| | | GCACCCTCCTCCAAGAGCACCTCTGGGGGCACAGCGGCCCTGGGCTGCCTGGT |
| | | CAAGGACTACTTCCCCGAACCTGTGACGGTCTCGTGGAACTCAGGCGCCCTGAC |
| | | CAGCGGCGTGCACACCTTCCCGGCTGTCCTACAGTCCTCAGGACTCTACTCCCT |
| | | CAGCAGCGTGGTGACCGTGCCCTCCAGCAGCTTGGGCACCCAGACCTACATCT |
| | | GCAACGTGAATCACAAGCCCAGCAACACCAAGGTGGACAAGAGAGTTGAGCCCA |
| | | AATCTTGTGACAAAACTCACACATGCCCACCGTGCCCAGCACCTGAACTCCTGG |
| | | GGGGACCGTCAGTCTTCCTCTTCCCCCCAAAACCCAAGGACACCCTCATGATCT |
| | | CCCGGACCCCTGAGGTCACATGCGTGGTGGTGGACGTGAN |
| 184 | 1C08_ | NNNNNNNNNNNNNNNNNNNCTGCACCTCGGTTCTATCGATTGAATTCCACCATG |
| | LC | GGATGGTCATGTATCATCCTTTTTCTAGTAGCAACTGCAACCGGTGTACATTCCG |
| | | ACATTGTGATGACCCAGTCTCCATCCTCCCTGAGTGTGTCAGCAGGAGAGAAGG |
| | | TCACTATGAGCTGCAAGTCCAGTCAGAGCCTGTTAAACAGTGGAAATCAAAAGAA |
| | | CTACTTGGCCTGGTACCAGCAGAAACCAGGGCAGCCTCCTAAACTGTTGATCTA |
| | | CGGGGCATCCACTAGGGAATCTGGGGTCCCTGATCGCTTCACAGGCAGTGGAT |
| | | CTGGAACCGATTTCACTCTTACCATCAGTAGTGTGCAGGCTGAAGACCTGGCAG |
| | | TTTATTACTGTCAGAATGATCATAGTTATCCGCTCACGTTCGGTGCTGGGACCAA |
| | | GCTGGAGCTGAAACGTACGGTGGCTGCACCATCTGTCTTCATCTTCCCGCCATC |
| | | TGATGAGCAGTTGAAATCTGGAACTGCCTCTGTTGTGTGCCTGCTGAATAACTTC |
| | | TATCCCAGAGAGGCCAAAGTACAGTGGAAGGTGGATAACGCCCTCCAATCGGGT |
| | | AACTCCCAGGAGAGTGTCACAGAGCAGGACAGCAAGGACAGCACCTACAGCCT |
| | | CAGCAGCACCCTGACGCTGAGCAAAGCAGACTACGAGAAACACAAAGTCTACGC |
| | | CTGCGAAGTCACCCATCAGGGCCTGAGCTCGCCCGTCACAAAGAGCTTCAACAG |
| | | GGGAGAGTGTTAGAAGCTTGATCCTCAACCTCTGGATTACAAAATTTGTGAAAGA |
| | | TTGACTGGTATTCTTAACTATGTTGCTCCTTTTACGCTATGTGGATACGCTGCTTT |
| | | AATGCCTTTGTATCATGCTATTGCTTCCCGTATGGCTTTCATTTTCTCCTCCTTGT |
| | | ATAAATCCTGGNTGCTGN |
| 185 | 1B02_ | NNNNNNNNNCNGNCACTGCACCTCGGTTCTATCGATTGAATTCCACCATGGGAT |
| | HC | GGTCATGTATCATCCTTTTTCTAGTAGCAACTGCAACCGGTGTACATTCCGAGGT |
| | | CCAACTGCAGCAGTCTGGAGCTGAGCTGGTGAGGCCTGGGTCCTCAGTGAAGA |
| | | TGTCCTGCAAGACTTCTGGATATACATTCACATTCTACGGTATAAACTGGGTGAA |
| | | GCAGAGGCCTGGACAGGGCCTGGAATGGATTGGATATATTTATGTTGGAAATGG |
| | | TTATTCTGAGTACAATGAGAAGTTCAAGGTCAAGGCCACACTGACTTCAGACACA |
| | | TCCTCCAGCACAGCCTACATGCAGCTCAGCGGCCTGACATCTGAGGACTCTGCA |
| | | ATCTATTTCTGTGCAAGATCAGATGGTTACAACTACTTTGACTACTGGGGCCAAG |
| | | GCACCACTCTCACAGTCTCCTCAACGTCGACCAAGGGCCCATCGGTCTTCCCCC |
| | | TGGCACCCTCCTCCAAGAGCACCTCTGGGGGCACAGCGGCCCTGGGCTGCCTG |
| | | GTCAAGGACTACTTCCCCGAACCTGTGACGGTCTCGTGGAACTCAGGCGCCCTG |
| | | ACCAGCGGCGTGCACACCTTCCCGGCTGTCCTACAGTCCTCAGGACTCTACTCC |
| | | CTCAGCAGCGTGGTGACCGTGCCCTCCAGCAGCTTGGGCACCCAGACCTACAT |
| | | CTGCAACGTGAATCACAAGCCCAGCAACACCAAGGTGGACAAGAGAGTTGAGCC |
| | | CAAATCTTGTGACAAAACTCACACATGCCCACCGTGCCCAGCACCTGAACTCCTG |
| | | GGGGGACCGTCAGTCTTCCTCTTCCCCCCAAAACCCAAGGACACCCTCATGATC |
| | | TCCCGGACCCCTGAGGNCN |
| 186 | 1B02_ | NNNNNNNNNNNNNNGNNNNTGCACCTCGGTTCTATCGATTGAATTCCACCATGG |
| | LC | GATGGTCATGTATCATCCTTTTTCTAGTAGCAACTGCAACCGGTGTACATTCCCA |
| | | AATTGTTCTCACCCAGTCTCCAGCAATCCTATCTGCATCTCCAGGGGAGAAAGTC |
| | | ACAATGACTTGCAGGGCCAGCTCAAGTTTACATTACATGCACTGGTACCAGCAGA |
| | | AGACAGGATCCTCCCCCAAACCCTGGATTTATGCCACATCCAACCTGGCTTCTG |
| | | GAGTCCCTGCTCGCTTCAGTGGCAGTGGGTCTGGGACCTCTTACTCTCTCACAA |
| | | TCAGCAGAGTGGAGGCTGAAGATACTGCCACTTATTACTGCCAGCAGTGGAGTA |
| | | GTAACCCACCCACGTTCGGTGCTGGGACCAAGCTGGAGCTGAAACGTACGGTG |
| | | GCTGCACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAAGTACA |
| | | GTGGAAGGTGGATAACGCCCTCCAATCGGGTAACTCCCAGGAGAGTGTCACAGA |
| | | GCAGGACAGCAAGGACAGCACCTACAGCNNNNNNNNCACCCTGACGCTGAGCA |
| | | AAGCAGACTACGAGAAACACAAAGTCTACGCCTGCGAAGTCACCCATCAGGGCC |
| | | TGAGCTCGCCCGTCACAAAGAGCTTCAACAGGGGAGAGTGTTAGAAGCTTGATC |
| | | CTCAACCTCTGGATTACAAAATTTGTGAAAGATTGACTGGTATTCTTAACTATGTT |
| | | GCTCCTTTTACGCTATGTGGATACGCTGCTTTAATGCCTTTGTATCATGCTATTGC |
| | | TTCCCGTATGGCTTTCATTTTCTCCTCCTTGTATAAATCCTGGTTGCTGTCTCTTT |
| | | ATGAGGAGTTGTGGNCCGTTGTCAGGNAACGTGGCGTGGTGTGCACTG |
| 187 | 1A09_ | NNNNNNNNNNNGNCACTGCACCTCGGTTCTATCGATTGAATTCCACCATGGGAT |
| | HC | GGTCATGTATCATCCTTTTTCTAGTAGCAACTGCAACCGGTGTACATTCCGAGGT |
| | | CCAACTGCAGCAGCCTGGGGCTGAGCTGGTAAAGCCTGGGGCTTCAGTGAAGT |
| | | TGTCCTGCAAGGCTTCTGGCTACACTTTCACCAGCTACTGGATGCACTGGGTGA |
| | | AGCAGAGGCCTGGACAAGGCCTTGAGTGGATTGGATTGATTCATCCTAATAGTG |
| | | GTAGTACTTACTACAATGAGAAGTTCAAGAACAAGGCCACACTGACTGTAGACAA |
| | | ATCCTCCAGCACAGCCTACATGCAACTCAGCAGCCTGACATCTGAGGACTCTGC |
| | | GGTCTATTACTGTGCAAGATGGGATGATTCCTACTGGTACTTCAAAGTCTGGGGC |
| | | ACAGGGACCACGGTCACCGTCTCCTCAGCGTCGACCAAGGGCCCATCGGTCTT |
| | | CCCCCTGGCACCCTCCTCCAAGAGCACCTCTGGGGGCACAGCGGCCCTGGGCT |
| | | GCCTGGTCAAGGACTACTTCCCCGAACCTGTGACGGTCTCGTGGAACTCAGGCG |
| | | CCCTGACCAGCGGCGTGCACACCTTCCCGGCTGTCCTACAGTCCTCAGGACTCT |
| | | ACTCCCTCAGCAGCGTGGTGACCGTGCCCTCCAGCAGCTTGGGCACCCAGACC |
| | | TACATCTGCAACGTGAATCACAAGCCCAGCAACACCAAGGTGGACAAGAGAGTT |
| | | GAGCCCAAATCTTGTGACAAAACTCACACATGCCCACCGTGCCCAGCACCTGAA |
| | | CTCCTGGGGGGACCGTCAGTCTTCCTCTTCCCCCCAAAACCCAAGGACACCCTC |
| | | ATGATCTCCCGGACCCCTGNNNCACATGCGTGGTGGTGGA |
| 188 | 1A09 | NNNNNNNNNNNNNNNNGNNNNNTGCACCTCGGTTCTATCGATTGAATTCCACCA |
| | LC | TGGGATGGTCATGTATCATCCTTTTTCTAGTAGCAACTGCAACCGGTGTACATTC |
| | | CCAAATTGTTCTCACCCAGTCTCCAGCAATCCTGTCTGCATCTCCAGGGGAGAAG |
| | | GTCACAATGACTTGCAGGGCCAGTTCAAGTGTAAGTTACATGCACTGGTACCAG |
| | | CAGAAGCCAGGATCCTCCCCCAAACCCTGGATTTATGCCACATCCAACCTGGCT |
| | | TCTGGAGTCCCTGCTCGCTTCAGTGGCAGTGGGTCTGGGACCTCTTACTCTCTC |
| | | ACAATCAGCAGAGTGGAGGCTGAAGATGCTGCCACTTATTACTGCCAGCAGTGG |
| | | AGTAGCAACCCGTACACGTTCGGAGGGGGGACCAAGCTGGAAATAAAACGTACG |
| | | GTGGCTGCACCATCTGTCTTCATCTTCCCGCCATCTGATGAGCAGTTGAAATCTG |
| | | GAACTGCCTCTGTTGTGTGCCTGCTGAATAACTTCTATCCCAGAGAGGCCAAAGT |
| | | ACAGTGGAAGGTGGATAACGCCCTCCAATCGGGTAACTCCCAGGAGAGTGTCAC |
| | | AGAGCAGGACAGCAAGGACAGCACCTACAGCCTCAGCAGCACCCTGACGCTGA |
| | | GCAAAGCAGACTACGAGAAACACAAAGTCTACGCCTGCGAAGTCACCCATCAGG |
| | | GCCTGAGCTCGCCCGTCACAAAGAGCTTCAACAGGGGAGAGTGTTAGAAGCTTG |
| | | ATCCTCAACCTCTGGATTACAAAATTTGTGAAAGATTGACTGGTATTCTTAACTAT |
| | | GTTGCTCCTTTTACGCTATGTGGATACGCTGCTTTAATGCCTTTGTATCATGCTAT |
| | | TGCTTCCCGTATGGCTTTCATTTTCTCCTCCTTGTATAAATCCTGGNTGCTN |
| 189 | 2A03_ | NNNNNNNNNNNNNNGNCACTGCACCTCGGTTCTATCGATTGAATTCCACCATGG |
| | HC | GATGGTCATGTATCATCCTTTTTCTAGTAGCAACTGCAACCGGTGTACATTCCGA |
| | | GGTCCAACTGCAGCAGTCTGGAGCTGAGCTGGTGAGGCCTGGGGCTTCAGTGA |
| | | AGCTGTCCTGCAAGGCTTCTGGCTACACTTTCACTGACTACTATATAAACTGGGT |
| | | GAAGCAGAGGCCTGGACAGGGACTCGAGTGGATTGCAAGGATTTATCCTGGAAG |
| | | TGGTAATACTTACTACAATGAGAAGTTCAAGGGCAAGGCCACACTGACTGCAGAA |
| | | AAATCCTCCAGCACTGCCTACATGCAGCTCAGCAGCCTGACATCTGAGGACTCT |
| | | GCTGTCTATTTCTGTGCAAGACGGCTAACTGCGGGATACTTCGATGTCTGGGGC |
| | | ACAGGGACCACGGTCACCGTCTCCTCAGCGTCGACCAAGGGCCCATCGGTCTT |
| | | CCCCCTGGCACCCTCCTCCAAGAGCACCTCTGGGGGCACAGCGGCCCTGGGCT |
| | | GCCTGGTCAAGGACTACTTCCCCGAACCTGTGACGGTCTCGTGGAACTCAGGCG |
| | | CCCTGACCAGCGGCGTGCACACCTTCCCGGCTGTCCTACAGTCCTCAGGACTCT |
| | | ACTCCCTCAGCAGCGTGGTGACCGTGCCCTCCAGCAGCTTGGGCACCCAGACC |
| | | TACATCTGCAACGTGAATCACAAGCCCAGCAACACCAAGGTGGACAAGAGAGTT |
| | | GAGCCCAAATCTTGTGACAAAACTCACACATGCCCACCGTGCCCAGCACCTGAA |
| | | CTCCTGGGGGGACCGTCAGTCTTCCTCTTCCCCCCAAAACCCAAGGACACCCTC |
| | | ATGATCTCCCGGACCCCTGANTCACATGCGTGGTGGTGGACNNN |
| 190 | 2A03_ | NNNNNNNNNNNGTCACTGCACCTCGGTTCTATCGATTGAATTCCACCATGGGAT |
| | LC | GGTCATGTATCATCCTTTTTCTAGTAGCAACTGCAACCGGTGTACATTCCGACAT |
| | | CAAGATGACCCAGTCTCCATCTTCCATGTTTGCATCTCTAGGAGAGAGAGTCACT |
| | | ATCACTTGCAAGGCGAGTCAGGACATTAATAGCTATTTATCCTGGTTCCAGCAGA |
| | | AACCAGGGAAATCTCCTAAGACCCTGATCTATCGTGCAAACAGATTGGTAGATGG |
| | | GGTCCCATCAAGGTTCAGTGGCAGTGGATCTGGGCAAGATTATTCTCTCACCATC |
| | | AGCAGCCTGGAGTATGAAGATATGGGAATTTATTATTGTCTACAGTATGATGAGT |
| | | TTCCGCTCACGTTCGGTGCTGGGACCAAGCTGGAGCTGAAACGTACGGTGGCT |
| | | GCACCATCTGTCTTCATCTTCCCGCCATCTGATGAGCAGTTGAAATCTGGAACTG |
| | | CCTCTGTTGTGTGCCTGCTGAATAACTTCTATCCCAGAGAGGCCAAAGTACAGTG |
| | | GAAGGTGGATAACGCCCTCCAATCGGGTAACTCCCAGGAGAGTGTCACAGAGCA |
| | | GGACAGCAAGGACAGCACCTACAGCCTCAGCAGCACCCTGACGCTGAGCAAAG |
| | | CAGACTACGAGAAACACAAAGTCTACGCCTGCGAAGTCACCCATCAGGGCCTGA |
| | | GCTCGCCCGTCACAAAGAGCTTCAACAGGGGAGAGTGTTAGAAGCTTGATCCTC |
| | | AACCTCTGGATTACAAAATTTGTGAAAGATTGACTGGTATTCTTAACTATGTTGCT |
| | | CCTTTTACGCTATGTGGATACGCTGCTTTAATGCCTTTGTATCATGCTATTGCTTC |
| | | CCGTATGGCTTTCATTTTCTCCTCNNTGTATAAATCCTGGTTGCTGTCTCTTTATG |
| | | AGGAGTTGTGGN |
| 191 | 2C09_ | NNNNNNNNNNNNNNGNNNNTGCACCTCGGTTCTATCGATTGAATTCCACCATGG |
| | HC | GATGGTCATGTATCATCCTTTTTCTAGTAGCAACTGCAACCGGTGTACATTCCGA |
| | | AGTGATGCTGGTGGAGTCTGGGGAAGACTTAGTGAAACCTGGAGGGTCCCTGAA |
| | | ACTCTCCTGTATAGCCTCTGGATTCACTTTCAGTAGCTATGCCATGTCTTGGGTT |
| | | CGCCAGACTCCAGAGAAGAGGCTGGAGTGGGTCGCATATATTAGTAGTGGTGGT |
| | | GATTACATCTACTATACAGACACTGTGAAGGGCCGATTCACCATCTCCAGAGACA |
| | | ATGCCAGGAACACCCTGTTCCTACAAATGAGCAGTCTGAAGTCTGAGGACACAG |
| | | CCATGTATTACTGTACAAGAGATACCGGTTACTACGTTTCTCGGTACTTCGATGT |
| | | CTGGGGCACAGGGACCACGGTCACCGTCTCCTCAGCGTCGACCAAGGGCCCAT |
| | | CGGTCTTCCCCCTGGCACCCTCCTCCAAGAGCACCTCTGGGGGCACAGCGGCC |
| | | CTGGGCTGCCTGGTCAAGGACTACTTCCCCGAACCTGTGACGGTCTCGTGGAAC |
| | | TCAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCAC |
| | | CCAGACCTACATCTGCAACGTGAATCACAAGCCCAGCAACACCAAGGTGGACAA |
| | | GAGAGTTGAGCCCAAATCTTGTGACAAAACTCACACATGCCCACCGTGCCCAGC |
| | | ACCTGAACTCCTGGGGGGACCGTCAGTCTTCCTCTTCCCCCCAAAACCCAAGGA |
| | | CACCCTCATGATCTCCCGGACCCCTGANGN |
| 192 | 2C09_ | NNNNNNNNNNNNNNGNNNNTGCACCTCGGTTCTATCGATTGAATTCCACCATGG |
| | LC | GATGGTCATGTATCATCCTTTTTCTAGTAGCAACTGCAACCGGTGTACATTCCGA |
| | | CATTGTGATGACCCAGTCTCAAAAATTCATGTCCACATCAGTTGGAGACAGGGTC |
| | | AGTGTCACCTGCAAGGCCAGTCAGAATGTGGGAACTAATGTAGCCTGGTATCAA |
| | | CAGAAACCAGGACAATCTCCTAAAGCACTGATTTACTCGGCATCCTTCCGGAACA |
| | | GTGGAGTCCCTGATCGCTTCACAGGCAGTGGATCTGGGACAGATTTCACTCTCA |
| | | CCATCAGCAATGTGCAGTCTGAAGACTTGGCAGAGTATTTCTGTCATCAATATAA |
| | | CAACTATCCTCTGACGTTCGGTGGAGGCACCAAGCTGGAAATCAAACGTACGGT |
| | | GGCTGCACCATCTGTCTTCATCTTCCCGCCATCTGATGAGCAGTTGAAATCTGGA |
| | | ACTGCCTCTGTTGTGTGCCTGCTGAATAACTTCTATCCCAGAGAGGCCAAAGTAC |
| | | AGTGGAAGGTGGATAACGCCCTCCAATCGGGTAACTCCCAGGAGAGTGTCACAG |
| | | AGCAGGACAGCAAGGACAGCACCTACAGCCTCAGCAGCACCCTGACGCTGAGC |
| | | AAAGCAGACTACGAGAAACACAAAGTCTACGCCTGCGAAGTCACCCATCAGGGC |
| | | CTGAGCTCGCCCGTCACAAAGAGCTTCAACAGGGGAGAGTGTTAGAAGCTTGAT |
| | | CCTCAACCTCTGGATTACAAAATTTGTGAAAGATTGACTGGTATTCTTAACTATGT |
| | | TGCTCCTTTTACGCTATGTGGATACGCTGCTTTAATGCCTTTGTATCATGCTATTG |
| | | CTTCCCGTATGGCTTTCATTTTCTCCTCCTTGTATAAATCCTGGTTGCTGTCTCTT |
| | | TATGAGGAGTTGTGGCCCNNN |
| 193 | 2G02_ | NNNNNNNNNNNGNCACTGCACCTCGGTTCTATCGATTGAATTCCACCATGGGAT |
| | HC | GGTCATGTATCATCCTTTTTCTAGTAGCAACTGCAACCGGTGTACATTCCGAGGT |
| | | CCAACTGCAGCAGTCTGGACCTGTGCTGGTGAAGCCTGGGCCTTCAGTGAAGAT |
| | | ATCCTGTAAGGCTTCTGGATTCACATTCACTGACTACTACATGCACTGGGTGAAG |
| | | CAGAGCCATGGAAAGAGCCTTGAATGGATTGGACTTGTTTATCCTTACAATGGTG |
| | | GTACTTATTACAACCAGAAGTTCAAGGGCAAGGCCACATTGACTGTAGACACATC |
| | | CTCCAGCACAGCCTACATGGAGCTAAACAGCCTGACTTCTGAGGACTCTGCGGT |
| | | CTATTACTGTGTACGATTAGGGAACGGTAGTAGCAACGAGTGGTACTTCGATGTC |
| | | TGGGGCACAGGGACCACGGTCACCGTCTCCTCAGCGTCGACCAAGGGCCCATC |
| | | GGTCTTCCCCCTGGCACCCTCCTCCAAGAGCACCTCTGGGGGCACAGCGGCCC |
| | | TGGGCTGCCTGGTCAAGGACTACTTCCCCGAACCTGTGACGGTCTCGTGGAACT |
| | | CAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCAC |
| | | CCAGACCTACATCTGCAACGTGAATCACAAGCCCAGCAACACCAAGGTGGACAA |
| | | GAGAGTTGAGCCCAAATCTTGTGACAAAACTCACACATGCCCACCGTGCCCAGC |
| | | ACCTGAACTCCTGGGGGGACCGTCAGTCTTCCTCNN |
| 194 | 2G02_ | NNNNNNNNNNNNNNGNNNNTGCACCTCGGTTCTATCGATTGAATTCCACCATGG |
| | LC | GATGGTCATGTATCATCCTTTTTCTAGTAGCAACTGCAACCGGTGTACATTCCCA |
| | | AATTGTTCTCACCCAGTCTCCAGCACTCATGGCTGCATCTCCAGGGGAGAAGGT |
| | | CACCATCACCTGCAGTGTCAGCTCAAGTATAAGTTCCTACAATTTGCACTGGTAC |
| | | CAGCAGAAGTCAGAAACCTCCCCCAAACCCTGGATTTATGGCACATCCAACCTG |
| | | GCTTCTGGAGTCCCTGTTCGCTTCAGTGGCAGTGGATCTGGGACCTCTTATTCTC |
| | | TCACAATCAGCAGTATGGAGGCTGAAGATGCTGCCACTTATTACTGTCAACAGTG |
| | | GAGAACCTACCCGTGGACGTTCGGTGGAGGCACCAAGCTGGAAATCAAACGTAC |
| | | GGTGGCTGCACCATCTGTCTTCATCTTCCCGCCATCTGATGAGCAGTTGAAATCT |
| | | GGAACTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAGTGTC |
| | | ACAGAGCAGGACAGCAAGGACAGCACCTACAGCCTCAGCAGCACCCTGACGCT |
| | | GAGCAAAGCAGACTACGAGAAACACAAAGTCTACGCCTGCGAAGTCACCCATCA |
| | | GGGCCTGAGCTCGCCCGTCACAAAGAGCTTCAACAGGGGAGAGTGTTAGAAGC |
| | | TTGATCCTCAACCTCTGGATTACAAAATTTGTGAAAGATTGACTGGTATTCTTAAC |
| | | TATGTTGCTCCTTTTACGCTATGTGGATACGCTGCTTTAATGCCTTTGTATCATGC |
| | | TATTGCTTCCCGTATGGCTTTCATTTTCTCCTCCTTGTATAAAN |
| 195 | 1D09_ | NNNNNNNNNNGTCACTGCACCTCGGTTCTATCGATTGAATTCCACCATGGGATG |
| | HC | GTCATGTATCATCCTTTTTCTAGTAGCAACTGCAACCGGTGTACATTCCGAGGTC |
| | | CAACTGCAGCAGCCTGGAGCTGAGCTTGTGAAGCCTGGGGCTTCAGTGAAGCT |
| | | GTCCTGCAAGGCTTCTGGCTACACCTTCACCAACTACTGGATACACTGGATGAAG |
| | | CAGAGGCCTGGACGAGGCCTTGAGTGGATTGGAAGGATTGAGCCTAATAGCGG |
| | | TGATACTAAATACAATGAGAAGATCAAGAGCAGGGCCACACTGACTGTAGACAAA |
| | | CCGTCCAGCACAGCCTACATGCAGCTCAGCAGCCTGACATCTGAGGACTCTGCG |
| | | GTCTATTATTGTGCAAGATCTGGGTATGATTACCCTGAGGCCTGGGGCCAAGGC |
| | | ACCACTCTCACAGTCTCCTCAGCGTCGACCAAGGGCCCATCGGTCTTCCCCCTG |
| | | GCACCCTCCTCCAAGAGCACCTCTGGGGGCACAGCGGCCCTGGGCTGCCTGGT |
| | | CAAGGACTACTTCCCCGAACCTGTGACGGTCTCGTGGAACTCAGGCGCCCTGAC |
| | | CAGCGGCGTGCACACCTTCCCGGCTGTCCTACAGTCCTCAGGACTCTACTCCCT |
| | | CAGCAGCGTGGTGACCGTGCCCTCCAGCAGCTTGGGCACCCAGACCTACATCT |
| | | GCAACGTGAATCACAAGCCCAGCAACACCAAGGTGGACAAGAGAGTTGAGCCCA |
| | | AATCTTGTGACAAAACTCACACATGCCCACCGTGCCCAGCACCTGAACTCCTGG |
| | | GGGGANCGTCAGTCTTCCTCTTCCCCCCAAAACCCAANNA |
| 196 | 1D09_ | NNNNNNNNNNNNNGTNACTGCACCTCGGTTCTATCGATTGAATTCCACCATGGG |
| | LC | ATGGTCATGTATCATCCTTTTTCTAGTAGCAACTGCAACCGGTGTACATTCCGAC |
| | | ATTGTGCTGACCCAATCTCCAGCTTCTTTGGCTGTGTCTCCAGGGCAGAGAGCC |
| | | ACCATCTCCTGCAGAGCCAGTGAAGGTGTTGAATATTATGGCACAACTTTAATGC |
| | | AGTGGTACCAACAGAAACCAGGACAGCCACCCAAACTCCTCATCTATGCTGCAT |
| | | CCAACGTAGAATCTGGGGTCCCTGCCAGGTTTAGTGGCAGTGGGTCTGGGACA |
| | | GACTTCAGCCTCAACATCCATCCTGTGGAGGAGGATGATATTGCAATGTATTTCT |
| | | GTCAGCAAAGTAGGAAGGTTTCTTGGACGTTCGGTGGAGGCACCAAGCTGGAAA |
| | | TCAAACGTACGGTGGCTGCACCATCTGTCTTCATCTTCCCGCCATCTGATGAGCA |
| | | GTTGAAATCTGGAACTGCCTCTGTTGTGTGCCTGCTGAATAACTTCTATCCCAGA |
| | | GAGGCCAAAGTACAGTGGAAGGTGGATAACGCCCTCCAATCGGGTAACTCCCAG |
| | | GAGAGTGTCACAGAGCAGGACAGCAAGGACAGCACCTACAGCCTCAGCAGCAC |
| | | CCTGACGCTGAGCAAAGCAGACTACGAGAAACACAAAGTCTACGCCTGCGAAGT |
| | | CACCCATCAGGGCCTGAGCTCGCCCGTCACAAAGAGCTTCAACAGGGGAGAGT |
| | | GTTAGAAGCTTGATCCTCAACCTCTGGATTACAAAATTTGTGAAAGATTGACTGGT |
| | | ATTCTTAACTATGTTGCTCCTTTTACGCTATGTGGATACGCTGCTTTAATGCCTTT |
| | | GTATCATGCTATTGCTTCCCGTATGGCTTTCATTTTCTCCTCCTTGTATAAATCCT |
| | | GGNTGCTGTCTCTTTATGAGNGTTGTGGGCCCGTTGTCNGGCAACNTGGCGNG |
| | | GNGTGCACTGTGTTTGCTGACGCAACCCCCACTGGTTGGGGCATNNNNACTCNN |
| | | CTGTCAGCNNCT |
| 197 | 2B06_ | NNNNNNNNNNNNGTCACTGCACCTCGGTTCTATCGATTGAATTCCACCATGGGA |
| | HC | TGGTCATGTATCATCCTTTTTCTAGTAGCAACTGCAACCGGTGTACATTCCGAGG |
| | | TCCAGCTGCAACAGCCTGGGGCTGAGCTTGTGAAGCCTGGGGCTTCAGTGAAGT |
| | | TGTCCTGCAAGGCTTCTGGCTATACCTTCACCAACTACTGGATGCACTGGGTGAA |
| | | ACACAGGCCTGGACGAGGCCTTGAGTGGATTGGAAGGATTGATCCTAATAGTGG |
| | | TGGTACTAAGTACAATGAGAAGTTCAAGAATAAGGCCACACTGACTGTAGACAAT |
| | | CCCTCCAGCACAGGCTACATGCAGCTCAGCAGCCTGACATCTGAGGACTCTGCG |
| | | GTCTATTATTGTACAAGATCTGGGTATGATTACCCTGACTACTGGGGCCAAGGCA |
| | | CCACTCTCACAGTCTCCTCAGCGTCGACCAAGGGCCCATCGGTCTTCCCCCTGG |
| | | CACCCTCCTCCAAGAGCACCTCTGGGGGCACAGCGGCCCTGGGCTGCCTGGTC |
| | | AAGGACTACTTCCCCGAACCTGTGACGGTCTCGTGGAACTCAGGCGCCCTGACC |
| | | AGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTG |
| | | CAACGTGAATCACAAGCCCAGCAACACCAAGGTGGACAAGAGAGTTGAGCCCAA |
| | | ATCTTGTGACAAAACTCACACATGCCCACCGTGCCCAGCACCTGAACTCCTGGG |
| | | GGGACCGTCAGTCTTCCTCTTCCCCCCAAAACCCAAGGACACCCTCATGATCT |
| 198 | 2B06_ | NNNNNNNNNNNNNNNNGNNNNNTGNACCTCGGTTCTATCGATTGAATTCCACCA |
| | LC | TGGGATGGTCATGTATCATCCTTTTTCTAGTAGCAACTGCAACCGGTGTACATTC |
| | | CGACATTGTGCTGACCCAATCTCCAGCTTCTTTGGCTGTGTCTCTAGGGCAGAGA |
| | | GCCACCATCTCCTGCAGAGCCAGTGAAAGTGTTGAATATTATGGCACAAGTTTAA |
| | | TGCAGTGGTACCAACAGAAACCAGGACAGCCACCCAAAGTCCTCATCTATGCTG |
| | | CATCCAACGTAGATTCTGGGGTCCCTCCCAGGTTTAGTGGCAGTGGGTCTGGGA |
| | | CAGACTTCAGCCTCAACTTCCATCCTGTGGAGGAGGATGATATTGCAATGTATTT |
| | | CTGTCAGCAAAGTAGGAAGGTTCCTTGGACGTTCGGTGGAGGCACCAAGCTGGA |
| | | AATCAAACGTACGGTGGCTGCACCATCTGTCTTCATCTTCCCGCCATCTGATGAG |
| | | CAGTTGAAATCTGGAACTGCCTCTGTTGTGTGCCTGCTGAATAACTTCTATCCCA |
| | | GAGAGGCCAAAGTACAGTGGAAGGTGGATAACGCCCTCCAATCGGGTAACTCCC |
| | | AGGAGAGTGTCACAGAGCAGGACAGCAAGGACAGCACCTACAGCCTCAGCAGC |
| | | ACCCTGACGCTGAGCAAAGCAGACTACGAGAAACACAAAGTCTACGCCTGCGAA |
| | | GTCACCCATCAGGGCCTGAGCTCGCCCGTCACAAAGAGCTTCAACAGGGGAGA |
| | | GTGTTAGAAGCTTGATCCTCAACCTCTGGATTACAAAATTTGTGAAAGATTGACTG |
| | | GTATTCTTAACTATGTTGCTCCTTTTACGCTATGTGGATACGCTGCTTTAATGCCT |
| | | TTGTATCATGCTATTGCTTCCCGTATGGCTTTCATTTTCTCCTCCTTGTATAAATCC |
| | | TGGTTGCTGTCTCTTTATGANGAGTTGTGGCCCGTTGTCAGGNACGTGGCGTGG |
| | | TGTGCACTGTGNTTGCTGACNCAACCCCCCACTGGTTGGGGCATTGCCACCNAC |
| | | CTGN |
| 199 | 2A02_ | NNNNNNNNCCNNGNNNNTGCACCTCGGTTCTATCGATTGAATTCCACCATGGGA |
| | HC | TGGTCATGTATCATCCTTTTTCTAGTAGCAACTGCAACCGGTGTACATTCCGAGG |
| | | TCCAACTGCAACAGCCTGGGGCTGAGCTTGTGAAGCCTGGGGCTTCAGTGAAGC |
| | | TGTCCTGCAAGGCTTCTGGCTACACCTTCACCAGCTACTGGATGCACTGGGTGA |
| | | AGCAGAGGCCTGGACGAGGCCTTGAGTGGATTGGAAGGATTGATCCTAATAGTG |
| | | GTGGTACTAAGTACAATGAGAAGTTCAAGAGCAAGGCCACACTGACTGTAGACA |
| | | AACCCTCCAGCACAGCCTACATGCAGCTCAGCAGCCTGACATCTGAGGACTCTG |
| | | CGGTCTATTATTGTGCAAGATCTGGGTATGATTACCCTGACTACTGGGGCCAAGG |
| | | CACCACTCTCACAGTCTCCTCAGCGTCGACCAAGGGCCCATCGGTCTTCCCCCT |
| | | GGCACCCTCCTCCAAGAGCACCTCTGGGGGCACAGCGGCCCTGGGCTGCCTGG |
| | | TCAAGGACTACTTCCCCGAACCTGTGACGGTCTCGTGGAACTCAGGCGCCCTGA |
| | | CCAGCGGCGTGCACACCTTCCCGGCTGTCCTACAGTCCTCAGGACTCTACTCCC |
| | | TCAGCAGCGTGGTGACCGTGCCCTCCAGCAGCTTGGGCACCCAGACCTACATCT |
| | | GCAACGTGAATCACAAGCCCAGCAACACCAAGGTGGACAAGAGAGTTGAGCCCA |
| | | AATCTTGTGACAAAACTCACACATGCCCACCGTGCCCAGCACCTGAACTCCTGG |
| | | GGGGACCGTCAGTCTTCCTCTTCCCCCCAAAACCCAAGGACACCCTCATGATCT |
| | | CCCGGACCCCTGAGGTCACATGCGTGGTGGTGGANGTGAGCCACGAAGANCCT |
| | | GAGGTN |
| 200 | 2A02_ | NNNNNNNNNNNGTCACTGCACCTCGGTTCTATCGATTGAATTCCACCATGGGAT |
| | LC | GGTCATGTATCATCCTTTTTCTAGTAGCAACTGCAACCGGTGTACATTCCGACATT |
| | | GTGCTGACCCAATCTCCAGCTCCTTTGGCTGTGTCTCTAGGGCAGAGAGCCACC |
| | | ATCTCCTGCAGAGCCAGTGAAGGTGTTGAATATTATGACACAAGTTTAATGCAGT |
| | | GGTACCAACAGAAACCAGGACAGCCACCCAAACTCCTCATCTATGCTGCATCCA |
| | | ACGTAGAACCTGGGGTTCCTGCCAGGTTTGGTGGCAGTGGGTCTGGGACAGAC |
| | | TTCAGCCTCAACATCCATCCTGTGGAGGAGGATGATATTGCAATGTATTTCTGTC |
| | | AGCAAAGTAGGAAGGTTCCTTGGACGTTCGGTGGAGGCACCAAGCTGGAAATCA |
| | | AACGTACGGTGGCTGCACCATCTGTCTTCATCTTCCCGCCATCTGATGAGCAGTT |
| | | GAAATCTGGAACTGCCTCTGTTGTGTGCCTGCTGAATAACTTCTATCCCAGAGAG |
| | | GCCAAAGTACAGTGGAAGGTGGATAACGCCCTCCAATCGGGTAACTCCCAGGAG |
| | | AGTGTCACAGAGCAGGACAGCAAGGACAGCACCTACAGCCTCAGCAGCACCCT |
| | | GACGCTGAGCAAAGCAGACTACGAGAAACACAAAGTCTACGCCTGCGAAGTCAC |
| | | CCATCAGGGCCTGAGCTCGCCCGTCACAAAGAGCTTCAACAGGGGAGAGTGTTA |
| | | GAAGCTTGATCCTCAACCTCTGGATTACAAAATTTGTGAAAGATTGACTGGTATTC |
| | | TTAACTATGTTGCTCCTTTTACGCTATGTGGATACGCTGCTTTAATGCCTTTGTAT |
| | | CATGCTATTGCTTCCCGTATGGCTTTCATTTTCTCCTCCTTGTATAAATCCTGGTT |
| | | GCTGTCTCTTTATGAGNAGTTGTGGCCCGTTGTCNNNAACGTGGCGTGGTGTGN |
| | | NACTGTGTTTGCTG |
| 201 | 1B07_ | NNNNNCNNNNNNNNNGTNACTGCACCTCGGTTCTATCGATTGAATTCCACCATG |
| | HC | GGATGGTCATGTATCATCCTTTTTCTAGTAGCAACTGCAACCGGTGTACATTCCG |
| | | AGGTCCAACTGCAACAGCCTGGGGCTGAGCTGGTAAAGCCTGGGACTTCAGTGA |
| | | AGTTGTCCTGCAAGGCTTCTGGCTACACTTTCACCAGCTACTGGATGCACTGGGT |
| | | GAAGCAGAGGCCTGGACAAGGCCTTGAGTGGATTGGAATGATTCATCCTAATAG |
| | | TGGTAGTACTAACTACAATGAGATGTTCGAGAGCAAGGCCTCACTGACTGTAGAC |
| | | AAATCCTCCAGCACAGCCTACATGCAACTCAGCAGCCTGACATCTGAGGACTCT |
| | | GCGGTCTATTACTGTGCAAGAATCGGATACTCCGGCTGGGGTCAAGGAACCTCA |
| | | GTCACCGTCTCCTCAGCGTCGACCAAGGGCCCATCGGTCTTCCCCCTGGCACCC |
| | | TCCTCCAAGAGCACCTCTGGGGGCACAGCGGCCCTGGGCTGCCTGGTCAAGGA |
| | | CTACTTCCCCGAACCTGTGACGGTCTCGTGGAACTCAGGCGCCCTGACCAGCG |
| | | GCGTGCACACCTTCCCGGCTGTCCTACAGTCCTCAGGACTCTACTCCCTCAGCA |
| | | GCGTGGTGACCGTGCCCTCCAGCAGCTTGGGCACCCAGACCTACATCTGCAAC |
| | | GTGAATCACAAGCCCAGCAACACCAAGGTGGACAAGAGAGTTGAGCCCAAATCT |
| | | TGTGACAAAACTCACACATGCCCACCGTGCCCAGCACCTGAACTCCTGGGGGGA |
| | | CCGTCAGTCTTCCTCTTCCCCCCAAAACCCAAGGACACCCTCATGATCTCCCGG |
| | | ACCCCTGAGGTCACATGCGTGGTGGTGGACG |
| 202 | 1B07_ | NNNNNNNNNNNNNNGTNACTGCACCTCGGTTCTATCGATTGAATTCCACCATGG |
| | LC | GATGGTCATGTATCATCCTTTTTCTAGTAGCAACTGCAACCGGTGTACATTCCGA |
| | | CATTGTGATGACACAGTCTCCATCCTCCCTGAGTGTGTCAGCAGGAGAGAAGGT |
| | | CACTATGAGCTGCAAGTCCAGTCAGAGTCTGTTAAATGGTGGAAATCAAAAGAAC |
| | | TACTTGGCCTGGTACCAGCAGAAACCAGGGCAGCCTCCTAAAGTGTTGATCAAC |
| | | GGGGCATCCACTAGGGAATCTGGGGTCCCTGATCGCTTCACAGGCAATGGATCT |
| | | GGAACCGATTTCACTCTTACCATCAGCAGTGTGCAGGCTGAAGACCTGGCAGTT |
| | | TATTACTGTCAGAATGATCATACTTATCCGCTCACGTTCGGTGCTGGGACCAAGC |
| | | TGGAGCTGAAACGTACGGTGGCTGCACCATCTGTCTTCATCTTCCCGCCATCTG |
| | | ATGAGCAGTTGAAATCTGGAACTGCCTCTGTTGTGTGCCTGCTGAATAACTTCTA |
| | | TCCCAGAGAGGCCAAAGTACAGTGGAAGGTGGATAACGCCCTCCAATCGGGTAA |
| | | CTCCCAGGAGAGTGTCACAGAGCAGGACAGCAAGGACAGCACCTACAGCCTCA |
| | | GCAGCACCCTGACGCTGAGCAAAGCAGACTACGAGAAACACAAAGTCTACGCCT |
| | | GCGAAGTCACCCATCAGGGCCTGAGCTCGCCCGTCACAAAGAGCTTCAACAGG |
| | | GGAGAGTGTTAGAAGCTTGATCCTCAACCTCTGGATTACAAAATTTGTGAAAGAT |
| | | TGACTGGTATTCTTAACTATGTTGCTCCTTTTACGCTATGTGGATACGCTGCTTTA |
| | | ATGCCTTTGTATCATGCTATTGCTTCCCGTATGGCTTTCATTTTCTCCTCCTTGTA |
| | | T |
| 203 | 2D04_ | NNNNNNNNNNNNNNNGTCACTGCACCTCGGTTCTATCGATTGAATTCCACCATG |
| | HC | GGATGGTCATGTATCATCCTTTTTCTAGTAGCAACTGCAACCGGTGTACATTCCG |
| | | AGGTCCAACTGCAGCAGCCTGGAGGTGAGCTGGTGAGGCCTGGGTCCTCAGTG |
| | | AAGATGTCCTGCAAGACTTCTGGATATACATTCACATTCTACGGTATAAACTGGG |
| | | TGAAGCAGAGGCCTGGACAGGGCCTGGAATGGATTGGATATATTTATGTTGGAA |
| | | ATGGTTATACTGAGTACAATGAGAAGTTCAAGGTCAAGGCCACACTGACTTCAGA |
| | | CACATCCTCCAGCACAGCCTACATGCAGCTCAGCGGCCTGACATCTGAGGACTC |
| | | TGCAATCTATTTCTGTGCAAGATCCGATGGTTACAACTACTTCGACTACTGGGGC |
| | | CAAGGCACCACTCTCACAGTCTCCTCAGCGTCGACCAAGGGCCCATCGGTCTTC |
| | | CCCCTGGCACCCTCCTCCAAGAGCACCTCTGGGGGCACAGCGGCCCTGGGCTG |
| | | CCTGGTCAAGGACTACTTCCCCGAACCTGTGACGGTCTCGTGGAACTCAGGCGC |
| | | CCTGACCAGCGGCGTGCACACCTTCCCGGCTGTCCTACAGTCCTCAGGACTCTA |
| | | CTCCCTCAGCAGCGTGGTGACCGTGCCCTCCAGCAGCTTGGGCACCCAGACCT |
| | | ACATCTGCAACGTGAATCACAAGCCCAGCAACACCAAGGTGGACAAGAGAGTTG |
| | | AGCCCAAATCTTGTGACAAAACTCACACATGCCCACCGTGCCCAGCACCTGAAC |
| | | TCCTGGGGGGACCGTCAGTCTTCCTCTTCCCCCCAAAACCCAAGGACACCCTCA |
| | | TGATCTC |
| 204 | 2D04_ | NNNNNNNNNNNNNNNNNNNCACTGCACCTCGGTTCTATCGATTGAATTCCACCA |
| | LC | TGGGATGGTCATGTATCATCCTTTTTCTAGTAGCAACTGCAACCGGTGTACATTC |
| | | CCAAATTGTTCTCACCCAGTCTCCAGCAATCCTGACTGCATCCCCAGGGGAGAA |
| | | AGTCACAATGACTTGCAGGGCCAGTTCAAGTGTACATTACATGCACTGGTACCAG |
| | | CAGAAGCCAGGATCCTCCCCCAAACCCTGGATTTATGCCACATCCAACCTGGCT |
| | | TCTGGAGTCCCTGCTCGCTTCAGTGGCAGTGGGTCTGGGACCTCTTACTCTCTC |
| | | ACAATCAGCAGAGTGGAGGCTGAAGATACTGCCACTTATTACTGCCATCAGTGG |
| | | AGTAGTAACCCACCCACGTTCGGTGCTGGGACAAAGTTGGAAATAAAACGTACG |
| | | GTGGCTGCACCATCTGTCTTCATCTTCCCGCCATCTGATGAGCAGTTGAAATCTG |
| | | GAACTGCCTCTGTTGTGTGCCTGCTGAATAACTTCTATCCCAGAGAGGCCAAAGT |
| | | ACAGTGGAAGGTGGATAACGCCCTCCAATCGGGTAACTCCCAGGAGAGTGTCAC |
| | | AGAGCAGGACAGCAAGGACAGCACCTACAGCCTCAGCAGCACCCTGACGCTGA |
| | | GCAAAGCAGACTACGAGAAACACAAAGTCTACGCCTGCGAAGTCACCCATCAGG |
| | | GCCTGAGCTCGCCCGTCACAAAGAGCTTCAACAGGGGAGAGTGTTAGAAGCTTG |
| | | ATCCTCAACCTCTGGATTACAAAATTTGTGAAAGATTGACTGGTATTCTTAACTAT |
| | | GTTGCTCCTTTTACGCTATGTGGATACGCTGCTTTAATGCCTTTGTATCATGCTAT |
| | | TGCTTCCCGTATGGCTTTCATTTTCTCCTCCTTGTATAAATCCTGGNTGCTGTCTC |
| | | TTTATGAGGAGTTGTGGGCCCGTTGTCNNNAACGTGGCGTGGTGTGCACTGTGT |
| | | TTGCTGACGCAACCCCCACTGGTTGGGGCATTGCCACCACCTGTCAGCTCCTTT |
| | | CCGGGACTTTCGC |
| 205 | 1E07_ | NNNNNNNNNNNGNCACTGCACCTCGGTTCTATCGATTGAATTCCACCATGGGAT |
| | HC | GGTCATGTATCATCCTTTTTCTAGTAGCAACTGCAACCGGTGTACATTCCGAGGT |
| | | CCAACTGCAGCAGCCTGGGGCTGAGCTGGTAAAGCCTGGGGCTTCAGTGAAGT |
| | | TGTCCTGCAAGGCTTCTGGCTACACTTTCACCAGCTACTGGATGCACTGGGTGA |
| | | AGCAGAGGCCTGGACAAGGCCTTGAGTGGATTGGATTGATTCATCCTAATAGTG |
| | | GTAGTACTTACTACAATGAGAAGTTCAAGAACAAGGCCACACTGACTGTAGACAA |
| | | ATCCTCCAGCACAGCCTACATGCAACTCAGCAGCCTGACATCTGAGGACTCTGC |
| | | GGTCTATTACTGTGCAAGATGGGATGATTCCTACTGGTACTTCAAAGTCTGGGGC |
| | | ACAGGGACCACGGTCACCGTCTCCTCAGCGTCGACCAAGGGCCCATCGGTCTT |
| | | CCCCCTGGCACCCTCCTCCAAGAGCACCTCTGGGGGCACAGCGGCCCTGGGCT |
| | | GCCTGGTCAAGGACTACTTCCCCGAACCTGTGACGGTCTCGTGGAACTCAGGCG |
| | | CCCTGACCAGCGGCGTGCACACCTTCCCGGCTGTCCTACAGTCCTCAGGACTCT |
| | | ACTCCCTCAGCAGCGTGGTGACCGTGCCCTCCAGCAGCTTGGGCACCCAGACC |
| | | TACATCTGCAACGTGAATCACAAGCCCAGCAACACCAAGGTGGACAAGAGAGTT |
| | | GAGCCCAAATCTTGTGACAAAACTCACACATGCCCACCGTGCCCAGCACCTGAA |
| | | CTCCTGGGGGGACCGTCAGTCTTCCTCTTCCCCCCAAAAN |
| 206 | 1E07_ | NNNNNNNNNNNNNNGTCACTGCNCCTCGGTTCTATCGATTGAATTCCACCATGG |
| | LC | GATGGTCATGTATCATCCTTTTTCTAGTAGCAACTGCAACCGGTGTACATTCCCA |
| | | AATTGTTCTCACCCAGTCTCCAGCAATCCTGTCTGCATCTCCAGGGGAGAAGGTC |
| | | ACAATGACTTGCAGGGCCAGTTCAAGTGTAAGTTACATGCACTGGTACCAGCAG |
| | | AAGCCAGGATCCTCCCCCAAACCCTGGATTTATGCCACATCCAACCTGGCTTCTG |
| | | GAGTCCCTGCTCGCTTCAGTGGCAGTGGGTCTGGGACCTCTTACTCTCTCACAA |
| | | TCAGCAGAGTGGAGGCTGAAGATGCTGCCACTTATTACTGCCAGCAGTGGAGTA |
| | | GCAACCCGTACACGTTCGGAGGGGGGACCAAGCTGGAAATAAAACGTACGGTG |
| | | GCTGCACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAAGTACA |
| | | GTGGAAGGTGGATAACGCCCTCCAATCGGGTAACTCCCAGGAGAGTGTCACAGA |
| | | GCAGGACAGCAAGGACAGCACCTACAGCCTCAGCAGCACCCTGACGCTGAGCA |
| | | AAGCAGACTACGAGAAACACAAAGTCTACGCCTGCGAAGTCACCCATCAGGGCC |
| | | TGAGCTCGCCCGTCACAAAGAGCTTCAACAGGGGAGAGTGTTAGAAGCTTGATC |
| | | CTCAACCTCTGGATTACAAAATTTGTGAAAGATTGACTGGTATTCTTAACTATGTT |
| | | GCTCCTTTTACGCTATGTGGATACGCTGCTTTAATGCCTTTGTATCATGCTATTGC |
| | | TTCCCGTATGGCTTTCATTTTCTCCTCCTTGTATAAATCCTGGNTGCTGTCTCTTT |
| | | ATGAGGAGTTGTGGCCCGTTGTCNNNAACGTGGGCGTGGNGTGCACTGTGTTTG |
| | | CTGA |
| 207 | 2D07_ | NNNNNNNNNNNNNNGTNACTGCACCTCGGTTCTATCGATTGAATTCCACCATGG |
| | HC | GATGGTCATGTATCATCCTTTTTCTAGTAGCAACTGCAACCGGTGTACATTCCGA |
| | | GGTCCAGCTGCAACAGTCTGGACCTGAGCTGGTGAGGCCTGGGGCTTCAGTGA |
| | | AGATATCCTGCAAGGCTTCTGGCTACACCTTCACTGACTACTATATACACTGGCT |
| | | GAAGCAGAGGCCTGGACAGGGACTTGAGTGGATTGGATTGATTTTTCCTGGAAG |
| | | TGGTAGTATTTACTGTAATGAGAAGTTCAAGGGCAAGGCTACACTTACTGTAGAC |
| | | AAATCCTCCACCACAGCCTACATGTTGCTCAGCAGCCTGACCTCTGAGGACTCT |
| | | GCGGTCTACTTCTGTGCAAGATGGGAGACTACGGCGTGGTACTTCGATGTCTGG |
| | | GGCACAGGGACCACGGTCACCGTCTCCTCAGCGTCGACCAAGGGCCCATCGGT |
| | | CTTCCCCCTGGCACCCTCCTCCAAGAGCACCTCTGGGGGCACAGCGGCCCTGG |
| | | GCTGCCTGGTCAAGGACTACTTCCCCGAACCTGTGACGGTCTCGTGGAACTCAG |
| | | GCGCCCTGACCAGCGGCGTGCACACCTTCCCGGCTGTCCTACAGTCCTCAGGA |
| | | CTCTACTCCCTCAGCAGCGTGGTGACCGTGCCCTCCAGCAGCTTGGGCACCCA |
| | | GACCTACATCTGCAACGTGAATCACAAGCCCAGCAACACCAAGGTGGACAAGAG |
| | | AGTTGAGCCCAAATCTTGTGACAAAACTCACACATGCCCACCGTGCCCAGCACC |
| | | TGAACTCCTGGGGGGANCGTCAGTCTTCCTCTTCCCCCCAAAACCCAAGGACAC |
| | | CCTCATGATCTCCCGGACCCCTGAGGTCACATGCGTGGTGGTGGACGTGA |
| 208 | 2D07_ | NNNNNNNNNNNNNNNCACTGCACCTCGGTTCTATCGATTGAATTCCACCATGGG |
| | LC | ATGGTCATGTATCATCCTTTTTCTAGTAGCAACTGCAACCGGTGTACATTCCCAAA |
| | | TTGTTCTCACCCAGTCTCCCCAAATTGTTCTCACCCAGTCTCCAGCAATCCTGTC |
| | | TGCATCTCCAGGGGAGAAGGTCACAATGACTTGCAGGGCCAGTTTAAGTGTAAG |
| | | TTACATGCACTGGTACCAGCAGAAGCCAGGATCCTCCCCCAAACCCTGGATTTAT |
| | | GCCACATCCAACCTGGCTTCTGGAGTCCCTGCCCGCTTCAGTGGCAGTGGGTCT |
| | | GGGACCTCTTACTCTCTCACAGTCAGCAGAGTGGAGGCCGAAGATGCTGCCACT |
| | | TATTACTGCCAGCAGTGGAGGAGTTACCCGTACACGTTCGGAGGGGGCACCAAG |
| | | CTGGAAATCAAACGTACGGTGGCTGCACCATCTGTCTTCATCTTCCCGCCATCTG |
| | | ATGAGCAGTTGAAATCTGGAACTGCCTCTGTTGTGTGCCTGCTGAATAACTTCTA |
| | | TCCCAGAGAGGCCAAAGTACAGTGGAAGGTGGATAACGCCCTCCAATCGGGTAA |
| | | CTCCCAGGAGAGTGTCACAGAGCAGGACAGCAAGGACAGCACCTACAGCCTCA |
| | | GCAGCACCCTGACGCTGAGCAAAGCAGACTACGAGAAACACAAAGTCTACGCCT |
| | | GCGAAGTCACCCATCAGGGCCTGAGCTCGCCCGTCACAAAGAGCTTCAACAGG |
| | | GGAGAGTGTTAGAAGCTTGATCCTCAACCTCTGGATTACAAAATTTGTGAAAGAT |
| | | TGACTGGTATTCTTAACTATGTTGCTCCTTTTACGCTATGTGGATACGCTGCTTTA |
| | | ATGCCTTTGTATCATGCTATTGCTTCCCGTATGGCTTTCATTTTCTCCTCCTTGTA |
| | | TAAATCCTGGTTGCTGTCTCTTTATGAGGAGTTGTGGGCCCGTTGTCAGNAACGT |
| | | GGNGTGGNGTGCACTGTGTTTGCTGACGCAA |
| 209 | 1A02_ | NNNNNNNNNNNNNGTNACTGCACCTCGGTTCTATCGATTGAATTCCACCATGGG |
| | HC | ATGGTCATGTATCATCCTTTTTCTAGTAGCAACTGCAACCGGTGTACATTCCGAG |
| | | GTCCAACTGCAACAGTCTGGACCTGTGCTGGTGAAGCCTGGGCCTTCAATGAAG |
| | | ATATCCTGTAAGGCTTCTGGATTCACATTCACTGACTACTACATACACTGGGTGA |
| | | GGCAGAGCCATGGAAAGAGCCTTGAGTGGATTGGACTTGTTTCTCCTTACAATG |
| | | GTGGTACTTACTACAACCAGAAGTTCAAGGGCAAGGCCACATTGACTGTAGACTC |
| | | ATCCTCCAGCACAGCCTACATGGAGCTAAGCAGCCTGACTTCTGAGGACTCTGC |
| | | GGTCTATTACTGTGCAAGATTGGGCTACTATGGTGACTGGTACTACTTTGACTAC |
| | | TGGGGCCAAGGCACCCCTCTCACAGTCTCCTCAGCGTCGACCAAGGGCCCATC |
| | | GGTCTTCCCCCTGGCACCCTCCTCCAAGAGCACCTCTGGGGGCACAGCGGCCC |
| | | TGGGCTGCCTGGTCAAGGACTACTTCCCCGAACCTGTGACGGTCTCGTGGAACT |
| | | CAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCAC |
| | | CCAGACCTACATCTGCAACGTGAATCACAAGCCCAGCAACACCAAGGTGGACAA |
| | | GAGAGTTGAGCCCAAATCTTGTGACAAAACTCACACATGCCCACCGTGCCCAGC |
| | | ACCTGAACTCCTGGGGGGACCGTCAGTCTTCCTCTTCCCCCCAAAACCCAAGGA |
| | | CACCCTCATGATCTCCCGGACCCCTGNNTCACATGCGTGGTGGTGGACGTGAGC |
| | | CACGAAGANCCTGAGNNNAGTTCAACTGGTACG |
| 210 | 1A02_ | NNNNNNNNNNNNNNNNTNAACTGCACCTCGGTTCTATCGATTGAATTCCACCAT |
| | LC | GGGATGGTCATGTATCATCCTTTTTCTAGTAGCAACTGCAACCGGTGTACATTCC |
| | | CAAATTGTTCTCACCCAGTCTCCAGCACTCATGGCTACATCTCCAGGGGAAAAGG |
| | | TCACCATCACCTGTAGTGTCAGCTCAAGTATAAGTTCCAGCAACTTGAACTGGTA |
| | | CCAGCAGAAGTCAGAAACCTCCCCCAAACCCTGGATTTATGGCACATCCAACCT |
| | | GGCTTCTGGAGTCCCTGTTCGCTTCAGTGGCAGTGGATCTGGGACCTCTTATTCT |
| | | CTCACAATCAGCAGCATGGAGGCTGAAGATGCTGCCACTTATTACTGTCAACAGT |
| | | GGAGTAGTTACCCATACACGTTCGGAGGGGGGACCAAGCTGGAAATAAAACGTA |
| | | CGGTGGCTGCACCATCTGTCTTCATCTTCCCGCCATCTGATGAGCAGTTGAAATC |
| | | TGGAACTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAGTGTC |
| | | ACAGAGCAGGACAGCAAGGACAGCACCTACAGCCTCAGCAGCACCCTGACGCT |
| | | GAGCAAAGCAGACTACGAGAAACACAAAGTCTACGCCTGCGAAGTCACCCATCA |
| | | GGGCCTGAGCTCGCCCGTCACAAAGAGCTTCAACAGGGGAGAGTGTTAGAAGC |
| | | TTGATCCTCAACCTCTGGATTACAAAATTTGTGAAAGANTGACTGGTATTCTTAAC |
| | | TATGTTGCTCCTTTTACGCTATGTGGATACGCTGCTTTAATGCCTTTGTATCATGC |
| | | TATTGCTTCCCGTATGGCTTTCATTTTCTCCTCCTTGTATAAATCCTGGTTGCTGT |
| | | CTCTTTATGAAGGAGTTGTGGCCCGTTGTCAGGNAACGTGGGCGNGGTNNGCAC |
| | | TGN |
| 211 | 2D10_ | NNNNNNNNNNNNNCACTGCACCTCGGTTCTATCGATTGAATTCCACCATGGGAT |
| | HC | GGTCATGTATCATCCTTTTTCTAGTAGCAACTGCAACCGGTGTACATTCCGAGGT |
| | | CCAGCTGCAACAGTCTGGGGCTGAGCTGGTGAAGCCTGGGGCTTCAGTGAAGT |
| | | TGTCCTGCAAGGCTTCTGGCTACACTTTCACCACCTACTGGATGCACTGGATGAA |
| | | GCAGTGGCCTGGACAAGGCCTTGAGTGGATTGGATTGATTCATCCTAATAGTGG |
| | | TAGTACTTACTACAATGAGAAGTTCAAGAGCAAGGCCACACTGACTGTAGACAAA |
| | | TCCTCCAGCACAGCCTACATGCAACTCAGCAGCCTGACATCTGAGGACTCTGCG |
| | | GTCTATTACTGTGCAAGATTTGAGGAGCTATGGGGAGGTTACTGGTACTTCGATG |
| | | TCTGGGGCACAGGGACCACGGTCACCGTCTCCTCAGCGTCGACCAAGGGCCCA |
| | | TCGGTCTTCCCCCTGGCACCCTCCTCCAAGAGCACCTCTGGGGGCACAGCGGC |
| | | CCTGGGCTGCCTGGTCAAGGACTACTTCCCCGAACCTGTGACGGTCTCGTGGAA |
| | | CTCAGGCGCCCTGACCAGCGGCGTGCACACCTTCCCGGCTGTCCTACAGTCCT |
| | | CANGACTCTACTCCCTCAGCANCGTGGTGACCGTGCCCTCCAGCAGCTTGGGCA |
| | | CCCAGACCTACATCTGCNACGTGAATCACAAGCCCAGCAACACCAAGGTGGACA |
| | | AGANAGTTGAGCCCAAATCTTGTGACAANNCTCACACATGCCCACCGTGCCCAN |
| | | CACCTGAACTCCTGGGGGGACCGNCAGTCTTCCTCTTCCCCCCAAAACCCANGA |
| | | CNNCCNN |
| 212 | 2D10_ | NNNNNNNNNNNNGTCACTGCACCTCGGTTCTATCGATTGAATTCCACCATGGGA |
| | LC | TGGTCATGTATCATCCTTTTTCTAGTAGCAACTGCAACCGGTGTACATTCCCAAAT |
| | | TGTTCTCACCCAGTCTCCAGCACTCATGGCTGCATCTCCAGGGGAGAAGGTCAC |
| | | CATCACCTGCAGTGTCAGCTCAAGTATAAGTTCCAGCAACTTGCACTGGTACCAG |
| | | CAGAAGTCAGAAACCTCCCCCAAACCCTGGATTTATGGCACATCCAACCTGGCTT |
| | | CTGGAGTCCCTGTTCGCTTCAGTGGCAGTGGATCTGGGACCTCTTATTCTCTCAC |
| | | AATCAGCAGCATGGAGGCTGAAGATGCTGCCACTTATTACTGTCAACAGTGGAG |
| | | TAGTTACCCGTACACGTTCGGAGGGGGGACCAAGCTGGAAATAAAACGTACGGT |
| | | GGCTGCACCATCTGTCTTCATCTTCCCGCCATCTGATGAGCAGTTGAAATCTGGA |
| | | ACTGCCTCTGTTGTGTGCCTGCTGAATAACTTCTATCCCAGAGAGGCCAAAGTAC |
| | | AGTGGAAGGTGGATAACGCCCTCCAATCGGGTAACTCCCAGGAGAGTGTCACAG |
| | | AGCAGGACAGCAAGGACAGCACCTACAGCCTCAGCAGCACCCTGACGCTGAGC |
| | | AAAGCAGACTACGAGAAACACAAAGTCTACGCCTGCGAAGTCACCCATCAGGGC |
| | | CTGAGCTCGCCCGTCACAAAGAGCTTCAACAGGGGAGAGTGTTAGAAGCTTGAT |
| | | CCTCAACCTCTGGATTACAAAATTTGTGAAAGATTGACTGGTATTCTTAACTATGT |
| | | TGCTCCTTTTACGCTATGTGGATACGCTGCTTTAATGCCTTTGTATCATGCTATTG |
| | | CTTCCCGTATGGCTTTCATTTTCTCCTCCTTGTATAAATCCTGGNTGCTGTCTCTT |
| | | TATGAGGAGTTGTGGCCCGTTGTCAGGNAACGTGGNGTG |
| 213 | 2E08_ | NNNNNNNNNNNNCNGGTNACTGCACCTCGGTTCTATCGATTGAATTCCACCATG |
| | HC | GGATGGTCATGTATCATCCTTTTTCTAGTAGCAACTGCAACCGGTGTACATTCCG |
| | | AGGTCCAGCTGCAGCAGCCTGGGGCTGAGCTGGCAAAGCCTGGGGCTTCAGTG |
| | | AAGTTGTCCTGCAAGGCTTCTGGCTACAATTTCACCAGCTACTGGATGCACTGGG |
| | | TGAAGCAGAGGCCTGGACAAGGCCTTGAGTGGATTGGATTGATTCATCCTAATA |
| | | GTGGTGGTACTTACTACAATGAGAAGTTCAAGAGCAAGGCCACACTGACTTTAGA |
| | | CAGATCCTCCAGCACAGCCTACATGCAACTCAGCAGCCTGACATCTGAGGACTC |
| | | TGCGGTCTATTATTGTGCAAGATTTGAGGAGGAATGGGGAGGGTATTGGTACTTC |
| | | GATGTCTGGGGCACGGGGACCACGGTCACCGTCTCCTCAGCGTCGACCAAGGG |
| | | CCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCTGTGACGGTCTCGT |
| | | GGAACTCAGGCGCCCTGACCAGCGGCGTGCACACCTTCCCGGCTGTCCTACAG |
| | | TCCTCAGGACTCTACTCCCTCAGCAGCGTGGTGACCGTGCCCTCCAGCAGCTTG |
| | | GGCACCCAGACCTACATCTGCAACGTGAATCACAAGCCCAGCAACACCAAGGTG |
| | | GACAAGAGAGTTGAGCCCAAATCTTGTGACAAAACTCACACATGCCCACCGTGC |
| | | CCAGCACCTGAACTCCTGGGGGGACCGTCAGTCTTCCTCTTCCCCCCAAAACCC |
| | | AAGGACACCCTCATGATCTCCCGGACCCCTGAGGTCACATGCGTGGTGGTGGAC |
| | | GTGAGCCACGAAGANCCTGAGGNCAAGTTCAACTGGTACGNNN |
| 214 | 2E08_ | NNNNNNNNNNNNNNNNNTGCACCTCGGTTCTATCGATTGAATTCCACCATGGGA |
| | LC | TGGTCATGTATCATCCTTTTTCTAGTAGCAACTGCAACCGGTGTACATTCCCAAAT |
| | | TGTTCTCACCCAGTCTCCAGCACTCATGGCTGCATCTCCAGGGGAAAAGGTCAC |
| | | CATCACCTGCAGTGTCAGTTCAAGTATAAGTTCCAGCAACTTGCACTGGTACCAA |
| | | CAGAAGTCAGAAACCTCCCCCAAACCCTGGATTTATGGCACTTCCAACCTGGCTT |
| | | CTGGAGTCCCTGTTCGCTTCAGTGGCAGTGGATCTGGGACCTCTTATTCTCTCAC |
| | | AATCAGCAGCATGGAGGCTGAAGATGCTGCCACTTATTACTGTCAACAGTGGAG |
| | | TAGTTACCCGTACACGTTCGGAGGGGGGACCAAACTGGAAATAAAACGTACGGT |
| | | GGCTGCACCATCTGTCTTCATCTTCCCGCCATCTGATGAGCAGTTGAAATCTGGA |
| | | ACTGCCTCTGTTGTGTGCCTGCTGAATAACTTCTATCCCAGAGAGGCCAAAGTAC |
| | | AGTGGAAGGTGGATAACGCCCTCCAATCGGGTAACTCCCAGGAGAGTGTCACAG |
| | | AGCAGGACAGCAAGGACAGCACCTACAGCCTCAGCAGCACCCTGACGCTGAGC |
| | | AAAGCAGACTACGAGAAACACAAAGTCTACGCCTGCGAAGTCACCCATCAGGGC |
| | | CTGAGCTCGCCCGTCACAAAGAGCTTCAACAGGGGAGAGTGTTAGAAGCTTGAT |
| | | CCTCAACCTCTGGATTACAAAATTTGTGAAAGATTGACTGGTATTCTTAACTATGT |
| | | TGCTCCTTTTACGCTATGTGGATACGCTGCTTTAATGCCTTTGTATCATGCTATTG |
| | | CTTCCCGTATGGCTTTCATTTTCTCCTCNTT |
| 215 | 2H04_ | NNNNNNNNNNNNNGTCACTGCACCTCGGTTCTATCGATTGAATTCCACCATGGG |
| | HC | ATGGTCATGTATCATCCTTTTTCTAGTAGCAACTGCAACCGGTGTACATTCCGAG |
| | | GTCCAACTGCAACAGTCTGGGGCTGAGCTGGTAAAGCCTGGGGCTTCAGTGAAG |
| | | TTGTCCTGCAAGGCTTCTGGCTACACTTTCACCAGCTACTGGATGCACTGGGTGA |
| | | AGCAGAGGCCTGGACAAGGCCTTGAGTGGATTGGACTGATTCATCCTAATAGTA |
| | | GTAGTACTTACTACAATGAGAAGTTCAAGACCAGGGCCACACTGACTGTAGACAA |
| | | GTCCTCCAGCACAGCCTACATGCAACTCAGCAGCCTGACATCTGAGGACTCTGC |
| | | GGTCTATTACTGTGCAAGATTGGGCTATGGTAACTCCTACTGGTACTTCGATGTC |
| | | TGGGGCACAGGGACCACGGTCACCGTCTCCTCAGCGTCGACCAAGGGCCCATC |
| | | GGTCTTCCCCCTGGCACCCTCCTCCAAGAGCACCTCTGGGGGCACAGCGGCCC |
| | | TGGGCTGCCTGGTCAAGGACTACTTCCCCGAACCTGTGACGGTCTCGTGGAACT |
| | | CAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCAC |
| | | CCAGACCTACATCTGCAACGTGAATCACAAGCCCAGCAACACCAAGGTGGACAA |
| | | GAGAGTTGAGCCCAAATCTTGTGACAAAACTCACACATGCCCACCGTGCCCAGC |
| | | ACCTGAACTCCTGGGGGGACCGTCAGTCTTCCTCTTCCCCCCAAAACCCAAGNC |
| | | ACCCTCATGATCTCCCGGACCCCTGAGGTCACATGCGTGGTGGTGGACGTGAG |
| | | CCNNN |
| 216 | 2H04_ | NNNNNNNNNNNCNGGTNNACTGCACCTCGGTTCTATCGATTGAATTCCACCATG |
| | LC | GGATGGTCATGTATCATCCTTTTTCTAGTAGCAACTGCAACCGGTGTACATTCCC |
| | | AAATTGTTCTCACCCAGTCTCCAGCACTCATGGCTGCATCTCCAGGGGAGAAGG |
| | | TCACCATCACCTGCAGTGTCAGCTCAAGTATTAGTTCCAGCAACTTGCACTGGTA |
| | | CCAGCAGAAGTCAGAAACCTCCCCCAAACCCTGGATTTATGGCACATCCAACCT |
| | | GGCTTCTGGAGTCCCTATTCGCTTCAGTGGCAGTGGATCTGGGACCTCTTATTCT |
| | | CTCACAATCAGCAGTTTGGAGGCTGAGGATGCTGCCACTTATTACTGTCAACAGT |
| | | GGAGAAGTTACCCGTGGACGTTCGGTGGAGGCACCAAGCTGGAAATCAAACGTA |
| | | CGGTGGCTGCACCATCTGTCTTCATCTTCCCGCCATCTGATGAGCAGTTGAAATC |
| | | TGGAACTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAGTGTC |
| | | ACAGAGCAGGACAGCAAGGACAGCACCTACAGCCTCAGCAGCACCCTGACGCT |
| | | GAGCAAAGCAGACTACGAGAAACACAAAGTCTACGCCTGCGAAGTCACCCATCA |
| | | GGGCCTGAGCTCGCCCGTCACAAAGAGCTTCAACAGGGGAGAGTGTTAGAAGC |
| | | TTGATCCTCAACCTCTGGATTACAAAATTTGTGAAAGATTGACTGGTATTCTTAAC |
| | | TATGTTGCTCCTTTTACGCTATGTGGATACGCTGCTTTAATGCCTTTGTATCATGC |
| | | TATTGCTTCCCGTATGGCTTTCATTTTCTCCTCCTTGTATAAATCCTGGNTGCTGT |
| | | CTCTTTATGAGGAGTTGTGGGCCCGTTGTCANGNACGNNGNNGTGGTGTGCACT |
| | | GTGTTTGCTGACGCANNNCCNNNNNGGN |
| 217 | 1B03_ | NNNNNNNNNNCNGGTCACTGCACCTCGGTTCTATCGATTGAATTCCACCATGGG |
| | HC | ATGGTCATGTATCATCCTTTTTCTAGTAGCAACTGCAACCGGTGTACATTCCGAG |
| | | GTCCAACTGCAGCAGTCTGGGGCTGAGCTGGTAAAGCCTGGGGCTTCAGTGAA |
| | | GTTGTCCTGCAAGGCTTCTGGCTACACTTTCACCAGCTACTGGATGCACTGGGT |
| | | GCAGCAGAGGCCTGGACAAGGCCTTGAGTGGATTGGATTGATTCATCCTATTGG |
| | | TGGTGGTACTCACTACAATGAGAAGTTCAAGAACAAGGCCACACTGACTGTAGAC |
| | | AAATCCTCCAGCACAGCCTACATGCAACTCAGCAGCCTGACATCTGAGGACTCT |
| | | GCGGTCTATTACTGTGCAAGACTCGGAACTGGTCCGTACTACTTTGACTACTGGG |
| | | GCCAAGGCACCACTCTCACAGTCTCCTCAGCGTCGACCAAGGGCCCATCGGTCT |
| | | TCCCCCTGGCACCCTCCTCCAAGAGCACCTCTGGGGGCACAGCGGCCCTGGGC |
| | | TGCCTGGTCAAGGACTACTTCCCCGAACCTGTGACGGTCTCGTGGAACTCAGGC |
| | | GCCCTGACCAGCGGCGTGCACACCTTCCCGGCTGTCCTACAGTCCTCAGGACTC |
| | | TACTCCCTCAGCAGCGTGGTGACCGTGCCCTCCAGCAGCTTGGGCACCCAGAC |
| | | CTACATCTGCAACGTGAATCACAAGCCCAGCAACACCAAGGTGGACAAGAGAGT |
| | | TGAGCCCAAATCTTGTGACAAAACTCACACATGCCCACCGTGCCCAGCACCTGA |
| | | ACTCCTGGGGGGACCGTCAGTCTTCCTCTTCCCCCCAAAACCCAAGGACACCCT |
| | | CATGATCTCCCGGACCCCTGANTCACATGCGTGGTGGTGGACGTGAGCCACGAA |
| | | GANCCTGA |
| 218 | 1B03_ | NNNNNNNNNNNNNNNNNNNNCTGCACCTCGGTTCTATCGATTGAATTCCACCAT |
| | LC | GGGATGGTCATGTATCATCCTTTTTCTAGTAGCAACTGCAACCGGTGTACATTCC |
| | | CAAATTGTTCTCACCCAGTCTCCAGCACTCATGGCTGCATCTCCAGGGGAGAAG |
| | | GTCACCATCACCTGCAGTGTCAGCTCAAGTATAAGTTCCAGCACCTTGGACTGGT |
| | | ACCAGCAGAAGTCAGAAACCTCCCCCAAACCCTGGGTTTATGGCACATCCAACC |
| | | TGGCTTCTGGAGTCCCTGTTCGCTTCAGTGGCAGTGGATCTGGGACCTCTTATTC |
| | | TCTCACAATCAGCAGCATGGAGGCTGAAGATGCTGCCACTTATTACTGTCAACAG |
| | | TGGAGTAGTTACCCGTGGACGTTCGGTGGAGGCACCAAGCTGGAAATCAAACGT |
| | | ACGGTGGCTGCACCATCTGTCTTCATCTTCCCGCCATCTGATGAGCAGTTGAAAT |
| | | CTGGAACTGCCTCTGTTGTGTGCCTGCTGAATAACTTCTATCCCAGAGAGGCCAA |
| | | AGTACAGTGGAAGGTGGATAACGCCCTCCAATCGGGTAACTCCCAGGAGAGTGT |
| | | CACAGAGCAGGACAGCAAGGACAGCACCTACAGCCTCAGCAGCACCCTGACGC |
| | | TGAGCAAAGCAGACTACGAGAAACACAAAGTCTACGCCTGCGAAGTCACCCATC |
| | | AGGGCCTGAGCTCGCCCGTCACAAAGAGCTTCAACAGGGGAGAGTGTTAGAAG |
| | | CTTGATCCTCAACCTCTGGATTACAAAATTTGTGAAAGATTGACTGGTATTCTTAA |
| | | CTATGTTGCTCCTTTTACGCTATGTGGATACGCTGCTTTAATGCCTTTGTATCATG |
| | | CTATTGCTTCCCGTATGGNTTTCATTTTCTCCTCCTTGTATAAATCCTGGNTGCTG |
| | | TCTCTTTNATGAGGAGTTGTGGCCCGTNGNCAGGNAACGNGGCGTGGTGTGCA |
| | | CTGNNTTTGCT |
| 219 | 2H07_ | NNNNNNNNNNNNGTCACTGCACCTCGGTTCTATCGATTGAATTCCACCATGGGA |
| | HC | TGGTCATGTATCATCCTTTTTCTAGTAGCAACTGCAACCGGTGTACATTCCGAGG |
| | | TCCAGCTGCAGCAGTCTGGACCTGTGCTGGTGAAGCCTGGGCCTTCAGTGAATA |
| | | TATCCTGTAAGGCTTCTGGATTCACATTCACTGACTACTACATTCACTGGGTGAAA |
| | | CAGAGCCATGGAAAGAGCCTTGAGTGGATTGGACTTGTTTATCCTTACAGTGGTG |
| | | GTACTTACTACAACCAGAAGTTCAAGGGCAAGGCCACATTGACTGTAGACGCATC |
| | | CTCCAGCACAGCCTACATGGAGCTAGGCAGCCTGACTTCTGAGGACTCTGCGGT |
| | | CTATTACTGTGCAAGATTGGGCGACAACTTCTACTACTTTGACTACTGGGGCCAA |
| | | GGCACCACTCTCACAGTCTCCTCAGCGTCGACCAAGGGCCCATCGGTCTTCCCC |
| | | CTGGCACCCTCCTCCAAGAGCACCTCTGGGGGCACAGCGGCCCTGGGCTGCCT |
| | | GGTCAAGGACTACTTCCCCGAACCTGTGACGGTCTCGTGGAACTCAGGCGCCCT |
| | | GACCAGCGGCGTGCACACCTTCCCGGCTGTCCTACAGTCCTCAGGACTCTACTC |
| | | CCTCAGCAGCGTGGTGACCGTGCCCTCCAGCAGCTTGGGCACCCAGACCTACA |
| | | TCTGCAACGTGAATCACAAGCCCAGCAACACCAAGGTGGACAAGAGAGTTGAGC |
| | | CCAAATCTTGTGACAAAACTCACACATGCCCACCGTGCCCAGCACCTGAACTCCT |
| | | GGGGGGACCGTCAGTCTTCCTCTTCCCCCCAAAACCCAAGGACACCCTCATGAT |
| | | CTCCCGGACCCCTGAGGTCACATGCGTGGTGGTGGACGTGAGCCN |
| 220 | 2H07_ | NNNNNNNNNNNNNNNNNNNTGCACCTCGGTTCTATCGATTGAATTCCACCATGG |
| | LC | GATGGTCATGTATCATCCTTTTTCTAGTAGCAACTGCAACCGGTGTACATTCCCA |
| | | AATTGTTCTCACCCAGTCTCCAACACTCATGGCTGCATCTCCAGGGGAGAAGGT |
| | | CACCATCACCTGCAGTGTCAGCTCAAGTATAAGTTCCAGCAACTTGCACTGGTAC |
| | | CAGCAGAAGTCAGAAACCTCCCCCAAACCCTGGATTTATGGCACATCCAACCTG |
| | | GCTTCTGGAGTCCCTGTTCGCTTCAGTGGCAGTGGATCTGGGACATCTTATTCTC |
| | | TCACAATCAGCAGCATGGAGGCTGAAGATGCTGCCACTTATTACTGTCAACAGTG |
| | | GAGAAGTTACCCGTACACGTTCGGAGGGGGGACCAAGCTGGAAATAAAACGTAC |
| | | GGTGGCTGCACCATCTGTCTTCATCTTCCCGCCATCTGATGAGCAGTTGAAATCT |
| | | GGAACTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAGTGTC |
| | | ACAGAGCAGGACAGCAAGGACAGCACCTACAGCCTCAGCAGCACCCTGACGCT |
| | | GAGCAAAGCAGACTACGAGAAACACAAAGTCTACGCCTGCGAAGTCACCCATCA |
| | | GGGCCTGAGCTCGCCCGTCACAAAGAGCTTCAACAGGGGAGAGTGTTAGAAGC |
| | | TTGATCCTCAACCTCTGGATTACAAAATTTGTGAAAGATTGACTGGTATTCTTAAC |
| | | TATGTTGCTCCTTTTACGCTATGTGGATACGCTGCTTTAATGCCTTTGTATCATGC |
| | | TATTGCTTCCCGTATGGCTTTCATTTTCTCCTCCTTGTATAAATCCTGGNNGCTGT |
| | | CTCTTTATGAGGAGTTGTGGGCCCGTTGTCAGGNAACGTGGCGTGGNGTGNN |
| 221 | 1C01_ | NNNNNNNNNNNNNNGTCACTGCACCTCGGTTCTATCGATTGAATTCCACCATGG |
| | HC | GATGGTCATGTATCATCCTTTTTCTAGTAGCAACTGCAACCGGTGTACATTCCGA |
| | | GGTCCAACTGCAACAGCCTGGACCTGTGCTGGTGAAGCCTGGGCCTTCAGTGAA |
| | | GATATCCTGTAAGGCTTCTGGATTCACATTCACTGACTACTACATGCACTGGGTG |
| | | AAACAGAGCCATGGAAAGAGCCTTGAGTGGATTGGTCTTGTTTCTCCTTACAATG |
| | | GTGGTACTTTCTACAACCAGAAGTTCAAGGGCAAGGCCACATTGACTGTAGACAC |
| | | ATCCTCCAGCACAGCCTACATGGAACTAAACAGCCTGACTTCTGAGGACTCTGC |
| | | GGTCTATTACTGTGCAAGAGTGGGTAATAGCTACGTCCATTATGCTATGGACTAC |
| | | TGGGGTCAAGGAACCTCAGTCACCGTCTCCTCAGCGTCGACCAAGGGCCCATC |
| | | GGTCTTCCCCCTGGCACCCTCCTCCAAGAGCACCTCTGGGGGCACAGCGGCCC |
| | | TGGGCTGCCTGGTCAAGGACTACTTCCCCGAACCTGTGACGGTCTCGTGGAACT |
| | | CAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCAC |
| | | CCAGACCTACATCTGCAACGTGAATCACAAGCCCAGCAACACCAAGGTGGACAA |
| | | GAGAGTTGAGCCCAAATCTTGTGACAAAACTCACACATGCCCACCGTGCCCAGC |
| | | ACCTGAACTCCTGGGGGGACCGTCAGTCTTCCTCTTCCCCCCAAAACCCAAGGA |
| | | CACCCTCATGATCTCCCGGACCCCTGANGTCACATGCGTGGNN |
| 222 | 1C01_ | NNNNNNNNNNNNNCCNGGTNACTGCACCTCGGTTCTATCGATTGAATTCCACCA |
| | LC | TGGGATGGTCATGTATCATCCTTTTTCTAGTAGCAACTGCAACCGGTGTACATTC |
| | | CCAAATTGTTCTCACCCAGTCTCCAGCACTCATGGCTGCATCTCCAGGGGAGAA |
| | | GGTCACCATCACCTGCAGTGTCAGCTCAAGTATAAGTTCCAGCAACTTGCACTGG |
| | | TACCAGCAGAAGTCAGAAACCTCCCCCAAACCCTGGATTTATGGCACATCCAACC |
| | | TGGCTTCTGGAGTCCCTGTTCGCTTCAGTGGCAGTGGATCTGGGACCTCTTATTC |
| | | TCTCACAATCAGCAGCATGGAGGCTGAAGATGCTGCCACTTATTACTGTCAACAG |
| | | TGGAGTAATTACCCGTACACGTTCGGAGGGGGGACCAAGCTGGAAATAAAACGT |
| | | ACGGTGGCTGCACCATCTGTCTTCATCTTCCCGCCATCTGATGAGCAGTTGAAAT |
| | | CTGGAACTGCCTCTGTTGTGTGCCTGCTGAATAACTTCTATCCCAGAGAGGCCAA |
| | | AGTACAGTGGAAGGTGGATAACGCCCTCCAATCGGGTAACTCCCAGGAGAGTGT |
| | | CACAGAGCAGGACAGCAAGGACAGCACCTACAGCCTCAGCAGCACCCTGACGC |
| | | TGAGCAAAGCAGACTACGAGAAACACAAAGTCTACGCCTGCGAAGTCACCCATC |
| | | AGGGCCTGAGCTCGCCCGTCACAAAGAGCTTCAACAGGGGAGAGTGTTAGAAG |
| | | CTTGATCCTCAACCTCTGGATTACAAAATTTGTGAAAGATTGACTGGTATTCTTAA |
| | | CTATGTTGCTCCTTTTACGCTATGTGGATACGCTGCTTTAATGCCTTTGTATCATG |
| | | CTATTGCTTCCCGTATGGCTTTCATTTTCTCCTCCTTGNATAAATCCTGGNTGCTG |
| | | TCTCTTTATGANGAGTTGTGGNCCCGTTGTCNGNNAACGTGGCGTGGTGTGCAC |
| | | TGTGNTTGCTGN |
| 223 | 2C07_ | NNNNNNNNNNNCNGNNNNTGCACCTCGGTTCTATCGATTGAATTCCACCATGGG |
| | HC | ATGGTCATGTATCATCCTTTTTCTAGTAGCAACTGCAACCGGTGTACATTCCGAA |
| | | GTGAAGCTGGTGGAGTCTGGGGAAGGCTTAGTGAAGCCTGGAGGGTCCCTGAA |
| | | ACTCTCCTGTGCAGCCTCTGGATTCACTTTCAGTAGCTATGCCATGTCTTGGGTT |
| | | CGCCAGACTCCAGAGAAGAGGCTGGACTGGGTCGCATACATTAGTAGTGGTGGT |
| | | GATCACATCTACTATGCAGACACTGTGAAGGGCCGATTCACCATCTCCAGAGACA |
| | | ATGCCAGGAACACCCTGTACCTGCAAATGAGCAGTCTGAAGTCTGAGGACACAG |
| | | CCATGTATTACTGTACAAGAGATACCGGTTACTACGTCTCTCGGTACTTCGATGT |
| | | CTGGGGCACAGGGACCACGGTCACCGTCTCCTCAGCGTCGACCAAGGGCCCAT |
| | | CGGTCTTCCCCCTGGCACCCTCCTCCAAGAGCACCTCTGGGGGCACAGCGGCC |
| | | CTGGGCTGCCTGGTCAAGGACTACTTCCCCGAACCTGTGACGGTCTCGTGGAAC |
| | | TCAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCAC |
| | | CCAGACCTACATCTGCAACGTGAATCACAAGCCCAGCAACACCAAGGTGGACAA |
| | | GAGAGTTGAGCCCAAATCTTGTGACAAAACTCACACATGCCCACCGTGCCCAGC |
| | | ACCTGAACTCCTGGGGGGACCGTCAGTCTTCCTCTTCCCCCCAAAACCCAAGGA |
| | | CACCCTCATGATCTCCCGGANCCCTGAGGTCACATGCGTGGTGGTGGACGTGA |
| | | GCCA |
| 224 | 2C07_ | NNNNNNNNNNNNNGNNNNTGCACCTCGGTTCTATCGATTGAATTCCACCATGGG |
| | LC | ATGGTCATGTATCATCCTTTTTCTAGTAGCAACTGCAACCGGTGTACATTCCGAC |
| | | ATTGTGATGACCCAGTCTCAAAAATTCATGTCCACATCAGTAGGAGACAGGGTCA |
| | | GCGTCACCTGCAAGGCCAGTCAGAATGTGGTTACTAATGTTGCCTGGTATCAACA |
| | | GAAATCAGGGCAATCTCCTAAAGTAGTGATTTATTCGGCATCCTTCCGGTCCAGT |
| | | GGAGTCCCTGATCGCTTCACAGGCAGTGGATCTGGGACAGATTTCACTCTCACC |
| | | ATCAGCAATGTGCAGTCTGAAGACTTGGCAGAGTATTTCTGTCACCAATATAACA |
| | | GCCATCCTCTGACGTTCGGTGGAGGCACCAAGCTGGAAATCAAACGTACGGTGG |
| | | CTGCACCATCTGTCTTCATCTTCCCGCCATCTGATGAGCAGTTGAAATCTGGAAC |
| | | TGCCTCTGTTGTGTGCCTGCTGAATAACTTCTATCCCAGAGAGGCCAAAGTACAG |
| | | TGGAAGGTGGATAACGCCCTCCAATCGGGTAACTCCCAGGAGAGTGTCACAGAG |
| | | CAGGACAGCAAGGACAGCACCTACAGCCTCAGCAGCACCCTGACGCTGAGCAA |
| | | AGCAGACTACGAGAAACACAAAGTCTACGCCTGCGAAGTCACCCATCAGGGCCT |
| | | GAGCTCGCCCGTCACAAAGAGCTTCAACAGGGGAGAGTGTTAGAAGCTTGATCC |
| | | TCAACCTCTGGATTACAAAATTTGTGAAAGATTGACTGGTATTCTTAACTATGTTG |
| | | CTCCTTTTACGCTATGTGGATACGCTGCTTTAATGCCTTTGTATCATGCTATTGCT |
| | | TCCCGTATGGCTTTCATTTTCTCCTCCTTGTATAAATCCTGGNTGCTGNCTCTTNA |
| | | NTNNNNGANNTGTGGCCCGTTGTCAGGNAAC |
| 225 | 2G04_ | NNNNNNNNNNNNNGTCACTGCACCTCGGTTCTATCGATTGAATTCCACCATGGG |
| | HC | ATGGTCATGTATCATCCTTTTTCTAGTAGCAACTGCAACCGGTGTACATTCCGAG |
| | | GTCCAGCTGCAGCAGCCTGGACCTGAATTGGTGAAGCCCGGGTCCTCACTGAA |
| | | GATATCCTGCAAGGCTTCTGGTTACACCTTCACTGACTACTTTATAAACTGGGTG |
| | | AAACAGAGGCCTGGACAGGGACTTGACTGGATTGGATGGATTTTTCCTGGAAGT |
| | | GGTAGTACTTACTACAATGACAAGTTCAAGGGCAAGGCCACACTTACTGTAGACA |
| | | AATCCTCCAGCACTGCCTACATGTTGCTCAGCAGCCTGACCTCTGAGGCCTCTG |
| | | CGGTCTATTTCTGTGCAAGATGGGACTCCGATAGTACCTACTGGTACTTCGATGT |
| | | CTGGGGCACAGGGACCACGGTCACCGTCTCCTCAGCGTCGACCAAGGGCCCAT |
| | | CGGTCTTCCCCCTGGCACCCTCCTCCAAGAGCACCTCTGGGGGCACAGCGGCC |
| | | CTGGGCTGCCTGGTCAAGGACTACTTCCCCGAACCTGTGACGGTCTCGTGGAAC |
| | | TCAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCAC |
| | | CCAGACCTACATCTGCAACGTGAATCACAAGCCCAGCAACACCAAGGTGGACAA |
| | | GAGAGTTGAGCCCAAATCTTGTGACAAAACTCACACATGCCCACCGTGCCCAGC |
| | | ACCTGAACTCCTGGGGGGACCGTCAGTCTTCCTCTTCCCCCCAAAACCCAAGGA |
| | | CACCCTCATGATCTCCCGGACCCCTGAGGTCACATGCGTGGTGGTGGACGTGA |
| | | GCCAC |
| 226 | 2G04_ | NNNNNNNNNNNNCNNNNNNNNNTGCACCTCGGTTCTATCGATTGAATTCCACCA |
| | LC | TGGGATGGTCATGTATCATCCTTTTTCTAGTAGCAACTGCAACCGGTGTACATTC |
| | | CCAAATTGTTCTCACCCAGTCTCCAGCACTCATGGCTGCATCTCCAGGGGAGAA |
| | | GGTCACCATCACCTGCAGTGTCAATTCAAGTATAAGTTCCAGCACCTTGCACTGG |
| | | TACCAGCAGAAGTCACAAACCTCCCCCAAACCCTGGATTTATGGTACATCCAATT |
| | | TGGCTTCTGGAGTCCCTATTCGCTTCAGTGGCAGTGGATCTGGGACCTCTTATTC |
| | | TCTCACAATCAGCAGCATGGAGGCTGAAGATGCTGCCACTTATTACTGTCAACAG |
| | | TGGAGTACTTACCCGTACACGTTCGGAGGTGGCACCAAGCTGGAAATCAAACGT |
| | | ACGGTGGCTGCACCATCTGTCTTCATCTTCCCGCCATCTGATGAGCAGTTGAAAT |
| | | CTGGAACTGCCTCTGTTGTGTGCCTGCTGAATAACTTCTATCCCAGAGAGGCCAA |
| | | AGTACAGTGGAAGGTGGATAACGCCCTCCAATCGGGTAACTCCCAGGAGAGTGT |
| | | CACAGAGCAGGACAGCAAGGACAGCACCTACAGCCTCAGCAGCACCCTGACGC |
| | | TGAGCAAAGCAGACTACGAGAAACACAAAGTCTACGCCTGCGAAGTCACCCATC |
| | | AGGGCCTGAGCTCGCCCGTCACAAAGAGCTTCAACAGGGGAGAGTGTTAGAAG |
| | | CTTGATCCTCAACCTCTGGATTACAAAATTTGTGAAAGATTGACTGGTATTCTTAA |
| | | CTATGTTGCTCCTTTTACGCTATGTGGATACGCTGCTTTAATGCCTTTGTATCATG |
| | | CTATTGCTTCCCGTATGGCTTTCATTTTCTCCTCCTTGTATAAATCCTGGTTGCTG |
| | | TCTCTTTATGAGGAGTTGTGGGCCCGTTGTCAGGCAACGTGGNGNGGNGTGCAC |
| | | TGTGTTTGCTGACGCAACCCCNCTGGTNGGGGCATTGCCACCACCTGTCAGCTC |
| | | CTTTCCGGGACTTTCGNTTNNNNNN |
| 227 | 1G01_ | NNNNNNNNNNNNNNGGTCACTGCACCTCGGTTCTATCGATTGAATTCCACCATG |
| | HC | GGATGGTCATGTATCATCCTTTTTCTAGTAGCAACTGCAACCGGTGTACATTCCG |
| | | AGGTCCAACTGCAACAGTCTGGAGCTGAGCTGATGAAGCCTGGGGCCTCAGTGA |
| | | AGCTTTCCTGCAAGGCTACTGGCTACACATTCACTGGCTACTGGATAGAGTGGTT |
| | | AAAGCAGAGGCCTGGACATGGCCTTGAGTGGATTGGAGAGATTTTACCTGGAAG |
| | | TGATAATACTAACTACAATGAGAAGTTCAGGGGCAAGGCCACATTCACTGCAGAT |
| | | ACATCCTCCAACACAGCCTACATGCACCTCAGCAGCCTGACAACTGAGGACTCT |
| | | GCCATCTATTACTGTGCAAGAGAAGGGGGTTTCTACTTTGACTACTGGGGCCAA |
| | | GGCACCACTCTCACAGTCTCCTCAGCGTCGACCAAGGGCCCATCGGTCTTCCCC |
| | | CTGGCACCCTCCTCCAAGAGCACCTCTGGGGGCACAGCGGCCCTGGGCTGCCT |
| | | GGTCAAGGACTACTTCCCCGAACCTGTGACGGTCTCGTGGAACTCAGGCGCCCT |
| | | GACCAGCGGCGTGCACACCTTCCCGGCTGTCCTACAGTCCTCAGGACTCTACTC |
| | | CCTCAGCAGCGTGGTGACCGTGCCCTCCAGCAGCTTGGGCACCCAGACCTACA |
| | | TCTGCAACGTGAATCACAAGCCCAGCAACACCAAGGTGGACAAGAGAGTTGAGC |
| | | CCAAATCTTGTGACAAAACTCACACATGCCCACCGTGCCCAGCACCTGAACTCCT |
| | | GGGGGGANCGTCAGTCTTCCTCTTCCCCCCAAAACCCAAGGACACCCTCATGAT |
| | | CTCCCGGACCCCTGAGGTCACATGCGTGGTGGTGGACGTGANCN |
| 228 | 1G01_ | NNNNNNNNNNNNNNNNNNNNNNNCCTCGGTTCTATCGATTGAATTCCACCATGG |
| | LC | GATGGTCATGTATCATCCTTTTTCTAGTAGCAACTGCAACCGGTGTACATTCCCA |
| | | GGCTGTTGTGACTCAGGAATCTGCACTCACCACATCACCTGGTGAAACAGTCAC |
| | | ACTCACTTGTCGCTCAAGTATTGGGGCTGTTACAACTAGTAACTACGCCAACTGG |
| | | GTCCAAGAAAAACCAGATCATTTATTCACTGGTCTAATAGGTGGTACCAACAACC |
| | | GAGCTCCAGGTGTTCCTGCCAGATTCTCAGGCTCCCTGATTGGAGACAAGGCTG |
| | | CCCTCACCATCACAGGGGCACAGACTGAGGATGAGGCAATATATTTCTGTGCTC |
| | | TATGGTACAGCAACCATTGGGTGTTCGGTGGAGGAACCAAACTGACTGTCCTAG |
| | | GCCAGCCCAAGTCTTCGCCATCAGTCACCCTGTTTCCGCCCTCGAGTGAGGAGC |
| | | TTCAAGCCAACAAGGCCACACTGGTGTGTCTCATAAGTGACTTCTACCCGGGAG |
| | | CCGTGACAGTGGCCTGGAAGGCAGATAGCAGCCCCGTCAAGGCGGGAGTGGAG |
| | | ACCACCACACCCTCCAAACAAAGCAACAACAAGTACGCGGCCAGCAGCTATCTG |
| | | AGCCTGACGCCTGAGCAGTGGAAGTCCCACAGAAGCTACAGCTGCCAGGTCAC |
| | | GCATGAAGGGAGCACCGTGGAGAAGACAGTGGCCCCTACAGAATGTTCATAGAA |
| | | GCTTGATCCTCAACCTCTGGATTACAAAATTTGTGAAAGATTGACTGGTATTCTTA |
| | | ACTATGTTGCTCCTTTTACGCTATGTGGATACGCTGCTTTAATGCCTTTGTATCAT |
| | | GCTATTGCTNCCCGTATGGCTTTCATTTTCTCCTCCTTGTATAAATCCTGGTTGCT |
| | | GTCTCTTTATGAGGAGTTGTGGCCCGTTGTCAGGNAACGTGGCGTGGNGTGCAC |
| | | TGNGTTTGCTGACGCAACCCCCACTGGTNGGGGCATTGCNNCCACCTGTCANCT |
| | | NCTTTCCGGGACTTTCGCTTN |
| 229 | 2A05_ | NNNNNNNNNNNNNCCNNGNNNACTGCACCTCGGTTCTATCGATTGAATTCCACC |
| | HC | ATGGGATGGTCATGTATCATCCTTTTTCTAGTAGCAACTGCAACCGGTGTACATT |
| | | CCGAGGTCCAGCTGCAACAGTCTGGACCTGTGCTGGTGAAGCCTGGGCCTTCA |
| | | GTGAAGATATCCTGTAAGGCTTCTGGATTCACATTCACTGACTACTACATGCACT |
| | | GGGTGAAGCAGAGCCATGGAAAGAGCCTTGAATGGATTGGACTTGTTTATCCTTA |
| | | CAATGGTGGTACTAGCTACAACCAGAAGTTCAAGGGCAAGGCCACATTGACTGT |
| | | AGACACATCCTCCACCACAGCCTACATGGAGCTAAACAGCCTGACTTCCGAGGA |
| | | CTCTGCGGTCTATTACTGTGCAAGATTGGGCTCAGGCTACGAGTACTACTTTGAC |
| | | TACTGGGGCCAAGGCACCTCTCTCACAGTCTCCTCAGCGTCGACCAAGGGCCCA |
| | | TCGGTCTTCCCCCTGGCACCCTCCTCCAAGAGCACCTCTGGGGGCACAGCGGC |
| | | CCTGGGCTGCCTGGTCAAGGACTACTTCCCCGAACCTGTGACGGTCTCGTGGAA |
| | | CTCAGGCGCCCTGACCAGCGGCGTGCACACCTTCCCGGCTGTCCTACAGTCCT |
| | | CAGGACTCTACTCCCTCAGCAGCGTGGTGACCGTGCCCTCCAGCAGCTTGGGC |
| | | ACCCAGACCTACATCTGCAACGTGAATCACAAGCCCAGCAACACCAAGGTGGAC |
| | | AAGAGAGTTGAGCCCAAATCTTGTGACAAAACTCACACATGCCCACCGTGCCCA |
| | | GCACCTGAACTCCTGGGGGGACCGTCAGTCTTCCTCTTCCCCCCAAAACCCAAG |
| | | GACACCCTCATGATCTCCCGGANCCCTGAGGTCACATGCGTGGTG |
| 230 | 2A05_ | NNNNNNNNNNNNNNGTNACTGCACCTCGGTTCTATCGATTGAATTCCACCATGG |
| | LC | GATGGTCATGTATCATCCTTTTTCTAGTAGCAACTGCAACCGGTGTACATTCCCA |
| | | AATTGTTCTCACCCAGTCTCCAGCACTCATGGCTGCATCTCCAGGGGAGAAGGT |
| | | CACCATCACCTGCAGTGTCAGCTCAAGTATAAGTTCCAGCAACTTGCACTGGTAC |
| | | CAGCAGAAGTCAGAAACCTCCCCCAAACCCTGGATTTATGGCACATCCAACCTG |
| | | GCTTCTGGAGTCCCTGTTCGCTTCAGTGGCAGTGGATCTGGGACCTCTTATTCTC |
| | | TCACAATCAGCAGCATGGAGGCTGAGGATGCTGCCACTTATTACTGTCAACAGT |
| | | GGAGAACTTACCCGTGGACGTTCGGTGGAGGCACCAAGCTGGAAATCAAACGTA |
| | | CGGTGGCTGCACCATCTGTCTTCATCTTCCCGCCATCTGATGAGCAGTTGAAATC |
| | | TGGAACTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAGTGTC |
| | | ACAGAGCAGGACAGCAAGGACAGCACCTACAGCCTCAGCAGCACCCTGACGCT |
| | | GAGCAAAGCAGACTACGAGAAACACAAAGTCTACGCCTGCGAAGTCACCCATCA |
| | | GGGCCTGAGCTCGCCCGTCACAAAGAGCTTCAACAGGGGAGAGTGTTAGAAGC |
| | | TTGATCCTCAACCTCTGGATTACAAAATTTGTGAAAGATTGACTGGTATTCTTAAC |
| | | TATGTTGCTCCTTTTACGCTATGTGGATACGCTGCTTTAATGCCTTTGTATCATGC |
| | | TATTGCTTCCCGTATGGNTTTCATTTTCTCCTCCTTGTATAAATCCTGGTTGCTGN |
| | | CTCTTTATNNNGNAGTTGNNGGCCCGTNNNNNNNANCGTNNN |
| 231 | 2H05_ | NNNNNNNNNNNNNGNCACTGCACCTCGGTTCTATCGATTGAATTCCACCATGGG |
| | HC | ATGGTCATGTATCATCCTTTTTCTAGTAGCAACTGCAACCGGTGTACATTCCGAG |
| | | GTCCAACTGCAGCAGCCTGGACCTGTGCTGGTGAAGCCTGGGCCTTCAGTGAA |
| | | GATATCCTGTAAGGCTTCTGGATTCACATTCACTGACTACTACATGCACTGGGTG |
| | | AAGCAGAGCCATGGAGAGAGCCTTGAGTGGATTGGACTTGTTTCTCCTTACAATG |
| | | GTGGTACTTTCTACAACCAGAAGTTCAAGGGCAAGGCCACATTGACTGTAGACAC |
| | | ATCCTCCAGCACAGCCTACATGGAACTAAACAGCCTGACTTCTGAGGACTCTGC |
| | | GGTCTATTACTGTGCAAGAGTGGGTAGTAGTTACGTCCATTATGCTATGGACTAC |
| | | TGGGGTCAAGGAACCTCAGTCACCGTCTCCTCAGCGTCGACCAAGGGCCCATC |
| | | GGTCTTCCCCCTGGCACCCTCCTCCAAGAGCACCTCTGGGGGCACAGCGGCCC |
| | | TGGGCTGCCTGGTCAAGGACTACTTCCCCGAACCTGTGACGGTCTCGTGGAACT |
| | | CAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCAC |
| | | CCAGACCTACATCTGCAACGTGAATCACAAGCCCAGCAACACCAAGGTGGANNG |
| | | AGAGTTGAGCCCAAATCTTGTGACAAACTCACACATGCCCACCGTGCCCAGCAC |
| | | CTGAACTCCTGGGGGGANCGTCNGTCTTCCTCTTCCCCCAAAACCCNANGANAC |
| | | CCTCATGATCTCCCGGACCCCTGAGGTCNCNTGCGTGGTGGTGGANGTGANNC |
| | | CNNNNAANNNCNNTNAN |
| 232 | 2H05_ | NNNNNNNNNNNCNNGTCACTGCACCTCGGTTCTATCGATTGAATTCCACCATGG |
| | LC | GATGGTCATGTATCATCCTTTTTCTAGTAGCAACTGCAACCGGTGTACATTCCCA |
| | | AATTGTTCTCACCCAGTCTCCAGCACTCACGGCTGCATCTCCAGGGGAGAGGGT |
| | | CACCATCACCTGCAGTGTCAGCTCAAGTATAAGTTCCAGCAACTTGCACTGGTAC |
| | | CAGCAGAAGTCAGAAACCTCCCCCAAACCCTGGATTTATGGCACATCCAACCTG |
| | | GCTTCTGGAGTCCCTGTTCGCTTCAGTGGCAGTGGATCTGGGACCTCTTATTCTC |
| | | TCACAATCAGCAGCATGGAGGCTGAAGATGCTGCCACTTATTACTGTCAACAGTG |
| | | GAGTAAGTACCCGTACACGTTCGGAGGGGGGACCAAGCTGGAAATAAAACGTAC |
| | | GGTGGCTGCACCATCTGTCTTCATCTTCCCGCCATCTGATGAGCAGTTGAAATCT |
| | | GGAACTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAGTGTC |
| | | ACAGAGCAGGACAGCAAGGACAGCACCTACAGCCTCAGCAGCACCCTGACGCT |
| | | GAGCAAAGCAGACTACGAGAAACACAAAGTCTACGCCTGCGAAGTCACCCATCA |
| | | GGGCCTGAGCTCGCCCGTCACAAAGAGCTTCAACAGGGGAGAGTGTTAGAAGC |
| | | TTGATCCTCAACCTCTGGATTACAAAATTTGTGAAAGATTGACTGGTATTCTTAAC |
| | | TATGTTGCTCCTTTTACGCTATGTGGATACGCTGCTTTAATGCCTTTGTATCATGC |
| | | TATTGCTTCCCGTATGGCTTTCATTTTCTCCTCCTTGTATAAATCCTGGTTGCTGT |
| | | CTCTTTATGA |
| 233 | 2B01_ | NNNNNNNNNNNNNNGNCACTGCACCTCGGTTCTATCGATTGAATTCCACCATGG |
| | HC | GATGGTCATGTATCATCCTTTTTCTAGTAGCAACTGCAACCGGTGTACATTCCGA |
| | | GGTCCAACTGCAACAGTCTGGGGCTGGGCTGGTAAAGCCTGGGGCTTCAGTGA |
| | | AGTTGCCTTGCAAGGCTTCTGGCTACACTTTCACCAGCTACTGGATGCACTGGGT |
| | | GCAGCAGAGGCCTGGACAAGGCCTTGAGTGGATTGGATTGATTCATCCTATTGG |
| | | TGGTGGTACTCACTACAATGAGAAGTTCAAGAACAAGGCCACACTGACTGTGGA |
| | | CAAATCCTCCAGCACAGCCTACATGCAACTCAGCAGCCTGACATCTGAGGACTC |
| | | TGCGGTCTATTACTGTGCAAGACTCGGAACTGGTCCGTACTACTTTGACTACTGG |
| | | GGCCAAGGCACCACTCTCACAGTCTCCTCAGCGTCGACCAAGGGCCCATCGGT |
| | | CTTCCCCCTGGCACCCTCCTCCAAGAGCACCTCTGGGGGCACAGCGGCCCTGG |
| | | GCTGCCTGGTCAAGGACTACTTCCCCGAACCTGTGACGGTCTCGTGGAACTCAG |
| | | GCGCCCTGACCAGCGGCGTGCACACCTTCCCGGCTGTCCTACAGTCCTCAGGA |
| | | CTCTACTCCCTCAGCAGCGTGGTGACCGTGCCCTCCAGCAGCTTGGGCACCCA |
| | | GACCTACATCTGCAACGTGAATCACAAGCCCAGCAACACCAAGGTGGACAAGAG |
| | | AGTTGAGCCCAAATCTTGTGACAAAACTCACACATGCCCACCGTGCCCAGCACC |
| | | TGAACTCCTGGGGGGACCGTCAGTCTTCCTCTTCCCCCCAAAACCCAAGGACAC |
| | | CCTCATGATCTCCCGGAN |
| 234 | 2B01_ | NNNNNNNNNNNNNNNNGNNNACTGCACCTCGGTTCTATCGATTGAATTCCACCA |
| | LC | TGGGATGGTCATGTATCATCCTTTTTCTAGTAGCAACTGCAACCGGTGTACATTC |
| | | CCAAATTGTTCTCACCCAGTCTCCAGCACTCATGGCTGCATCTCCAGGGGAGAA |
| | | GGTCACCATCACCTGCAGTGTCAGCTCAAGTATAAGTTCCAGCAACTTGCACTGG |
| | | TACCAGCAGAAGTCAGAGACCTCCCCCAAACCCTGGATTTATGGCACATCCAAC |
| | | CTGGCTTCTGGAGTCCCTGTTCGCTTCAGTGGCAGTGGATCTGGGACCTCTTATT |
| | | CTCTCACAATCAGCAGCATGGAGGCTGAAGATGCTGCCACTTATTACTGTCAACA |
| | | GTGGAGTAGTTACCCGTGGACGTTCGGTGGAGGCACCAAGCTGGAAATCAAACG |
| | | TACGGTGGCTGCACCATCTGTCTTCATCTTCCCGCCATCTGATGAGCAGTTGAAA |
| | | TCTGGAACTGCCTCTGTTGTGTGCCTGCTGAATAACTTCTATCCCAGAGAGGCCA |
| | | AAGTACAGTGGAAGGTGGATAACGCCCTCCAATCGGGTAACTCCCAGGAGAGTG |
| | | TCACAGAGCAGGACAGCAAGGACAGCACCTACAGCCTCAGCAGCACCCTGACG |
| | | CTGAGCAAAGCAGACTACGAGAAACACAAAGTCTACGCCTGCGAAGTCACCCAT |
| | | CAGGGCCTGAGCTCGCCCGTCACAAAGAGCTTCAACAGGGGAGAGTGTTAGAA |
| | | GCTTGATCCTCAACCTCTGGATTACAAAATTTGTGAAAGATTGACTGGTATTCTTA |
| | | ACTATGTTGCTCCTTTTACGCTATGTGGATACGCTGCTTTAATGNCTTTGTATCAT |
| | | GCTATTGCTTCCCGTATGGCTTTCATTTTCTCCTCCTTGNNNNNATCCTGGNTGC |
| | | TGTCTCTTTATGAGGAGTTGTGGGCCCGTTGTCAGGCAACNNNGCGTGGNGTGC |
| | | ACTGTGTTTGCTGACGCAACCCCCNACTGGNNNNNNNATTGCCACCNNNCNGTC |
| | | AGCNNCTTTNCGGGACTTTCGCTTTNCCCNTNCNTATTNNNNNNNN |
| 235 | 1D01_ | NNNNNNNNNNNNNGTCACTGCACCTCGGTTCTATCGATTGAATTCCACCATGGG |
| | HC | ATGGTCATGTATCATCCTTTTTCTAGTAGCAACTGCAACCGGTGTACATTCCGAG |
| | | GTCCAGCTGCAGCAGCCTGGACCTGACGTGGTGAAGCCTGGGGCTTCAGTGAA |
| | | GATTTCCTGCAAGGCTTCTGGCTACACCTTCACTGACTACTATATAAACTGGGTG |
| | | AAGCAGAGGCCTGGACAGGGACTTGAGTGGATTGGATGGATTTTTCCTGGAAGT |
| | | GGTAGTAGTTATTACAATGAGAAGTTCAAGGACAAGGCCACATCTACTGTAGACA |
| | | AATCCTCCAGCACAGCCTACATGTTGCTCAGCAGCCTGACCTCTGAGGACTCTG |
| | | CGGTCTATTTCTGTGCAAAATGGAAGGATTACGGGTGGTACTTCGATGTCTGGG |
| | | GCACAGGGACCACGGTCACCGTCTCCTCAGCGTCGACCAAGGGCCCATCGGTC |
| | | TTCCCCCTGGCACCCTCCTCCAAGAGCACCTCTGGGGGCACAGCGGCCCTGGG |
| | | CTGCCTGGTCAAGGACTACTTCCCCGAACCTGTGACGGTCTCGTGGAACTCAGG |
| | | CGCCCTGACCAGCGGCGTGCACACCTTCCCGGCTGTCCTACAGTCCTCAGGACT |
| | | CTACTCCCTCAGCAGCGTGGTGACCGTGCCCTCCAGCAGCTTGGGCACCCAGA |
| | | CCTACATCTGCAACGTGAATCACAAGCCCAGCAACACCAAGGTGGACAAGAGAG |
| | | TTGAGCCCAAATCTTGTGACAAAACTCACACATGCCCACCGTGCCCAGCACCTG |
| | | AACTCCTGGGGGGACCGTCAGTCTTCCTCTTCCCCCCAAAACCCAAGGACACCC |
| | | TCATGATCTCCCGGANCCCTGNNTCACATGCGTGGTGGTGGACGTGAGCCACGA |
| | | AGACCTNA |
| 236 | 1D01_ | NNNNNNNNNNNNNNNNNGNNNNCTGNACCTCGGTTCTATCGATTGAATTCCACC |
| | LC | ATGGGATGGTCATGTATCATCCTNNTTTCTAGTAGCAACTGCAACCGGTGTACAT |
| | | TCCCAAATTGTTCTCACCCAGTCTCCAGCACTCATGGCTGCATCTCCAGGGGAG |
| | | AAGGTCACCCTCACCTGCAATGTCAGCTCAAGTCTAAGTTCCAGCAACTTGCACT |
| | | GGTACCAGCAGAAGTCAGAAACCTCCCCCAAACCCTGGATTTATGGCACATCCA |
| | | ACCTGGCTTCTGGAGTCCCTGTTCGCTTCAGTGGCAGTGGATCTGGGACCTCTT |
| | | ATTCTCTCACAATCAGCAGCATGGAGGCTGAAGATGCTGCCACTTATTACTGTCA |
| | | ACAGTGGAGGACTTACCCTTACACGTTCGGAGGGGGGACCAAGCTGGAAATAAA |
| | | ACGTACGGTGGCTGCACCATCTGTCTTCATCTTCCCGCCATCTGATGAGCAGTTG |
| | | AAATCTGGAACTGCCTCTGTTGTGTGCCTGCTGAATAACTTCTATCCCAGAGAGG |
| | | CCAAAGTACAGTGGAAGGTGGATAACGCCCTCCAATCGGGTAACTCCCAGGAGA |
| | | GTGTCACAGAGCAGGACAGCAAGGACAGCACCTACAGCCTCAGCAGCACCCTG |
| | | ACGCTGAGCAAAGCAGACTACGAGAAACACAAAGTCTACGCCTGCGAAGTCACC |
| | | CATCAGGGCCTGAGCTCGCCCGTCACAAAGAGCTTCAACAGGGGAGAGTGTTAG |
| | | AAGCTTGATCCTCAACCTCTGGATTACAAAATTTGTGAAAGANTGACTGGTATTCT |
| | | TAACTATGTTGCTCCTTTTACGCTATGTGGATACGCTGCTTTAATGCCTTTGTATC |
| | | ATGCTATTGCTTCCCGTATGGCTTTCATTTTCTCCTCCTTGTATAATCCTGGTTGC |
| | | TGTCTCTTTATGAGGAGTTGTGGNCCGTTGTCAGGCAACGTGGCGTGGTGTGCA |
| | | CTGTGTTTGCNNGACNNCACCCCCNNCTGGTTGGGGNATTGCCACCNNCCTGTC |
| | | AGCTNNTTTCCGGGANNNTN |
| 237 | 2E02_ | NNNNNNNNNNNNGTCACTGCACCTCGGTTCTATCGATTGAATTCCACCATGGGA |
| | HC | TGGTCATGTATCATCCTTTTTCTAGTAGCAACTGCAACCGGTGTACATTCCGAGG |
| | | TCCAACTGCAGCAGTCTGGACCTGTGCTGGTGAAGCCTGGGCCTTCAGTGAAGA |
| | | TATCCTGTAAGGCTTCTGGATTCACATTCACTGACTACTACATTCACTGGGTGAA |
| | | GCGGAGCCATGGAAAGAGCCTTGAGTGGATTGGACTTGTTTCTCCTTACAATGG |
| | | TGGTACTTTCTACAACCAGAAGTTCAAGGGCAAGGCCACATTGACTGTAGATACA |
| | | TCCTCCAATACAGCCTACATGGAGCTAACCAGCCTGACTTCTGAGGACTCTGCG |
| | | GTCTATTACTGTGCAAGATTGGGGGTTAACTGGTACTTCGATGTCTGGGGCACA |
| | | GGGACCACGGTCACCGTCTCCTCAGCGTCGACCAAGGGCCCATCGGTCTTCCC |
| | | CCTGGCACCCTCCTCCAAGAGCACCTCTGGGGGCACAGCGGCCCTGGGCTGCC |
| | | TGGTCAAGGACTACTTCCCCGAACCTGTGACGGTCTCGTGGAACTCAGGCGCCC |
| | | TGACCAGCGGCGTGCACACCTTCCCGGCTGTCCTACAGTCCTCAGGACTCTACT |
| | | CCCTCAGCAGCGTGGTGACCGTGCCCTCCAGCAGCTTGGGCACCCAGACCTAC |
| | | ATCTGCAACGTGAATCACAAGCCCAGCAACACCAAGGTGGACAAGAGAGTTGAG |
| | | CCCAAATCTTGTGACAAAACTCACNNNTGCCCACCGTGCCCAGCACCTGAACTC |
| | | CTGGGGGGANCGTCAGTCTTCCTCTTCCCCCCAAAACCCAAGGACACCCTCATG |
| | | ATCTC |
| 238 | 2E02_ | NNNNNNNNNNNNNNCNNGTCACTGCACCTCGGTTCTATCGATTGAATTCCACCA |
| | LC | TGGGATGGTCATGTATCATCCTTTTTCTAGTAGCAACTGCAACCGGTGTACATTC |
| | | CCAAATTGTTCTCACCCAGTCTCCAGCACTCATGGCTGCATCTCCAGGGGAAAA |
| | | GGTCACCCTCACCTGCAGTGTCAGCTCAAGTATAAGTTCCAGCACCTTGCACTG |
| | | GTACCAGCAGAAGTCAGAAACCTCCCCCAAACCCTGGATTTATGGCACATCCAA |
| | | CCTGGCTTCTGGAGTCCCTGTTCGCTTCAGTGGCAGTGGATCTGGGACCTCTTA |
| | | TTCTCTCACAATCAGCAGCATGGAGGCTGAAGATGCTGCCACTTATTACTGTCAA |
| | | CAGTGGAGTACTTACCCATACACGTTCGGAGGGGGGACCAAGCTGGAAATAAAA |
| | | CGTACGGTGGCTGCACCATCTGTCTTCATCTTCCCGCCATCTGATGAGCAGTTGA |
| | | AATCTGGAACTGCCTCTGTTGTGTGCCTGCTGAATAACTTCTATCCCAGAGAGGC |
| | | CAAAGTACAGTGGAAGGTGGATAACGCCCTCCAATCGGGTAACTCCCAGGAGAG |
| | | TGTCACAGAGCAGGACAGCAAGGACAGCACCTACAGCCTCAGCAGCACCCTGA |
| | | CGCTGAGCAAAGCAGACTACGAGAAACACAAAGTCTACGCCTGCGAAGTCACCC |
| | | ATCAGGGCCTGAGCTCGCCCGTCACAAAGAGCTTCAACAGGGGAGAGTGTTAGA |
| | | AGCTTGATCCTCAACCTCTGGATTACAAAATTTGTGAAAGATTGACTGGTATTCTT |
| | | AACTATGTTGCTCCTTTTACGCTATGTGGATACGCTGCTTTAATGCCTTTGTATCA |
| | | TGCTATTGCTTCCCGTATGGCTTTCATTTTCTCCTCCTTGTATAAATCCTGGNTGC |
| | | TGTCTCTTTATGAAGGAGTTGTGGGCCCGTTGTCNNNAACGTGGGCGTGGTGTG |
| 239 | 1A05_ | NNNNNNNNNNNNNNGNNNNTGCACCTCGGTTCTATCGATTGAATTCCACCATGG |
| | HC | GATGGTCATGTATCATCCTTTTTCTAGTAGCAACTGCAACCGGTGTACATTCCGA |
| | | GGTCCAACTGCAACAGCCTGGACCTGTACTGGTGAAGCCTGGGCCTTCAATGAA |
| | | GATATCCTGTAAGGCTTCTGGGTACGCTTTCACTGACTACTTCATACACTGGGTG |
| | | AGACAGAGCCATGGAAGGAGCCTTGAGTGGATTGGACTTGTTTCTCCTTATAATG |
| | | GTGGTACTTACTACAACCAGAAGTTCAAGGGCAAGGCCTCATTGACTGTAGACAC |
| | | ATCCTCCAGCACAGCCTACATGGAACTAAGCAGCCTGACTTCTGAGGACTCTGC |
| | | GGTCTATTACTGTGCAAGATTGGGCTACTATGGTAACTGGTACTTCTTTGACTACT |
| | | GGGGCCAAGGCACCCCTCTCACAGTCTCCTCAGCGTCGACCAAGGGCCCATCG |
| | | GTCTTCCCCCTGGCACCCTCCTCCAAGAGCACCTCTGGGGGCACAGCGGCCCT |
| | | GGGCTGCCTGGTCAAGGACTACTTCCCCGAACCTGTGACGGTCTCGTGGAACTC |
| | | AGGCGCCCTGACCAGCGGCGTGCACACCTTCCCGGCTGTCCTACAGTCCTCAG |
| | | GACTCTACTCCCTCAGCAGCGTGGTGACCGTGCCCTCCAGCAGCTTGGGCACC |
| | | CAGACCTACATCTGCAACGTGAATCACAAGCCCAGCAACACCAAGGTGGACAAG |
| | | AGAGTTGAGCCCAAATCTTGTGACAAAACTCACACATGCCCACCGTGCCCAGCA |
| | | CCTGAACTCCTGGGGGGANCGTCAGTCTTCCTCTTCCCCCAAAACCCAAGGACA |
| | | CCCTCATGATCTCCCGGACCCCTGAGTCACATGCGTGGTGGTGGACGTGAGCCA |
| | | CGANNA |
| 240 | 1A05_ | NNNNNNNNNNNNNNNNNNCTGCACCTCGGTTCTATCGATTGAATTCCACCATGG |
| | LC | GATGGTCATGTATCATCCTTTTTCTAGTAGCAACTGCAACCGGTGTACATTCCCA |
| | | AATTGTTCTCACCCAGTCTCCAGCACTCATGGCTACATCTCCAGGGGAAAAGGTC |
| | | ACCATCACCTGTAGTGTCAGCTCAAGTATAAGTTCCAGCAACTTGAACTGGTACC |
| | | AGCAGAAGTCAGAAACCTCCCCCAAACCCTGGATTTATGGCACATCCAACCTGG |
| | | CTTCTGGAGTCCCTGTTCGCTTCAGTGGCAGTGGATCTGGGACCTCTTATTCTCT |
| | | CACAATCAGCAGCATGGAGGCTGAAGATGCTGCCACTTATTACTGTCAACAGTG |
| | | GAGTAGTTACCCATACACGTTCGGAGGGGGGACCAAGCTGGAAATAAAACGTAC |
| | | GGTGGCTGCACCATCTGTCTTCATCTTCCCGCCATCTGATGAGCAGTTGAAATCT |
| | | GGAACTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAGTGTC |
| | | ACAGAGCAGGACAGCAAGGACAGCACCTACAGCCTCAGCAGCACCCTGACGCT |
| | | GAGCAAAGCAGACTACGAGAAACACAAAGTCTACGCCTGCGAAGTCACCCATCA |
| | | GGGCCTGAGCTCGCCCGTCACAAAGAGCTTCAACAGGGGAGAGTGTTAGAAGC |
| | | TTGATCCTCAACCTCTGGATTACAAAATTTGTGAAAGATTGACTGGTATTCTTAAC |
| | | TATGTTGCTCCTTTTACGCTATGTGGATACGCTGCTTTAATGCCTTTGTATCATGC |
| | | TATTGCTTCCCGTATGGCTTTCATTTTCTCCTCCNTGTATAAATCCTGNNGCTGNC |
| | | TCTTTATGAGGANTTGNGGCCCGTTGTCAGGNAACGTGGCGNGGTGTGCACTGT |
| | | GNTTGCNNGACNNNNNNCN |

In various other aspects, the monoclonal antibody to FimH comprises a heavy chain protein variable region and a light chain protein variable region configured to bind to and deactivate the binding of the FimH lectin domain protein of E. coli to exposed mannose residues on the surface of epithelial cells. SEQ ID Nos of the amino acid and nucleotide sequences of heavy and light chains of the E. coli-derived monoclonal antibody sequences re summarized in Table 5 below.

In some aspects, the heavy chain protein variable region comprises the amino acid sequence selected from SEQ ID NOS: 241, 243, 245, 247, 249, 251, 253, 255, and 257 and the light chain protein variable region comprises the amino acid sequence selected from SEQ ID NOS: 242, 244, 246, 248, 250, 252, 254, 256, and 258 as listed in Table 5 below. In other aspects, the heavy chain protein variable region is encoded by a nucleotide sequence selected from SEQ ID NOS: 259, 261, 263, 265, 267, 269, 271, 273, and 275, and the light chain protein variable region is encoded by a nucleotide sequence selected from SEQ ID NOS: 260, 262, 264, 266, 268, 270, 272, 274, and 276, shown listed in Table 5 below.

TABLE 5
E. coli-Derived Antibody Sequences

| | E. coli | |
|---|---|---|
| SEQ ID | Sequence | |
| NO | Label | Sequence |
| 241 | A2 HC | TGVHSEVQLQQSGAEQVRPGASVKMSCKASGYTFTTYNMNWMKQT |
| | | PGQGLEWIGVIYPGNGDISYNQKFKGKATLTVDKSSSTAYMQLSSLTSE |
| | | DSAVYFCAREGDYGPWFAYWGQGTLVTVSAAS (SEQ_ID_NO: 241) |
| 242 | A2 LC | TGVHSQIVLTQSPAIMSASPGEKVTMTCRASSSVSSSYLHWYQQKSG |
| | | ASPKFWIYSTSKLASGVPARFSGSGSGTSYSLTISSVEAEDAATYYCQH |
| | | YSRYPLTFGGGTKLEIKRT (SEQ_ID_NO: 242) |
| 243 | A11 HC | TGVHSEVQLQQSGPVLVKPGPSVKISCKASGFTFTDYYIHWVKQSHGK |
| | | SPEWIGLVSPYNGGTYYNQKFMGRATLTEDTSSNTAYMELNSLTSEDS |
| | | AVYYCARLLRGYWYFDVWGTGTTVTVSSAS (SEQ_ID_NO: 243) |
| 244 | A11 LC | TGVHSQIVLTQSPALMAASPGEMVTITCSVSSSISSSNLHWYQQKSET |
| | | SPKPWIYGTSNLASGVPVRFSGSGSGTSYSLTISSMEAEDAATYYCQQ |
| | | WRTYPYTFGGGTKLEIKRT (SEQ_ID_NO: 244) |
| 245 | B7 HC | TGVHSEVQLQQPGAELVRPGASVKMSCKASGYTFTIYNLHWVKQTPR |
| | | QGLEWIGTIYPGDGDTSYNQKFKGKATLTVDKSSSTAYMQLSNLTSED |
| | | SAVYFCAREGDYGPWFAYWGQGTLVTVSAAS (SEQ_ID_NO: 245) |
| 246 | B7 LC | TGVHSQIVLTQSPAIMSASPGEKVTMTCRASSSVSSSYLHWYQQKSG |
| | | ASPKLWIYSSSNLASGVPARFSGSGSGTSYSLTINSVEAEDAATYYCQH |
| | | YGSYPLTFGGGTKLEIKRT (SEQ_ID_NO: 246) |
| 247 | C7 HC | TGVHSEVQLQQSGPELVKPGASVKISCKASGYAFSSSWMNWVKQRP |
| | | GKGLKWIGRIYPGDGDTNYNGKFKGKATLTADKSSXTAYMQLSSLTSE |
| | | DSAVYFCAREGNLXFDYWGQGTTLTVSSAS (SEQ_ID_NO: 247) |
| 248 | C7 LC | TGVHSDIVLTQSPASLDVSLGQRATISCRASQSVSTSTYSYMHWYQQK |
| | | PGQSPKVLIKYASNLEAGVPARVSGSGSGTDFTLNIHPVEEEDTATYYC |
| | | QHSWEIPPTFGGGTKLEIKRT (SEQ_ID_NO: 248) |
| 249 | C12 HC | TGVHSQVQLKQSGPGLVAPSQSLSITCTVSGFSLTNYAISWVRQPPGK |
| | | GLEWLGIIWTGGGTLYNSALKSRLSISKDNSKSQVFLKMNSLQTDDTA |
| | | RYYCARAYYSNYDWYFDVWDTGTTVTVSSAS (SEQ_ID_NO: 249) |
| 250 | C12 LC | TGVHSDIQMTQSPSSLSASLGDRVTISCSASQGISNYLNWYQQKPDGT |
| | | VKLLIYYTSSLHSGVPSRFSGSGSGTDYSLTISNLEPEDIATYYCQQYS |
| | | QVPYTFGGGTKLEIKRT (SEQ_ID_NO: 250) |
| 251 | E7 HC | TGVHSEVQLQQSGAELVKPGASVKVSCKASGYSFTNYWVHWVKQRP |
| | | GQGLEWIGRIYSSDGDSNYNQKFKGKATLTVDKSSSTAYMQLSSLTSE |
| | | DSAVYYCAIEGNLYFDYWGQGTTLTVSSAS (SEQ_ID_NO: 251) |
| 252 | E7 LC | TGVHSDIVLTQSPASLAVSLGQRATISCRASQSVSTSSYSYMHWYQQK |
| | | PGQPPKFLIKYASNLESGVPARFSGSGSGTDFTLNIHPVEEEDTATYYC |
| | | QHSWEIPLTFGAGTKLKLKRT (SEQ_ID_NO: 252) |
| 253 | F7 HC | TGVHSEVQLQQPGPVLVKPGPSVKISCKASGFTFTDYFIHWVKQSHGK |
| | | SLEWIGLVSPYNGGTYYNQQFKGKATLTVDTSSSTAYMDLNSLTSEDS |
| | | AVYYCTRLLRGYWYFDVWGTGTTVTVSSAS (SEQ_ID_NO: 253) |
| 254 | F7 LC | TGVHSQIVLTQSPALMAASPGEKVTITCSVSSSIGSSNLHWYQQKSETS |
| | | PKPWIYGTSNLASGVPVRFSGSGSGTSYSLTISSMEAEDAATYYCQQ |
| | | WSTYPYTFGGGTKLEIKRT (SEQ_ID_NO: 254) |
| 255 | F11 HC | TGVHSQVQLKESGPGLVAPSQSLSITCTVSGFSLTNYAISWVRQPPGK |
| | | GLEWLGIIWTGGGTLYNSALKSRPSISKDNSKSQVFLKMNSLQTDDTA |
| | | RYYCARAYYSNYDWYFDVWDTGTTVTVSSAS (SEQ_ID_NO: 255) |
| 256 | F11 LC | TGVHSDIQMTQSPSSLSASLGDRVTISCSASQGISNYLNWYQQKPDGT |
| | | VKLLIYYTSSLHSGVPSRFSGSGSGTDYSLTISNLEPEDIATYYCQQYS |
| | | QVPYTFGGGTKLEIKRT (SEQ_ID_NO: 256) |
| 257 | H9 HC | TGVHSEVQLQQSGPVLVKPGPSVKISCKASGFTFTDYFVHWVKQSHG |
| | | KSLEWIGLVSPYNGGTYYNQQFKGKATLTVDTSSSTAYMDLNSLTSED |
| | | SAVYYCARLLRGYWYFDVWGTGTTVTVSSAS (SEQ_ID_NO: 257) |
| 258 | H9 LC | TGVHSQIVLTQSPALMAASPGEKVTITCSVSSSISSSTLHWYQQKSETS |
| | | PKPWIYGTSNLASGVPVRFGGSGSGTSYSLTISSMEAEDAATYYCQQ |
| | | WSTYPYTFGGGTKLEIKRT (SEQ_ID_NO: 258) |
| 259 | A2 HC | ACCGGTGTACATTCCGAGGTCCAACTGCAACAGTCTGGGGCTGAG |
| | | CAGGTGAGGCCTGGGGCCTCAGTGAAGATGTCCTGCAAGGCTTCT |
| | | GGCTACACATTTACCACTTACAATATGAACTGGATGAAGCAGACACC |
| | | TGGACAGGGCCTGGAATGGATTGGAGTTATTTATCCAGGAAATGGT |
| | | GATATTTCCTACAATCAGAAGTTCAAGGGCAAGGCCACACTGACTGT |
| | | AGACAAATCCTCCAGCACAGCCTACATGCAGCTCAGCAGCCTGACA |
| | | TCTGAAGACTCTGCGGTCTATTTCTGTGCAAGAGAGGGAGATTATG |
| | | GGCCCTGGTTTGCTTACTGGGGCCAAGGGACTCTGGTCACTGTCT |
| | | CTGCAGCGTCGAC (SEQ_ID_NO: 259) |
| 260 | A2 LC | ACCGGTGTACATTCCCAAATTGTTCTCACCCAGTCTCCAGCAATCAT |
| | | GTCTGCATCTCCAGGGGAAAAGGTCACCATGACCTGCAGGGCCAG |
| | | CTCAAGTGTAAGTTCCAGTTACTTGCACTGGTACCAGCAGAAGTCA |
| | | GGTGCCTCCCCCAAATTCTGGATTTATAGTACATCCAAGTTGGCTTC |
| | | TGGAGTCCCTGCTCGCTTCAGTGGCAGTGGGTCTGGGACCTCTTA |
| | | CTCTCTCACAATCAGCAGTGTGGAGGCTGAAGATGCTGCCACTTATT |
| | | ACTGCCAGCACTACAGTCGTTACCCACTCACGTTCGGAGGGGGGA |
| | | CCAAGCTGGAAATAAAACGTACG (SEQ_ID_NO: 260) |
| 261 | A11 HC | ACCGGTGTACATTCCGAGGTCCAGCTGCAACAGTCTGGACCTGTGC |
| | | TGGTGAAGCCTGGGCCTTCAGTGAAGATATCCTGTAAGGCTTCTGG |
| | | ATTCACATTCACTGACTACTACATACACTGGGTGAAGCAGAGCCATG |
| | | GAAAGAGCCCTGAGTGGATTGGACTTGTTTCTCCTTACAATGGTGG |
| | | TACTTACTACAACCAGAAGTTCATGGGCAGGGCCACATTGACTGAA |
| | | GACACATCCTCCAACACAGCCTACATGGAGCTAAACAGCCTGACTT |
| | | CTGAGGACTCTGCGGTCTATTACTGTGCGAGGTTACTACGGGGCTA |
| | | CTGGTACTTCGATGTCTGGGGCACAGGGACCACGGTCACCGTCTC |
| | | CTCAGCGTCGAC (SEQ_ID_NO: 261) |
| 262 | A11 LC | ACCGGTGTACATTCCCAAATTGTTCTCACCCAGTCTCCAGCACTCAT |
| | | GGCTGCATCTCCAGGGGAGATGGTCACCATCACCTGCAGTGTCAG |
| | | CTCAAGTATAAGTTCCAGCAACTTGCACTGGTACCAGCAGAAGTCA |
| | | GAAACCTCCCCCAAACCCTGGATTTATGGCACATCCAACCTGGCTT |
| | | CTGGAGTCCCTGTTCGCTTCAGTGGCAGTGGATCTGGGACCTCTTA |
| | | TTCTCTCACAATCAGCAGCATGGAGGCTGAAGATGCTGCCACTTATT |
| | | ACTGTCAACAGTGGCGTACTTACCCGTACACGTTCGGAGGGGGGA |
| | | CCAAGCTGGAAATAAAACGTACG (SEQ_ID_NO: 262) |
| 263 | B7 HC | ACCGGTGTACATTCCGAGGTCCAGCTGCAACAGCCTGGGGCTGAG |
| | | CTGGTGAGGCCTGGGGCCTCAGTGAAGATGTCCTGCAAGGCTTCT |
| | | GGCTACACATTTACCATTTACAATTTGCACTGGGTAAAGCAGACACC |
| | | TAGACAGGGCCTGGAATGGATTGGAACTATTTATCCAGGAGATGGTG |
| | | ATACTTCCTACAATCAGAAGTTCAAGGGCAAGGCCACACTGACTGTA |
| | | GACAAATCCTCCAGCACAGCCTACATGCAGCTCAGCAACCTGACAT |
| | | CTGAAGACTCTGCGGTCTATTTCTGTGCAAGAGAGGGAGATTATGG |
| | | GCCCTGGTTTGCTTACTGGGGCCAAGGGACTCTGGTCACTGTCTCT |
| | | GCAGCGTCGAC (SEQ_ID_NO: 263) |
| 264 | B7 LC | ACCGGTGTACATTCCCAAATTGTTCTCACCCAGTCTCCAGCAATCAT |
| | | GTCTGCATCTCCAGGGGAAAAGGTCACCATGACCTGCAGGGCCAG |
| | | CTCAAGTGTAAGTTCCAGTTATTTGCACTGGTACCAGCAGAAGTCAG |
| | | GTGCCTCCCCCAAACTCTGGATCTATAGCTCATCCAACTTGGCTTCT |
| | | GGAGTCCCTGCTCGCTTCAGTGGCAGTGGGTCTGGGACCTCTTAC |
| | | TCTCTCACAATCAACAGTGTGGAGGCTGAAGATGCAGCCACTTATTA |
| | | CTGCCAGCACTACGGTAGTTACCCACTCACGTTCGGAGGGGGGAC |
| | | CAAGCTGGAAATAAAACGTACG (SEQ_ID_NO: 264) |
| 265 | C7 HC | ACCGGTGTACATTCCGAGGTCCAACTGCAACAGTCTGGACCTGAGC |
| | | TGGTGAAGCCTGGGGCCTCAGTGAAGATTTCCTGCAAGGCTTCTG |
| | | GCTACGCATTCAGTAGCTCCTGGATGAACTGGGTGAAGCAGAGGCC |
| | | TGGAAAGGGTCTTAAGTGGATTGGACGGATTTATCCTGGAGATGGA |
| | | GATACTAATTACAATGGGAAGTTCAAGGGCAAGGCCACACTGACTG |
| | | CAGACAAATCCTCCNGCACAGCCTACATGCAACTCAGCAGCCTGAC |
| | | ATCTGAGGACTCTGCGGTCTACTTCTGTGCAAGAGAGGGTAACTTG |
| | | NATTTTGACTACTGGGGCCAAGGCACCACTCTCACAGTCTCCTCAG |
| | | CGTCGAC (SEQ_ID_NO: 265) |
| 266 | C7 LC | ACCGGTGTACATTCCGACATTGTGCTGACCCAATCTCCTGCTTCCTT |
| | | AGATGTATCTCTGGGGCAGAGGGCCACCATCTCATGCAGGGCCAG |
| | | CCAAAGTGTCAGTACATCTACCTATAGTTATATGCACTGGTACCAACA |
| | | GAAACCAGGACAGTCACCCAAAGTCCTCATCAAGTATGCATCCAAC |
| | | CTAGAAGCTGGGGTCCCTGCCAGGGTCAGTGGCAGTGGGTCTGGG |
| | | ACAGACTTCACCCTCAACATCCATCCTGTGGAGGAGGAGGATACTG |
| | | CAACATATTACTGTCAGCACAGTTGGGAGATTCCTCCGACGTTCGGT |
| | | GGAGGCACCAAGCTGGAAATCAAACGTACG (SEQ_ID_NO: 266) |
| 267 | C12 HC | ACCGGTGTACATTCCCAGGTGCAGCTGAAGCAGTCAGGACCTGGC |
| | | CTGGTGGCGCCCTCACAGAGCCTGTCCATCACATGCACTGTCTCTG |
| | | GGTTCTCATTAACCAACTATGCTATAAGCTGGGTTCGCCAGCCACCA |
| | | GGAAAGGGTCTGGAGTGGCTTGGAATAATATGGACTGGTGGAGGCA |
| | | CACTTTATAATTCAGCTCTCAAATCCAGACTGAGCATCAGCAAAGAC |
| | | AACTCCAAGAGTCAAGTTTTCTTAAAAATGAACAGTCTGCAAACTGA |
| | | TGACACAGCCAGGTACTACTGTGCCAGAGCCTACTATAGTAACTACG |
| | | ACTGGTACTTCGATGTCTGGGACACAGGGACCACGGTCACCGTCTC |
| | | CTCAGCGTCGAC (SEQ_ID_NO: 267) |
| 268 | C12 LC | ACCGGTGTACATTCCGACATCCAGATGACTCAGTCTCCATCCTCCCT |
| | | GTCTGCCTCTCTGGGAGACAGAGTCACCATCAGTTGCAGTGCAAGT |
| | | CAGGGCATTAGCAATTATTTAAACTGGTATCAGCAGAAACCAGATGG |
| | | AACTGTTAAACTCCTGATCTATTACACATCAAGTTTACACTCAGGAGT |
| | | CCCATCAAGGTTCAGTGGCAGTGGGTCTGGGACAGATTATTCTCTC |
| | | ACCATCAGCAACCTGGAACCTGAAGATATTGCCACTTACTATTGTCA |
| | | GCAATATAGTCAGGTTCCGTACACGTTCGGAGGGGGGACCAAGCTG |
| | | GAAATAAAACGTACG (SEQ_ID_NO: 268) |
| 269 | E7 HC | ACCGGTGTACATTCCGAGGTCCAACTGCAGCAGTCTGGGGCTGAA |
| | | CTGGTGAAGCCTGGGGCTTCAGTGAAGGTGTCCTGCAAGGCTTCT |
| | | GGCTACTCCTTCACCAACTACTGGGTGCACTGGGTGAAGCAGAGG |
| | | CCTGGCCAAGGCCTTGAGTGGATTGGAAGGATTTATTCTTCTGATG |
| | | GTGATTCTAATTACAATCAAAAGTTCAAGGGCAAGGCCACATTGACT |
| | | GTAGACAAATCCTCCAGCACAGCCTACATGCAGCTCAGCAGCCTGA |
| | | CATCTGAGGACTCTGCGGTCTATTACTGTGCAATAGAGGGAAACTTG |
| | | TACTTTGACTACTGGGGCCAAGGCACCACTCTCACAGTCTCCTCAG |
| | | CGTCGAC (SEQ_ID_NO: 269) |
| 270 | E7 LC | ACCGGTGTACATTCCGACATTGTGCTGACCCAATCTCCTGCTTCCTT |
| | | AGCTGTATCTCTGGGGCAGAGGGCCACCATCTCATGCAGGGCCAG |
| | | CCAAAGTGTCAGTACATCTAGCTATAGTTATATGCACTGGTACCAACA |
| | | GAAACCAGGACAGCCGCCCAAATTCCTCATCAAGTATGCATCCAAC |
| | | CTAGAATCTGGGGTCCCTGCCAGGTTCAGTGGCAGTGGGTCTGGG |
| | | ACAGACTTCACCCTCAATATCCATCCTGTGGAGGAGGAGGATACTG |
| | | CAACATATTACTGTCAGCACAGTTGGGAGATTCCTCTCACGTTCGGT |
| | | GCTGGGACCAAGCTGAAGCTGAAACGTACG (SEQ_ID_NO: 270) |
| 271 | F7 HC | ACCGGTGTACATTCCGAGGTCCAACTGCAACAGCCTGGACCTGTGC |
| | | TGGTGAAGCCTGGGCCTTCAGTGAAGATATCCTGTAAGGCTTCTGG |
| | | ATTTACATTCACTGACTACTTCATACACTGGGTGAAGCAGAGCCATG |
| | | GAAAGAGCCTTGAGTGGATTGGACTTGTTTCTCCTTACAATGGTGGT |
| | | ACTTACTACAACCAGCAGTTCAAGGGCAAGGCCACATTGACTGTAG |
| | | ACACATCCTCCAGCACAGCCTACATGGACCTAAACAGCCTGACTTCT |
| | | GAGGACTCTGCGGTCTATTACTGTACGAGGTTACTACGGGGCTACT |
| | | GGTACTTCGATGTCTGGGGCACAGGGACCACGGTCACCGTCTCCT |
| | | CAGCGTCGAC (SEQ_ID_NO: 271) |
| 272 | F7 LC | ACCGGTGTACATTCCCAAATTGTTCTCACCCAGTCTCCAGCGCTCAT |
| | | GGCTGCATCTCCAGGGGAGAAGGTCACCATCACCTGCAGTGTCAG |
| | | CTCGAGTATAGGTTCCAGCAACTTGCATTGGTACCAGCAGAAGTCA |
| | | GAAACCTCCCCCAAACCCTGGATTTATGGCACATCCAACCTGGCTT |
| | | CTGGAGTCCCTGTTCGCTTCAGTGGCAGTGGATCTGGGACCTCTTA |
| | | TTCTCTCACAATCAGCAGCATGGAGGCTGAAGATGCTGCCACTTATT |
| | | ACTGTCAACAGTGGAGTACTTACCCGTACACGTTCGGAGGGGGGA |
| | | CCAAGCTGGAAATAAAACGTACG (SEQ_ID_NO: 272) |
| 273 | F11 HC | ACCGGTGTACATTCCCAGGTGCAGCTGAAGGAGTCAGGACCTGGC |
| | | CTGGTGGCGCCCTCACAGAGCCTGTCCATCACATGCACTGTCTCTG |
| | | GGTTCTCATTAACCAACTATGCTATAAGCTGGGTTCGCCAGCCACCA |
| | | GGAAAGGGTCTGGAGTGGCTTGGAATAATATGGACTGGTGGAGGCA |
| | | CACTTTATAATTCAGCTCTCAAATCCAGACCGAGCATCAGTAAAGAC |
| | | AACTCCAAGAGTCAAGTTTTCTTAAAAATGAACAGTCTGCAAACTGA |
| | | TGACACAGCCAGGTACTACTGTGCCAGAGCCTACTATAGTAACTACG |
| | | ACTGGTACTTCGATGTCTGGGACACAGGGACCACGGTCACCGTCTC |
| | | CTCAGCGTCGAC (SEQ_ID_NO: 273) |
| 274 | F11 LC | ACCGGTGTACATTCCGACATCCAGATGACTCAGTCTCCATCCTCCCT |
| | | GTCTGCCTCTCTGGGAGACAGAGTCACCATCAGTTGCAGTGCAAGT |
| | | CAGGGCATTAGCAATTATTTAAACTGGTATCAGCAGAAACCAGATGG |
| | | AACTGTTAAACTCCTGATCTATTACACATCAAGTTTACACTCAGGAGT |
| | | CCCATCAAGGTTCAGTGGCAGTGGGTCTGGGACAGATTATTCTOTTA |
| | | CCATCAGCAACCTGGAACCTGAAGATATTGCCACTTACTATTGTCAG |
| | | CAATATAGTCAGGTTCCGTACACGTTCGGAGGGGGGACCAAGCTGG |
| | | AAATAAAACGTAC (SEQ_ID_NO: 274) |
| 275 | H9 HC | ACCGGTGTACATTCCGAGGTCCAGCTGCAACAGTCTGGACCTGTGC |
| | | TGGTGAAGCCTGGGCCTTCAGTGAAGATATCCTGTAAGGCTTCTGG |
| | | ATTCACATTCACTGACTACTTCGTACACTGGGTGAAGCAGAGCCATG |
| | | GAAAGAGCCTTGAGTGGATTGGACTTGTTTCTCCTTACAATGGTGGT |
| | | ACTTACTACAACCAGCAGTTCAAGGGCAAGGCCACATTGACTGTAG |
| | | ACACATCCTCCAGCACAGCCTACATGGACCTAAACAGCCTGACTTCT |
| | | GAGGACTCTGCGGTCTATTACTGTGCGAGGTTACTACGGGGCTACT |
| | | GGTACTTCGATGTCTGGGGCACAGGGACCACGGTCACCGTCTCCT |
| | | CAGCGTCGAC (SEQ_ID_NO: 275) |
| 276 | H9 LC | ACCGGTGTACATTCCCAAATTGTTCTCACCCAGTCTCCAGCGCTCAT |
| | | GGCTGCATCTCCAGGGGAGAAGGTCACCATCACCTGCAGTGTCAG |
| | | CTCGAGTATTAGTTCCAGCACCTTGCACTGGTACCAGCAGAAGTCA |
| | | GAAACCTCCCCCAAACCCTGGATTTATGGCACATCCAACCTGGCTT |
| | | CTGGAGTCCCTGTTCGCTTCGGTGGCAGTGGATCTGGGACCTCTTA |
| | | TTCTCTCACAATCAGCAGCATGGAGGCTGAAGATGCTGCCACTTATT |
| | | ACTGTCAACAGTGGAGTACTTACCCGTACACGTTCGGAGGGGGGA |
| | | CCAAGCTGGAAATAAAACGTACG (SEQ_ID_NO: 276) |

A FimH modulating agent can be an agent that induces or inhibits progenitor cell differentiation into FimH expressing cells (e.g., by blocking FimH). For example, anti-FimH antibodies can be used to block FimH.

FimH Signal Reduction, Elimination, or Inhibition by Small Molecule Inhibitors, shRNA, siRNA, or ASOs

As described herein, a FimH modulation agent can be used for use in UTI and other bacterial infection therapy. A FimH modulation agent can be used to reduce/eliminate or enhance/increase FimH signals. For example, a FimH modulation agent can be a small molecule inhibitor of FimH. As another example, a FimH modulation agent can be a short hairpin RNA (shRNA). As another example, a FimH modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

### FimH Inhibiting Agent

One aspect of the present disclosure provides for targeting of FimH, its receptor, or its downstream signaling. The present disclosure provides methods of treating or preventing UTIs and other bacterial infections based on the discovery that treatment with FimH inhibiting antibodies protects against UTI in vivo.

As described herein, inhibitors of FimH (e.g., antibodies, fusion proteins, small molecules) can reduce or prevent UTIs and other bacterial infections. A FimH inhibiting agent can be any agent that can inhibit FimH, downregulate FimH, or knockdown FimH.

As an example, a FimH inhibiting agent can inhibit FimH signaling.

For example, the FimH inhibiting agent can be an anti-FimH antibody. As an example, the anti-FimH antibody can be any anti-FimH antibody identified by ELISA binding assays that bind with high affinity to antigenic FimH. Furthermore, the anti-FimH antibody can be a murine antibody, a humanized murine antibody, or a human antibody.

As another example, the FimH inhibiting agent can be an anti-FimH antibody, wherein the anti-FimH antibody prevents binding of FimH to mannose or prevents activation of FimH and downstream signaling.

As another example, the FimH inhibiting agent can be a fusion protein. For example, the fusion protein can be a decoy receptor for FimH. Furthermore, the fusion protein can comprise a mouse or human Fc antibody domain fused to the ectodomain of FimH.

As another example, a FimH inhibiting agent can be the anti-FimH antibodies identified in the present disclosure, which has been shown to be a potent and specific inhibitor of FimH signaling.

As another example, a FimH inhibiting agent can be an inhibitory protein that antagonizes FimH. For example, the FimH inhibiting agent can be a viral protein, which has been shown to antagonize FimH.

As another example, a FimH inhibiting agent can be a short hairpin RNA (shRNA) or a short interfering RNA (siRNA) targeting FimH or associated biological machinery.

As another example, a FimH inhibiting agent can be an sgRNA targeting FimH of associated machinery.

Methods for preparing a FimH inhibiting agent (e.g., an agent capable of inhibiting FimH signaling) can comprise the construction of a protein/Ab scaffold containing the natural FimH receptor as a FimH neutralizing agent; developing inhibitors of the FimH receptor “down-stream”; or developing inhibitors of the FimH production “up-stream”.

Inhibiting FimH can be performed by genetically modifying FimH in a subject or genetically modifying a subject to reduce or prevent expression of the FimH gene, such as through the use of CRISPR-Cas9 or analogous technologies, wherein, such modification reduces or prevents UTIs and other bacterial infections.

### Chemical Agent:

Examples of FimH inhibiting agents are described herein. FimH inhibiting agents can be of a formula that binds to the mannose-binding domain of the FimH adhesin proteins.

R groups can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include chlorine, chloro, Cl; fluorine, fluoro, F; bromine, bromo, Br; or iodine, iodo, or I.

The term “acetamide”, as used herein, is an organic compound with the formula CH₃CONH_{Z}. The “acetamide” can be optionally substituted.

The term “aryl”, as used herein, unless otherwise indicated, includes a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH2-cyclopropyl, —O—CH2-cyclobutyl, —O—CH2-cyclopentyl, —O—CH2-cyclohexyl, —O—CH2-cycloheptyl, —O—CH2-cyclooctyl, —O— CH2-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C3-10 cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH2-cyclopropyl, —CH2-cyclobutyl, —CH2-cyclopentyl, —CH2-cyclopentadienyl, —CH2-cyclohexyl, —CH2-cycloheptyl, or —CH2-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “hetreocyclic” can be optionally substituted.

The term “indole”, as used herein, is an aromatic heterocyclic organic compound with the formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates the transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects the expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid that is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated into the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or DNA generated externally, such as a DNA sequence containing an antisense message of a gene, or a DNA sequence encoding a synthetic or modified version of a gene.

Host strains developed according to the approaches described herein can be evaluated by any number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, FimH signals can be modulated (e.g., reduced, eliminated, or enhanced) using genome editing. Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of FimH by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for UTIs and other bacterial infections to target cells by the removal of FimH signals.

For example, the methods as described herein can comprise a method for altering a target polynucleotide sequence in a cell comprising contacting the polynucleotide sequence with a clustered regularly interspaced short palindromic repeats-associated (Cas) protein.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of the agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process of treating, preventing, or reversing UTIs and other bacterial infections in a subject in need of administration of a therapeutically effective amount of a FimH inhibiting agent, so as to protect against UTIs and other bacterial infections.

Methods described herein are generally performed on a subject in need thereof. A subject in need of the therapeutic methods described herein can be a subject having, diagnosed with, suspected of having, or at risk for developing a UTI and other bacterial infection. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a FimH inhibiting agent is, for example, an amount that would cause the desired therapeutic effect in a subject while minimizing undesired side effects. In various embodiments, an effective amount of a FimH inhibiting agent described herein can substantially inhibit UTIs and other bacterial infections, slow the progress of UTIs and other bacterial infections, or limit the development of UTIs and other bacterial infections.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a FimH inhibiting agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to protect against a UTI and other bacterial infections.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a FimH inhibiting agent can occur as a single event or over a time course of treatment. For example, a FimH inhibiting agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for UTIs and other bacterial infections.

A FimH inhibiting agent can be administered simultaneously or sequentially with another agent, such as an antibiotic, an anti-inflammatory, or another agent. For example, a FimH inhibiting agent can be administered simultaneously with another agent, such as an antibiotic or an anti-inflammatory. Simultaneous administration can occur through the administration of separate compositions, each containing one or more of a FimH inhibiting agent, an antibiotic, an anti-inflammatory, or another agent. Simultaneous administration can occur through the administration of one composition containing two or more of a FimH inhibiting agent, an antibiotic, an anti-inflammatory, or another agent. A FimH inhibiting agent can be administered sequentially with an antibiotic, an anti-inflammatory, or another agent. For example, a FimH inhibiting agent can be administered before or after administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to, a FimH inhibiting antibody, antibiotics, solubilizing agents, and salts. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

The methods and algorithms of the invention may be enclosed in a controller or processor. Furthermore, methods and algorithms of the present invention can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Monoclonal Antibodies Targeting FimH Adhesin Protect Against Uropathogenic E. coli UTI

In this example, monoclonal antibodies targeting the FimH adhesin protein are developed and identified. Their use in the treatment of UTI in vitro and in vivo is described, which showed protection against UTI in a murine model.

### Introduction

Urinary tract infections (UTIs) affect over 400 million individuals worldwide yearly, leading to $2.8 billion in healthcare and productivity-related costs annually in the US alone. Around 25% of individuals will suffer from recurrent UTIs which severely impairs their quality of life. Further, 27% of all sepsis cases can be traced to urinary origin. This is aggravated by the increased prevalence of multidrug-resistant uropathogens, such as uropathogenic Escherichia coli (UPEC), responsible for 70-90% of UTIs, and Klebsiella pneumoniae, one of the most prevalent non-UPEC uropathogens. UTIs represent the fourth leading cause of death attributed to or associated with antibiotic resistance. Thus, developing antibiotic-sparing strategies to prevent UTIs caused by these difficult-to-treat uropathogens is crucial. One promising approach for new antibacterial treatments is to neutralize key extracellular adhesins to prevent bacterial colonization and invasion into tissue and biofilm formation.

UPEC and K. pneumoniae express chaperone-usher pathway (CUP) type 1 pili that are tipped with the mannose-binding FimH adhesin essential in i) bladder colonization, ii) ascension to cause pyelonephritis, iii) invasion into terminally differentiated umbrella cells of the bladder, iv) the formation of intracellular biofilms in luminal bladder cells, and v) causing an epigenetic imprint in the bladder that predisposes to recurrent UTI. FimH is a two-domain protein with an N-terminal lectin domain containing a deep binding pocket that recognizes mannose with stereochemical specificity and a C-terminal pilin domain linking the adhesin to the pilus. At the tip of the assembled type 1 pilus, FimH exists in a conformational equilibrium between a high-affinity relaxed state and a low-affinity tense state controlled by structural interactions between the FimH lectin and pilin domains. In the high-affinity relaxed state, the FimH lectin domain is highly mobile with respect to the pilin domain. In contrast, in the low-affinity tense state, the pilin domain constrains the lectin domain and allosterically deforms the mannose-binding pocket. UPEC FimH occupies both tense and relaxed conformations whereas the equilibrium in the highly invariant and conserved K. pneumoniae FimH is primarily shifted towards the tense low-binding conformation, explaining its poor mannose binding properties despite an identical mannose binding site.

A vaccine against UPEC FimCH has revealed an 73% reduction in recurrent UTIs caused by UPEC or Klebsiella spp. in a phase 1A/1B clinical trial, showing potential to prevent the two most common UTI pathogens with a FimH targeted therapeutic. The effectiveness of FimH vaccination is associated with antibody responses that inhibit FimH binding. Here, we characterized monoclonal antibodies (mAb) from mice immunized with E. coli and K. pneumoniae FimH lectin domains and discovered cross-reactive antibodies that bind to four distinct FimH structural epitopes (Class 1-4), which block FimH binding in vitro. Using cryo-EM, we discovered that the mAbs are selectively bound to the epitopes displayed in the high-affinity relaxed conformation of FimH. Using binding studies and mouse UTI models, we identified Class 1 mAbs that blocked FimH binding to mannose through steric interference leading to protection against UPEC in mouse UTI models. From structure to an antibiotic alternative therapeutic, these results guide future optimization of FimH mAbs and vaccination strategies to treat E. coli and K. pneumoniae UTIs, two of the most prominent uropathogens with increasing antibiotic resistance.

### Results

FimH_{LD} mAbs can Cross-React with E. coli and K. pneumoniae FimH

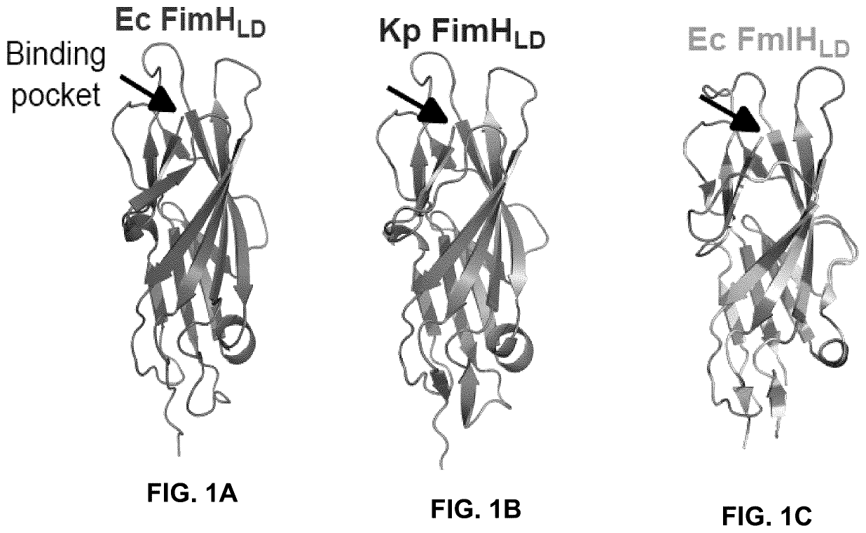

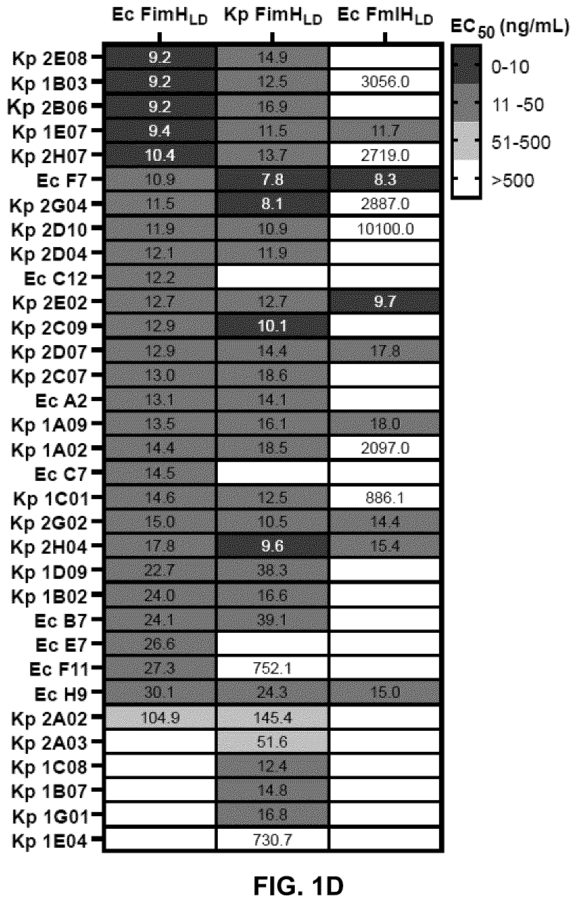

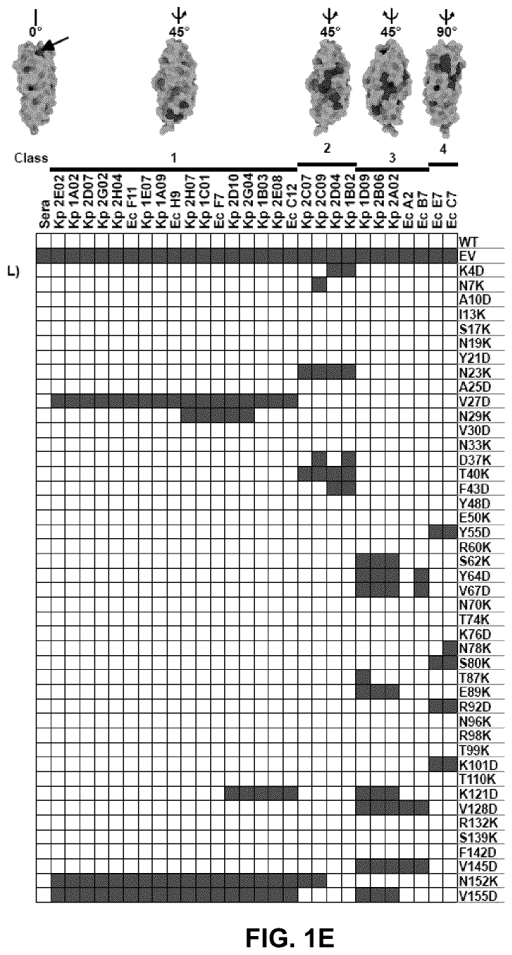

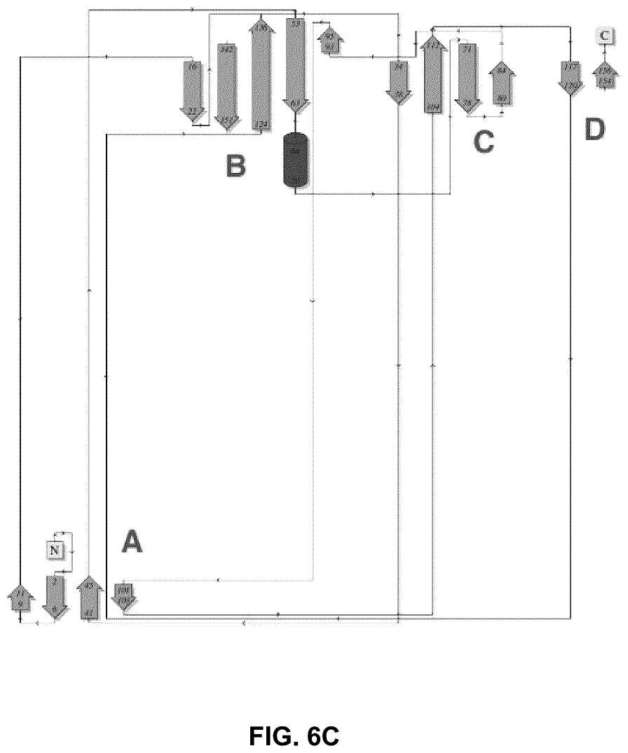

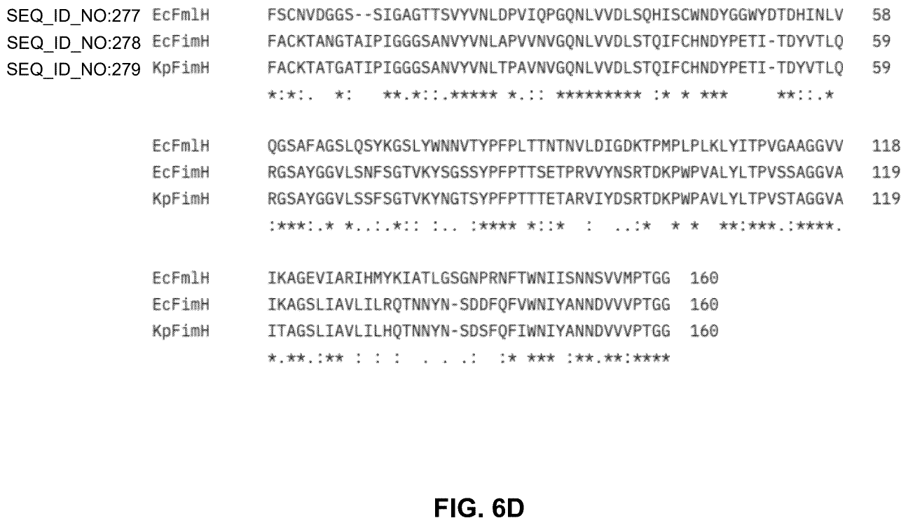

Female C57BL/6J mice were immunized with the lectin domain truncates of FimH (FimH_{LD}) from either E. coli (UT189) or K. pneumoniae (TOP52). Heavy chain V-D-J and light chain V-J fragments from sorted plasmablasts isolated from draining lymph nodes were cloned into human IgG expression vectors to create chimeric murine/human mAbs as previously described. In total, 33 clonally distinct mAbs were generated from E. coli (8 mAbs) and K. pneumoniae (25 mAbs) that bound the respective FimH_{LD} (referred to as Ec or Kp mAbs respectively). The FimH_{LD} structures of E. coli and K. pneumoniae are highly homologous (RMSD=0.420) with an 86% amino acid sequence identity, including an identical binding pocket (FIG. 1A, FIG. 1B, FIG. 6A, FIG. 6B). By ELISA, most mAbs bound to their respective FimH_{LD} antigen with half-maximal effective binding concentrations (EC₅₀) ranging from 8 to 50 ng/mL, while some bound more weakly with EC₅₀ values from 50 to 751 ng/mL (FIG. 1D). Half (4 of 8) of the Ec mAbs also reacted with K. pneumoniae FimH_{LD} and 80% (20 of 25) of Kp mAbs reacted with E. coli FimH_{LD}. Further, eight mAbs (2 Ec and 6 Kp mAbs) that reacted with both FimH antigens also bound with high affinity to a third structurally similar (RMSD=0.699 to Ec FimH) E. coli adhesin, FmIH lectin domain (FmIH_{LD}), an adhesin that binds to exposed galactose residues on bladder tissue during chronic cystitis infections (FIG. 1C, FIG. 1D, FIG. FIG. 6C).

FimH_{LD} mAbs Bind Diverse Epitopes

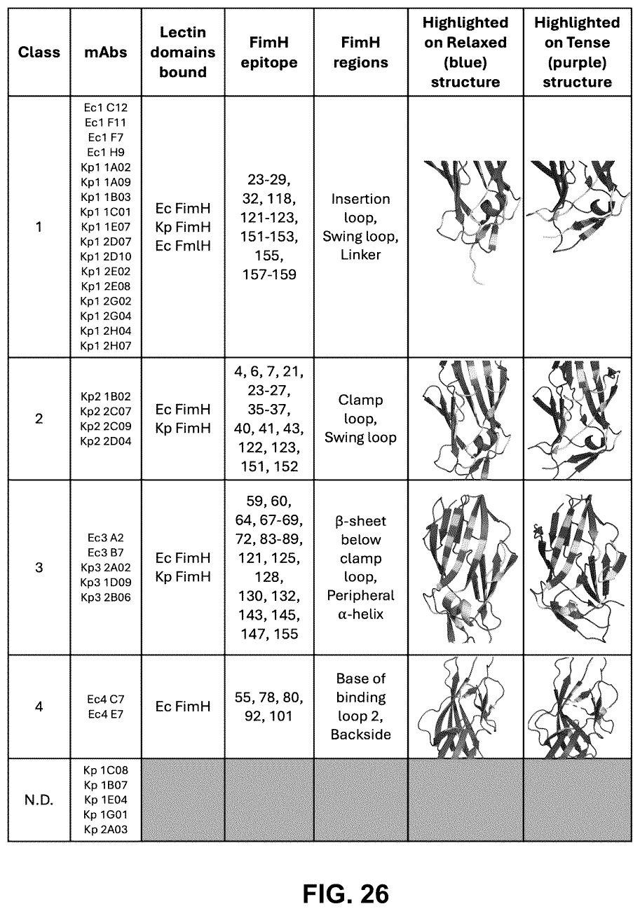

To determine the structural regions of FimH_{LD} that were recognized by each mAb, we generated a mutant library consisting of 45 surface-exposed mutations to bulky charged residues across the lectin domain of full-length E. coli FimH. Binding of the mAbs to the stable mutant FimH proteins was determined by ELISA, which revealed four binding site classes (FIG. 1E, structural regions of FimH are defined in FIG. 6D and FIG. 26). Class 1 mAbs (4 Ec and 13 Kp mAbs), bind with high affinity to both E. coli and K. pneumoniae FimH_{LD} and FmIH_{LD}. Class 1 mAbs were unable to bind E. coli FimH Class 1 epitope residue mutants V27D, N152K, and V155D located in the insertion and swing loops, and in the part of the linker between the pilin and receptor binding domains, suggesting they bind to the base of the lectin domain. Class 2 mAbs (4 Kp mAbs) bound to E. coli and K. pneumoniae FimH_{LD}, but did not react with FmIH_{LD}, consistent with the sequence differences between E. coli FimH_{LD} and E. coli FmIH_{LD} (FIG. 6D). Class 2 mAbs shared the inability to bind to the Class 2 epitope residue mutants N23K and T40K in E. coli FimH suggesting that they bind to the side of the FimH_{LD} body between the base of the binding clamp loop and basal swing loop. A majority of Class 3 mAbs (2 Ec mAbs and 3 Kp mAbs), bound to both E. coli and K. pneumoniae FimH_{LD}, but not to FmIH_{LD}. Class 3 mAbs were unable to bind to Class 3 epitope residue mutants S62K, Y64D, V67D, E89K, K121 D, V128D, V145D, and V155D in E. coli FimH, suggesting that they bind below the binding pocket to the opposite lateral side of the FimH_{LD} body from the Class 2 epitope, covering regions on β-sheet B below the clamp loop and peripheral alpha-helix. Class 4 mAbs (2 Ec mAbs) were only able to bind E. coli FimH_{LD}, and not to K. pneumoniae FimH_{LD} or E. coli FmIH_{LD} and were unable to bind to Class 4 epitope residue mutants Y55D, S80K, R92D, and K101 D in E. coli FimH, suggesting that they bound at the base of binding loop two and backside of FimH_{LD}. Five Kp mAbs were not mapped as they did not bind E. coli FimH. Thus, immunization of E. coli and K. pneumoniae FimH_{LD} antigens generated mAbs bound to four unique surfaces (Class 1-4 epitopes) on E. coli FimH. Below, mAb nomenclature denotes the epitope class recognized by the mAb after the Ec or Kp designation. Kp FimH_{LD} mAbs whose epitopes could not be determined are left unnumbered.

### Structural Basis of FimH mAb Recognition

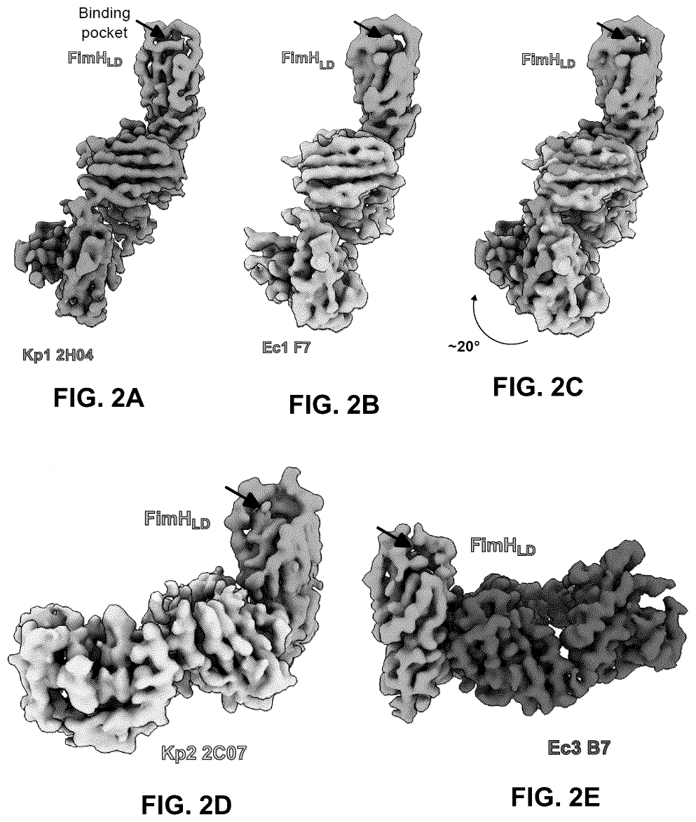

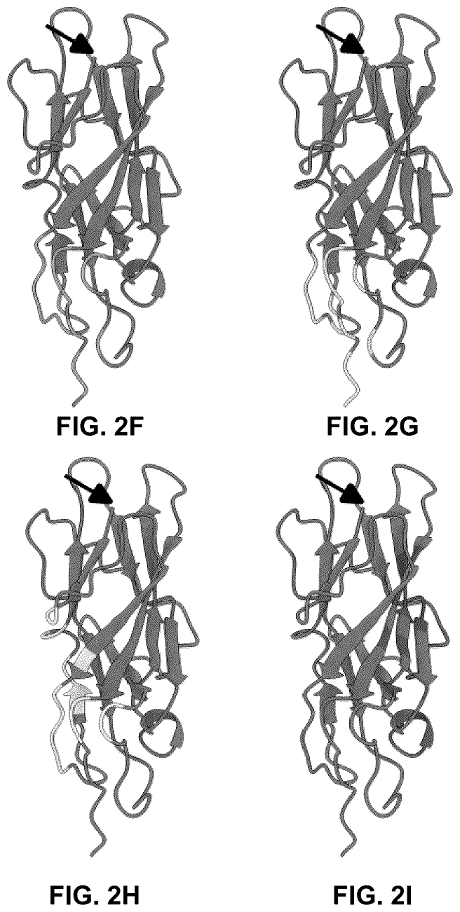

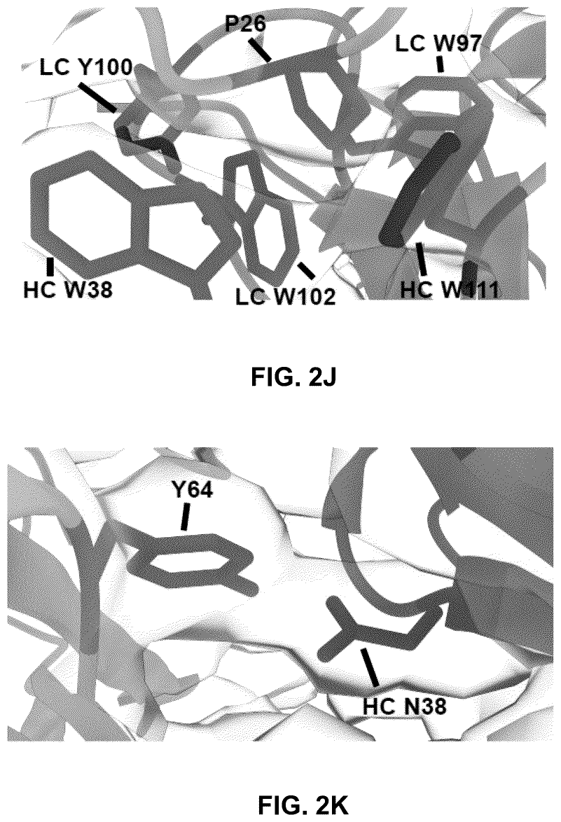

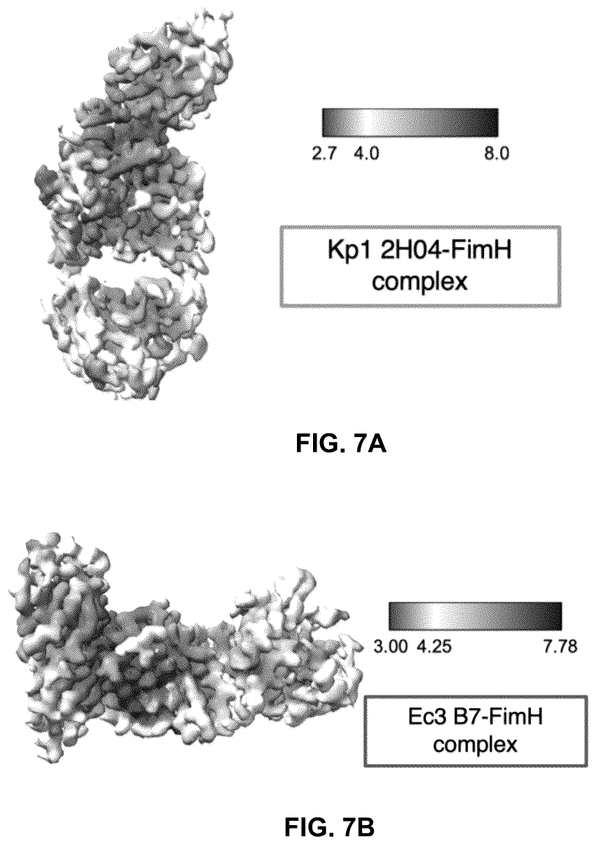

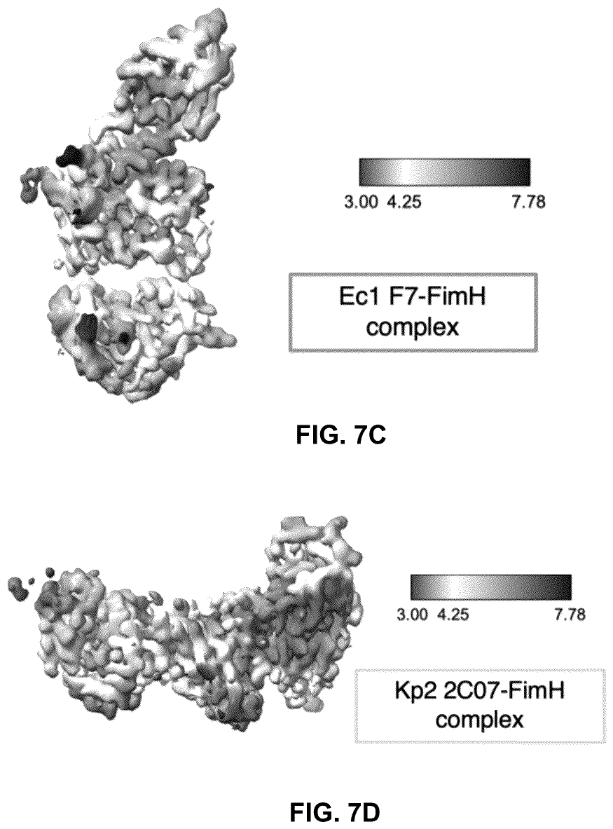

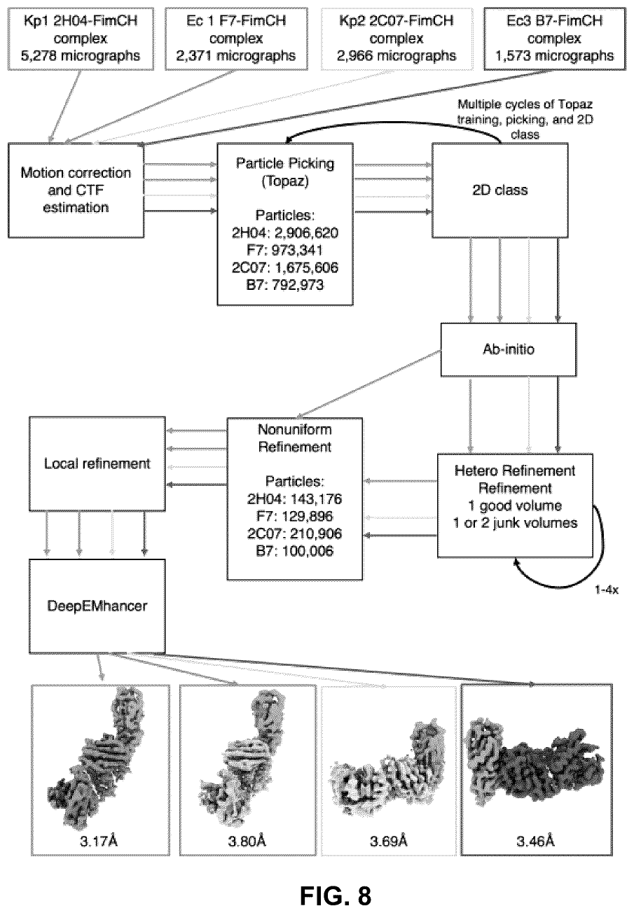

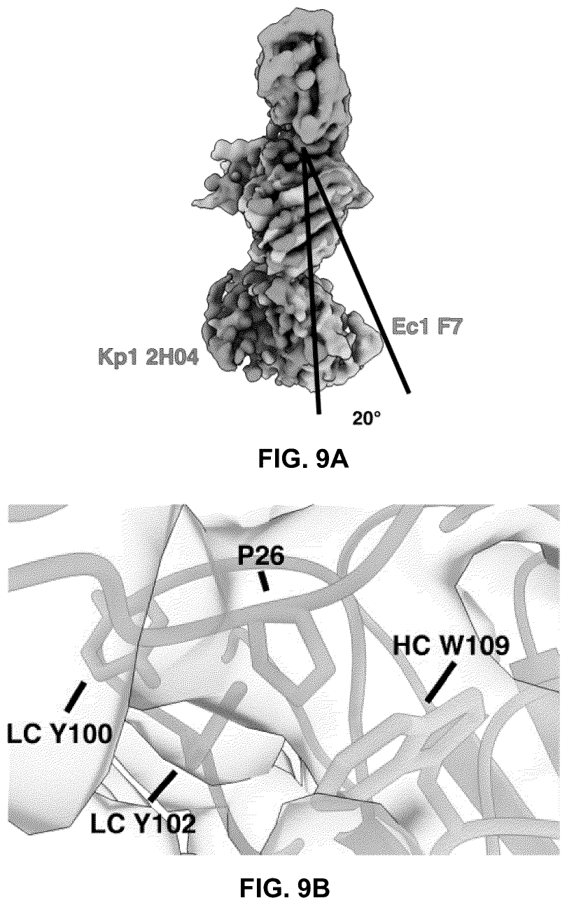

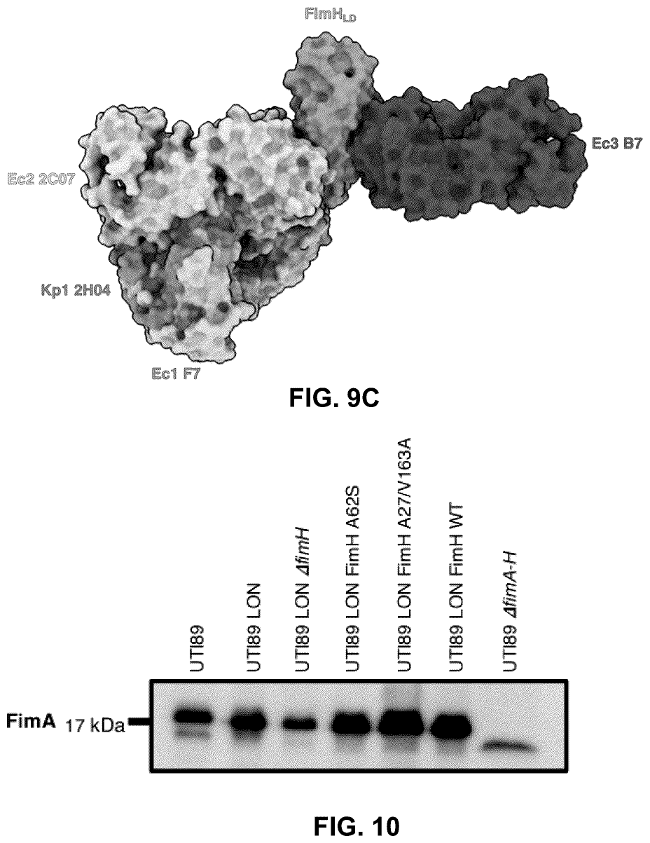

The structural basis of Class 1-3 mAbs binding to FimH_{LD} was determined by cryo-electron microscopy (cryo-EM) of FimCH complexed with fragment antigen-binding regions (Fabs) of Kp1 2H04, Ec1 F7, Kp2 2C07, and Ec3 B7 (FIG. 2A, FIG. 2B, FIG. 2D, FIG. 2E, FIG. 7A, FIG. 7B, FIG. 7C, FIG. 7D, FIG. 8, FIG. 9C). In each case, the observed interaction between FimH_{LD} and Fabs confirmed and extended the epitope mapping described above. The structural basis of Kp1 2H04 Fab and Ec1 F7 Fab binding to Class 1 epitope of FimH revealed that they both interacted with the FimH_{LD} swing loop and linker regions (Class 1 epitope residues A24 to N29 and N151 to T158) and coordinated multiple aromatic residues around FimH_{LD} Class 1 residue P26 (FIG. 2A, FIG. 2B, FIG. 2C, FIG. 2F, FIG. 2G). However, Kp1 2H04 and Ec1 F7 Fabs bound to the E. coli FimH_{LD} at differing angles. Ec1 F7 Fab was rotated ˜20 degrees relative to Kp1 2H04 Fab bound to FimH_{LD} (FIG. 2C, FIG. 9A, FIG. 9B, FIG. 9C). The structure of the Class 2 Kp2 2C07 Fab-FimH_{LD} complex revealed strong binding to the Class 2 epitope residues Y21 to A27 and N151 to D153 (FIG. 2D, FIG. 2H). Class 3 Ec3 B7 Fab bound to β-sheet B of E. coli FimH including Class 3 residue Y64 (FIG. 2I, FIG. 2F), which has been identified as a “toggle switch” between tense and relaxed FimH conformation, with the residue undergoing a major solvent-accessible surface area change in the conformational transition. This binding mode suggests a role of our Class 3 mAbs specifically binding and stabilizing the relaxed FimH conformation, as mutagenesis of Class 3 residue Y64 abolished Class 3 mAb binding in the epitope mapping (FIG. 1E). Altogether, the high-resolution cryo-EM structures identified critical FimH interactions of the mAb epitope classes.

FimH_{LD} mAbs Bind Preferentially to the Relaxed Conformation

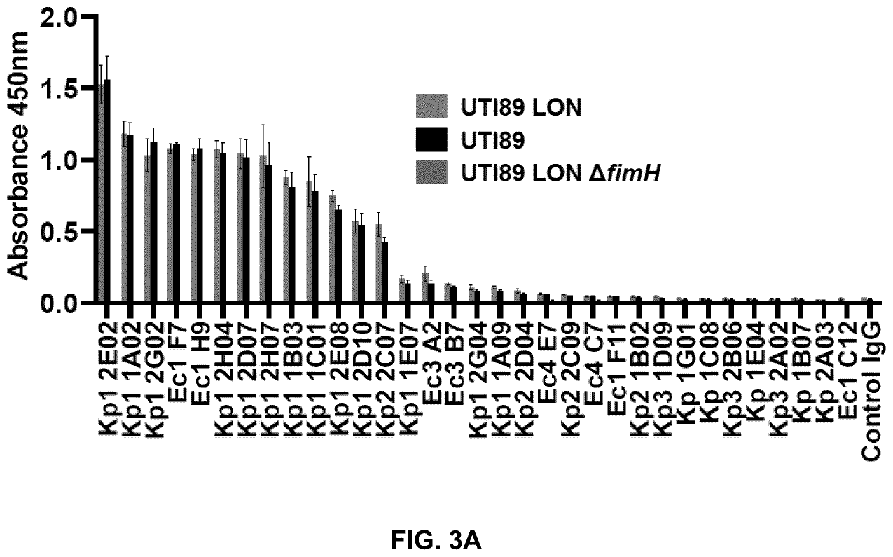

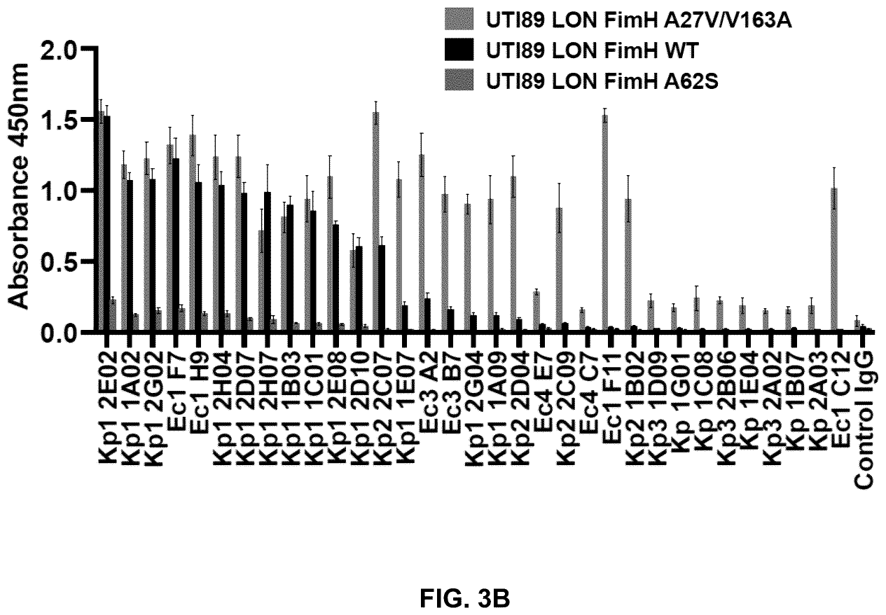

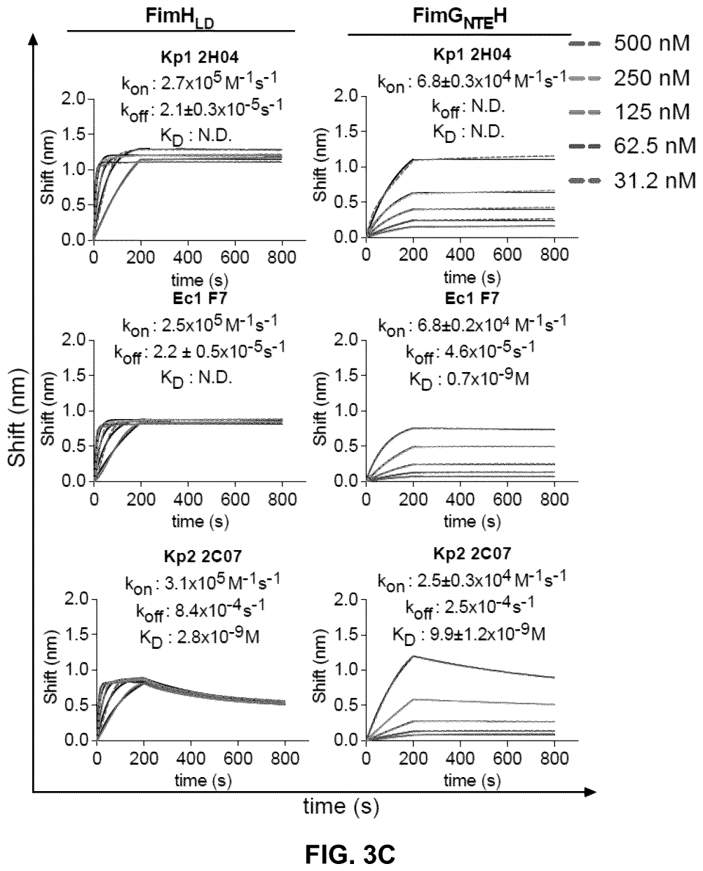

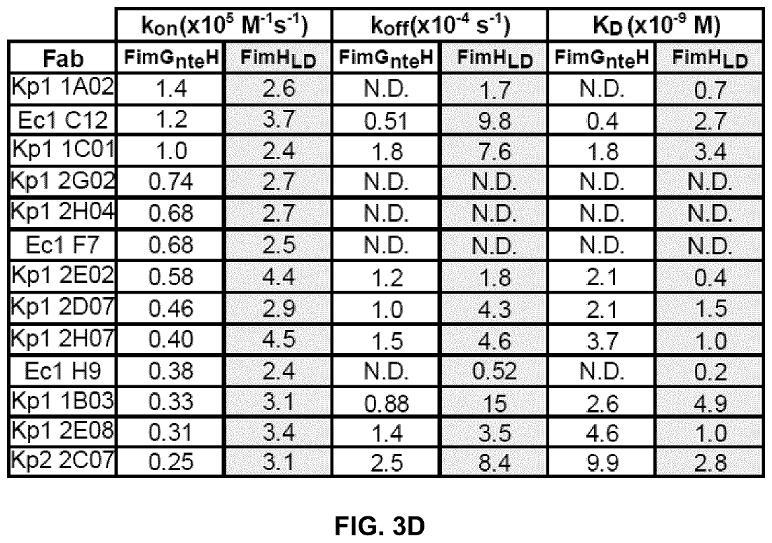

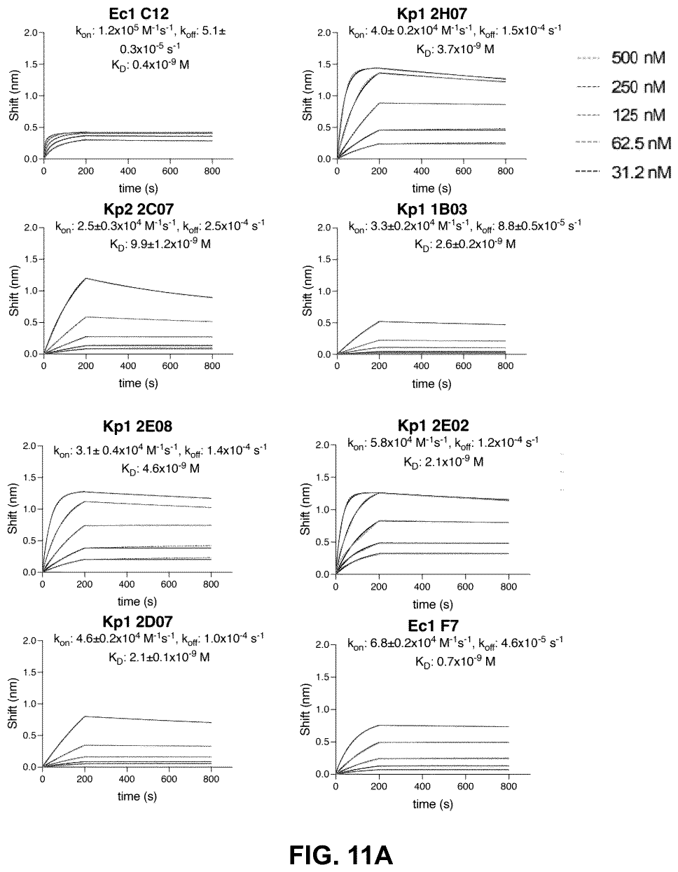

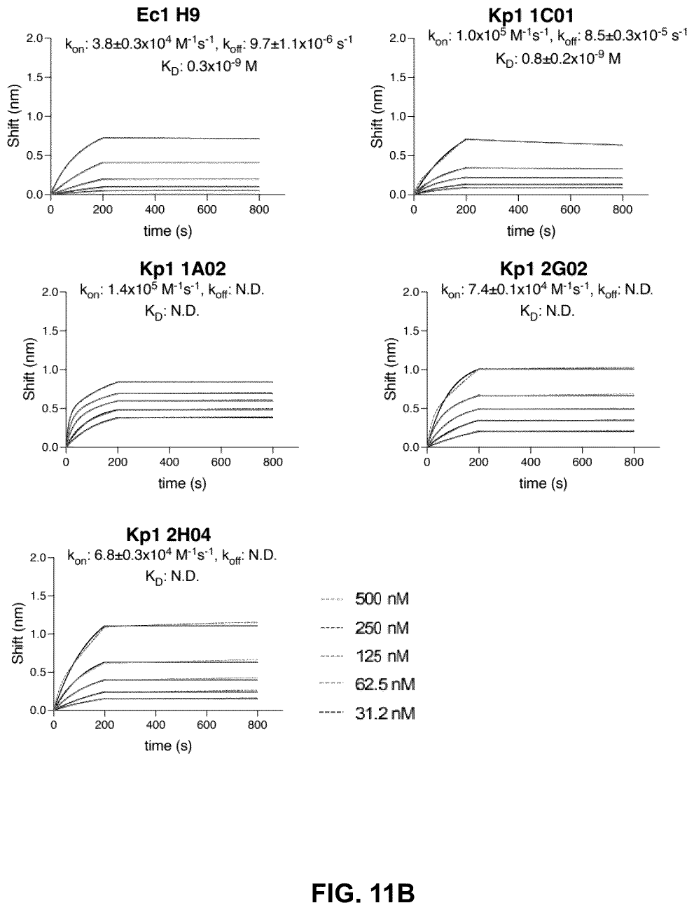

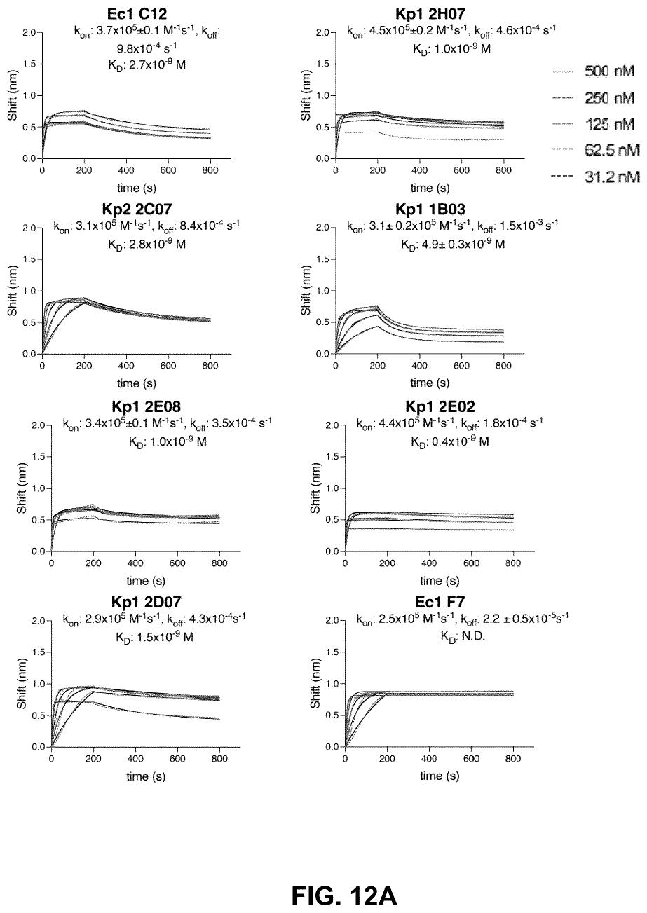

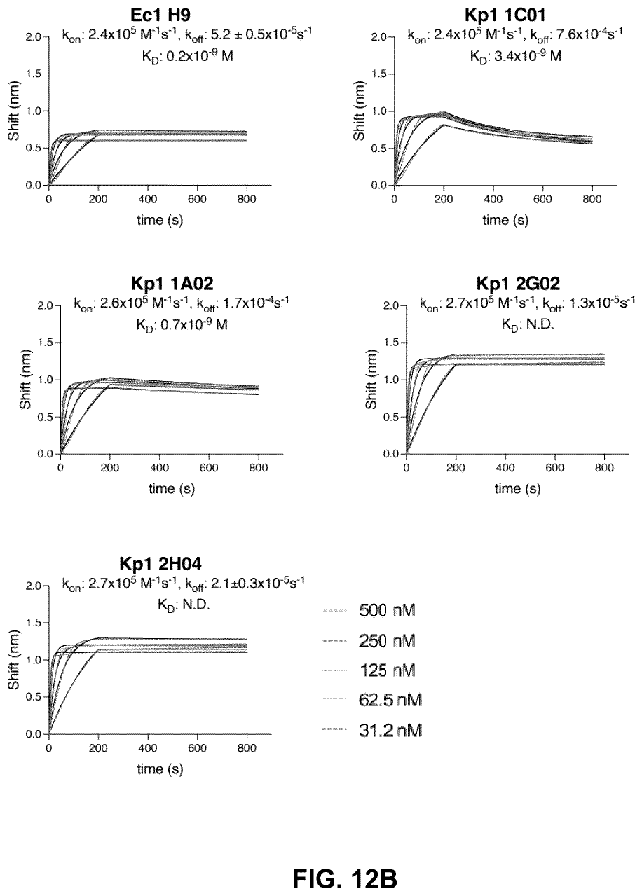

When FimH is incorporated at the tip of type 1 pili, the receptor binding domain samples a conformational equilibrium between low-affinity tense and high-affinity relaxed conformations (FIG. 6A, FIG. 6B). The identified mAb binding epitopes of FimH_{LD}, particularly Class 1-3, are in regions that vary extensively between tense and relaxed conformational states (FIG. 6A, FIG. 6B, FIG. 26). Thus, we investigated whether the conformational dynamics influence the epitopes recognized by Class 1-4 mAbs by measuring binding to FimH-tipped piliated E. coli bacteria using ELISA. Class 1 mAbs displayed the highest reactivity, while Class 2-4 mAbs displayed greatly diminished reactivity to FimH tipped type 1 pili of E. coli (FIG. 3A). Thus, we tested Class 1-4 mAbs against E. coli expressing conformational FimH variants. A majority of mAbs from Classes 1-4 had increased binding to the relax-shifted A27V/V163A E. coli FimH mutant tipping type 1 pili and very weak binding to the tense-shifted A62S E. coli FimH mutant, despite similar levels of type 1 pili expression as measured by western blot (FIG. 3B, FIG. 10). To investigate binding of the highest affinity Class 1-2 mAbs to FimH in a tip-like state, we used Bio-Layer Interferometry (BLI) to measure binding to recombinant FimH. We prepared “tip-like” recombinant full-length FimH by incubating E. coli FimCH with the N-terminal extension (Nte) peptide of FimG resulting in a donor strand exchange reaction where the FimG Nte displaces the FimC chaperone to produce E. coli FimGₙₜₑH complex that samples dynamic conformations like that of FimH tipping type 1 pili. Class 1-2 mAbs had varied binding affinity to E. coli FimGₙₜₑH, with Kₒₙ rates lower than binding to E. coli FimH_{LD} (Kₒₙ rates between 2×10⁴ to 1×10⁵ M⁻¹ s⁻¹) (FIG. 3C, FIG. 3D, FIG. 11A, FIG. 11B, FIG. 12A, FIG. 12B). This finding indicated that Class 1-2 mAbs had a binding preference for the relaxed FimH conformation likely due to FimH_{LD} being the immunizing antigen.

FimH_{LD} mAbs Inhibit FimH Mannosylated Protein Binding

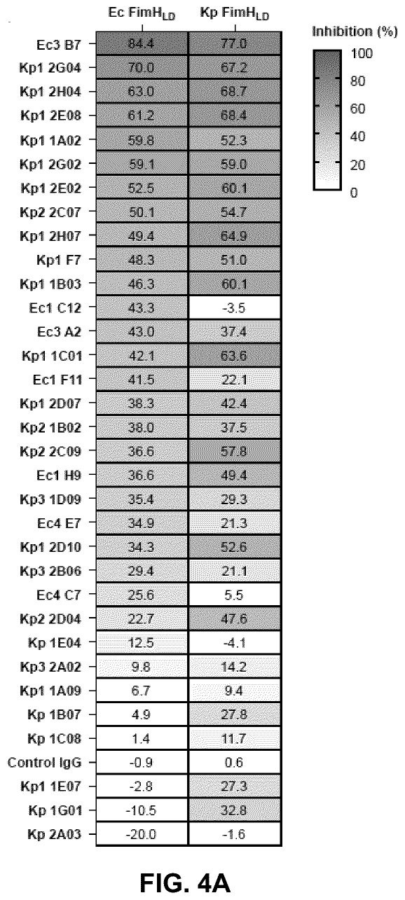

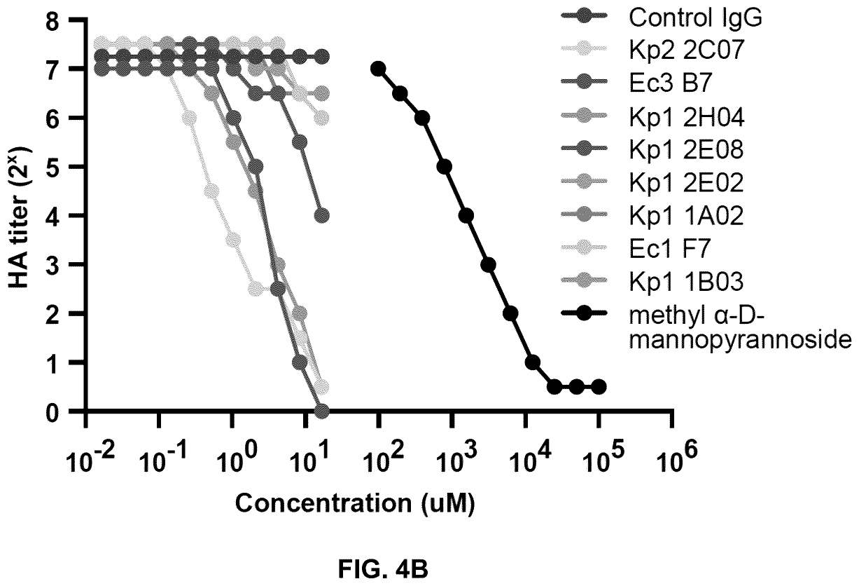

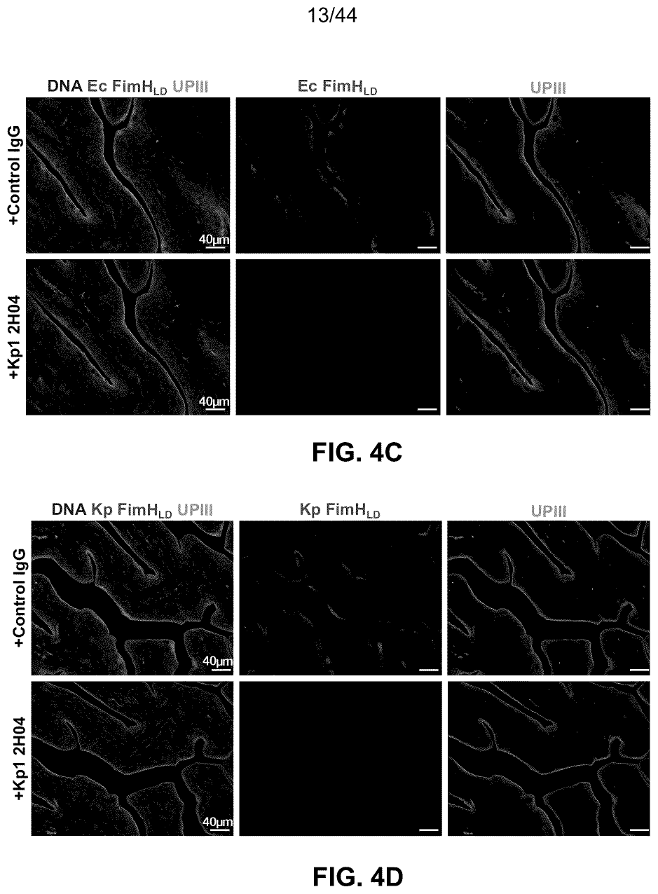

The structural analysis showed mAbs recognizing epitopes near the mannose binding pocket suggesting mAbs may interfere with FimH binding. Thus, FimH_{LD} mAbs were tested for their ability to block E. coli and K. pneumoniae FimH_{LD} binding to highly mannosylated glycoprotein bovine submaxillary mucin (BSM). At a 5:1 molar ratio of mAb to FimH_{LD}, assays measuring mAbs that inhibit binding to BSM in ELISA assays ranged from no inhibition to 85% inhibition compared to untreated control (FIG. 4A). Only eight mAbs (representing epitope Classes 1-3) inhibited both FimH_{LD} proteins at greater than 50% at a 5:1 molar ratio. We selected mAbs from Classes 1-3 from both E. coli and K. pneumoniae antigens with the highest inhibition to FimH_{LD} and the strongest binding to FimH tipping type 1 pili on the surface of bacteria to test for the ability to inhibit mannose-dependent E. coli bacterial hemagglutination of guinea pig erythrocytes. When present in high concentrations (17 uM), we found all mAbs tested can inhibit hemagglutination with more potency than α-D-mannopyranoside. However, the FimH mAbs displayed greatly variable inhibition potency with 50% inhibition concentration ranging from 700 nM to above 17 uM (FIG. 4B). We selected Kp1 2H04 to further test for ability to block FimH_{LD} binding to mouse bladder tissue, as Kp1 2H04 had high inhibition in the FimH BSM ELISA and E. coli hemagglutination. At a 10:1 molar ratio of mAb to FimH_{LD} protein, Ec FimH_{LD} and Kp FimH_{LD} mixed with control IgG bound strongly to the bladder epithelial cells. However, Kp1 2H04 mAb treatment completely blocked Ec FimH_{LD} and Kp FimH_{LD} binding to mouse bladder tissue (FIG. 4C).

FimH_{LD} mAbs Protect Against UTI

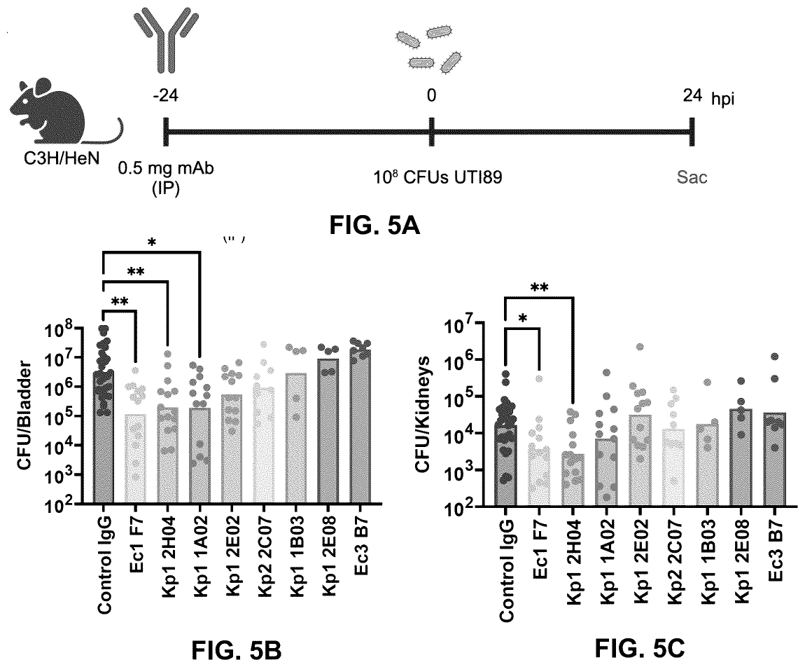

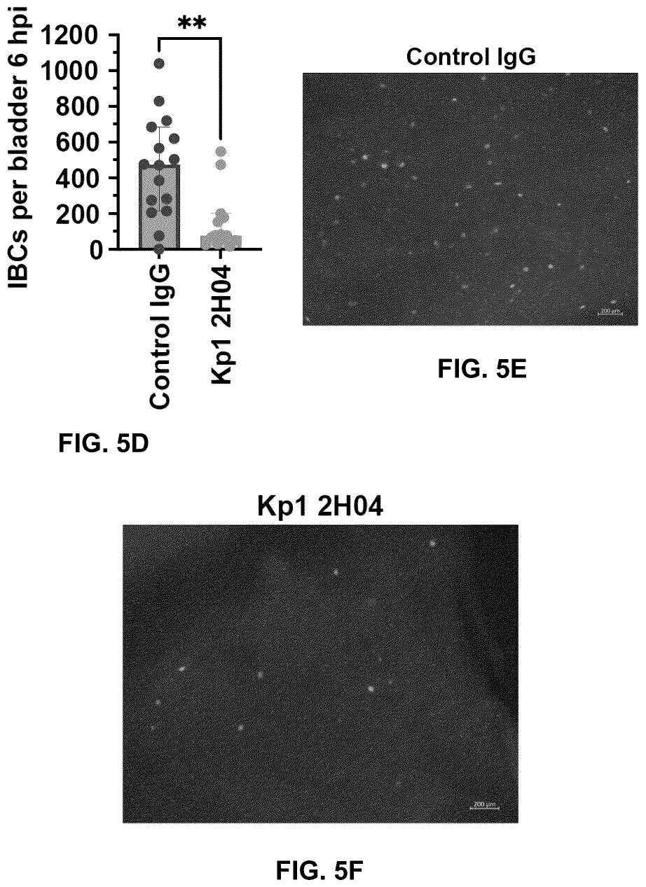

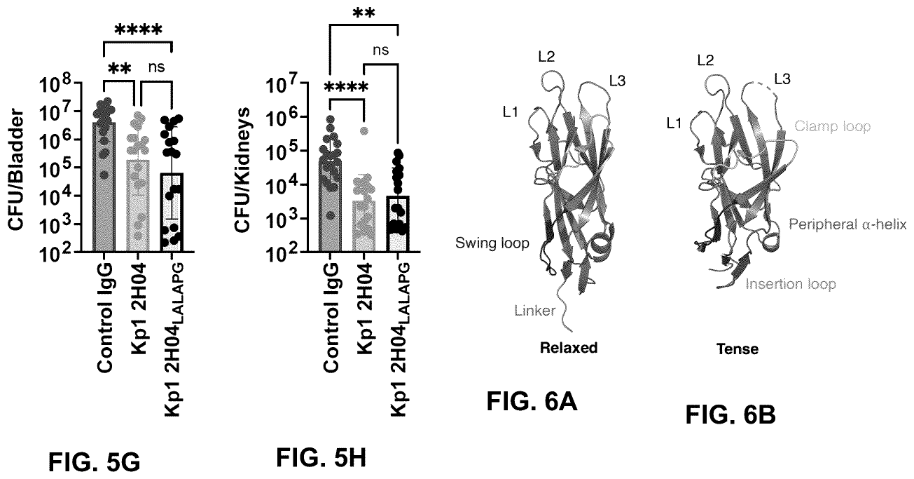

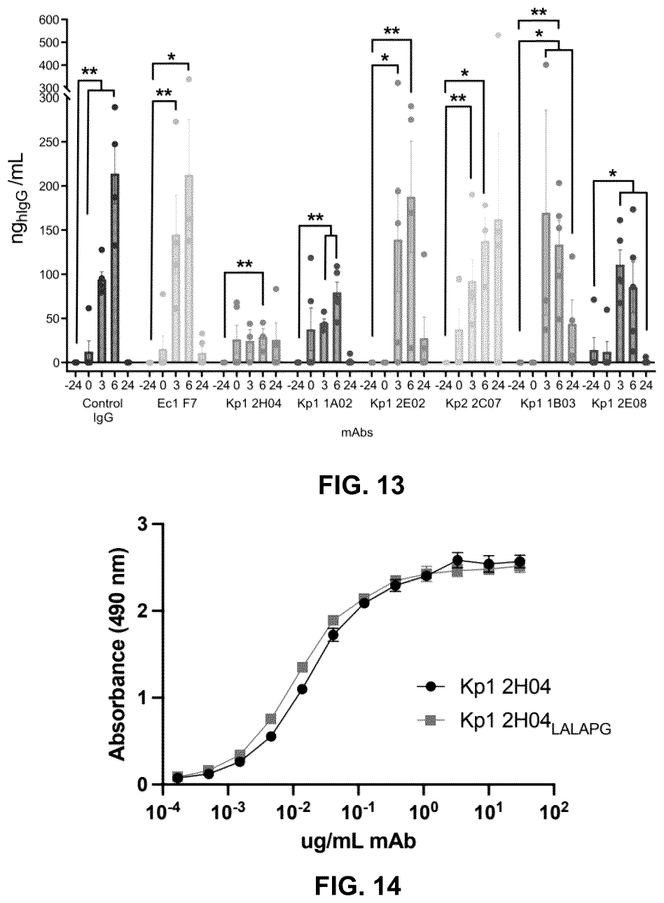

To determine the ability of the mAbs to prevent UTI, we screened eight mAbs in a prophylactic model. We chose Class 1-3 mAbs Ec1 F7, Kp1 2H04, Kp1 1A02, Kp1 2E02, Kp1 1B03, Kp1 2E08 mAbs, Kp2 2C07, and Ec3 B7 mAb for these assays since they bind with high-affinity to tip-like FimH, inhibit bacterial E. coli FimH binding, and represent three epitope classes from E. coli and K. pneumoniae FimH antigens. Each mAb was administered via intraperitoneal injection at 0.5 mg per mouse 24 hours before infection with E. coli UT189. Bladder and kidney titers were enumerated 24 hours post-infection (FIG. 5A). There was no detectable IgG in urine before infection (24 hours after IP injection of mAbs) but mAbs were detected in the bladder approximately 3 to 6 hours after infection, consistent with bladder damage being necessary for antibodies to reach the urine as previously suggested (FIG. 13). Three of the mAbs from epitope Class 1, (Ec1 F7, Kp1 2H04, and Kp1 1A02), resulted in significantly decreased bladder titers (˜1 log; P<0.05) and two Class 1 mAbs (Ec1 F7 and Kp1 2H04) also significantly decreased kidney titers (˜0.5 log; P<0.05) (FIG. 5B, FIG. 5C). The effect of prophylactic administration of Kp1 2H04, one of the strongest inhibitors of bladder titers at 24 hpi, on formation of intracellular bacterial communities (IBCs) at 6 hpi was assessed. Kp1 2H04 significantly decreased the amount of bladder IBCs compared to the control IgG at 6 hpi (FIG. 5D, FIG. 5E, FIG. 5F).

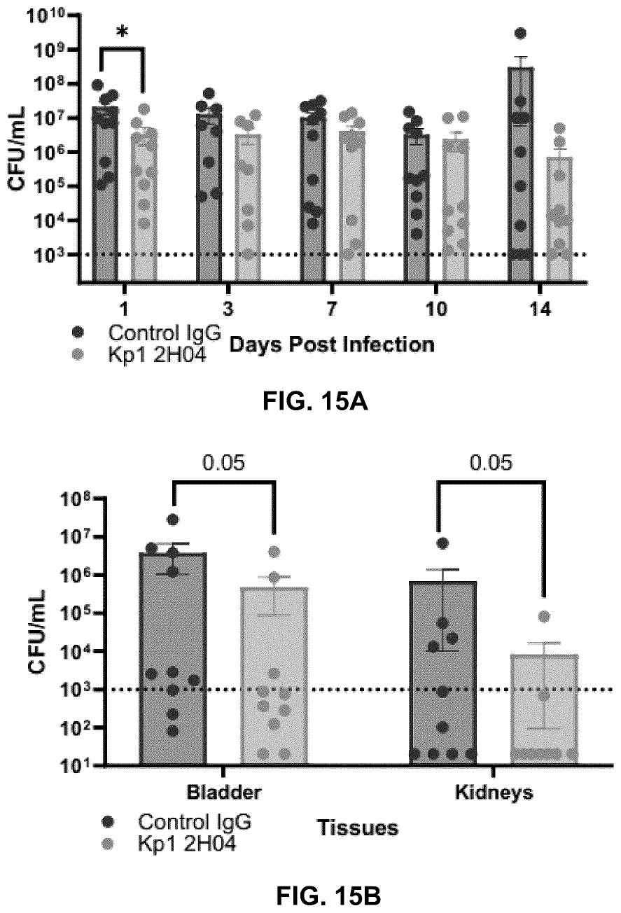

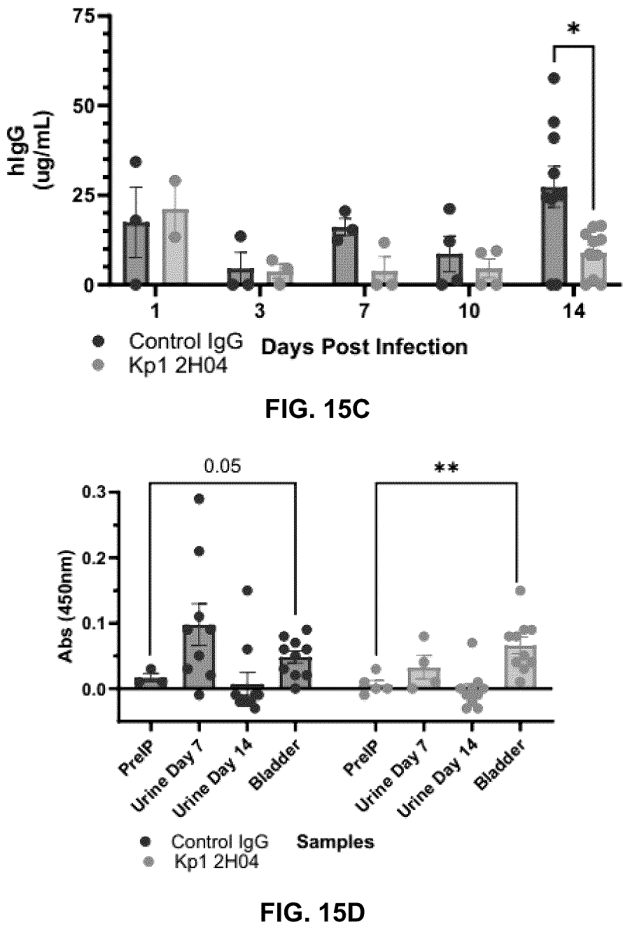

To test if immune system signaling functions associated with the fragment crystallizable (Fc) region of mAbs were needed for protection, we created a LALAPG Fc variant of Kp1 2H04 (2H04_{LALAPG}) which inhibits the ability of the mAb to bind Fc receptors or fix complement. 2H04_{LALAPG} had a comparable binding affinity to FimH_{LD} as 2H04 when assayed by ELISA (FIG. 14). When tested in the prophylactic model, 2H04_{LALAPG} inhibited UT189 infection to the same degree as Kp1 2H04 in the bladder and kidney (FIG. 5G, FIG. 5H), suggesting that Kp1 2H04 prevents infection primarily by directly inhibiting FimH rather than through Fc receptor functions. To further test this, we repeated the prophylactic model but monitored the infection over 14 days. Kp1 2H04 treated mice resulted in lower urine and bladder titers over the 14 days. Still, this difference did not increase over time despite detectable amounts of mAb in the serum and bladder homogenates up to 14 days post-infection (FIG. 15A, FIG. 15B, FIG. 15C, FIG. 15D) suggesting that the primary mechanism of the mAb is blocking initial FimH attachment to the bladder epithelium.

### Discussion

Carbapenem-resistant Enterobacteriaceae (CRE) and extended-spectrum beta-lactamase (ESBL) producing Enterobacteriaceae are listed as urgent and serious threats by the Centers for Diseases Control and Prevention (CDC) as they are resistant to numerous antibiotics needed to treat common infections, including UTIs. New antibiotic-sparing strategies are needed to treat these and other Enterobacteriaceae infections. mAbs have been exceptional drugs in treating cancer and viral infections but few mAb therapies have been notably developed for treating or preventing bacterial infections. Here, we generated mAbs to neutralize FimH, the critical adhesin used by UPEC and K. pneumoniae to mediate UTI pathogenesis. We: i) generated mAbs that define four classes of anti-FimH mAbs that bind to distinct FimH epitopes; ii) identified cross-reactive mAbs with high affinity to E. coli and K. pneumoniae FimH; and iii) characterized the most potent FimH blocking antibodies in a structural and functional analysis. When we tested these mAbs in vivo, we found significant quantities of antibody could be detected in the urine of mice after (but not prior to) infection, suggesting that mAbs penetrate the urinary tract in the context of infection. Two Class 1 Kp and Ec mAbs (Kp1 2H04 and Ec1 F7) significantly reduced bacterial titers in the bladder and kidneys when administered before UPEC infection in a robust mouse model of UTI through direct inhibition of FimH function. This is in contrast to previous studies suggesting that mAbs to the relaxed FimH conformation may stabilize a high-affinity conformation thereby increasing affinity to the bladder. These data suggest that some mAbs, which bind outside the binding pocket to a high-affinity relaxed-conformation by our structural analysis, inhibit FimH binding through steric hindrance preventing access to the binding pocket.

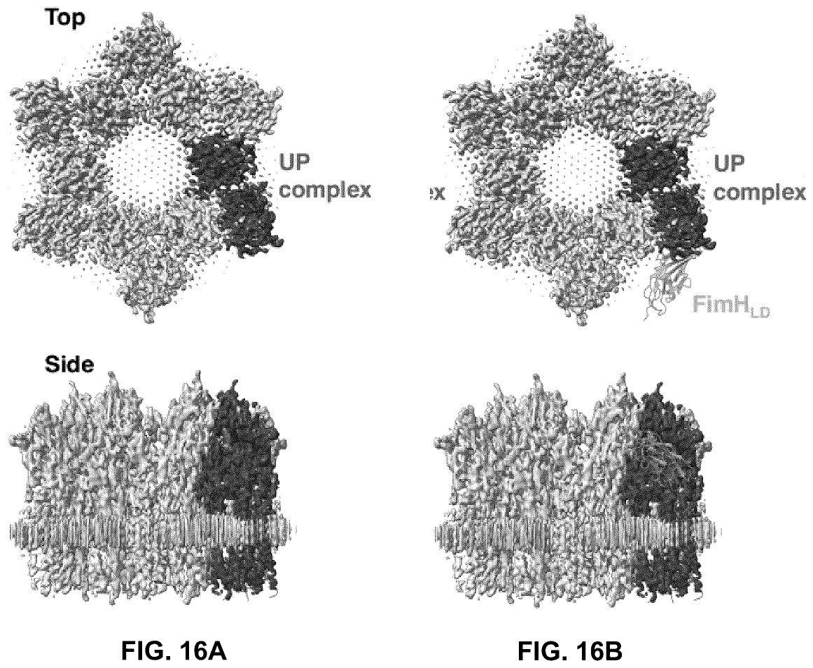

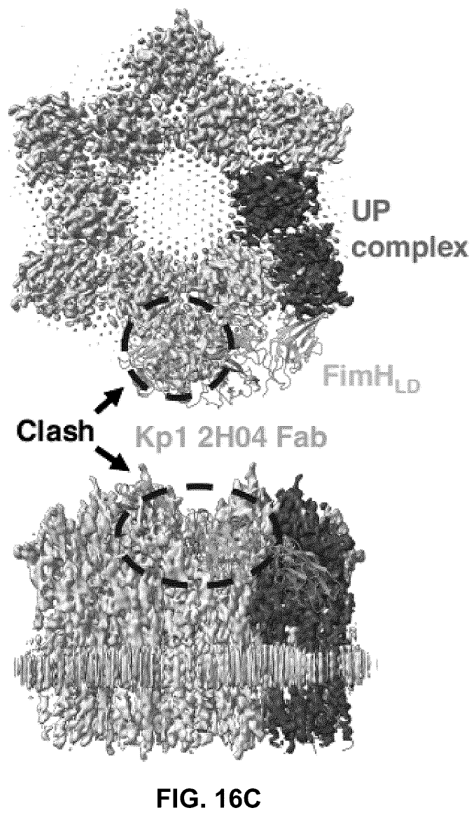

In the bladder, FimH binds uroplakin Ia, which forms an oligomeric complex with other uroplakin proteins in a dense crystalline lipid superstructure burying the mannose glycan deep in the complex. The structure of uroplakin plaques requires FimH to reach into a tight spatial pocket to bind mannose which may be sterically hindered by the binding of mAbs (FIG. 16A, FIG. 16B, FIG. 16C). However, this proposed mechanism does not preclude the additional possibility of stabilizing the high-affinity conformation as shifting the conformational equilibrium towards the high-affinity state via mutations can also result in virulence attenuation. Bacteria with low-affinity tense-shifted FimH alleles may be able to evade vaccination or mAb therapy targeting the relaxed FimH conformation; however, bacteria with tense-shifted FimH alleles are attenuated in infection due to decreased ability to bind to the bladder epithelium. Nevertheless, the highly variable ability of antibodies to bind to tense or relaxed states of FimH suggests that the conformational equilibrium may provide an advantage in vivo by helping UPEC avoid antibodies that effectively bind to only one of the two states. Notably, we did not identify a mAb that directly binds to the FimH mannose-binding pocket, suggesting that vaccination of FimH in the relaxed state may function by providing antibodies that sterically inhibit FimH rather than directly blocking the mannose-binding pocket. Future studies are needed to determine the antigenicity of a tense state FimH and if tense state-specific anti-FimH mAbs are effective at protecting from UTI.

Our results show that FimH-inhibiting mAbs have promising antibiotic-sparing therapeutic potential to treat both UPEC and K. pneumoniae UTIs, lay the groundwork for identifying FimH mAbs with increased efficacy, and provide a roadmap to leverage mAbs to inhibit other bacterial uropathogenic CUP adhesins. While there have been few mAbs developed to treat bacterial infection, mAbs directed at treating UTI could offer unique advantages over antibiotics since they both avoid selection of antibiotic resistance and would have a sustained period of effectiveness. These mAbs may be deployed effectively in patients with highly recurrent UTI, who are often administered prophylactic antibiotics, or in hospital settings with high-risk UTI patients, such as patient populations requiring catheterization, in which UPEC and K. pneumoniae can repeatedly colonize catheters over many months.

### Materials and Methods

### Protein Generation and Purification

FimCH, FimHLD and FmIH lectin domain truncated proteins were purified. Briefly, proteins were expressed and isolated from crude periplasmic preparations using affinity chromatography. In vitro DSE using FimGNTE peptide was performed and purified. FimHLD labeled with EZ-Link™ NHS-PEG4 Biotinylation Kit (ThermoFisher) or Alexa Fluor 647 with NHS Ester conjugation (Invitrogen) was generated according to the manufacturer's instructions. Surface topology diagram of FimH was generated using PDBsum.

Mouse Immunization for Monoclonal Antibody (mAb) Generation

All procedures involving animals were performed in accordance with the guidelines of the Institutional Animal Care and Use Committee (IACUC) of Washington University in Saint Louis. Female C57BL/6J mice (Jackson Laboratories) were immunized intramuscularly with 30 μg E. coli FimHLD or 25 μg K. pneumoniae FimHLD emulsified with AddaVax (InvivoGen). Four weeks later, mice were boosted with a second dose of FimHLD emulsified with AddaVax. One control mouse received PBS emulsified with AddaVax according to the same schedule. Draining iliac and inguinal lymph nodes were harvested 5 days after the boost for plasmablast sorting.

### Cell Sorting for mAb Generation

Staining for sorting was performed using fresh lymph node single cell suspensions in PBS supplemented with 2% FBS and 1 mM EDTA (P2). Cells were stained for 30 min on ice with CD138-BV421 (281-2, 1:200), CD4-PerCP (GK1.5, 1:100), CD19-PE (6D5, 1:200), B220-PE-D594 (RA3-6B2, 1:200), CD38-PE-Cy7 (90, 1:200), Fas-APC (SA367H8, 1:400), IgD-APC-Cy7 (11-26c.2a, 1:100), and Zombie Aqua (all Biolegend) diluted in P2. Cells were washed twice and single plasmablasts (B220lo CD138+ IgDlo CD19+ CD4− live singlet lymphocytes) were sorted using a FACSAria II into 96-well plates containing 2 μL Lysis Buffer (Clontech) supplemented with 1 U/μL RNase inhibitor (NEB) and immediately frozen on dry ice.

Monoclonal Antibody (mAb) and Fragment Antigen Binding (Fab) Generation

Antibodies were cloned. In brief, VH, Vκ, and VA genes were amplified by reverse transcriptase-polymerase chain reaction (RT-PCR) and nested PCR from singly-sorted plasmablasts using cocktails of primers specific for IgG, IgM/A, IgK, and IgA using first round and nested primer sets (Table 6) and then sequenced. Clonally related cells were identified by the same length and composition of IGHV, IGHJ and heavy-chain CDR3 and shared somatic hypermutation at the nucleotide level. To generate recombinant antibodies, heavy chain V-D-J and light chain V-J fragments were PCR-amplified from 1st round PCR products with mouse variable gene forward primers and joining gene reverse primers having 5′ extensions for cloning by Gibson assembly as previously described (Table 6), and were cloned into pABVec6W antibody expression vectors in frame with either human IgG, IgK, or IgL constant domain. Plasmids were co-transfected at a 1:2 heavy to light chain ratio into Expi293F cells using the Expifectamine 293 Expression Kit (Thermo Fisher), and antibodies were purified with protein A agarose (Invitrogen). For monovalent Fab generation, the VH segment of selected antibodies were cloned into a Fab expression vector with a thrombin cleavage site preceding a 6×His tag by GenScript. Fab and light chain plasmids were co-transfected into Expi293F cells for expression and purified with HisPur Ni-NTA resin (Thermo Scientific). For controls in experiments, IgG mAb 2B04, specific for SARS-CoV-2 receptor binding domain was used.

TABLE 6
Primers used for mAb generation
1st Round PCR Primers
IgG, IgK, IgL primers
IgM/A
Forward
VH/Outer: GGGAATTCGAGGTGCAGCTGCAGGAGTCTGG (SEQ_ID_NO: 280)
Reverse
3′Cμ outer: AGGGGGCTCTCGCAGGAGACGAGG (SEQ_ID_NO: 281)
3′Cα outer: GAAAGTTCACGGTGGTTATATCC (SEQ_ID_NO: 282)
Nested PCR primers
IgG, IgK, IgL primers
IgM/A
Forward
VH/Outer: GGGAATTCGAGGTGCAGCTGCAGGAGTCTGG (SEQ_ID_NO: 283)
Reverse
3′Cμ inner: AGGGGGAAGACATTTGGGAAGGAC (SEQ_ID_NO: 284)
3′Cα inner: TGCCGAAAGGGAAGTAATCGTGAAT (SEQ_ID_NO: 285)
Gibson cloning primers
IgH
Forward
VH01:
ATCCTTTTTCTAGTAGCAACTGCAACCGGTGTACATTCCGAGGTCCARCTGCARC
AGYCTGG (SEQ_ID_NO: 286)
VH02:
CCTTTTTCTAGTAGCAACTGCAACCGGTGTACATTCCCAGGTGCAGCTGAAGSAG
TC (SEQ_ID_NO: 287)
VH06:
CCTTTTTCTAGTAGCAACTGCAACCGGTGTACATTCCGAAGTGAAGCTTGARGWG
TCTG (SEQ_ID_NO: 288)
VH14:
CCTTTTTCTAGTAGCAACTGCAACCGGTGTACATTCCGAGGTTCAGCTGCAGCAG
(SEQ_ID_NO: 289)
Reverse
JH01: GAAGACCGATGGGCCCTTGGTCGACGCTGAGGAGACGGTGACCGTG
(SEQ_ID_NO: 290)
JH02: GAAGACCGATGGGCCCTTGGTCGACGCTGAGGAGACTGTGAGA
(SEQ_ID_NO: 291)
JH03: GAAGACCGATGGGCCCTTGGTCGACGCTGCAGAGACAGTGACCAGAG
(SEQ_ID_NO: 292)
JH04: GAAGACCGATGGGCCCTTGGTCGACGCTGAGGAGACGGTGACTGAG
(SEQ_ID_NO: 293)
IgK

### ELISAs

To generate ELISA binding curves to FimHLD and FmIHLD adhesin truncates, plates were first coated with the antigen (0.1 ug/mL) overnight. Plates were washed once with PBS supplemented with Tween-20 (PBS-T, 0.05%) and blocked for 2 h with PBS-T with 10% FBS. mAbs were serially diluted starting at 30 ug/mL and added to the plate to bind for 1 h. Plates were washed 3× in PBS-T. mAb binding was detected with anti-human IgG (Jackson ImmunoResearch, 1:2,500 dilution) for 1 h before washing 3 times in PBS-T and developed with O-phenylenediamine dihydrochloride in citrate buffer (Sigma). Reactions were quenched with 1 M HCl and absorbance was read at 490 nm.

To measure inhibition of FimHLD binding, plates were coated with BSM (10 ug/mL) overnight. Plates were blocked for 2 h with 1×PBS with 1% BSA. FimHLD (2.5 ug/mL) was mixed with a serial curve of mAb (to ensure dose-dependent inhibition) for 1 h. FimHLD mAb mixtures were then added to the plate to let bind for 1 h at RT. Plates were washed 3 times with PBS-T and incubated with anti-streptavidin-HRP (BD Pharmagen) for 1 h before washing 3 times with PBS-T and development with TMB substrate (BD Pharmagen). Reactions were quenched with 1 M H2SO4 and absorbance at 450 nm was recorded.

For measurements of the reactivity of mAbs to bacteria, bacteria were grown statically 2×24 (grown for 24 h and subcultured 1:1000 for another 24 h growth period) in LB and normalized to OD600=1.0 in 1×PBS. Bacteria (100 uL) were added to the plate, spun down 5 min at 3000 rcf, and allowed to bind for 1 h. The supernatant was decanted, and formalin (10%) was added to the wells to fix bacteria for 10 min. Plates were washed 3× in PBS-T and then assayed and developed with the same protocol as the ELISA binding curves to adhesin truncates. To measure levels of humanized IgG (mAbs) in serum, urines and bladder homogenates, plates were coated with diluted 1:100 serum, 1:10 mouse urine, or 1:2 bladder homogenates along with a standard curve of hIgG isotype overnight at 4° C. Plates were then blocked for 2 hours at room temperature with 1×PBS with 1% BSA. Then, plates were washed three times with PBS-T. To measure hIgG levels, plates were then incubated with 1:10,000 dilution of goat anti-Human IgG H&L (HRP) preabsorbed IgG (Abcam, ab97175) for 1 hr at room temperature. For plate development, TMB substrate reagent was added and incubated for approximately 5 minutes at room temperature. Reactions were quenched with 3M HCl and absorbance at 450 nm was recorded.

### Generation of FimH Surface Mutants

Surface FimH (J96) mutants were generated via one-step mutagenesis (19055817) using a pBAD33.1 vector plasmid encoding for FimH (J96 strain) template, Pfu Ultra HF polymerase (Agilent, NC9666083), and the primers listed in Table 6. After PCR amplification, reactions were digested with DpnI (NEB Biolabs, R0176S) to remove the original template from the reaction products. PCR reaction products were transformed into E. coli DH5a for ligation. Plasmids confirmed by Sanger sequencing were then transformed into Escherichia coli C600 Δfim expression strain.

### Epitope Mapping

Epitope mapping of FimH mAbs to E. coli FimChisH was performed using a modified ELISA technique. E. coli strain C600 Δfim carrying pBAD33 plasmids encoding FimH mutants and a ptrc99a plasmid encoding FimChis, were grown in LB to an OD of 0.6-0.8 and then induced with 0.1 mM IPTG and 0.05% arabinose for 1 h. Cells were harvested and periplasm extracts containing FimChisH variants were obtained. FimChisH periplasmic extracts were titrated using anti-FimH sera to normalize the amount of FimH. Normalized FimChisH periplasm was used to coat plates for 1 h at room temperature. Plates were blocked for 2 h with 1×PBS with 1% BSA and mAbs (0.1 ug/mL, except for low-binding mAb 2A02 1 ug/ml was used) were allowed to bind for 1 h. Plates were washed in 1×PBS-T before detection with anti-human IgG (Jackson ImmunoResearch, 1:1000 dilution) and developed with TMB substrate (BD Pharmagen) and H2SO4 as outlined above. mAb binding to each FimChisH mutant was normalized to WT FimChisH binding. Variants that decreased binding below 10% of WT binding and clustered together (3 or more residues) were considered an epitope. Mapping data was visualized and clustered (one-minus Pearson correlation) in Morpheus.

### Western Blotting

Western blotting to detect FimA in bacterial lysates was performed. Bacteria grown 2×24 statically was normalized to optical density at 600 nm of 1.0 and were acid treated with HCl and boiled to disrupt FimA DSE interactions. To measure FimA, rabbit antitype 1 pili (1:2000) was used. A secondary antibody of goat anti-rabbit-HRP (1:10,000, KPL) was used to detect followed by development with SuperSignal™ West Femto Maximum Sensitivity Substrate (Thermo Fisher). Images were obtained on a BioRad ChemiDoc system. A colorimetric image (to view protein size ladder) was overlaid on the chemiluminescent image (detection signal) to create the figure reported in this study.

### Biolayer Interferometry (BLI) Studies

Kinetic binding studies were performed on an Octet Red instrument (ForteBio). Avi-tag biotinylated antibody fragments (Fab) were loaded up to 2.5 nm onto Streptavidin sensor tips (Sartorius) that were pre-equilibrated in HEPES Buffered Saline (HBS) with 0.05% Tween-20 and 1% BSA (kinetic buffer A). Diluted antigens (Ec FimHLD, Ec FimGnteH) were monitored for 200 s of association and 600 s of dissociation in kinetic buffer A. Loaded sensor tips dipping in kinetic buffer A were used as reference sensors. Reference subtracted kinetic traces were used to calculate kinetic rate constants (kon, koff) and equilibrium dissociation rate constant (KD) using a Langmuir 1:1 binding model. Resulting binding traces and fits were plotted with GraphPad Prism v10.

### Hemagglutination Inhibition (HAI) Assay

E. coli guinea pig erythrocyte hemagglutination inhibition assays were performed. Briefly, mAbs were serially diluted in microtiter plates, and 25 uL of bacterial suspension (serially diluted from OD600=10.0 in 1×PBS) was added to each well. After incubation for 10 min at room temperature, 25 ul (OD640=2.0) guinea pig erythrocytes in 1×PBS were added for a final volume of 50 ul. The plates were incubated at 4° C. overnight. For each mAb concentration, the HA titer was defined as the greatest dilution of bacteria that caused hemagglutination.

### Cryo-EM Data Collection and Analysis

Fab and FimCH protein were mixed at a ratio of 1.2:1 and dialyzed into 20 mM HEPES pH 7.5 with 50 mM NaCl. Complexes were flash frozen on EM grids in liquid ethane using an FEI Vitrobot (ThermoFisher) and imaged on Titan Krios (2H04 complex) or Glacios (F7, B7 and 2C07 complexes) microscopes using a Falcon 4 electron detector (Thermo Fisher). Movies were processed in Cryosparc v4.4.1 and particles were picked using Topaz. Densities were post-processed using DeepEMhancer for model building. The collection parameters and workflow are described in more detail in FIG. 8. Initial Fab models were built using homology models in SwissModel. FimHLD model was generated by trimming and threading the UT189 FimH sequence on PDB 1KLF. Rough models were initially docked in ChimeraX before multiple rounds of real space refinement in Phenix v1.20.1 with manual editing in COOT v0.9.6. Refinement statistics are shown in Table 7.

TABLE 7
CryoEM model refinement and validation statistics

| | Kp1 2H04-FimH | Ec1 F7-FimH | Ec3 B7-FimH | Kp2 2C07-FimH |
|---|---|---|---|---|
| Composition (#) | | | | |
| Chains | 3 | 3 | 3 | 3 |
| Atoms | 4532 (Hydrogens: 0) | 4501 (Hydrogens: 0) | 4491 (Hydrogens: 0) | 4558 (Hydrogens: 0) |
| Residues | Protein: 597 | Protein: 595 | Protein: 595 | Protein: 601 |
| | Nucleotide: 0 | Nucleotide: 0 | Nucleotide: 0 | Nucleotide: 0 |
| Water | 0 | 0 | 0 | 0 |
| Ligands | 0 | 0 | 0 | 0 |
| Bonds (RMSD) | | | | |
| Length (Å) (# > | 0.005 (0) | 0.005 (0) | 0.004 (0) | 0.004 (0) |
| 4σ) | | | | |
| Angles (°) (# > | 0.852 (1) | 0.911 (2) | 0.707 (3) | 0.809 (4) |
| 4σ) | | | | |
| MolProbity score | 2.26 | 2.5 | 2.08 | 2.19 |
| Clash score | 15.01 | 15.67 | 11.64 | 12.25 |
| Ramachandran | | | | |
| plot (%) | | | | |
| Outliers | 0.34 | 0.34 | 0.34 | 0.34 |
| Allowed | 10.83 | 16.81 | 8.15 | 11.43 |
| Favored | 88.83 | 82.85 | 91.51 | 88.24 |
| Rama-Z | | | | |
| (Ramachandran | | | | |
| plot Z-score, | | | | |
| RMSD) | | | | |
| whole (N = 591) | −3.25 (0.32) | −3.22 (0.33) | −1.79 (0.35) | −3.03 (0.32) |
| helix (N = 22) | −2.39 (0.70) | −3.52 (0.92) | −1.60 (1.00) | −1.32 (1.92) |
| sheet (N = 220) | −2.27 (0.30) | −1.22 (0.35) | −0.51 (0.35) | −1.41 (0.36) |
| loop (N = 349) | −2.07 (0.33) | −2.78 (0.32) | −1.72 (0.34) | −2.47 (0.29) |
| Rotamer outliers | 0.78 | 1.38 | 0 | 0 |
| (%) | | | | |
| Cβ outliers (%) | 0 | 0 | 0 | 0 |
| Peptide plane | | | | |
| (%) | | | | |
| Cis | 9.1/0.0 | 8.6/0.0 | 8.8/0.0 | 10.3/0.0 |
| proline/general | | | | |
| Twisted | 3.0/0.2 | 0.0/0.0 | 0.0/0.0 | 3.4/0.0 |
| proline/general | | | | |
| CaBLAM outliers | 5.13 | 8.92 | 5.49 | 7.3 |
| (%) | | | | |
| ADP (B-factors) | | | | |
| Iso/Aniso (#) | 4532/0 | 4501/0 | 4491/0 | 4558/0 |
| min/max/mean | | | | |
| Protein | 18.79/129.80/74.18 | 61.50/146.72/91.65 | 25.70/128.99/62.80 | 49.62/132.53/75.87 |
| Nucleotide | — | — | — | — |
| Ligand | — | — | — | — |
| Water | — | — | — | — |
| Occupancy | | | | |
| Mean | 1 | 1 | 1 | 1 |
| occ = 1 (%) | 100 | 100 | 100 | 100 |
| 0 < occ < 1 (%) | 0 | 0 | 0 | 0 |
| occ > 1 (%) | 0 | 0 | 0 | 0 |
| CC (mask) | 0.65 | 0.61 | 0.77 | 0.58 |
| CC (box) | 0.67 | 0.66 | 0.78 | 0.66 |
| CC (peaks) | 0.64 | 0.61 | 0.77 | 0.59 |
| CC (volume) | 0.65 | 0.61 | 0.78 | 0.58 |
| Resolution (Å) | 3.2 | 3.8 | 3.5 | 3.7 |

### Immunofluorescence Studies

C3H/HeN 7-8 week old female mice (Envigo) bladders were fixed in formalin, embedded in paraffin, and sectioned. Tissue sections were heat deparaffinized and rehydrated in xylene, followed by stepwise hydration in 100% ethanol, 90% ethanol, 75% ethanol, 50% ethanol to 30% ethanol (each step having a 5 min incubation in fresh solution). Slides were rinsed in 1×PBS followed by blocking solution (1×PBS with 5% fetal bovine serum). Primary mouse antibody to uroplakin IIIa (Progen, 1:50) was allowed to bind overnight at 4° C. Slides were washed in 1×PBS and a secondary anti-mouse Alexa fluor 488 antibody (Invitrogen, 1:1000) was allowed to bind for 2 h, and then washed again. FimH_{LD}-Alexa Fluor 647 (588 nM) was mixed with mAb (6 uM) for 20 min at room temperature in 1×PBS. FimH_{LD} mAb mixtures were applied to the section along with Hoechst DNA dye (8 uM) for 20 min at room temperature. The slides were washed again in 1×PBS and allowed to dry. ProLong™ gold antifade mountant (Invitrogen) was added and slides were imaged using the confocal function of a Zeiss Cell Observer Spinning Disk Confocal Microscope with a 10× air objective lens.

Splayed bladders were analyzed with a Zeiss Axio Observer D1 inverted fluorescence microscope equipped with an X-Cite120 mini LED light source (Excelitis Technologies) and DAPI, GFP, DsRed, and Cy5 filter sets. EC Plan-Neofluar (NA 0.075) 2.5× and EC Plan-Neofluar (NA 0.15) 5× objectives (Zeiss), an Axiocam 503 color camera (Zeiss), and ZEN 2 (blue version) software were used for image acquisition.

### Mouse Infection Experiments

For acute and 2-week infection models, 7-8 week old female C3H/HeN mice (Envigo) were infected with 2×10⁸ CFUs of UT189. Intraperitoneal injections of mAb were given in 1×PBS buffer 24 h before infection. Urines were taken by clean catch at specified time points. For obtaining serum, 3 to 4 mice per treatment group were bled at specific time points during the length of the experiment via submental bleeding method. At the conclusion of the experimental time points, mice were humanely sacrificed, and bladder and kidney organs were homogenized and tittered. For screening mAbs in the prophylactic infection model with UT189, if a phenotype was observed after 1 replicate, the experiment was repeated an additional 1-2 times.

TABLE 8
Primers for FimH Mutagenesis

| | | Mutant | Primer | |
|---|---|---|---|---|
| AA | Codon | Codon | Name | Primer Sequence |
| pBAD | N/A | N/A | pBAD33 F | ATGCCATAGCATTTTTTATCC |
| | | | | (SEQ_ID_NO: 294) |
| | | | pBAD33 R | GATTTAATCTGTATCAGG (SEQ_ID_NO: 295) |
| 4K | AAA | GAT (D) | J96_FimH | GCCTGTGATACCGCCAATGGTACC |
| | (K) | | 4K F | (SEQ_ID_NO: 296) |
| | | | J96_FimH | GGCGGTATCACAGGCGAATGACC |
| | | | 4KR | (SEQ_ID_NO: 297) |
| 7N | AAT | AAA (K) | J96_FimH | GCCTGTAAAACCGCCAAAGGTACC |
| | (N) | | 7N F | (SEQ_ID_NO: 298) |
| | | | J96_FimH | GATAGCGGTACCTTTGGCGGTTTT |
| | | | 7N R | (SEQ_ID_NO: 299) |
| 10A | GCT | GAT (D) | J96_FimH | GGTACCGATATCCCTATTGGCGGTG |
| | (A) | | 10A F | (SEQ_ID_NO: 300) |
| | | | J96_FimH | GCCAATAGGGATATCGGTACCATTGGC |
| | | | 10A R | (SEQ_ID_NO: 301) |
| 13I | ATT | AAA (K) | J96_FimH | GCTATCCCTAAAGGCGGTGGCAGC |
| | (I) | | 13I_F | (SEQ_ID_NO: 302) |
| | | | J96 FimH | CCACCGCCTTTAGGGATAGCGG |
| | | | 13I_R | (SEQ_ID_NO: 303) |
| 17S | AGC | AAA (K) | J96_FimH | GGTGGCAAAGCCAATGTTTATGTAAACCT |
| | (S) | | 17S F | TGCG (SEQ ID_NO: 304) |
| | | | J96_FimH | GTTTACATAAACATTGGCTTTGCCACCGC |
| | | | 17S R | CAATAGG (SEQ_ID_NO: 305) |
| 19N | AAT | AAA (K) | J96_FimH | GCAGCGCCAAAGTTTATGTAAACCTTGC |
| | (N) | | 19N F | (SEQ_ID_NO: 306) |
| | | | J96_FimH | GTTTACATAAACTTTGGCGCTGCCACC |
| | | | 19N R | (SEQ_ID_NO: 307) |
| 21Y | TAT | GAT (D) | J96_FimH | GCCAATGTTGATGTAAACCTTGCGCCC |
| | (Y) | | 21Y F | (SEQ_ID_NO: 308) |
| | | | J96_FimH | GTTTACATCAACATTGGCGCTGCCACC |
| | | | 21Y R | (SEQ_ID_NO: 309) |
| 23N | AAC | AAA (K) | J96_FimH | GTTTATGTAAAACTTGCGCCCGTCGTG |
| | (N) | | 23N F | (SEQ_ID_NO: 310) |
| | | | J96_FimH | GACGGGCGCAAGTTTTACATAAACATTGG C |
| | | | 23N R | (SEQ_ID_NO: 311) |
| 25A | GCG | GAT (D) | J96_FimH | GTAAACCTTGATCCCGTCGTGAATGTGGG G |
| | (A) | | 25A-2 F | (SEQ_ID_NO: 312) |
| | | | J96 FimH | CACGACGGGATCAAGGTTTACATAAACAT |
| | | | 25A-2 R | TGGC (SEQ_ID_NO: 313) |
| 27V | GTC | GAC (D) | J96_FimH | CCTTGCGCCCGACGTGAATG |
| | (V) | | 27V F | (SEQ_ID_NO: 314) |
| | | | J96_FimH | CCACATTCACGTCGGGCGC |
| | | | 27V R | (SEQ_ID_NO: 315) |
| 29N | AAT | AAA (K) | J96_FimH | GCCCGTCGTGAAAGTGGGG |
| | (N) | | 29N F | (SEQ_ID_NO: 316) |
| | | | J96_FimH | GGTTTTGCCCCACTTTCACGAC |
| | | | 29N R | (SEQ_ID_NO: 317) |
| 30V | GTG | GAT (D) | J96_FimH | CCCGTCGTGAATGATGGGCAAAAC |
| | (V) | | 30V F | (SEQ_ID_NO: 318) |
| | | | J96_FimH | CGACCAGGTTTTGCCCATCATTCAC |
| | | | 30V R | (SEQ_ID_NO: 319) |
| 33N | AAC | AAA (K) | J96_FimH | CGTGAATGTGGGGCAAAAACTGGTCG |
| | (N) | | 33N F | (SEQ_ID_NO: 320) |
| | | | J96_FimH | CGAAAGATCCACGACCAGTTTTTGCCC |
| | | | 33N R | (SEQ_ID_NO: 321) |
| 37D | GAT | AAA (K) | J96_FimH | CCTGGTCGTGAAACTTTCG |
| | (D) | | 37D F | (SEQ_ID_NO: 322) |
| | | | J96_FimH | GCGTCGAAAGTTTCACGAC |
| | | | 37D R | (SEQ_ID_NO: 323) |
| 40T | ACG | AAG (K) | J96_FimH | CGTGGATCTTTCGAAGCAAATC |
| | (T) | | 40T F | (SEQ_ID_NO: 324) |
| | | | J96_FimH | GGCAAAAGATTTGCTTCGAAAG |
| | | | 40T R | (SEQ_ID_NO: 325) |
| 43F | TTT | GAT (D) | J96_FimH | CGCAAATCGATTGCCATAACGATT |
| | (F) | | 43F F | (SEQ_ID_NO: 326) |
| | | | J96_FimH | GGCAATCGATTTGCGTCGAAAGAT |
| | | | 43F R | (SEQ_ID_NO: 327) |
| 48Y | TAT | GAT (D) | J96_FimH | GCCATAACGATGATCCGGAAACC |
| | (Y) | | 48Y F | (SEQ_ID_NO: 328) |
| | | | J96_FimH | CTGTAATGGTTTCCGGATCATCGTTATGG |
| | | | 48Y R | (SEQ_ID_NO: 329) |
| 50E | GAA | AAA (K) | J96_FimH | GCCATAACGATTATCCGAAAACCATTAC |
| | (E) | | 50E F | (SEQ_ID_NO: 330) |
| | | | J96_FimH | GTCTGTAATGGTTTTCGGATAATCG |
| | | | 50E R | (SEQ_ID_NO: 331) |
| 55Y | TAT | GAT (D) | J96_FimH | CCATTACAGACGATGTCACACTGC |
| | (Y) | | 55Y F | (SEQ_ID_NO: 332) |
| | | | J96_FimH | GTGACATCGTCTGTAATGGTTTCC |
| | | | 55Y R | (SEQ_ID_NO: 333) |
| 60R | CGA | AAA (K) | J96_FimH | CACTGCAAAAAGGCTCGGCTTATGGCGG |
| | (R) | | 60R F | (SEQ_ID_NO: 334) |
| | | | J96_FimH | GCCGAGCCTTTTTGCAGTGTGACATAGTC |
| | | | 60R R | (SEQ_ID_NO: 335) |
| 62S | TCG | AAG (K) | J96_FimH | GCAACGAGGCAAGGCTTATGGC |
| | (S) | | 62S F | (SEQ_ID_NO: 336) |
| | | | J96_FimH | GCCGTTCCTCGTTGCAGTGTGACA |
| | | | 62S R | (SEQ_ID_NO: 337) |
| 64Y | TAT | GAT (D) | J96_FimH | CTCGGCTGATGGCGGCGTGTTAT |
| | (Y) | | 64Y F | (SEQ_ID_NO: 338) |
| | | | J96_FimH | CCATCAGCCGAGCCTCGTTGCA |
| | | | 64Y R | (SEQ_ID_NO: 339) |
| 67V | GTG | GAT (D) | J96_FimH | GGCGGCGATTTATCTAATTTTTCCGGG |
| | (V) | | 67V F | (SEQ_ID_NO: 340) |
| | | | J96_FimH | TTAGATAAATCGCCGCCATAAGCCGAG |
| | | | 67V R | (SEQ_ID_NO: 341) |
| 70N | AAT | AAA (K) | J96_FimH | GTTATCTAAATTTTCCGGGACCG |
| | (N) | | 70N F | (SEQ_ID_NO: 342) |
| | | | J96_FimH | GGAAAATTTAGATAACACGCCGC |
| | | | 70N R | (SEQ_ID_NO: 343) |
| 74T | ACC | AAA (K) | J96_FimH | CCGGGAAAGTAAAATATAGTGGC |
| | (T) | | 74T F | (SEQ_ID_NO: 344) |
| | | | J96_FimH | CTATATTTTACTTTCCCGGAAAAATTAG |
| | | | 74T R | (SEQ_ID_NO: 345) |
| 76K | AAA | GAT (D) | J96_FimH | CCGGGACCGTAGATTATAGTGGCAGTAGC |
| | (K) | | 76K F | (SEQ_ID_NO: 346) |
| | | | J96_FimH | GCCACTATAATCTACGGTCCCGGAAAAAT |
| | | | 76K R | TAG (SEQ_ID_NO: 347) |
| 78N | AGT | AAA (K) | J96_FimH | CGTAAAATATAAAGGCAGTAGCTATCC |
| | (S) | | 78N F | (SEQ_ID_NO: 348) |
| | | | J96_FimH | CTGCCTTTATATTTTACGGTCCC |
| | | | 78N R | (SEQ_ID_NO: 349) |
| 80S | AGT | AAA (K) | J96_FimH | GTGGCAAAAGCTATCCATTTCC |
| | (S) | | 80S F | (SEQ_ID_NO: 350) |
| | | | J96_FimH | GGATAGCTTTTGCCACTATATTTTACGG |
| | | | 80S R | (SEQ_ID_NO: 351) |
| 87T | ACC | AAA (K) | J96_FimH | CCTACCAAAAGCGAAACGCCG |
| | (T) | | 87T F | (SEQ_ID_NO: 352) |
| | | | J96_FimH | CGTTTCGCTTTTGGTAGGAAATGG |
| | | | 87T R | (SEQ_ID_NO: 353) |
| 89E | GAA | AAA (K) | J96_FimH | CCTACCAAAAGCGAAACGCCGC |
| | (E) | | 89E F | (SEQ_ID_NO: 354) |
| | | | J96_FimH | CGGCGTTTTGCTGGTGGTAGGA |
| | | | 89E R | (SEQ_ID_NO: 355) |
| 92R | CGC | GAC (D) | J96_FimH | CGCCGGACGTTGTTTATAATTCG |
| | (C) | | 92R F | (SEQ_ID_NO: 356) |
| | | | J96_FimH | TAAACAACGTCCGGCGTTTCGC |
| | | | 92R R | (SEQ_ID_NO: 357) |
| 96N | AAT | AAA (K) | J96_FimH | GCGTTGTTTATAAATCGAGAACGG |
| | (N) | | 96N F | (SEQ_ID_NO: 358) |
| | | | J96_FimH | CCGTTCTCGATTTATAAACAACGCG |
| | | | 96N R | (SEQ_ID_NO: 359) |
| 98R | AGA | AAA (K) | J96_FimH | GTTTATAATTCGAAAACGGATAAGCCG |
| | (R) | | 98R F | (SEQ_ID_NO: 360) |
| | | | J96_FimH | GCTTATCCGTTTTCGAATTATAAACAACG |
| | | | 98R R | (SEQ_ID_NO: 361) |
| 99T | ACG | AAG (K) | J96_FimH | GTTTATAATTCGAGAAAGGATAAGCCG |
| | (T) | | 99T F | (SEQ_ID_NO: 362) |
| | | | J96_FimH | CGGCTTATCCTTTCTCGAATTATAAAC |
| | | | 99T R | (SEQ_ID_NO: 363) |
| 101K | AAG | GAT (D) | J96_FimH | GAGAACGGATGATCCGTGGCCGGTG |
| | (K) | | 101K F | (SEQ_ID_NO: 364) |
| | | | J96_FimH | CCGGCCACGGATCATCCGTTCTCGAA |
| | | | 101K R | (SEQ_ID_NO: 365) |
| 110T | ACG | AAG (K) | J96_FimH | GGTGGCGCTTTATTTGAAGCCTGTGAGC |
| | (T) | | 110T F | (SEQ_ID_NO: 366) |
| | | | J96_FimH | CGCACTGCTCACAGGCTTCAAATAAAGC |
| | | | 110T R | (SEQ_ID_NO: 367) |
| 121K | AAA | GAT (D) | J96_FimH | GGCGATTGATGCTGGCTCATTAATTGC |
| | (K) | | 121K F | (SEQ_ID_NO: 368) |
| | | | J96_FimH | GAGCCAGCATCAATCGCCACC |
| | | | 121K R | (SEQ_ID_NO: 369) |
| 128V | GTG | GAT (D) | J96_FimH | CATTAATTGCCGATCTTATTTTGCGAC |
| | (V) | | 128V F | (SEQ_ID_NO: 370) |
| | | | J96_FimH | CAAAATAAGATCGGCAATTAATGAGCC |
| | | | 128V R | (SEQ_ID_NO: 371) |
| 132R | CGA | AAA (K) | J96_FimH | GCTTATTTTGAAACAGACCAACAAC |
| | (R) | | 132R F | (SEQ_ID_NO: 372) |
| | | | J96_FimH | GGTCTGTTTCAAAATAAGCACGGC |
| | | | 132R R | (SEQ_ID_NO: 373) |
| 139S | AGC | AAA (K) | J96_FimH | CAACTATAACAAAGATGATTTCCAG |
| | (S) | | 139S F | (SEQ_ID_NO: 374) |
| | | | J96_FimH | GGAAATCATCTTTGTTATAGTTGTTGG |
| | | | 139S R | (SEQ_ID_NO: 375) |
| 142F | TTC | GAC (D) | J96_FimH | CTATAACAGCGATGATGACCAGTTTGTGT GG |
| | (F) | | 142F F | (SEQ_ID_NO: 376) |
| | | | J96_FimH | CACAAACTGGTCATCATCGCTGTTATAGT TG |
| | | | 142F R | (SEQ_ID_NO: 377) |
| 145V | GTG | GAT (D) | J96_FimH | TTTCCAGTTTGATTGGAATATTTACG |
| | (V) | | 145V F | (SEQ_ID_NO: 378) |
| | | | J96_FimH | GTAAATATTCCAATCAAACTGGAAATC |
| | | | 145V R | (SEQ_ID_NO: 379) |
| 152N | AAT | AAA (K) | J96_FimH | CGCCAATAAAGATGTGGTGGTG |
| | (N) | | 152N F | (SEQ_ID_NO: 380) |
| | | | J96_FimH | CACCACATCTTTATTGGCGTAA |
| | | | 152N R | (SEQ_ID_NO: 381) |
| 155V | GTG | GAT (D) | J96_FimH | CCAATAATGATGTGGATGTGCCTACTGGC |
| | (V) | | 155V F | (SEQ_ID_NO: 382) |
| | | | J96_FimH | GTAGGCACATCCACATCATTATTGGCG |
| | | | 155V R | (SEQ_ID_NO: 383) |

### Example 2—Monoclonal Antibodies Targeting FimH Adhesin Protect Against UTI in a Murine Model

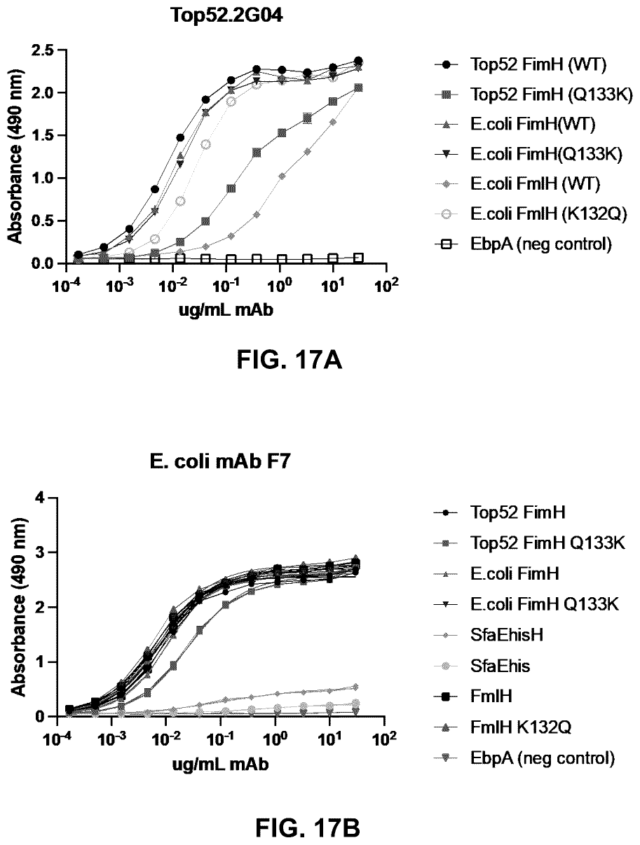

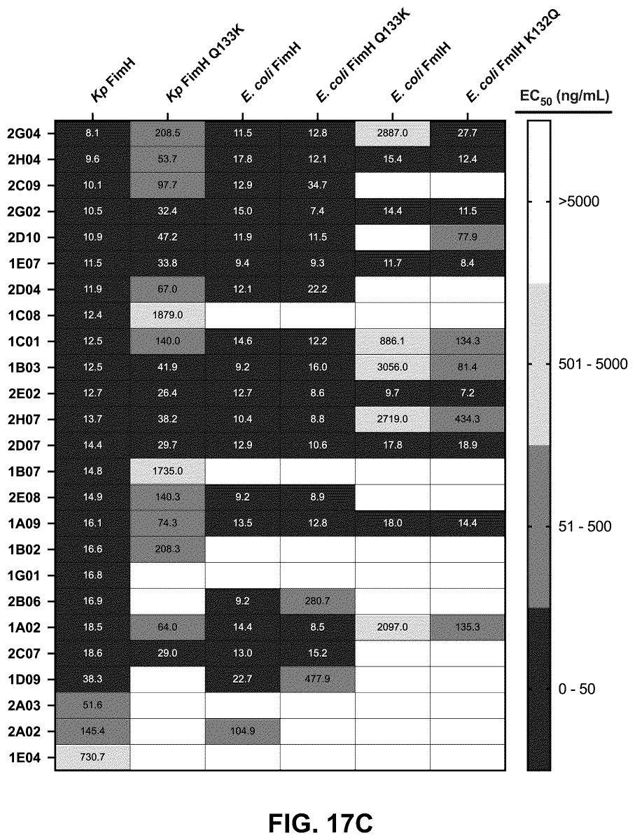

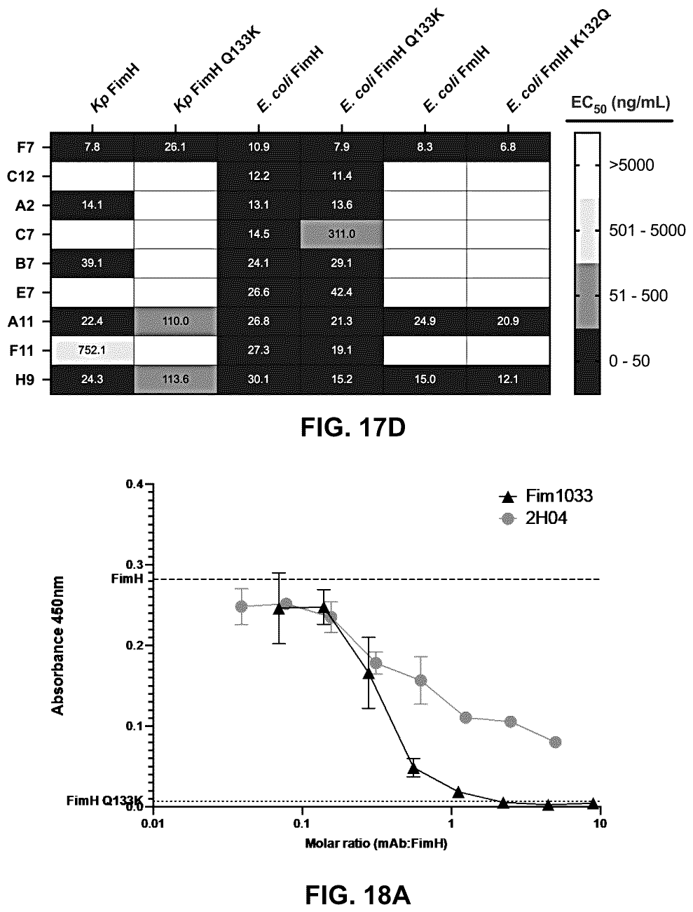

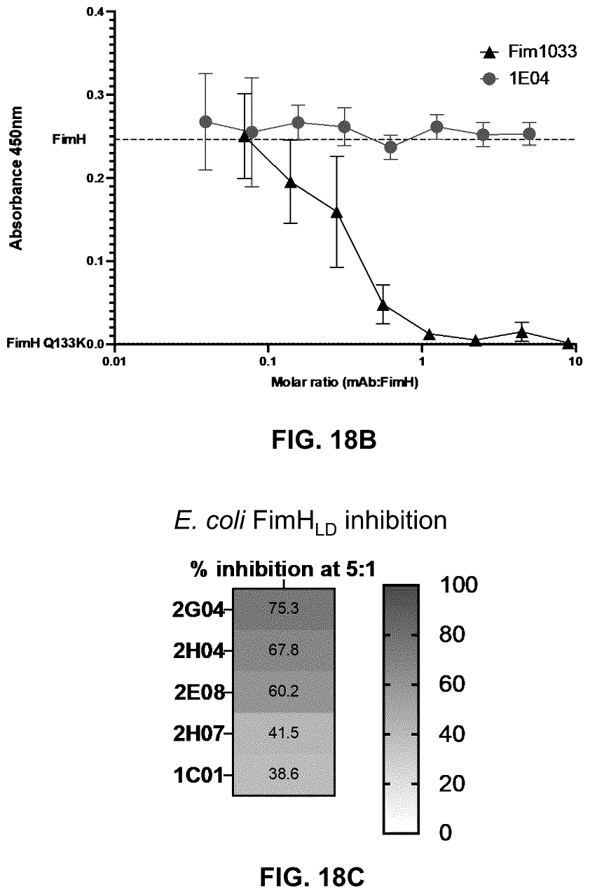

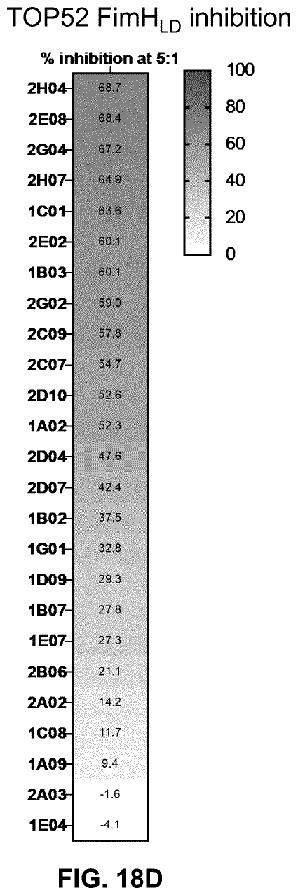

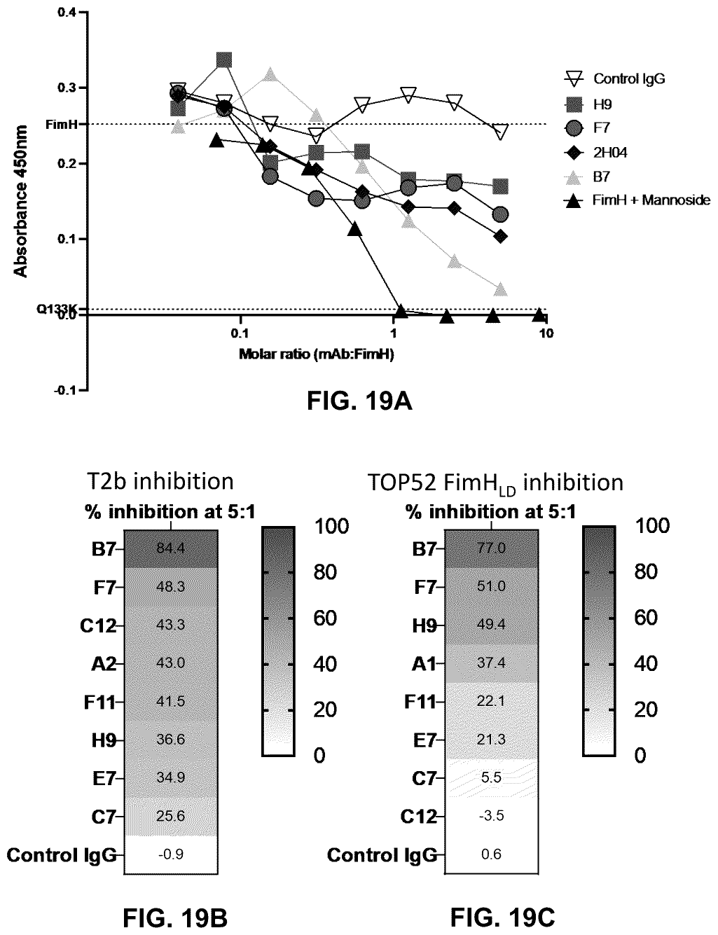

The K. pneumoniae FimH amino acid sequence is 86% similar to that of uropathogenic Escherichia coli (UPEC) and is highly conserved among K. pneumoniae isolates. FimH consists of an N-terminal lectin mannose binding domain and a pilin domain that connects it to the pilus. We generated monoclonal antibodies (mAbs) to UPEC and K. pneumoniae FimH lectin domains to identify mAbs that inhibit FimH binding and prevent infection in vivo. Using ELISA binding assays, we identified mAbs that bind with high affinity to the antigenic FimH (FIG. 17A, FIG. 17B, FIG. 17C, FIG. 17D). In addition, a subset of mAbs cross-reacted to both UPEC and K. pneumoniae FimH proteins and related chaperone usher pili galactose binding adhesin FmIH, which contributes to UPEC attachment in chronic UTI. Further, we identified monoclonal antibodies that inhibited K. pneumoniae and UPEC FimH lectin domain binding in vitro by performing binding inhibition ELISAs (FIG. 18A, FIG. 18B, FIG. 18C, FIG. 18D).

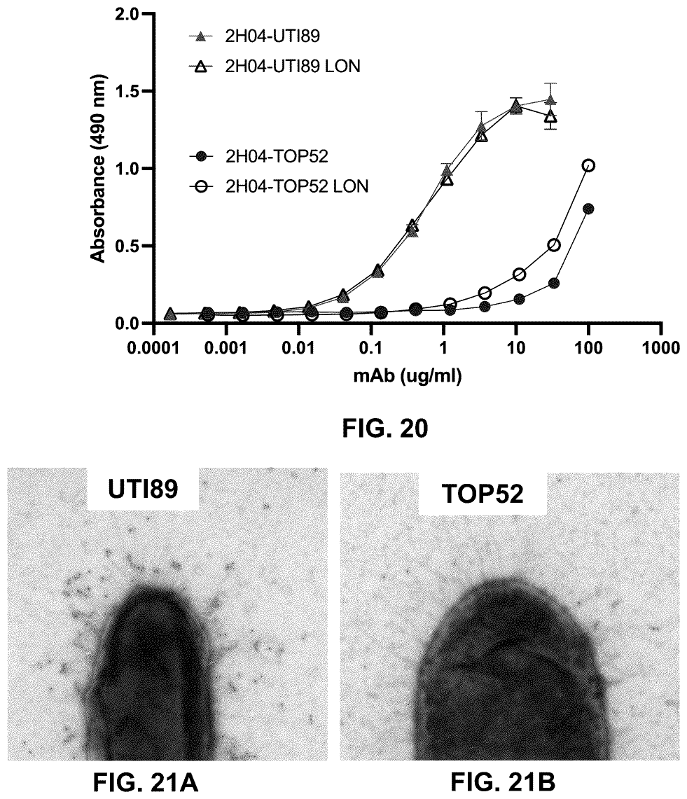

On the tip of a pilus, FimH is a two-domain protein that samples a conformational equilibrium between a high-affinity relaxed state and low-affinity tense state. The FimH lectin domain antigen is exclusively in the relaxed state. When tested for binding to UT189 bacteria in an ELISA assay, we found that mAbs differentially bind to the bacteria and not all mAbs bind with high affinity (FIG. 3A; LON designates fim locked on an overexpression strain as a positive control). In UPEC, naturally occurring FimH variants can strongly shift the conformational equilibrium towards either state. In tip-like two-domain FimH, the mutations A27V/V163 strongly shift FimH towards the relaxed conformation and A62S strongly shifts FimH towards tense. mAbs were tested for binding to these FimH conformational variants overexpressed on the surface of UT189 (FIG. 3B). We found that there were two subsets of mAbs: 1) a set that binds with slight preference to the relaxed conformational mutant and II) a set that binds with a strong preference to the relaxed state. 2H04 mAb was also tested for binding to K. pneumoniae isolate TOP52 by ELISA and immunogold and we found that it bound more strongly to UT189 corroborating previous work that shows K. pneumoniae FimH to be strongly skewed towards a low-affinity conformation (FIG. 20, FIG. 21A, FIG. 21B).

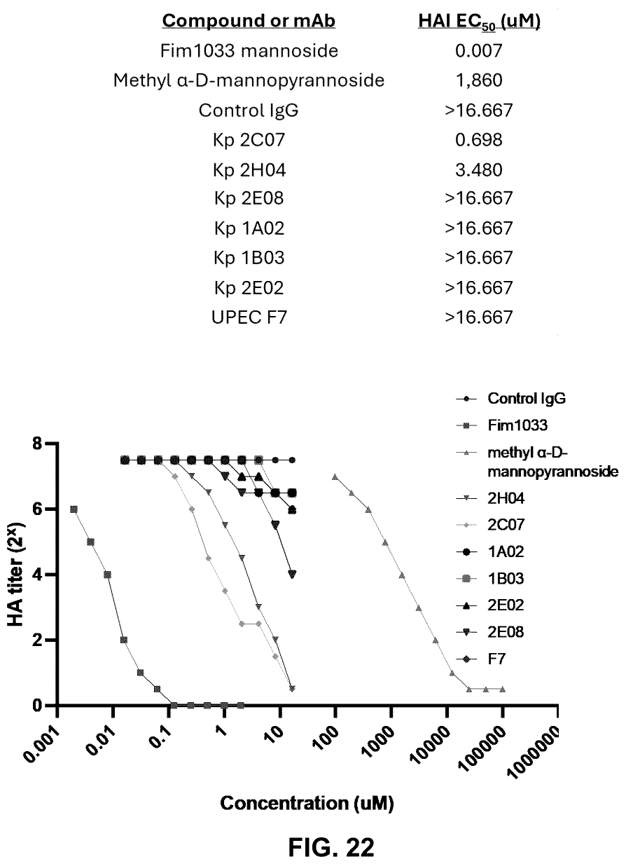

Using the subset of mAbs that bound with high-affinity to UT189 wild type bacteria, we tested 7 different representative mAbs from this subset for ability to block type 1 pili red blood cell hemagglutination. We found that most mAbs inhibited type 1 pili binding slightly at the highest concentration, but two mAbs (2H04 and 2C07) had a strong titratable inhibitory effect (FIG. 22)

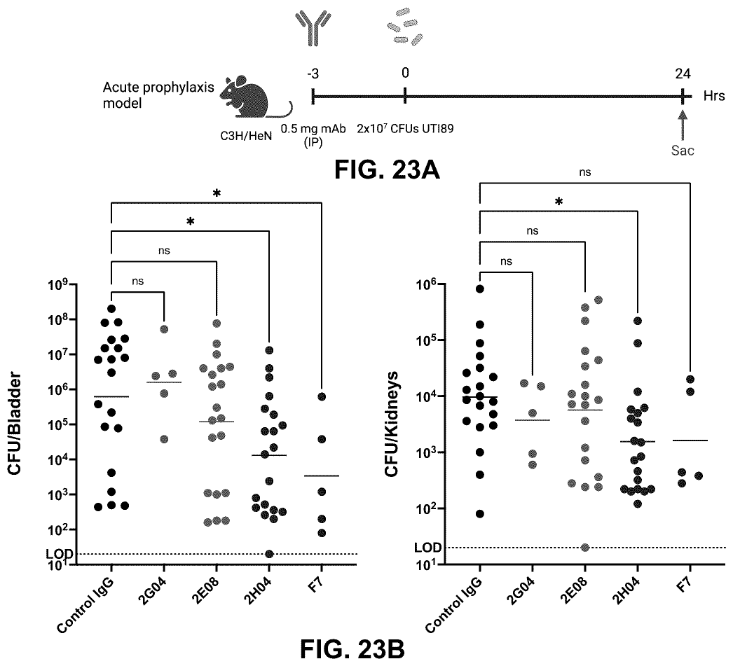

We first tested our highest inhibiting mAbs from our FimH lectin domain protein assays for ability to protect in an acute murine model of UPEC UTI with an infection of 107 CFUs of UT189 (FIG. 23A, FIG. 23B). Two out of four mAbs (2H04 and F7) tested significantly reduced bacterial titers in the urine and bladders of infected mice. The two mAbs that did not affect infection were 2G04 and 2E08 which do not bind to UT189 bacteria as well as 2H04 and F7. These results showed that the ability to bind to native FimH on the pilus of bacteria is important for protection.

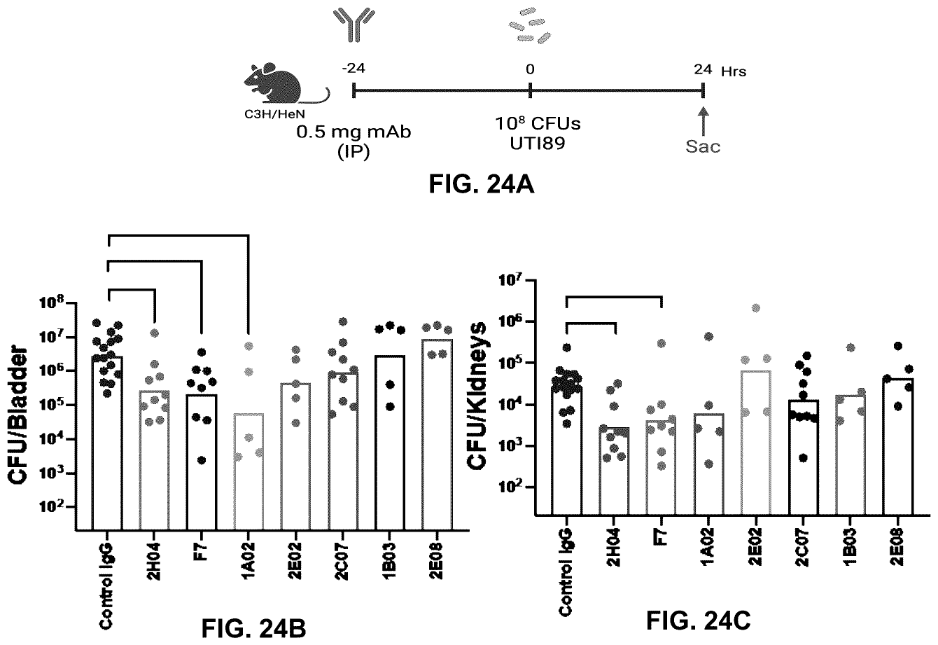

We then tested 7 different representative mAbs that bound well to UT189 bacteria and were tested in the hemagglutination assay in an acute model of infection. mAbs were given before infection to test preventative ability. We found that again 2H04 and F7 significantly protected mice from infection along with one other mAb 1A02 (FIG. 24A, FIG. 24B, FIG. 24C). Interestingly, 2C07 with the highest inhibitory power did not appear to significantly protect against infection suggesting that the ability to recognize and bind to native tip-like FimH drives protection.

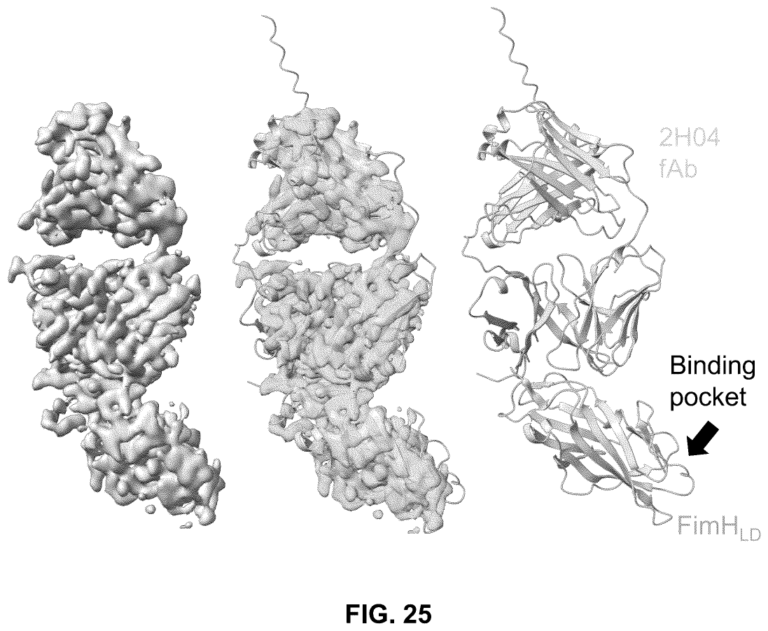

Using cryo-EM, we obtained the structure of 2H04 fAb to FimH lectin domain (FIG. 25). We found that 2H04 bound to the base of the lectin domain, in an area that does not change dramatically between conformational states. Further, this suggests that the mechanism of inhibiting FimH binding is by steric hindrance and preventing FimH from reaching the mannose ligands, as opposed to the mAb binding directly to the mannose-binding pocket.

These results demonstrate that monoclonal antibodies inhibiting FimH function are an antibiotic-sparing therapeutic strategy for K. pneumoniae and UPEC UTIs.

### Methods

### Monoclonal Antibody Generation

Monoclonal antibodies against TOP52 and E. coli FimH truncates were generated. Briefly, C57BL/6 mice were immunized with 25 ug of purified lectin mixed 1:1 with the squalene oil-in-water adjuvant Addavax (Invivogen). 3 weeks post-immunization, mice were boosted with a second dose of the same protein. Mice were sacrificed at 5 days post-boost and B cells from draining lymph nodes were stained and sorted into 96 well plates. B cell RNA was converted to cDNA and the VDJ regions amplified and cloned into human IgG1 plasmid expression vectors.

### Mab Expression and Purification

mAbs were expressed using the Expi293 protein expression system and purified on a Protein A column.

What is claimed is:

1. A composition to treat a bacterial infection, the composition comprising an agent that targets a FimH adhesin protein.

2. The composition of claim 1, wherein the agent is an antibody.

3. The composition of claim 2, wherein the antibody targets the FimH adhesin protein of K. pneumonia and E. coli bacteria.

4. The composition of claim 3, wherein the antibody targets lectin domains of the FimH adhesin protein.

5. The composition of claim 3, wherein the antibody comprises:
a. a heavy chain protein variable region comprising an amino acid sequence selected from SEQ ID NOS: 61, 63, 65, 67, 69, 71, 73, 75, 77, 79, 81, 83, 85, 87, 89, 91, 93, 95, 97, 99, 101, 103, 105, 107, 109, 111, 113, 115, 117, and 119; and
b. light chain protein comprising an amino acid sequence independently selected from SEQ ID NOS: 62, 64, 66, 68, 70, 72, 74, 76, 78, 80, 82, 84, 86, 88, 90, 92, 94, 96, 98, 100, 102, 104, 106, 108, 110, 112, 114, 116, 118, and 120.

6. The composition of claim 3, wherein the antibody comprises:
a. a heavy chain protein variable region encoded by a nucleotide sequence selected from SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, and 59; and
b. a light chain protein encoded by a nucleotide sequence independently selected from SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, and 60.

7. The composition of claim 3, wherein the antibody comprises:
a. a heavy chain protein encoded by a nested nucleotide sequence selected from SEQ ID NOS: 121, 123, 125, 127, 129, 131, 133, 135, 137, 139, 141, 143, 145, 147, 149, 151, 153, 155, 157, 159, 161, 163, 165, 167, 169, 171, 173, 175, 177, and 179, and
b. a light chain protein encoded by a nested nucleotide sequence independently selected from SEQ ID NOS: 122, 124, 126, 128, 130, 132, 134, 136, 138, 140, 142, 144, 146, 148, 150, 152, 154, 156, 158, 160, 162, 164, 166, 168, 170, 172, 174, 176, 178, and 180.

8. The composition of claim 3, wherein the antibody comprises:
a. a heavy chain protein encoded by a plasmid nucleotide sequence selected from SEQ ID NOS: 181, 183, 185, 187, 189, 191, 193, 195, 197,199, 201, 203, 205, 207, 209, 211, 213, 215, 217, 219, 221, 223, 225, 227, 229, 231, 233, 235, 237, and 239; and
b. a light chain protein encoded by a plasmid nucleotide sequence independently selected from SEQ ID NOS: 182, 184, 186, 188, 190, 192, 194, 196, 198, 200, 202, 204, 206, 208, 210, 212, 214, 216, 218, 220, 222, 224, 226, 228, 230, 232, 234, 236, 238, and 240.

9. The composition of claim 1, wherein the composition is used to prevent and treat a urinary tract infection (UTI).

10. A method of identifying at least one antibody to treat a bacterial infection, the method comprising performing an ELISA binding assay comprising a plurality of candidate monoclonal antibodies to identify the at least one antibody from the plurality of the candidate monoclonal antibodies that inhibit FimH binding in vitro.

11. The method of claim 10, wherein the ELISA assay further comprises at least one antigen selected from E. coli and K. pneumonia FimH proteins.

12. The method of claim 10, wherein the bacterial infection is a UTI.

13. The method of claim 11, further comprising administering the at least one antibody identified in vitro to a murine UTI model to characterize protection against UTI in vivo.

14. A method of treating a bacterial infection, the method comprising administering a therapeutically effective amount of a compound that targets and inhibits a FimH protein.

15. The method of claim 14, wherein the bacterial infection is a UTI.

16. The method of claim 14, wherein the compound is an antibody.

17. The method of claim 16, wherein the antibody binds to a lectin domain of FimH.

18. The method of claim 17, wherein the antibody comprises:
a. a heavy chain protein variable region comprising an amino acid sequence selected from SEQ ID NOS: 241, 243, 245, 247, 249, 251, 253, 255, and 257 and the light chain protein variable region comprises an amino acid sequence independently selected from SEQ ID NOS: 242, 244, 246, 248, 250, 252, 254, 256, and 258.

19. The method of claim 17, wherein the antibody comprises a heavy chain protein variable region encoded by a nucleotide sequence selected from 259, 261, 263, 265, 267, 269, 271, 273, and 275, and the light chain protein variable region is encoded by a nucleotide sequence independently selected from SEQ ID NOS: 260, 262, 264, 266, 268, 270, 272, 274, and 276.
